(19) United States
(12) Patent Application Publication
(10) Pub. No.: US 2024/0358826 A1
(43) Pub. Date: Oct. 31, 2024

(54) INTERLEUKIN-1 ALPHA CHIMERIC PROTEIN

(71) Applicants: Orionis Biosciences, Inc., Waltham, MA (US); Orionis Biosciences BV, Ghent (BE)

(72) Inventors: Nikolai KLEY, Waltham, MA (US); Erik DEPLA, Ghent (BE); Joris WAUMAN, Ghent (BE); Enkeleda NAKUCI, Waltham, MA (US); Yen-Ching HO, Waltham, MA (US); Alexander Lee KLEY, Waltham, MA (US)

(21) Appl. No.: 18/574,857

(22) PCT Filed: Jul. 8, 2022
(86) PCT No.: PCT/US22/73559

§ 371 Date: Dec. 28, 2023

Related U.S. Application Data
(60) Provisional application No. 63/246,875, filed on Sep. 22, 2021.
(60) Provisional application No. 63/219,475, filed on Jul. 8, 2021.

(51) Int. Cl. A61K 39/39; A61K 38/00; A61K 39/00; A61K 39/145; A61K 39/215; A61K 39/395; C07K 14/545; C07K 16/30

(52) U.S. Cl. CPC A61K 39/39; A61K 39/145; A61K 39/215; A61K 39/39558; A61K 39/4635; C07K 14/545; C07K 16/30; A61K 38/00; A61K 2039/53; A61K 2039/54; A61K 2039/544; A61K 2039/55505; A61K 2039/55566; A61K 2039/55577; C07K 2317/569; C07K 2319/30

(57) ABSTRACT

The present invention relates, in part, to chimeric proteins, chimeric protein complexes, vaccine compositions, and adjuvants that include IL-1α or pro-IL-1α and their use as therapeutic agents or vaccines. The present invention further relates to methods of treatment of various diseases, such as infectious diseases and cancer and methods of vaccination.

### FIELD

The present invention relates, in part, to chimeric proteins, chimeric protein complexes, adjuvants, or vaccine compositions, including IL-1α or pro-IL-1α, and their use in the treatment of various infectious diseases, cancers, and other therapeutic indications.

### SEQUENCE LISTING

The contents of the computer readable Sequence Listing in XML format (“XML Document”) submitted electronically herewith are incorporated herein by reference in their entirety. A computer readable format copy of the Sequence Listing (filename: ORN-082PC.xml, date produced: Jul. 6, 2022; size: 708,215 bytes) is submitted per 37 C.F.R. §§ 1.831-1.835.

### BACKGROUND

Interleukins are mostly secreted proteins that act as cytokines to mediate inter-cellular communication during immune responses. IL-1α is a proinflammatory molecule and plays a central role in mediating immune responses due to its broad spectrum of biological functions and range of cells that it targets. IL-1α is a decision-making molecule used by the cell for gauging the magnitude of stress or damage or severity of infection to launch either the tissue or the whole body into action through initiation of inflammation or reparative fibrosis. Aberration of these sequelae can produce devastating disruption of tissue homeostasis and underlies the pathology of numerous human diseases.

Under homeostatic conditions, IL-1α is expressed by multiple hematopoietic and non-hematopoietic cells. It can be upregulated by a diverse array of inflammatory stimuli and it does not depend on proteolytic processing for its bioactivity. Therefore, cell death caused by injury due to sterile or infectious insults leads to release of bioactive IL-1α that signals via IL-1R to induce inflammatory responses. IL-1R is expressed constitutively by a broad range of cell types and NF-κB and MAPK activation downstream of IL-1R induces production of pro-inflammatory mediators such as cyclooxygenase type-2 (COX-2), IL-6, tumor necrosis factor (TNF), that further promote production of IL-1α and IL-1β, amplifying the inflammatory stimuli provided by the initial release of IL-1α. Physiological manifestations of IL-1 signaling include fever, hypotension, vasodilation and increased sensitivity to pain.

Dysregulation of IL-1α production is associated with numerous auto-inflammatory disorders, in instigating host defense against multiple infectious agents. Malignant cells, tumor-infiltrating immune cells and stromal cells can express IL-1α, IL-1β and IL-1R. IL-1 signaling in the tumor tissue and its microenvironment can an affect tumor progression in different ways. Even after several decades of research, many critical questions related to IL-1α remain unanswered. Deregulated activity of IL-1α or the interleukin family members is a major cause of inflammatory and autoimmune disorders. Both IL-1α and IL-13, while having the potential for exhibiting differentiated biological activities, can be potent activators of T cells and NK cells, cell types that play important roles in defense against cancer and infectious diseases, for example. Such responses can include induction of T cells, memory T cells, and T-cell mediated immunity. Many other cell types are targets of IL-1 with relevance to therapeutic applications in cancer, infectious diseases as well as other diseases. Thus, members of the IL-1 family, represent attractive targets for therapeutic manipulation. Selective activation of desired target cells, e.g., T and NK cells, by IL-1α has remained an unaddressed challenge in the quest of harnessing the therapeutic potential of IL-1α.

Accordingly, there remains a need for target-selective, safe, and effective IL1-α based therapeutics with improved pharmacokinetic and therapeutic properties with minimal toxicity.

### SUMMARY

In some aspects, the present invention relates to chimeric proteins and chimeric protein complexes, including Fc-based chimeric protein complexes, comprising an interleukin-1α (IL-1α), pro-IL-1α, or a mutant thereof as a signaling agent. The term variants as used herein includes IL-1α mutants or pro-IL-1α mutants.

In some aspects, the present invention is related to a chimeric protein comprising: (a) an interleukin-1α (IL-1α), pro-IL-1α, or a mutant thereof, and (b) one or more targeting moieties, said targeting moieties comprising recognition domains which specifically bind to an antigen or receptor of interest; wherein the IL-1α, pro-IL-1α, or the mutant thereof, and the one or more targeting moieties are optionally connected with one or more linkers.

In some aspects, the present invention is related to a chimeric protein complex (e.g., a Fc-based chimeric protein complex) comprising: a) an interleukin-1α (IL-1α), pro-IL-1α, or a mutant thereof, and b) one or more targeting moieties, said targeting moieties comprising recognition domains which specifically bind to an antigen or receptor of interest; and c) a Fc domain, the Fc domain optionally having one or more mutations that reduces or eliminates one or more effector functions of the Fc domain, promotes Fc chain pairing in the Fc domain, and/or stabilizes a hinge region in the Fc domain.

In one aspect, the present invention provides a chimeric protein or chimeric protein complex (e.g., a Fc-based chimeric protein complex) comprising: (a) a mutant interleukin-1α (IL-1α) or pro-IL-1α, wherein the mutation is a deletion of amino acids 1-6 (delta 1-6) with respect to any one of SEQ ID NOs: 1-4, and (b) one or more targeting moieties, said targeting moieties comprising recognition domains which specifically bind to an antigen or receptor of interest; and (c) a connector between (a) and (b), the connector being: (i) a flexible linker that connects (a) and (b), and/or (ii) an Fc domain that connects (a) and (b), the Fc domain optionally having one or more mutations that reduces or eliminates one or more effector functions of the Fc domain, promotes Fc chain pairing in the Fc domain, and/or stabilizes a hinge region in the Fc domain.

The present disclosure concerns, in part, findings that chimeric proteins or chimeric protein complexes comprising an IL-1α, pro-IL-1α, or a mutant thereof, exhibit substantially reduced or increased IL-1R (IL-1 Receptor) activation signaling activity compared to wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118), respectively. Reduced IL-1R-activation signaling activity, however, can be induced and/or restored at a target cell when directed to such a cell through a targeting moiety. Surprisingly, the induced and/or restored IL-1α activity at a target cell, achieved through targeting of chimeric proteins or chimeric protein complexes comprising IL-1α, or variants thereof, may be similar or greater at the target cell than that of wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). Furthermore, and equally surprising, the targeted IL-1α activity of the chimeric protein or chimeric protein complexes comprising IL-1α, or variants thereof, may be similar to or even greater than that of wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). Importantly, the IL-1α chimeric proteins and chimeric protein complexes comprising IL-1α described herein, exhibit substantial and surprising selectivity for target cells versus non-target cells compared to wild type IL-1α, and can be substantially more than, for example, achieved with chimeric proteins comprising a targeted IL-1α having the deletion of amino acids 1-6 (delta 1-6) with respect to SEQ ID NO: 1 or 3. In summary, a unique combination of highly potent and highly cell target-selective IL-1R-signaling activation can be achieved with IL-1α compositions, and variants thereof, described herein.

The present disclosure also concerns, in part, findings that chimeric proteins or chimeric protein complexes comprising an IL-1α, pro-IL-1α, or a mutant thereof, exhibit increased (rather than reduced) IL-1R activation signaling activity compared to wild type IL-1α or wild type pro-IL-1α and/or a comparable IL-1α with a deletion of amino acids 1-6 (delta 1-6) with respect to SEQ ID NO: 1 or 3 or a comparable pro-IL-1α with a deletion of amino acids 113-118 (delta 113-118) with respect to SEQ ID NO: 2 or 4 but no additional mutations other than the delta 1-6 deletion in IL-1α or the delta 113-118 deletion in pro-IL-1α. Importantly, and equally surprising, such chimeric proteins and chimeric protein complexes comprising IL-1α, pro-IL-1α, or a mutant thereof described herein, can exhibit substantial and surprising selectivity for target cells versus non-target cells compared to wild type IL-1α or wild type pro-IL-1α and/or a comparable IL-1α with a deletion of amino acids 1-6 (delta 1-6) with respect to SEQ ID NO: 1 or 3 or a comparable pro-IL-1α with a deletion of amino acids 113-118 (delta 113-118) with respect to SEQ ID NO: 2 or 4 but no additional mutations other than the delta 1-6 deletion in IL-1α or the delta 113-118 deletion in pro-IL-1α.

In some embodiments, the IL-1α or pro-IL-1α is modified, i.e., is a variant and comprises one or more mutations in IL-1α (or pro-IL-1α). In some embodiments, the one or more mutations reduce or increase the biological activity of the IL-1α or pro-IL-1α. For example, the one or more mutations may reduce or increase the affinity and/or activity of the IL-1α or pro-IL-1α for a therapeutic receptor. In an embodiment, the modified IL-1α or pro-IL-1α comprises one or more mutations that reduce or increase its affinity and/or activity for IL-1R. In another embodiment, the modified IL-1α or pro-IL-1α comprises one or more mutations that reduce or increase its affinity and/or activity for IL-1R or IL-1RAcP. In some embodiments, the loss in affinity and/or activity of the modified IL-1α or pro-IL-1α for a therapeutic receptor, e.g., IL-1R or IL-1RAcP, can be induced and/or restored upon directing or targeting of the chimeric protein or chimeric protein complex comprising the modified IL-1α or pro-IL-1α to a target cell through a targeting moiety. In some embodiments, the modified IL-1α or pro-IL-1α variant that comprises one or more mutations that reduce or increase its affinity and/or activity for IL-1R or IL-1RAcP, further comprises one or more mutations that reduce undesired disulphide pairings to improve product homogeneity and pharmaceutical properties of the chimeric protein or chimeric protein complexes, while simultaneously maintaining or avoiding substantial loss of induction and/or restoration of IL-1R-activation activity by the modified IL-1α or pro-IL-1α when directed/targeted to a target cell through a targeting moiety. In some embodiments the incorporation of wild type IL-1α in a chimeric protein or chimeric protein complex, such as, for example, through genetic fusion or attachment (e.g. the formation of a complex), reduces the biological activity of IL-1α. For example, wild type IL-1α incorporated in chimeric proteins or chimeric protein complexes may have reduced affinity and/or activity compared to wild type IL-1α for a therapeutic receptor. In some embodiments, the loss in affinity and/or activity of wild type IL-1α for a therapeutic receptor, e.g., IL-1R, can be induced and/or restored upon directing or targeting of the chimeric protein or chimeric protein complex comprising IL-1α to a target cell through a targeting moiety. In some embodiments, the induction and/or restoration of IL-1α-mediated IL-1R-activation at a target cell may reach a level that is similar to or higher than IL-1α-activation achieved with wild type (non-chimeric) IL-1α. In some embodiments, the IL-1α is a variant that comprises one or more mutations which reduce undesired disulphide pairings to improve product homogeneity and pharmaceutical properties of the chimeric protein or chimeric protein complexes, while simultaneously maintaining or avoiding substantial loss of IL-1R-activation of the modified IL-1α compared to wild type IL-1α in the context of chimeric proteins or chimeric protein complexes, including maintaining or avoiding substantial loss of restoration and/or induction of IL-1R-activation by the modified IL-1α when directed or targeted to a target cell through a targeting moiety.

In some embodiments, the chimeric proteins and chimeric protein complexes, including Fc-based chimeric protein complexes, comprises one or more additional signaling agents, e.g., without limitation, an interferon, an interleukin, and a tumor necrosis factor, that may be modified. In various embodiments, the chimeric proteins and chimeric protein complexes, including Fc-based chimeric protein complexes, of the invention provides improved safety and/or therapeutic activity and/or pharmacokinetic profiles (e.g., increased serum half-life) compared to an untargeted and/or unmodified IL-1α or an unmodified, wild type IL-1α.

In various embodiments, the chimeric proteins and chimeric protein complexes, including Fc-based chimeric protein complexes, comprise one or more targeting moieties which have recognition domains (e.g. antigen recognition domains, including without limitation various antibody formats, inclusive of single-domain antibodies) which specifically bind to a target (e.g. antigen, receptor) of interest. In various embodiments, the targeting moieties have recognition domains that specifically bind to a target (e.g. antigen, receptor) of interest, including those found on one or more immune cells, which can include, without limitation, T cells, cytotoxic T lymphocytes, T helper cells, T regulatory cells (Tregs), natural killer (NK) cells, natural killer T (NKT) cells, anti-tumor and tumor macrophages (e.g. M1 and M2 macrophages), B cells, B regulatory (Breg) cells, neutrophils, monocytes, myeloid derived cells, and dendritic cells. In various embodiments, the targeting moieties have recognition domains that specifically bind to a target (e.g. antigen, receptor) of interest, including those found on one or more tumor cells, endothelial cells, epithelial cells, mesenchymal cells, stromal cells or other cell types that are characteristic of and/or unique for specific organs and/or tissues, including those specifically associated with disease. In some embodiments, the recognition domains specifically bind to a target (e.g. antigen, receptor) of interest and effectively recruit one or more immune cells. In some embodiments, the targets (e.g. antigens, receptors) of interest can be found on one or more tumor cells. In some embodiments, the present chimeric proteins, chimeric protein complexes, including Fc-based chimeric protein complexes, may recruit an immune cell, e.g., an immune cell that can kill and/or suppress a tumor cell, or modulate other immune cells, to a site of action (such as, by way of non-limiting example, the tumor microenvironment). In some embodiments, the present chimeric proteins, chimeric protein complexes, including Fc-based chimeric protein complexes, may modulate an immune cell at a site of action, or recruit an immune cell to a site of action that is associated with an autoimmune disease, inflammatory disease, infection, metabolic and/or cardiovascular disease (such as, by way of non-limiting example, the disease microenvironment). In some embodiments, the recognition domains specifically bind to a target (e.g. antigen, receptor) of interest that is part of a non-cellular structure.

In some embodiments, the chimeric proteins or the chimeric protein complexes described herein comprise IL-1α or pro-IL-1α mutants (which is an example of a cytokine/signaling agent that may also be used in the present invention) with reduced or increased biological activity that is coupled to one or more targeting moieties. In some embodiments, the chimeric proteins or chimeric protein complexes of the present invention include AcTakines (Activity-on-Target cytokines) that have one or more mutated cytokines that remain inactive en route through the body and only reveal their full agonistic activity upon target cell binding. In some embodiments, the chimeric proteins or chimeric protein complexes target a mutant IL-1α to CD8⁺ T cells. In vivo, such chimeric proteins or chimeric protein complexes can act as an adjuvant that potently promotes the CD8⁺ T cell response to antigens, with a significantly reduced toxicity profile compared to wild-type (WT) IL-1α. In some embodiments, the chimeric proteins or chimeric protein complexes target a mutant IL-1α to NK cells or other immune or alternative cell types.

In various embodiments, the present chimeric proteins and chimeric protein complexes, including Fc-based chimeric protein complexes find use in the treatment of various diseases or disorders such as cancer, infections, immune disorders, autoimmune diseases, cardiovascular diseases, wound healing, ischemia-related diseases, neurodegenerative diseases, metabolic diseases and many other diseases and disorders, and the present invention encompasses various methods of treatment. In various embodiments, the adjuvants, the chimeric proteins, and the chimeric protein complexes find use in the vaccination against or treatment of various diseases or disorders, such as infections. The present invention encompasses various methods of treatment or various methods of vaccination against such diseases or disorders.

Another aspect of the invention is related to a method for treating a subject afflicted with an infectious disease, comprising administering a chimeric protein or chimeric protein complex as described herein. In some embodiments, the chimeric protein or chimeric protein complex comprises: (i) an IL-1α, pro-IL-1α, or a mutant thereof, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) and/or (2) a flexible linker that connects (i) and (ii); wherein the mutant IL-1α or pro-IL-1α is characterized by low or high affinity or activity at the IL-1 receptor.

Yet another aspect of the invention is related to a method for treating a subject afflicted with a cancer, comprising administering a chimeric protein or chimeric protein complex as described herein. In some embodiments, the chimeric protein or chimeric protein complex comprises: (i) an IL-1α, pro-IL-1α, or a mutant thereof, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) and/or (2) a flexible linker that connects (i) and (ii); wherein the mutant IL-1α or pro-IL-1α is characterized by low or high affinity or activity at the IL-1 receptor.

One aspect of the present application is related to a vaccine composition comprising the chimeric proteins or chimeric protein complexes as described herein. In some embodiments, the vaccine composition comprises: (a) an adjuvant, and (b) an antigen that is suitable for inducing an immune response. The adjuvant comprises a chimeric protein or chimeric protein complex comprising: (i) an IL-1α, pro-IL-1α, or a mutant thereof (which is an example of a signaling agent as described herein), (ii) one or more targeting moieties, said targeting moieties comprising recognition domains which specifically bind to an antigen or receptor of interest; and (iii) a connector between (i) and (ii). The connector comprises: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii); and/or (2) a flexible linker that connects (i) and (ii), wherein the mutant IL-1α or mutant pro-IL-1α is characterized by low or high affinity or activity at the IL-1 receptor. In some embodiments, the present vaccine adjuvants as described herein have the capacity to safely promote activation, expansion and memory differentiation of CD8⁺ T cells. The present disclosure also concerns, in part, to the finding that adjuvants, chimeric proteins, or chimeric protein complexes comprising an IL-1α, pro-IL-1α, or a mutant thereof, exhibit substantially reduced or increased IL-1α activity compared to wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). In some embodiments, reduced IL-1α-activation signaling activity, however, can be induced and/or restored at a target cell when directed to such a cell through a targeting moiety, which binds to an antigen or receptor of interest. Surprisingly, the induced and/or restored IL-1α activity at a target cell, achieved through targeting of vaccine compositions, adjuvants, chimeric proteins, or chimeric protein complexes comprising IL-1α, pro-IL-1α, or variants thereof, may be similar or greater at the target cell than that of wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118).

Importantly, the adjuvants, chimeric proteins, and chimeric protein complexes comprising mutant IL-1α or pro-IL-1α described herein, exhibit substantial and surprising selectivity for target cells versus non-target cells, and substantially more than, for example, achieved with wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). In summary, a unique combination of highly potent and highly cell target-selective signaling activation can be achieved with the adjuvants, chimeric proteins, or chimeric protein complexes described herein.

In some embodiments, the loss in affinity and/or activity of IL-1α or pro-IL-1α for its receptor, e.g., IL-1 receptor, can be induced and/or restored upon directing or targeting of the adjuvant, the chimeric protein, or the chimeric protein complex comprising IL-1α or pro-IL-1α to a target cell through a targeting moiety. In some embodiments, the induction and/or restoration of IL-1α-mediated activation at a target cell may reach a level that is similar to or higher than activation achieved with wild type (non-chimeric) IL-1α.

In an embodiment, the modified IL-1α or pro-IL-1α comprises one or more mutations that reduce or increase its affinity and/or activity for IL-1 receptor. In another embodiment, the modified IL-1α or pro-IL-1α comprises one or more mutations that reduce or increase its affinity and/or activity for IL-1R1 or IL-1RAcP (co-receptor). In an embodiment, the modified IL-1α or pro-IL-1α comprises one or more mutations that reduce or increase its affinity and/or activity for IL-1R1 and comprises one or more mutations that reduce or increase its affinity and/or activity for IL-1RAcP. In some embodiments, a loss in affinity and/or activity of the modified IL-1α or pro-IL-1α for a receptor, e.g., IL-1R1, IL-1RAcP, can be induced and/or restored upon directing or targeting of the adjuvant, the chimeric protein, or the chimeric protein complex comprising the modified IL-1α or pro-IL-1α to a target cell through a targeting moiety.

In some embodiments, the adjuvant, the chimeric protein, and the chimeric protein complex, including Fc-based chimeric protein complex, comprises one or more additional signaling agents or cytokines, e.g., without limitation, an interferon, an interleukin, and a tumor necrosis factor, that may be modified. In various embodiments, the adjuvant, the chimeric protein, or chimeric protein complex, including Fc-based chimeric protein complex, of the invention provides improved safety and/or therapeutic activity and/or pharmacokinetic profiles (e.g., increased serum half-life) compared to an untargeted and/or unmodified IL-1α or pro-IL-1α or an unmodified, wild type IL-1α or pro-IL-1α.

In various embodiments, the adjuvants, the chimeric proteins, or the chimeric protein complexes, including Fc-based chimeric protein complexes, comprise one or more targeting moieties which have recognition domains (e.g. antigen recognition domains, including without limitation various antibody formats, inclusive of single-domain antibodies) which specifically bind to a target (e.g. antigen, receptor) of interest. In various embodiments, the targeting moieties have recognition domains that specifically bind to a target (e.g. antigen, receptor) of interest, including those found on one or more cancer cells, immune cells, which can include, without limitation, T cells, cytotoxic T lymphocytes, T helper cells, T regulatory cells (Tregs), natural killer (NK) cells, natural killer T (NKT) cells, macrophages (e.g. M1 and M2 macrophages), B cells, B regulatory (Breg) cells, neutrophils, monocytes, myeloid derived cells, and dendritic cells. In various embodiments, the targeting moieties have recognition domains that specifically bind to a target (e.g. antigen, receptor) of interest, including those found on one or more cancer cells, endothelial cells, epithelial cells, mesenchymal cells, stromal cells or other cell types that are characteristic of and/or unique for specific organs and/or tissues, including those specifically associated with disease. In some embodiments, the recognition domains specifically bind to a target (e.g. antigen, receptor) of interest and effectively recruit one or more immune cells.

In some embodiments, the adjuvants, the chimeric proteins, or the chimeric protein complexes, including Fc-based chimeric protein complexes, may recruit an immune cell, e.g., an immune cell that can cause an anti-infective effect, or modulate other immune cells, to a site of action. In some embodiments, the adjuvants, the chimeric proteins, or the chimeric protein complexes, including Fc-based chimeric protein complexes, may modulate an immune cell at a site of action, or recruit an immune cell to a site of action.

In some aspects, the present invention is related to a method for vaccinating a subject against an infectious disease, comprising administering: (a) an adjuvant comprising a chimeric protein or chimeric protein complex, comprising: (1) an IL-1α, pro-IL-1α, or a mutant thereof (which is an example of a signaling agent as described herein), (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) and/or (2) a flexible linker that connects (i) and (ii); wherein the mutant IL-1 or the mutant pro-IL-1α is characterized by low or high affinity or activity at the IL-1 receptor; and (b) an antigen which is suitable for inducing an immune response.

### BRIEF DESCRIPTION OF THE DRAWINGS

FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, and 19A-F show various non-limiting illustrative schematics of the chimeric protein complexes (e.g., a Fc-based chimeric protein complex) of the present invention. In embodiments, each schematic is a composition of the present invention. Where applicable in the figures, “TM” refers to a “targeting moiety” as described herein, “SA” refers to a “signaling agent” as described herein, [symbol] is an optional “linker” as described herein, the two long parallel rectangles are human Fc domains, e.g. from IgG1, from IgG2, or from IgG4, as described herein and optionally with effector knock-out and/or stabilization mutations as also described herein, and the two long parallel rectangles with one having a protrusion and the other having an indentation are human Fc domains, e.g. from IgG1, from IgG2, or from IgG4 as described herein, with knob-in-hole and/or ionic pair (a/k/a charged pairs, ionic bond, or charged residue pair) mutations as described herein and optionally with effector knock-out and/or stabilization mutations as also described herein.

FIGS. 1A-F show illustrative homodimeric 2-chain complexes. These figures show illustrative configurations for the homodimeric 2-chain complexes.

FIGS. 2A-H show illustrative homodimeric 2-chain complexes with two targeting moieties (TM) (as described herein, more targeting moieties may be present in some embodiments). In embodiments, the position of TM1 and TM2 are interchangeable. In embodiments, the constructs shown in the box (i.e., FIGS. 2G and 2H) have signaling agent (SA) between TM1 and TM2 or between TM1 and Fc.

FIGS. 3A-H show illustrative homodimeric 2-chain complexes with two signaling agents (as described herein, more signaling agents may be present in some embodiments). In embodiments, the position of SA1 and SA2 are interchangeable. In embodiments, the constructs shown in the box (i.e., FIGS. 3G and 3H) have TM between SA1 and SA2 or TM at N- or C-terminus.

FIGS. 4A-D show illustrative heterodimeric 2-chain complexes with split TM and SA chains, namely the TM on the knob chain of the Fc and the SA on hole chain of the Fc.

FIGS. 5A-F show illustrative heterodimeric 2-chain complexes with split TM and SA chains, namely with both TMs on the knob chain of the Fc and with SA on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 6A-J show illustrative heterodimeric 2-chain complexes with split TM and SA chains, namely with TM on the knob chain of the Fc and with a SA on the hole chain of the Fc, with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 7A-D show illustrative heterodimeric 2-chain complexes with split TM and SA chains, namely the SA on the knob chain of the Fc and the TM on hole chain of the Fc.

FIGS. 8A-F show illustrative heterodimeric 2-chain complexes with split TM and SA chains, namely with SA on the knob chain of the Fc and both TMs on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 9A-J show illustrative heterodimeric 2-chain complexes with split TM and SA chains, namely with SA on the knob chain of the Fc and TM on hole chain of the Fc, with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 10A-F show illustrative heterodimeric 2-chain complexes with TM and SA on the same chain, namely the SA and TM both on the knob chain of the Fc.

FIGS. 11A-L show illustrative heterodimeric 2-chain complexes with a TM and a SA on the same chain, namely with SA and with TM both on the knob chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 12A-L show illustrative heterodimeric 2-chain complexes with a TM and a SA on the same chain, namely with SA and with TM both on the knob chain of the Fc, with two signaling agents (as described herein, more signaling agents may be present in some embodiments). In embodiments, the position of SA1 and SA2 are interchangeable.

FIGS. 13A-F show illustrative heterodimeric 2-chain complexes with TM and SA on the same chain, namely the SA and TM both on the hole chain of the Fc.

FIGS. 14A-L show illustrative heterodimeric 2-chain complexes with a TM and a SA on the same chain, namely with SA and with TM both on the hole chain of the Fc, with two targeting moieties (as described herein, more targeting moieties are present in some embodiments). In embodiments, the position of TM1 and TM2 are interchangeable. In embodiments, TM1 and TM2 can be identical.

FIGS. 15A-L show illustrative heterodimeric 2-chain complexes with a TM and a SA on the same chain, namely with SA and with TM both on the hole chain of the Fc, with two signaling agents (as described herein, more signaling agents may be present in some embodiments). In embodiments, the position of SA1 and SA2 are interchangeable.

FIGS. 16A-J show illustrative heterodimeric 2-chain complexes with two targeting moieties (as described herein, more targeting moieties may be present in some embodiments) and with SA on knob Fc and TM on each chain. In embodiments, TM1 and TM2 can be identical.

FIGS. 17A-J show illustrative heterodimeric 2-chain complexes with two targeting moieties (as described herein, more targeting moieties may be present in some embodiments) and with SA on hole Fc and TM on each chain. In embodiments, TM1 and TM2 can be identical.

FIGS. 18A-F show illustrative heterodimeric 2-chain complexes with two signaling agents (as described herein, more signaling agents may be present in some embodiments) and with split SA and TM chains: SA on knob and TM on hole Fc.

FIGS. 19A-F show illustrative heterodimeric 2-chain complexes with two signaling agents (as described herein, more signaling agents may be present in some embodiments) and with split SA and TM chains: TM on knob and SA on hole Fc.

FIG. 20 depicts NF-KB reporter activity in HEK293T cells treated with recombinant IL1a or recombinant Fc-IL1a.

FIG. 21 depicts NF-KB reporter activity in HEK293T cells with and without CD8 expression treated with recombinant IL1α or recombinant Fc-IL1α. Note that the anti-CD8 VHH-Fc-IL1α more potent than recombinant IL1α at ˜0.01 nM (boxed area).

FIGS. 22A-C shows NF-KB reporter activity in HEK293T cells with and without CD8 expression treated with recombinant anti-CD8 VHH-Fc-IL1α with C141 mutations

FIGS. 23A-D shows NF-KB reporter activity in HEK293T cells with and without CD8 expression treated with recombinant anti-CD8 VHH-Fc-IL1α with A44T, N29A, or N29G mutations

FIGS. 24A-C depicts NF-KB reporter activity in HEK293T cells with and without CD8 expression treated with recombinant anti-CD8 VHH-Fc-IL1α with D151 mutations.

FIGS. 25A-C shows NF-KB reporter activity in HEK293T cells with and without CD8 expression treated with recombinant anti-CD8 VHH-Fc-IL1α with M15G, R16G, or 118G mutations.

FIGS. 26A-H depicts NF-KB reporter activity in HEK293T cells with and without CD8 expression treated with recombinant anti-CD8 VHH-Fc-IL1α with R16A, 118A, H46A, H46G, A58H, A58N, 168G, and D64G/D65A mutations.

FIGS. 27A-F shows biological activity of CD8-targeted or -untargeted human IL-1α Fc-construct with a del1-6 mutation, with or without C141 mutation.

FIGS. 28A-H shows biological activity of CD8 targeted human IL-1α Fc-construct with a del1-6 mutation with additional mutations at C141, N29 or S31 (PC=parental HekBlue-IL-1β cell line; hCD8=HekBlue-IL-1β cell line stably transfected with CD8).

FIGS. 29A-J depicts biological activity of CD8 targeted human IL-1α Fc-construct with a del1-6 mutation and N29 or S31 mutation with or without C141 mutation.

FIG. 30 depicts biological activity of CD8-targeted Fc-IL-1α variants with C- or N-terminally fused IL-1α on parental (PC) and CD8+ HekBlue-IL-1β cells.

FIG. 31 shows biological activity of CD8-targeted single-peptide IL-1α variants on parental (PC) and CD8+ HekBlue-IL-1β cells.

FIG. 32 shows biological activity of CD8-targeted Fc-IL-1α (delta1-6) C141H or M15G, on mock or CD8 transiently transfected HEK293T cells, using different CD8 targeting domains (VHHs or OKT8 scFv).

FIG. 33 depicts biological activity of monovalent and bivalent CD8-targeted Fc-IL-1α variants on parental (PC) and CD8+ HekBlue-IL-1β cells.

FIG. 34 shows biological activity of NKp46-targeted Fc-IL-1α variants on isolated NK cells.

### DETAILED DESCRIPTION

In one aspect, the present invention provides a chimeric protein, a chimeric protein complex, a vaccine composition, or an adjuvant, that includes an interleukin-1α (IL-1α), pro-IL-1α or a mutant thereof. IL-1α is a proinflammatory cytokine and an important immune system regulator. IL-1α is constitutively expressed as a 31-kDa precursor by epithelial cells, endothelial cells, and keratinocytes. Unlike IL-1β, IL-1α exhibits a basal amount of activity in its immature unprocessed form (i.e., pro-IL-1α exhibits basal amount of activity). IL-1α is released from damaged cells, and binds to IL-1R1 receptor (see Afonina et al., Immunity 42, 991-1004, 2015). IL-1α expression is rapidly upregulated by a wide variety of danger- and pathogen-associated molecular patterns. IL-1α is very promiscuous in its sub-cellular localization and can function as an IL-1R agonist upon secretion from the cell or as a cell membrane bound molecule. While the other IL-1R ligand, IL-1β, requires inflammasome activation for its maturation and pyroptosis for its secretion, IL-1α is bioavailable in a much wider set of cellular scenarios. IL-1α is bioactive in both the pro-form and mature form, and therefore both pyroptotic and necrotic cell death yields bioactive IL-1α.

IL-1α is translated as pro-IL-1α and has several post-translational modifications of this precursor form. Specifically, pro-IL-1α is phosphorylated at Ser90, myristoylated on Lys82, and acetylated on Lys82 (Di Paolo et al., Nat Immunol. 2016 Jul. 19; 17 (8): 906-913). Cleavage of human pro-IL-1α at Phe118 is mediated by the calcium-dependent neutral protease calpain. Calpain can cleave pro-IL-1α inside the cell or under cell-free conditions.

In various embodiments, the IL-1α of present invention includes truncated forms of pro-IL-1α that exhibit activity or bind to IL-1α's receptor. For instance, in one embodiment, the IL-1α of the present invention includes amino acids 128 to 267 of pro-IL-1α having the amino acid sequence of SEQ ID NO: 2 or 4 (as described, e.g., by Mosley, Bruce, et al., “Determination of the minimum polypeptide lengths of the functionally active sites of human interleukins 1 alpha and 1 beta,” PNAS 84.13 (1987): 4572-4576, which is hereby incorporated by reference in its entirety). In some embodiments, the truncated form of pro-IL-1α is cleaved, resulting in a truncated IL-1α with an N-terminus at e.g., at N102, S104, S117, or L119 of the amino acid sequence of SEQ ID NO: 2 or 4 (as described, e.g., by Afonina, et al., “Granzyme B-dependent Proteolysis Acts as a Switch to Enhance the Proinflammatory Activity of IL-1α,” Mol Cell. 44:265-278 (2011) and Afonina et al., “Proteolytic Processing of Interleukin-1 Family Cytokines: Variations on a Common Theme,” Immunity 42.6 (2015): 991-1004 (2015), which are hereby incorporated by reference in their entirety).

In various embodiments, the present invention provides a chimeric protein or chimeric protein complex, such as Fc-based chimeric protein complexes, that includes an IL-1α or a mutant thereof fused to one or more targeting moieties. In some embodiments, the present invention provides a chimeric protein or chimeric protein complex, such as Fc-based chimeric protein complexes, that include a pro-IL-1α or a mutant thereof fused to one or more targeting moieties.

In some embodiments, the mutant IL-1α or the mutant pro-IL-1α is human IL-1α or human pro-IL-1α. In some embodiments, the mutant IL-1α or mutant pro-IL-1α has low or high affinity and/or activity for IL-1 receptor. In some embodiments, the mutant IL-1α or mutant pro-IL-1α has substantially reduced or ablated or increased affinity and/or activity for IL-1 receptor. In some embodiments, a low affinity or activity of the mutant IL-1α or mutant pro-IL-1α at the IL-1 receptor is restorable and/or inducible by attachment to one or more targeting moieties or upon inclusion in the chimeric protein complex.

In an embodiment, the wild type IL-1α has the amino acid sequence of:

IL-1α (wild type)
(SEQ ID NO: 1)
SAPFSFLSNVKYNFMRIIKYEFILNDALNQSIIRANDQYLTAAALHNL
DEAVKFDMGAYKSSKDDAKITVILRISKTQLYVTAQDEDQPVLLKEMP
EIPKTITGSETNLIFFWETHGTKNYFTSVAHPNLFIATKQDYWVCLAG
GPPSITDFQILENQA.

In an embodiment, the wild type pro-IL-1α has the amino acid sequence of:

Pro-IL-1α (wild type)
(SEQ ID NO: 2)
MAKVPDMFEDLKNCYSENEEDSSSIDHLSLNQKSFYHVSYGPLHEGCMDQ
SVSLSISETSKTSKLTFKESMVWATNGKVLKKRRLSLSQSITDDDLEAIA
NDSEEEIIKPRSAPFSFLSNVKYNFMRIIKYEFILNDALNQSIIRANDQY
LTAAALHNLDEAVKFDMGAYKSSKDDAKITVILRISKTQLYVTAQDEDQP
VLLKEMPEIPKTITGSETNLIFFWETHGTKNYFTSVAHPNLFIATKQDYW
VCLAGGPPSITDFQILENQA.

In an embodiment, the wild type IL-1α has the amino acid sequence of:

IL-1α (wild type)
(SEQ ID NO: 3)
SAPFSFLSNVKYNFMRIIKYEFILNDALNQSIIRANDQYLTAAALHNLDE
AVKFDMGAYKSSKDDAKITVILRISKTQLYVTAQDEDQPVLLKEMPEIPK
TITGSETNLLFFWETHGTKNYFTSVAHPNLFIATKQDYWVCLAGGPPSIT
DFQILENQA.

In an embodiment, the wild type pro-IL-1α has the amino acid sequence of:

Pro-IL-1α (wild type)
(SEQ ID NO: 4)
MAKVPDMFEDLKNCYSENEEDSSSIDHLSLNQKSFYHVSYGPLHEGCMDQ
SVSLSISETSKTSKLTFKESMVVVATNGKVLKKRRLSLSQSITDDDLEAI
ANDSEEEIIKPRSAPFSFLSNVKYNFMRIIKYEFILNDALNQSIIRANDQ
YLTAAALHNLDEAVKFDMGAYKSSKDDAKITVILRISKTQLYVTAQDEDQ
PVLLKEMPEIPKTITGSETNLLFFWETHGTKNYFTSVAHPNLFIATKQDY
WVCLAGGPPSITDFQILENQA.

In some embodiments, the mutant human IL-1α has an amino acid sequence of at least 95%, or 96%, or 97%, or 98%, or 99% identity to SEQ ID NO: 1 or 3. In some embodiments, the mutant human pro-IL-1α has an amino acid sequence of at least 95%, or 96%, or 97%, or 98%, or 99% identity to SEQ ID NO: 2 or 4.

In some embodiments, the human IL-1α has an amino acid sequence of wild-type human IL-1α.

In some embodiments, the mutant IL-1α has a deletion mutation of del1-6 (or “delta 1-6”) with respect to SEQ ID NO: 1 or 3, wherein the deletion mutation of del1-6 is a deletion of amino acid residues numbered 1-6 of SEQ ID NO: 1 or 3.

In some embodiments, the mutant pro-IL-1α has a deletion mutation of del113-118 (or “delta 113-118”) with respect to SEQ ID NO: 2 or 4, wherein the deletion mutation of del113-118 is a deletion of amino acid residues numbered 113-118 of SEQ ID NO: 2 or 4.

In some embodiments, the mutant IL-1α has one or more substitution mutations selected from C141, N29, S31, P3, M15, R16, 117, 118, L24, N25, D26, L28, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, L142, D151, F152, Q153 where the residue numbering is based on SEQ ID NO: 1 or 3. In some embodiments, one or more of these mutations produce a modified human IL-1α with reduced or increased binding affinity for type I IL-1 and reduced or increased biological activity. In some embodiments, the mutant IL-1α has one or more substitution mutations selected C141A, C141S, N29A, N29D, N29G, S31A, S31G, M15A, M15G, M15S, R16A, R16K, R16G, 118A, 118G, 118L, L24K, L24S, N25A, N25G, D26V, L28A, L28G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141H, D151A, D151K, D151Y, F152Q, F152N, F152S, Q153A, and Q153G where the residue numbering is based on SEQ ID NO: 1 or 3.

In some embodiments, the mutant pro-IL-1α has one or more substitution mutations selected N141, S143, P115, M127, R128, 1129, 1130, L136, N137, D138, L140, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 where the residue numbering is based on SEQ ID NO: 2 or 4. In some embodiments, one or more of these mutations produce a modified human pro-IL-1α with reduced or increased binding affinity for type I IL-1 and reduced or increased biological activity. In some embodiments, the mutant pro-IL-1α has one or more substitution mutations selected from N141A, N141D, N141G, S143A, S143G, M127S, R128A, R128K, 1130A, 1130L, L136K, L136S, N137A, N137G, D138V, L140A, L140G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G where the residue numbering is based on SEQ ID NO: 2 or 4.

In embodiments, the one or more additional mutations confer reduced or increased activity as compared to an IL-1α having the amino acid sequence of SEQ ID NO: 1 or 3 having a deletion of amino acids 1-6 (delta 1-6), or compared to a pro-IL-1α having the amino acid sequence of SEQ ID NO: 2 or 4 having a deletion of amino acids 113-118 (delta 113-118). In such embodiments, the one or more mutations of IL-1α or pro-IL-1α confer reduced or increased activity that is restorable and/or inducible by attachment to one or more targeting moieties or upon inclusion in the chimeric protein or chimeric protein complex. In other embodiments the one or more additional mutations confer increased activity as compared to an IL-1α having the amino acid sequence of SEQ ID NO: 1 or 3 having a deletion of amino acids 1-6 (delta 1-6), optionally wherein the one or more additional mutations are selected from an amino acid substitution at a position selected from N29 and S31 with respect to SEQ ID NO: 1 or 3, optionally wherein the substitution is selected from N29A, N29D, N29G, S31A, and S31G. 25 In other embodiments the one or more additional mutations confer increased activity as compared to a pro-IL-1α having the amino acid sequence of SEQ ID NO: 2 or 4 having a deletion of amino acids 113-118 (delta 113-118), optionally wherein the one or more additional mutations are selected from an amino acid substitution at a position selected from N141 and S143 with respect to SEQ ID NO: 2 or 4, optionally wherein the substitution is selected from N141A, N141D, N141G, S143A, and S143G.

In some embodiments, the one or more additional mutations confer increased activity to the chimeric proteins and chimeric protein complexes of the present application. In such embodiments, the present application provides chimeric proteins and chimeric protein complexes that have a bioactivity that is at least about 2-fold greater, about 3-fold greater, about 4-fold greater, about 5-fold greater, about 6-fold greater, about 7-fold greater, about 8-fold greater, about 9-fold greater, at least about 10-fold greater, at least about 15-fold greater, at least about 20-fold greater, at least about 25-fold greater, at least about 30-fold greater, at least about 35-fold greater, at least about 40-fold greater, at least about 45-fold greater, at least about 50-fold greater, at least about 100-fold greater, at least about 150-fold greater, or about 10-50-fold greater, about 50-100-fold greater, about 100-150-fold greater, about 150-200-fold greater, or more than 200-fold greater when comparing the bioactivity on a plurality of cells that express or overexpress the antigen to which the targeting moiety is directed against a plurality of cells that do not express or minimally express the antigen to which the targeting moiety is directed.

In various embodiments, the mutations allow for IL-1α or pro-IL-1α to have one or more of attenuated or reduced activity such as one or more of reduced binding affinity, reduced endogenous activity, and reduced specific bioactivity relative to unmodified or unmutated, i.e., the wild type form of IL-1α or pro-IL-1α (e.g. comparing IL-1α in a wild type form versus a modified (e.g. mutant) form) or compared to wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). In some embodiments, the mutations that attenuate or reduce binding or affinity include those mutations that substantially reduce or ablate binding or activity. In some embodiments, the mutations that attenuate or reduce binding or affinity are different than those mutations which substantially reduce or ablate binding or activity. Consequentially, in various embodiments, the mutations allow for IL-1α to have improved safety, e.g. have reduced systemic toxicity, reduced side effects, and reduced off-target effects relative to unmutated, i.e. wild type, IL-1α (e.g. comparing IL-1α in a wild type form versus a modified (e.g. mutant) form) or compared to wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118).

In various embodiments, the mutations allow for IL-1α or pro-IL-1α to have increased activity such as one or more of increased binding affinity, increased endogenous activity, and increased specific bioactivity relative to unmodified or unmutated, i.e., the wild type form of IL-1α or pro-IL-1α (e.g. comparing IL-1α in a wild type form versus a modified (e.g. mutant) form) or compared to wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). Consequentially, in various embodiments, the mutations allow for IL-1α to have improved safety, e.g. have reduced systemic toxicity, reduced side effects, and reduced off-target effects relative to unmutated, i.e. wild type, IL-1α (e.g. comparing IL-1α in a wild type form versus a modified (e.g. mutant) form) or compared to wild type IL-1α or wild type pro-IL-1a or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118).

In various embodiments, IL-1α or pro-IL-1α is modified to have one or more mutations that reduce or increase its binding affinity or activity for one or more of its receptors. In some embodiments, IL-1α or pro-IL-1α is modified to have one or more mutations that substantially reduce or ablate or increase binding affinity or activity for the receptors. In some embodiments, the activity provided by the wild type IL-1α or pro-IL-1α is agonism at the receptor (e.g. activation of a cellular effect at a site of therapy). For example, the wild type IL-1α or pro-IL-1α may activate its receptor. In such embodiments, the mutations result in the modified IL-1α or pro-IL-1α to have reduced or ablated or increased activating activity at the receptor. For example, the mutations may result in the modified IL-1α or pro-IL-1α to deliver a reduced or increased activating signal to a target cell or the activating signal could be ablated.

In some embodiments, the activity provided by the wild type IL-1α (or pro-IL-1α) is antagonism at the receptor (e.g. blocking or dampening of a cellular effect at a site of therapy). For example, the wild type IL-1α or pro-IL-1α may antagonize or inhibit the receptor. In these embodiments, the mutations result in the modified IL-1α or pro-IL-1α to have a reduced or ablated or increased antagonizing activity at the receptor. For example, the mutations may result in the modified IL-1α or pro-IL-1α to deliver a reduced or increased inhibitory signal to a target cell or the inhibitory signal could be ablated.

In some embodiments, a reduced affinity or activity of IL-1α or pro-IL-1α at the receptor is restorable and/or inducible by attachment with one or more of the targeting moieties. In other embodiments, the reduced affinity or activity of IL-1α or pro-IL-1α at the receptor is not substantially restorable and/or inducible by the activity of one or more of the targeting moieties.

In various embodiments, the chimeric proteins or chimeric protein complexes of the present invention reduce off-target effects because the IL-1α or pro-IL-1α has mutations that weaken or ablate binding affinity or activity at a receptor. In various embodiments, this reduction in side effects is observed relative with, for example, the wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). In various embodiments, the IL-1α or pro-IL-1α is active on target cells because the targeting moiety(ies) compensates for the missing/insufficient binding (e.g., without limitation and/or avidity) required for substantial activation. In various embodiments, the modified IL-1α or pro-IL-1α is substantially inactive en route to the site of therapeutic activity and has its effect substantially on specifically targeted cell types that greatly reduces undesired side effects.

In various embodiments, substantially reducing or ablating binding or activity at the receptor causes the therapeutic effect of IL-1α or pro-IL-1α to improve as there is a reduced or eliminated sequestration of the therapeutic chimeric proteins away from the site of therapeutic action. For instance, in some embodiments, this obviates the need of high doses of the present vaccine compositions that compensate for loss at the other receptor. Such ability to reduce dose further provides a lower likelihood of side effects.

In various embodiments, the modified IL-1α or pro-IL-1α comprises one or more mutations that cause IL-1α or pro-IL-1α to have reduced, substantially reduced, or ablated affinity, e.g. binding (e.g. K_{D}) and/or activation. In various embodiments, the reduced affinity at IL-1α's or pro-IL-1α's receptor allows for attenuation of activity (inclusive of agonism or antagonism). In such embodiments, the modified IL-1α or pro-IL-1α has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor relative to the wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118).

In various embodiments, the modified IL-1α or pro-IL-1α comprises one or more mutations that cause IL-1α or pro-IL-1α to have increased or substantially increased affinity, e.g. binding (e.g. K_{D}) and/or activation. In various embodiments, the increased affinity at IL-1α's or pro-IL-1α's receptor allows for enhancement of activity (inclusive of agonism or antagonism). In such embodiments, the modified IL-1α or pro-IL-1α has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% greater affinity for the receptor relative to the wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). In some embodiments, the activity and/or binding affinity is at least about 2-fold higher, about 3-fold higher, about 4-fold higher, about 5-fold higher, about 6-fold higher, about 7-fold higher, about 8-fold higher, about 9-fold higher, at least about 10-fold higher, at least about 15-fold higher, at least about 20-fold higher, at least about 25-fold higher, at least about 30-fold higher, at least about 35-fold higher, at least about 40-fold higher, at least about 45-fold higher, at least about 50-fold higher, at least about 100-fold higher, at least about 150-fold higher, or about 10-50-fold higher, about 50-100-fold higher, about 100-150-fold higher, about 150-200-fold higher, or more than 200-fold higher relative to the wild type IL-1α or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118).

In various embodiments, the modified IL-1α or pro-IL-1α comprises one or more mutations that reduce or increase the endogenous activity of IL-1α or pro-IL-1α by about 75%, or about 70%, or about 60%, or about 50%, or about 40%, or about 30%, or about 25%, or about 20%, or about 10%, or about 5%, or about 3%, or about 1%, e.g., relative to the wild type IL-1 or wild type pro-IL-1α or Fc IL-1α or Fc pro-IL-1α or Fc IL-1α (delta 1-6) or Fc pro-IL-1α (delta 113-118) or targeted Fc IL-1α (delta 1-6) or targeted Fc pro-IL-1α (delta 113-118). In some embodiments, the modified IL-1α or pro-IL-1α comprises one or more mutations that cause IL-1α (or pro-IL-1α) to have reduced or increased affinity for its receptor that is lower or higher than the binding affinity of the targeting moiety(ies) for its (their) receptor(s). In some embodiments, this binding affinity differential is between IL-1α/receptor and targeting moiety/receptor on the same cell. In some embodiments, this binding affinity differential allows for mutant IL-1α or pro-IL-1α to have localized, on-target effects and to minimize off-target effects that underlie side effects that are observed with wild type IL-1α or wild type pro-IL-1α. In some embodiments, this binding affinity is at least about 2-fold, or at least about 5-fold, or at least about 10-fold, or at least about 15-fold lower or higher, or at least about 25-fold, or at least about 50-fold, or at least about 100-fold, or at least about 150-fold lower or higher.

Receptor binding activity may be measured using methods known in the art. For example, affinity and/or binding activity may be assessed by Scatchard plot analysis and computer-fitting of binding data (e.g. Scatchard, 1949) or by reflectometric interference spectroscopy under flow through conditions, as described by Brecht et al. (1993), the entire contents of all of which are hereby incorporated by reference.

In various embodiments the modified IL-1α or pro-IL-1α comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known wild type IL-1α or wild type pro-IL-1α (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments the modified IL-1α comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any of amino acid sequences of the IL-1α or pro-IL-1α disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments, the modified IL-1α or pro-IL-1α comprises an amino acid sequence having one or more amino acid mutations. In some embodiments, the one or more amino acid mutations may be independently selected from substitutions, insertions, deletions, and truncations. In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In some embodiments, a reduced affinity or activity of the modified IL-1α or pro-IL-1α at the therapeutic receptor is inducible and/or restorable by attachment to a targeting moiety or upon inclusion of a targeting moiety in a chimeric protein or a chimeric protein complex, e.g., a Fc-based chimeric protein complex as disclosed herein. In some embodiments, the activity of IL-1α or pro-IL-1α is reduced or attenuated by virtue of its fusion with another protein, including, in some instances, by fusion with targeting moieties as described herein. In other embodiments, the activity of IL-1α or pro-IL-1α is reduced or attenuated by modifying the IL-1α or pro-IL-1α, e.g., by introducing mutations as described herein. In some embodiments, attenuation of the activity can be restored and/or induced by attaching the IL-1α or pro-IL-1α to a targeting moiety or by the action of the attached targeting moiety. In embodiments, the targeting moiety—by virtue of its attachment or by its activity-induces IL-1α's activity. In some embodiments, the reduced affinity or activity at the receptor is inducible and/or restorable by attachment with one or more of the targeting moieties as described herein or upon inclusion in the chimeric protein complexes, such as Fc-based chimeric protein complex disclosed herein.

### Targeting Moiety

In various embodiments, chimeric protein, chimeric protein complex, vaccine composition, or adjuvant of the present invention additionally comprise one or more targeting moieties having recognition domains, which specifically bind to a target (e.g. antigen, receptor) of interest. In some embodiments, chimeric protein, chimeric protein complex, vaccine composition, or adjuvant may comprise two, three, four, five, six, seven, eight, nine, ten or more targeting moieties. In illustrative embodiments, chimeric protein, chimeric protein complex, vaccine composition, or adjuvant of the invention comprise two or more targeting moieties. In such embodiments, chimeric protein, chimeric protein complex, vaccine composition, or adjuvant can target two different cells (e.g. to make a synapse) or the same cell (e.g. to get a more concentrated signaling agent effect). In some embodiments, chimeric protein, chimeric protein complex, vaccine composition, or adjuvant of the invention comprise IL-1α, pro-IL-1α, or a variant thereof, a targeting moiety that is Flt3L and one targeting moiety that recognizes PD-1 or PD-L1. In some embodiments, chimeric protein, chimeric protein complex, vaccine composition, or adjuvant of the invention comprise IL-1α, pro-IL-1α, or a variant thereof, a targeting moiety that is Flt3L and two targeting moieties that recognizes PD-1 or PD-L1.

In some embodiments, chimeric protein, chimeric protein complex, vaccine composition, or adjuvant of the invention comprise IL-1α, pro-IL-1α, or a variant thereof, a targeting moiety that is FIt3L and one targeting moiety that recognizes PD-1 or PD-L1. In some embodiments, chimeric protein, chimeric protein complex, vaccine composition, or adjuvant of the invention comprise IL-1α, pro-IL-1α, or a variant thereof, a targeting moiety that is Flt3L and two targeting moieties that recognizes PD-1 or PD-L1.

In various embodiments, the target (e.g. antigen, receptor) of interest can be found on one or more immune cells, which can include, without limitation, T cells, cytotoxic T lymphocytes, T helper cells, natural killer (NK) cells, natural killer T (NKT) cells, anti-tumor or tumor-associated macrophages (e.g. M1 or M2 macrophages), B cells, Breg cells, dendritic cells, or subsets thereof. In some embodiments, the recognition domains specifically bind to a target (e.g. antigen, receptor) of interest and effectively, directly or indirectly, recruit one of more immune cells. In some embodiments, the target (e.g. antigen, receptor) of interest can be found on one or more tumor cells. In some embodiments, chimeric protein, chimeric protein complex, vaccine composition, or adjuvant may directly or indirectly recruit an immune cell, e.g., in some embodiments, to a therapeutic site (e.g. a locus with one or more disease cell or cell to be modulated for a therapeutic effect). In some embodiments, chimeric protein, chimeric protein complex, vaccine composition, or adjuvant may directly or indirectly recruit an immune cell, e.g. an immune cell that can kill and/or suppress a tumor cell, to a site of action (such as, by way of non-limiting example, the tumor microenvironment).

In various embodiments, chimeric protein, chimeric protein complex, vaccine composition, or adjuvant have targeting moieties having recognition domains, which specifically bind to a target (e.g. antigen, receptor) which is part of a non-cellular structure. In some embodiments, the antigen or receptor is not an integral component of an intact cell or cellular structure. In some embodiments, the antigen or receptor is an extracellular antigen or receptor. In some embodiments, the target is a non-proteinaceous, non-cellular marker, including, without limitation, nucleic acids, inclusive of DNA or RNA, such as, for example, DNA released from necrotic tumor cells or extracellular deposits such as cholesterol.

In some embodiments, the target (e.g. antigen, receptor) of interest is part of the non-cellular component of the stroma or the extracellular matrix (ECM) or the markers associated therewith. As used herein, stroma refers to the connective and supportive framework of a tissue or organ. Stroma may include a compilation of cells such as fibroblasts/myofibroblasts, glial, epithelia, fat, immune, vascular, smooth muscle, and immune cells along with the extracellular matrix (ECM) and extracellular molecules. In various embodiments, the target (e.g. antigen, receptor) of interest is part of the non-cellular component of the stroma such as the extracellular matrix and extracellular molecules. As used herein, the ECM refers to the non-cellular components present within all tissues and organs. The ECM is composed of a large collection of biochemically distinct components including, without limitation, proteins, glycoproteins, proteoglycans, and polysaccharides. These components of the ECM are usually produced by adjacent cells and secreted into the ECM via exocytosis. Once secreted, the ECM components often aggregate to form a complex network of macromolecules. In various embodiments, chimeric protein, chimeric protein complex, vaccine composition, or adjuvant of the invention comprises a targeting moiety that recognizes a target (e.g., an antigen or receptor or non-proteinaceous molecule) located on any component of the ECM. Illustrative components of the ECM include, without limitation, the proteoglycans, the non-proteoglycan polysaccharides, fibers, and other ECM proteins or ECM non-proteins, e.g. polysaccharides and/or lipids, or ECM associated molecules (e.g. proteins or non-proteins, e.g. polysaccharides, nucleic acids and/or lipids).

In some embodiments, the targeting moiety recognizes a target (e.g. antigen, receptor) on ECM proteoglycans. Proteoglycans are glycosylated proteins. The basic proteoglycan unit includes a core protein with one or more covalently attached glycosaminoglycan (GAG) chains. Proteoglycans have a net negative charge that attracts positively charged sodium ions (Na+), which attracts water molecules via osmosis, keeping the ECM and resident cells hydrated. Proteoglycans may also help to trap and store growth factors within the ECM. Illustrative proteoglycans that may be targeted by chimeric protein, chimeric protein complex, vaccine composition, or adjuvant of the invention include, but are not limited to, heparan sulfate, chondroitin sulfate, and keratan sulfate. In an embodiment, the targeting moiety recognizes a target (e.g. antigen, receptor) on non-proteoglycan polysaccharides such as hyaluronic acid.

In some embodiments, the targeting moiety recognizes a target (e.g. antigen, receptor) on ECM fibers. ECM fibers include collagen fibers and elastin fibers. In some embodiments, the targeting moiety recognizes one or more epitopes on collagens or collagen fibers. Collagens are the most abundant proteins in the ECM. Collagens are present in the ECM as fibrillar proteins and provide structural support to resident cells. In one or more embodiments, the targeting moiety recognizes and binds to various types of collagens present within the ECM including, without limitation, fibrillar collagens (types I, II, III, V, XI), facit collagens (types IX, XII, XIV), short chain collagens (types VIII, X), basement membrane collagens (type IV), and/or collagen types VI, VII, or XIII. Elastin fibers provide elasticity to tissues, allowing them to stretch when needed and then return to their original state. In some embodiments, the target moiety recognizes one or more epitopes on elastins or elastin fibers.

In some embodiments, the targeting moiety recognizes one or more ECM proteins including, but not limited to, a tenascin, a fibronectin, a fibrin, a laminin, or a nidogen/entactin.

In an embodiment, the targeting moiety recognizes and binds to tenascin. The tenascin (TN) family of glycoproteins includes at least four members, tenascin-C, tenascin-R, tenascin-X, and tenascin W. The primary structures of tenascin proteins include several common motifs ordered in the same consecutive sequence: amino-terminal heptad repeats, epidermal growth factor (EGF)-like repeats, fibronectin type III domain repeats, and a carboxyl-terminal fibrinogen-like globular domain. Each protein member is associated with typical variations in the number and nature of EGF-like and fibronectin type III repeats. Isoform variants also exist particularly with respect to tenascin-C. Over 27 splice variants and/or isoforms of tenascin-C are known. In a particular embodiment, the targeting moiety recognizes and binds to tenascin-CA1. Similarly, tenascin-R also has various splice variants and isoforms. Tenascin-R usually exists as dimers or trimers. Tenascin-X is the largest member of the tenascin family and is known to exist as trimers. Tenascin-W exists as trimers. In some embodiments, the targeting moiety recognizes one or more epitopes on a tenascin protein. In some embodiments, the targeting moiety recognizes the monomeric and/or the dimeric and/or the trimeric and/or the hexameric forms of a tenascin protein.

In an embodiment, the targeting moieties recognize and bind to fibronectin. Fibronectins are glycoproteins that connect cells with collagen fibers in the ECM, allowing cells to move through the ECM. Upon binding to integrins, fibronectins unfolds to form functional dimers. In some embodiments, the targeting moiety recognizes the monomeric and/or the dimeric forms of fibronectin. In some embodiments, the targeting moiety recognizes one or more epitopes on fibronectin. In illustrative embodiments, the targeting moiety recognizes fibronectin extracellular domain A (EDA) or fibronectin extracellular domain B (EDB). Elevated levels of EDA are associated with various diseases and disorders including psoriasis, rheumatoid arthritis, diabetes, and cancer. In some embodiments, the targeting moiety recognizes fibronectin that contains the EDA isoform and may be utilized to target chimeric protein, chimeric protein complex, vaccine composition, or adjuvant to diseased cells including cancer cells. In some embodiments, the targeting moiety recognizes fibronectin that contains the EDB isoform. In various embodiments, such targeting moieties may be utilized to target chimeric protein, chimeric protein complex, vaccine composition, or adjuvant to tumor cells including the tumor neovasculature.

In an embodiment, the targeting moiety recognizes and binds to fibrin. Fibrin is another protein substance often found in the matrix network of the ECM. Fibrin is formed by the action of the protease thrombin on fibrinogen, which causes the fibrin to polymerize. In some embodiments, the targeting moiety recognizes one or more epitopes on fibrin. In some embodiments, the targeting moiety recognizes the monomeric as well as the polymerized forms of fibrin.

In an embodiment, the targeting moiety recognizes and binds to laminin. Laminin is a major component of the basal lamina, which is a protein network foundation for cells and organs. Laminins are heterotrimeric proteins that contain an α-chain, a β-chain, and a γ-chain. In some embodiments, the targeting moiety recognizes one or more epitopes on laminin. In some embodiments, the targeting moiety recognizes the monomeric, the dimeric as well as the trimeric forms of laminin.

In an embodiment, the targeting moiety recognizes and binds to a nidogen or entactin. Nidogens/entactins are a family of highly conserved, sulfated glycoproteins. They make up the major structural component of the basement membranes and function to link laminin and collagen IV networks in basement membranes. Members of this family include nidogen-1 and nidogen-2. In various embodiments, the targeting moiety recognizes an epitope on nidogen-1 and/or nidogen-2.

In various embodiments, the targeting moiety comprises an antigen recognition domain that recognizes an epitope present on any of the targets described herein. In an embodiment, the antigen-recognition domain recognizes one or more linear epitopes present on the protein. As used herein, a linear epitope refers to any continuous sequence of amino acids present on the protein. In another embodiment, the antigen-recognition domain recognizes one or more conformational epitopes present on the protein. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the targeting moiety may bind to the full-length and/or mature forms and/or isoforms and/or splice variants and/or fragments and/or any other naturally occurring or synthetic analogs, variants, or mutants of any of the targets described herein. In various embodiments, the targeting moiety may bind to any forms of the proteins described herein, including monomeric, dimeric, trimeric, tetrameric, heterodimeric, multimeric and associated forms. In various embodiments, the targeting moiety may bind to any post-translationally modified forms of the proteins described herein, such as glycosylated and/or phosphorylated forms.

In various embodiments, the targeting moiety comprises an antigen recognition domain that recognizes extracellular molecules such as DNA. In some embodiments, the targeting moiety comprises an antigen recognition domain that recognizes DNA. In an embodiment, the DNA is shed into the extracellular space from necrotic or apoptotic tumor cells or other diseased cells.

In various embodiments, the targeting moiety comprises an antigen recognition domain that recognizes one or more non-cellular structures associated with atherosclerotic plaques. Two types of atherosclerotic plaques are known. The fibro-lipid (fibro-fatty) plaque is characterized by an accumulation of lipid-laden cells underneath the intima of the arteries. Beneath the endothelium there is a fibrous cap covering the atheromatous core of the plaque. The core includes lipid-laden cells (macrophages and smooth muscle cells) with elevated tissue cholesterol and cholesterol ester content, fibrin, proteoglycans, collagen, elastin, and cellular debris. In advanced plaques, the central core of the plaque usually contains extracellular cholesterol deposits (released from dead cells), which form areas of cholesterol crystals with empty, needle-like clefts. At the periphery of the plaque are younger foamy cells and capillaries. A fibrous plaque is also localized under the intima, within the wall of the artery resulting in thickening and expansion of the wall and, sometimes, spotty localized narrowing of the lumen with some atrophy of the muscular layer. The fibrous plaque contains collagen fibers (eosinophilic), precipitates of calcium (hematoxylinophilic) and lipid-laden cells. In some embodiments, the targeting moiety recognizes and binds to one or more of the non-cellular components of these plaques such as the fibrin, proteoglycans, collagen, elastin, cellular debris, and calcium or other mineral deposits or precipitates. In some embodiments, the cellular debris is a nucleic acid, e.g. DNA or RNA, released from dead cells.

In various embodiments, the targeting moiety comprises an antigen recognition domain that recognizes one or more non-cellular structures found in the brain plaques associated with neurodegenerative diseases. In some embodiments, the targeting moiety recognizes and binds to one or more non-cellular structures located in the amyloid plaques found in the brains of patients with Alzheimer's disease. For example, the targeting moiety may recognize and bind to the peptide amyloid beta, which is a major component of the amyloid plaques. In some embodiments, the targeting moiety recognizes and binds to one or more non-cellular structures located in the brain plaques found in patients with Huntington's disease. In various embodiments, the targeting moiety recognizes and binds to one or more non-cellular structures found in plaques associated with other neurodegenerative or musculoskeletal diseases such as Lewy body dementia and inclusion body myositis.

In some embodiments, chimeric protein, chimeric protein complex, vaccine composition, or adjuvant of the invention may have two or more targeting moieties that bind to non-cellular structures. In some embodiments, there are two targeting moieties and one targets a cell while the other targets a non-cellular structure. In various embodiments, the targeting moieties can directly or indirectly recruit cells, such as disease cells and/or effector cells. In some embodiments, chimeric protein, chimeric protein complex, vaccine composition, or adjuvant are capable of, or find use in methods involving, shifting the balance of immune cells in favor of immune attack of a tumor. For instance, chimeric protein, chimeric protein complex, vaccine composition, or adjuvant can shift the ratio of immune cells at a site of clinical importance in favor of cells that can kill and/or suppress a tumor (e.g. T cells, cytotoxic T lymphocytes, T helper cells, natural killer (NK) cells, natural killer T (NKT) cells, anti-tumor macrophages (e.g. M1 macrophages), B cells, dendritic cells, or subsets thereof) and in opposition to cells that protect tumors (e.g. myeloid-derived suppressor cells (MDSCs), regulatory T cells (Tregs); tumor associated neutrophils (TANs), M2 macrophages, tumor associated macrophages (TAMs), or subsets thereof). In some embodiments, chimeric protein, chimeric protein complex, vaccine composition, or adjuvant are capable of increasing a ratio of effector T cells to regulatory T cells.

For example, in some embodiments, the recognition domains specifically bind to a target (e.g. antigen, receptor) associated with T cells. In some embodiments, the recognition domains directly or indirectly recruit T cells. In an embodiment, the recognition domains specifically bind to effector T cells. In some embodiments, the recognition domain directly or indirectly recruits effector T cells, e.g., in some embodiments, to a therapeutic site (e.g. a locus with one or more disease cell or cell to be modulated for a therapeutic effect). Illustrative effector T cells include cytotoxic T cells (e.g. αβ TCR, CD3⁺, CD8⁺, CD45RO⁺); CD4⁺ effector T cells (e.g. αβ TCR, CD3⁺, CD4⁺, CCR7⁺, CD62Lhi, IL-7R/CD127⁺); CD8⁺ effector T cells (e.g. αβ TCR, CD3⁺, CD8⁺, CCR7⁺, CD62Lhi, IL-7R/CD127⁺); effector memory T cells (e.g. CD62Llow, CD44⁺, TCR, CD3⁺, IL-7R/CD127⁺, IL-15R+, CCR7low); central memory T cells (e.g. CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27+CD62L⁻) and late effector memory T cells (CD27⁻ CD62L⁻) (TemE and TemL, respectively); CD127(⁺) CD25(low/−) effector T cells; CD127 (⁻) CD25 (⁻) effector T cells; CD8⁺ stem cell memory effector cells (TSCM) (e.g. CD44(low) CD62L(high) CD122(high)sca(⁺)); TH1 effector T-cells (e.g. CXCR3⁺, CXCR6⁺ and CCR5⁺; or αβ TCR, CD3⁺, CD4⁺, IL-12R⁺, IFNγR⁺, CXCR3⁺), TH2 effector T cells (e.g. CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g. αβ TCR, CD3⁺, CD4⁺); TH17 effector T cells (e.g. αβ TCR, CD3⁺, CD4⁺, IL-23R⁺, CCR6⁺, IL-1R⁺); CD4⁺CD45RO⁺CCR7⁺ effector T cells, ICOS⁺ effector T cells; CD4⁺CD45RO⁺CCR7(⁻) effector T cells; and effector T cells secreting IL-2, IL-4 and/or IFN-γ.

Illustrative T cell antigens of interest include, for example (and inclusive of the extracellular domains, where applicable): CD8, CD3, SLAMF4, IL-2Rα, 4-1BB/TNFRSF9, IL-2 R β, ALCAM, B7-1, IL-4 R, B7-H3, BLAME/SLAMFS, CEACAM1, IL-6 R, CCR3, IL-7 Ra, CCR4, CXCRI/IL-S RA, CCR5, CCR6, IL-10R α, CCR 7, IL-1 0 R β, CCRS, IL-12 R β 1, CCR9, IL-12 R β 2, CD2, IL-13 R α 1, IL-13, CD3, CD4, ILT2/CDS5j, ILT3/CDS5k, ILT4/CDS5d, ILT5/CDS5a, lutegrin α 4/CD49d, CDS, Integrin α E/CD103, CD6, Integrin α M/CD 11 b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, CXCR3, SIRP β 1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R. In various embodiments, a targeting moiety of chimeric protein, chimeric protein complex, vaccine composition, or adjuvant binds one or more of these illustrative T cell antigens.

By way of non-limiting example, in various embodiments, chimeric protein, chimeric protein complex, vaccine composition, or adjuvant have a targeting moiety directed against a checkpoint marker expressed on a T cell, e.g. one or more of PD-1, CD28, CTLA4, ICOS, BTLA, KIR, LAG3, CD137, OX40, CD27, CD40L, TIM3, and A2aR.

For example, in some embodiments, the recognition domains specifically bind to a target (e.g. antigen, receptor) associated with B cells. In some embodiments, the recognition domains directly or indirectly recruit B cells, e.g., in some embodiments, to a therapeutic site (e.g. a locus with one or more disease cell or cell to be modulated for a therapeutic effect). Illustrative B cell antigens of interest include, for example, CD10, CD19, CD20, CD21, CD22, CD23, CD24, CD37, CD38, CD39, CD40, CD70, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138, CDw150, CS1, and B-cell maturation antigen (BCMA).

In various embodiments, a targeting moiety of the vaccine compositions, adjuvants, chimeric proteins or chimeric protein complexes binds one or more of these illustrative B cell antigens.

By way of further example, in some embodiments, the recognition domains specifically bind to a target (e.g. antigen, receptor) associated with Natural Killer cells. In some embodiments, the recognition domains directly or indirectly recruit Natural Killer cells, e.g., in some embodiments, to a therapeutic site (e.g. a locus with one or more disease cell or cell to be modulated for a therapeutic effect). Illustrative Natural Killer cell antigens of interest include, for example TIGIT, 2B4/SLAMF4, KIR2DS4, CD155/PVR, KIR3DL1, CD94, LMIR1/CD300A, CD69, LMIR2/CD300c, CRACC/SLAMF7, LMIR3/CD300LF, DNAM-1, LMIR5/CD300LB, Fc-epsilon RII, LMIR6/CD300LE, Fc-γ RI/CD64, MICA, Fc-γ RIIB/CD32b, MICB, Fc-γ RIIC/CD32c, MULT-1, Fc-γ RIIA/CD32a, Nectin-2/CD112, Fc-γ RIII/CD16, NKG2A, FcRH1/IRTA5, NKG2C, FcRH2/IRTA4, NKG2D, FcRH4/IRTA1, NKp30, FcRH5/IRTA2, NKp44, Fc-Receptor-like 3/CD16-2, NKp46/NCR1, NKp80/KLRF1, NTB-A/SLAMF6, Rae-1, Rae-1 α, Rae-1 β, Rae-1 delta, H60, Rae-1 epsilon, ILT2/CD85j, Rae-1 γ, ILT3/CD85k, TREM-1, ILT4/CD85d, TREM-2, ILT5/CD85a, TREM-3, KIR/CD158, TREML1/TLT-1, KIR2DL1, ULBP-1, KIR2DL3, ULBP-2, KIR2DL4/CD158d and ULBP-3.

In various embodiments, a targeting moiety of the vaccine compositions, adjuvants, chimeric proteins or chimeric protein complexes binds one or more of these illustrative NK cell antigens.

Also, in some embodiments, the recognition domains specifically bind to a target (e.g. antigen, receptor) associated with macrophages/monocytes. In some embodiments, the recognition domains directly or indirectly recruit macrophages/monocytes, e.g., in some embodiments, to a therapeutic site (e.g. a locus with one or more disease cell or cell to be modulated for a therapeutic effect). Illustrative macrophages/monocyte antigens of interest include, for example SIRP1a, B7-1/CD80, ILT4/CD85d, B7-H1, ILT5/CD85a, Common β Chain, Integrin α 4/CD49d, BLAME/SLAMF8, Integrin α X/CDIIc, CCL6/C10, Integrin β 2/CD18, CD155/PVR, Integrin β 3/CD61, CD31/PECAM-1, Latexin, CD36/SR-B3, Leukotriene B4 R1, CD40/TNFRSF5, LIMPIIISR-B2, CD43, LMIR1/CD300A, CD45, LMIR2/CD300c, CD68, LMIR3/CD300LF, CD84/SLAMF5, LMIR5/CD300LB, CD97, LMIR6/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc-γ RI/CD64, Osteopontin, Fc-γ RIIB/CD32b, PD-L2, Fc-γ RIIC/CD32c, Siglec-3/CD33, Fc-γ RIIA/CD32a, SIGNR1/CD209, Fc-γ RIII/CD16, SLAM, GM-CSF R α, TCCR/WSX-1, ICAM-2/CD102, TLR3, IFN-γ RI, TLR4, IFN-γ R2, TREM-I, IL-I RII, TREM-2, ILT2/CD85j, TREM-3, ILT3/CD85k, TREML1/TLT-1, 2B4/SLAMF 4, IL-10 R α, ALCAM, IL-10 R β, AminopeptidaseN/ANPEP, ILT2/CD85j, Common β Chain, ILT3/CD85k, Clq R1/CD93, ILT4/CD85d, CCR1, ILT5/CD85a, CCR2, Integrin α 4/CD49d, CCR5, Integrin α M/CDII b, CCR8, Integrin α X/CDIIc, CD155/PVR, Integrin β 2/CD18, CD14, Integrin β 3/CD61, CD36/SR-B3, LAIR1, CD43, LAIR2, CD45, Leukotriene B4-R1, CD68, LIMPIIISR-B2, CD84/SLAMF5, LMIR1/CD300A, CD97, LMIR2/CD300c, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc-γ RI/CD64, PSGL-1, Fc-γ RIIIICD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-I, IL-6 R, TREM-2, CXCRI/IL-8 RA, TREM-3 and TREMLI/TLT-1. In various embodiments, a targeting moiety of the vaccine compositions, adjuvants, chimeric proteins or chimeric protein complexes binds one or more of these illustrative macrophage/monocyte antigens.

Also, in some embodiments, the recognition domains specifically bind to a target (e.g. antigen, receptor) associated with dendritic cells. In some embodiments, the recognition domains directly or indirectly recruit dendritic cells, e.g., in some embodiments, to a therapeutic site (e.g. a locus with one or more disease cell or cell to be modulated for a therapeutic effect). Illustrative dendritic cell antigens of interest include, for example, CLEC9A, XCR1, RANK, CD36/SRB3, LOX-1/SR-E1, CD68, MARCO, CD163, SR-A1/MSR, CD5L, SREC-1, CL-PI/COLEC12, SREC-II, LIMPIIISRB2, RP105, TLR4, TLR1, TLR5, TLR2, TLR6, TLR3, TLR9, 4-IBB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, lutegrin α 4/CD49d, Aag, Integrin β 2/CD18, AMICA, Langerin, B7-2/CD86, Leukotriene B4 RI, B7-H3, LMIR1/CD300A, BLAME/SLAMF8, LMIR2/CD300c, Clq R1/CD93, LMIR3/CD300LF, CCR6, LMIR5/CD300LB CCR7, LMIR6/CD300LE, CD40/TNFRSF5, MAG/Siglec-4-a, CD43, MCAM, CD45, MD-1, CD68, MD-2, CD83, MDL-1/CLEC5A, CD84/SLAMF5, MMR, CD97, NCAMLI, CD2F-10/SLAMF9, Osteoactivin GPNMB, Chern 23, PD-L2, CLEC-1, RP105, CLEC-2, CLEC-8, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC/CLEC4C, SLAM, EMMPRIN/CD147, TCCR/WSX-1, Fc-γ R1/CD64, TLR3, Fc-γ RIIB/CD32b, TREM-1, Fc-γ RIIC/CD32c, TREM-2, Fc-γ RIIA/CD32a, TREM-3, Fc-γ RIII/CD16, TREML1/TLT-1, ICAM-2/CD102 and Vanilloid R1. In various embodiments, a targeting moiety of the vaccine compositions, adjuvants, chimeric proteins or chimeric protein complexes binds one or more of these illustrative DC antigens.

In some embodiments, the recognition domains specifically bind to a target (e.g. antigen, receptor) on immune cells selected from, but not limited to, megakaryocytes, thrombocytes, erythrocytes, mast cells, basophils, neutrophils, myeloid cells, monocytes, eosinophils, or subsets thereof. In some embodiments, the recognition domains directly or indirectly recruit megakaryocytes, thrombocytes, erythrocytes, mast cells, basophils, neutrophils, myeloid cells, monocytes, eosinophils, or subsets thereof, e.g., in some embodiments, to a therapeutic site (e.g. a locus with one or more disease cell or cell to be modulated for a therapeutic effect). In some embodiments, the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof.

In some embodiments, the recognition domains specifically bind to a target (e.g. antigen, receptor) associated with megakaryocytes and/or thrombocytes. Illustrative megakaryocyte and/or thrombocyte antigens of interest include, for example, GP Ilb/IIIa, GPIb, vWF, PF4, and TSP. In various embodiments, a targeting moiety of the vaccine compositions, adjuvants, chimeric proteins or chimeric protein complexes binds one or more of these illustrative megakaryocyte and/or thrombocyte antigens.

In some embodiments, the recognition domains specifically bind to a target (e.g. antigen, receptor) associated with erythrocytes. Illustrative erythrocyte antigens of interest include, for example, CD34, CD36, CD38, CD41a (platelet glycoprotein IIb/IIIa), CD41b (GPIIb), CD71 (transferrin receptor), CD105, glycophorin A, glycophorin C, c-kit, HLA-DR, H2 (MHC-II), and Rhesus antigens. In various embodiments, a targeting moiety of the vaccine compositions, adjuvants, chimeric proteins or chimeric protein complexes binds one or more of these illustrative erythrocyte antigens.

In some embodiments, the recognition domains specifically bind to a target (e.g. antigen, receptor) associated with mast cells. Illustrative mast cells antigens of interest include, for example, SCFR/CD117, Fc_{ε}RI, CD2, CD25, CD35, CD88, CD203c, C5R1, CMAI, FCERIA, FCER2, TPSABI. In various embodiments, a targeting moiety of the vaccine compositions, adjuvants, chimeric proteins or chimeric protein complexes binds one or more of these mast cell antigens.

In some embodiments, the recognition domains specifically bind to a target (e.g. antigen, receptor) associated with basophils. Illustrative basophils antigens of interest include, for example, Fc_{ε}RI, CD203c, CD123, CD13, CD107a, CD107b, and CD164. In various embodiments, a targeting moiety of the vaccine compositions, adjuvants, chimeric proteins or chimeric protein complexes binds one or more of these basophil antigens.

In some embodiments, the recognition domains specifically bind to a target (e.g. antigen, receptor) associated with neutrophils. Illustrative neutrophils antigens of interest include, for example, 7D5, CD10/CALLA, CD13, CD16 (FcRIII), CD18 proteins (LFA-1, CR3, and p150, 95), CD45, CD67, and CD177. In various embodiments, a targeting moiety of the vaccine compositions, adjuvants, chimeric proteins or chimeric protein complexes binds one or more of these neutrophil antigens.

In some embodiments, the recognition domains specifically bind to a target (e.g. antigen, receptor) associated with eosinophils. Illustrative eosinophils antigens of interest include, for example, CD35, CD44 and CD69. In various embodiments, a targeting moiety of the vaccine compositions, adjuvants, chimeric proteins or chimeric protein complexes binds one or more of these eosinophil antigens.

In various embodiments, the recognition domain may bind to any appropriate target, antigen, receptor, or cell surface markers known by the skilled artisan. In some embodiments, the antigen or cell surface marker is a tissue-specific marker. Illustrative tissue-specific markers include, but are not limited to, endothelial cell surface markers such as ACE, CD14, CD34, CDH5, ENG, ICAM2, MCAM, NOS3, PECAMI, PROCR, SELE, SELP, TEK, THBD, VCAMI, VWF; smooth muscle cell surface markers such as ACTA2, MYHIO, MYHI 1, MYH9, MYOCD; fibroblast (stromal) cell surface markers such as ALCAM, CD34, COLIAI, COL1A2, COL3A1, FAP, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (αvβ3), E-selectin; and adipocyte surface markers such as ADIPOQ, FABP4, and RETN. In various embodiments, a targeting moiety of the vaccine compositions, adjuvants, chimeric proteins or chimeric protein complexes binds one or more of these antigens. In various embodiments, a targeting moiety of the vaccine compositions, adjuvants, chimeric proteins or chimeric protein complexes binds one or more of cells having these antigens.

In some embodiments, the recognition domains specifically bind to a target (e.g. antigen, receptor) associated with tumor cells. In some embodiments, the recognition domains directly or indirectly recruit tumor cells. For instance, in some embodiments, the direct or indirect recruitment of the tumor cell is to one or more effector cell (e.g. an immune cell as described herein) that can kill and/or suppress the tumor cell.

Tumor cells or cancer cells refer to an uncontrolled growth of cells or tissues and/or an abnormal increase in cell survival and/or inhibition of apoptosis, which interferes with the normal functioning of bodily organs and systems. For example, tumor cells include benign and malignant cancers, polyps, hyperplasia, as well as dormant tumors or micrometastases. Illustrative tumor cells include, but are not limited to cells of: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Tumor cells, or cancer cells also include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g. astrocytoma, oligodendroglioma, and ependymoma), meningioma, pituitary adenoma, and neuromas, and spinal cord tumors (e.g. meningiomas and neurofibroma).

Illustrative tumor antigens include, but are not limited to, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, lg-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD20, CD22, CD30, CD33, CD37, CD47, CS1, CD38, ASGPR, CD56, CD70, CD74, CD138, AGS16, MUC1, GPNMB, Ep-CAM, PD-L1, PD-L2, PMSA, and BCMA (TNFRSF17). In various embodiments, a targeting moiety of the vaccine compositions, adjuvants, chimeric proteins or chimeric protein complexes binds one or more of these tumor antigens. In an embodiment, the vaccine compositions, adjuvants, chimeric proteins or chimeric protein complexes binds to HER2. In another embodiment, the vaccine compositions, adjuvants, chimeric proteins or chimeric protein complexes binds to PD-L2.

In some embodiments, the present chimeric proteins or chimeric protein complexes such as Fc-based chimeric protein complexes have (i) one or more of the targeting moieties which is directed against an immune cell selected from a T cell, a B cell, a dendritic cell, a macrophage, a NK cell, or subsets thereof and (ii) one or more of the targeting moieties which is directed against a tumor cell, along with any of the signaling agents (e.g., IL-1α or a variant thereof) described herein. In one embodiment, the present chimeric proteins or chimeric protein complexes such as Fc-based chimeric protein complexes have (i) a targeting moiety directed against a T cell (including, without limitation an effector T cell) and (ii) a targeting moiety is directed against a tumor cell, along with any of the signaling agents described herein. In one embodiment, the present chimeric proteins or chimeric protein complexes such as Fc-based chimeric protein complexes have (i) a targeting moiety directed against a B cell and (ii) a targeting moiety is directed against a tumor cell, along with any of the signaling agents described herein. In one embodiment, the present chimeric proteins or chimeric protein complexes such as Fc-based chimeric protein complexes have (i) a targeting moiety directed against a dendritic cell and (ii) a targeting moiety is directed against a tumor cell, along with any of the signaling agents described herein. In one embodiment, the present chimeric proteins or chimeric protein complexes such as Fc-based chimeric protein complexes have (i) a targeting moiety directed against a macrophage and (ii) a targeting moiety is directed against a tumor cell, along with any of the signaling agents described herein. In one embodiment, the present chimeric proteins or chimeric protein complexes such as Fc-based chimeric protein complexes have (i) a targeting moiety directed against a NK cell and (ii) a targeting moiety is directed against a tumor cell, along with any of the signaling agents described herein.

By way of non-limiting example, in various embodiments, the present chimeric proteins or chimeric protein complexes such as Fc-based chimeric protein complexes have (i) a targeting moiety directed against a T cell, for example, mediated by targeting to CD8, SLAMF4, IL-2 R α, 4-1BB/TNFRSF9, IL-2 R β, ALCAM, B7-1, IL-4 R, B7-H3, BLAME/SLAMFS, CEACAM1, IL-6 R, CCR3, IL-7 Ra, CCR4, CXCRI/IL-S RA, CCR5, CCR6, IL-10R α, CCR 7, IL-1 0 R β, CCRS, IL-12 R $1, CCR9, IL-12 R $2, CD2, IL-13 R α 1, IL-13, CD3, CD4, ILT2/CDS5j, ILT3/CDS5k, ILT4/CDS5d, ILT5/CDS5a, lutegrin α 4/CD49d, CDS, Integrin α E/CD103, CD6, Integrin α M/CD 11 b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, CXCR3, SIRP β 1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1, or TSLP R; and (ii) a targeting moiety is directed against a tumor cell, along with any of the signaling agents (e.g., IL-1α or a variant thereof) described herein.

By way of non-limiting example, in various embodiments, the present chimeric proteins or chimeric protein complexes such as Fc-based chimeric protein complexes have a targeting moiety directed against (i) a checkpoint marker expressed on a T cell, e.g. one or more of PD-1, CD28, CTLA4, ICOS, BTLA, KIR, LAG3, CD137, OX40, CD27, CD40L, TIM3, and A2aR and (ii) a targeting moiety is directed against a tumor cell, along with any of the signaling agents described herein.

In various embodiments, the present chimeric proteins or chimeric protein complexes such as Fc-based chimeric protein complexes have one or more targeting moieties directed against PD-1. In some embodiments, the vaccine compositions, adjuvants, chimeric proteins or chimeric protein complexes have one or more targeting moieties, which selectively bind a PD-1 polypeptide. In some embodiments, the vaccine compositions, adjuvants, chimeric proteins or chimeric protein complexes comprise one or more antibodies, antibody derivatives or formats, peptides or polypeptides, or fusion proteins that selectively bind a PD-1 polypeptide.

In an embodiment, the targeting moiety comprises the anti-PD-1 antibody pembrolizumab (aka MK-3475, KEYTRUDA), or fragments thereof. Pembrolizumab and other humanized anti-PD-1 antibodies are disclosed in Hamid, et al. (2013) New England Journal of Medicine 369 (2): 134-44, U.S. Pat. No. 8,354,509, and WO 2009/114335, the entire disclosures of which are hereby incorporated by reference. In illustrative embodiments, pembrolizumab or an antigen-binding fragment thereof for use in the methods provided herein comprises a heavy chain comprising the amino acid sequence of (SEQ ID NO: 7) and/or a light chain comprising the amino acid sequence of (SEQ ID NO: 8).

In an embodiment, the targeting moiety comprises the anti-PD-1 antibody, nivolumab (aka BMS-936558, MDX-1106, ONO-4538, OPDIVO), or fragments thereof. Nivolumab (clone 5C4) and other human monoclonal antibodies that specifically bind to PD-1 are disclosed in U.S. Pat. No. 8,008,449 and WO 2006/121168, the entire disclosures of which are hereby incorporated by reference. In illustrative embodiments, nivolumab or an antigen-binding fragment thereof comprises a heavy chain comprising the amino acid sequence of (SEQ ID NO: 9) and/or a light chain comprising the amino acid sequence of (SEQ ID NO: 10).

In an embodiment, the targeting moiety comprises the anti-PD-1 antibody pidilizumab (aka CT-011, hBAT or hBAT-1), or fragments thereof. Pidilizumab and other humanized anti-PD-I monoclonal antibodies are disclosed in US 2008/0025980 and WO 2009/101611, the entire disclosures of which are hereby incorporated by reference. In illustrative embodiments, the anti-PD-1 antibody or an antigen-binding fragment thereof for use in the methods provided herein comprises a light chain variable regions comprising an amino acid sequence selected from SEQ ID NOS: 15-18 of US 2008/0025980: SEQ ID No: 15 of US 2008/0025980 (SEQ ID NO: 11); SEQ ID No: 16 of US 2008/0025980 (SEQ ID NO: 12); SEQ ID No: 17 of US 2008/0025980 (SEQ ID NO: 13); and SEQ ID No: 18 of US 2008/0025980 (SEQ ID NO: 14); and/or a heavy chain comprising an amino acid sequence selected from SEQ ID NOS: 20-24 of US 2008/0025980: SEQ ID No: 20 of US 2008/0025980 (SEQ ID NO: 15); SEQ ID No: 21 of US 2008/0025980 (SEQ ID NO: 16); SEQ ID No: 22 of US 2008/0025980 (SEQ ID NO: 17); SEQ ID No: 23 of US 2008/0025980 (SEQ ID NO: 18); and SEQ ID No: 24 of US 2008/0025980 (SEQ ID NO: 19).

In an embodiment, the targeting moiety comprises a light chain comprising SEQ ID NO: 18 of US 2008/0025980 (SEQ ID NO: 14) and a heavy chain comprising SEQ ID NO: 22 of US 2008/0025980 (SEQ ID NO: 17).

In an embodiment, the targeting moiety comprises AMP-514 (aka MEDI-0680).

In an embodiment, the targeting moiety comprises the PD-L2-Fc fusion protein AMP-224, which is disclosed in WO2010/027827 and WO 2011/066342, the entire disclosures of which are hereby incorporated by reference. In such an embodiment, the targeting moiety may include a targeting domain which comprises SEQ ID NO:4 of WO2010/027827 (SEQ ID NO:20) and/or the B7-DC fusion protein which comprises SEQ ID NO:83 of WO2010/027827 (SEQ ID NO: 21).

In an embodiment, the targeting moiety comprises the peptide AUNP 12 or any of the other peptides disclosed in US 2011/0318373 or 8,907,053. For example, the targeting moiety may comprise AUNP 12 (i.e., Compound 8 or SEQ ID NO:49 of US 2011/0318373) which has the sequence of:

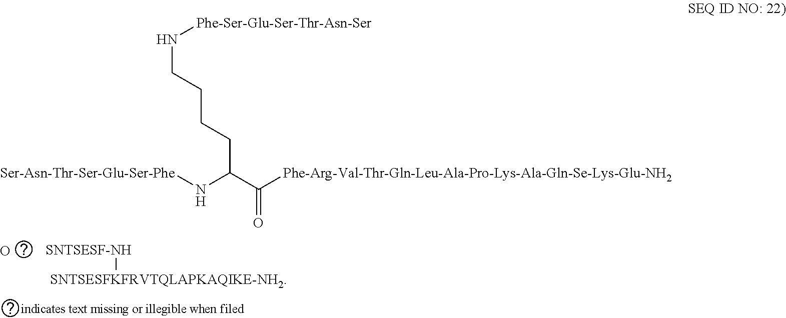

In an embodiment, the targeting moiety comprises the anti-PD-1 antibody 1E3, or fragments thereof, as disclosed in US 2014/0044738, the entire disclosures of which are hereby incorporated by reference. In illustrative embodiments, 1E3 or an antigen-binding fragment thereof for use in the methods provided herein comprises a heavy chain variable region comprising the amino acid sequence of (SEQ ID NO: 23); and/or a light chain variable region comprising the amino acid sequence of (SEQ ID NO: 24).

In an embodiment, the targeting moiety comprises the anti-PD-1 antibody 1E8, or fragments thereof, as disclosed in US 2014/0044738, the entire disclosures of which are hereby incorporated by reference. In illustrative embodiments, 1E8 or an antigen-binding fragment thereof for use in the methods provided herein comprises a heavy chain variable region comprising the amino acid sequence of SEQ ID NO: 25) and/or a light chain variable region comprising the amino acid sequence of SEQ ID NO: 26.

In an embodiment, the targeting moiety comprises the anti-PD-1 antibody 1H3, or fragments thereof, as disclosed in US 2014/0044738, the entire disclosures of which are hereby incorporated by reference. In illustrative embodiments, 1H3 or an antigen-binding fragment thereof for use in the methods provided herein comprises a heavy chain variable region comprising the amino acid sequence of (SEQ ID NO: 27) and/or light chain variable region comprising the amino acid sequence of (SEQ ID NO: 28).

In an embodiment, the targeting moiety comprises a VHH directed against PD-1 as disclosed, for example, in U.S. Pat. No. 8,907,065 and WO 2008/071447, the entire disclosures of which are hereby incorporated by reference. In illustrative embodiments, the VHHs against PD-1 comprise SEQ ID NOS: 347-351 of U.S. Pat. No. 8,907,065 (SEQ ID No: 347 of U.S. Pat. No. 8,907,065 (SEQ ID NO: 29); SEQ ID No: 348 of U.S. Pat. No. 8,907,065 (SEQ ID NO: 30); SEQ ID No: 349 of U.S. Pat. No. 8,907,065 (SEQ ID NO:31); SEQ ID No: 350 of U.S. Pat. No. 8,907,065 (SEQ ID NO: 32); and SEQ ID No: 351 of U.S. Pat. No. 8,907,065 (SEQ ID NO:33).

In an embodiment, the targeting moiety comprises any one of the anti-PD-1 antibodies, or fragments thereof, as disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID NOS: 25-29 of US2011/0271358 (SEQ ID No: 25 of US2011/0271358 (SEQ ID NO:34); SEQ ID No: 26 of US2011/0271358 (SEQ ID NO:35); SEQ ID No: 27 of US2011/0271358 (SEQ ID NO:36); SEQ ID No: 28 of US2011/0271358 (SEQ ID NO:37); and SEQ ID No: 29 of US2011/0271358 (SEQ ID NO:38)); and/or a light chain comprising an amino acid sequence selected from SEQ ID NOS: 30-33 of US2011/0271358 (SEQ ID No: 30 of US2011/0271358 (SEQ ID NO: 39); SEQ ID No: 31 of US2011/0271358 (SEQ ID NO:40); SEQ ID No: 32 of US2011/0271358 (SEQ ID NO: 41); and SEQ ID No: 33 of US2011/0271358 (SEQ ID NO:42)).

In various embodiments, the present chimeric proteins or chimeric protein complexes such as Fc-based chimeric protein complexes comprise one or more antibodies directed against PD-1, or antibody fragments thereof, selected from TSR-042 (Tesaro, Inc.), REGN2810 (Regeneron Pharmaceuticals, Inc.), PDR001 (Novartis Pharmaceuticals), and BGB-A317 (BeiGene Ltd.)

In various embodiments, the present chimeric proteins or chimeric protein complexes such as Fc-based chimeric protein complexes have one or more targeting moieties directed against PD-L1. In some embodiments, the vaccine compositions, adjuvants, chimeric proteins or chimeric protein complexes have one or more targeting moieties, which selectively bind a PD-L1 polypeptide. In some embodiments, the vaccine compositions, adjuvants, chimeric proteins or chimeric protein complexes comprise one or more antibodies, antibody derivatives or formats, peptides or polypeptides, or fusion proteins that selectively bind a PD-L1 polypeptide.

In an embodiment, the targeting moiety comprises the anti-PD-L1 antibody MEDI4736 (aka durvalumab), or fragments thereof. MEDI4736 is selective for PD-L1 and blocks the binding of PD-L1 to the PD-1 and CD80 receptors. MEDI4736 and antigen-binding fragments thereof for use in the methods provided herein comprises a heavy chain and a light chain or a heavy chain variable region and a light chain variable region. The sequence of MEDI4736 is disclosed in WO/2016/06272, the entire contents of which are hereby incorporated by reference. In illustrative embodiments, MEDI4736 or an antigen-binding fragment thereof for use in the methods provided herein comprises a heavy chain comprising the amino acid sequence of (SEQ ID NO:43); and/or a light chain comprising the amino acid sequence of (SEQ ID NO: 44).

In illustrative embodiments, the MEDI4736 or an antigen-binding fragment thereof for use in the methods provided herein comprises a heavy chain variable region comprising the amino acid sequence of SEQ ID NO: 4 of WO/2016/06272 (SEQ ID NO:45); and/or a light chain variable region comprising the amino acid sequence of SEQ ID NO:3 of WO/2016/06272 (SEQ ID NO:46).

In an embodiment, the targeting moiety comprises the anti-PD-L1 antibody atezolizumab (aka MPDL3280A, RG7446), or fragments thereof. In illustrative embodiments, atezolizumab or an antigen-binding fragment thereof for use in the methods provided herein comprises a heavy chain comprising the amino acid sequence of (SEQ ID NO:47); and/or a light chain comprising the amino acid sequence of (SEQ ID NO:48).

In an embodiment, the targeting moiety comprises the anti-PD-L1 antibody avelumab (aka MSB0010718C), or fragments thereof. In illustrative embodiments, avelumab or an antigen-binding fragment thereof for use in the methods provided herein comprises a heavy chain comprising the amino acid sequence of (SEQ ID NO:49); and/or a light chain comprising the amino acid sequence of (SEQ ID NO: 50).

In an embodiment, the targeting moiety comprises the anti-PD-L1 antibody BMS-936559 (aka 12A4, MDX-1105), or fragments thereof, as disclosed in US 2013/0309250 and WO2007/005874, the entire disclosures of which are hereby incorporated by reference. In illustrative embodiments, BMS-936559 or an antigen-binding fragment thereof for use in the methods provided herein comprises a heavy chain variable region comprising the amino acid sequence of: (SEQ ID NO:51); and/or a light chain variable region comprising the amino acid sequence of (SEQ ID NO:52).

In an embodiment, the targeting moiety comprises the anti-PD-L1 antibody 3G10, or fragments thereof, as disclosed in US 2013/0309250 and WO2007/005874, the entire disclosures of which are hereby incorporated by reference. In illustrative embodiments, 3G10 or an antigen-binding fragment thereof for use in the methods provided herein comprises a heavy chain variable region comprising the amino acid sequence of (SEQ ID NO: 53); and/or a light chain variable region comprising the amino acid sequence of (SEQ ID NO: 54).

In an embodiment, the targeting moiety comprises the anti-PD-L1 antibody 10A5, or fragments thereof, as disclosed in US 2013/0309250 and WO2007/005874, the entire disclosures of which are hereby incorporated by reference. In illustrative embodiments, 10A5 or an antigen-binding fragment thereof for use in the methods provided herein comprises a heavy chain variable region comprising the amino acid sequence of (SEQ ID NO: 55); and/or a light chain variable region comprising the amino acid sequence of (SEQ ID NO: 56).

In an embodiment, the targeting moiety comprises the anti-PD-L1 antibody 5F8, or fragments thereof, as disclosed in US 2013/0309250 and WO2007/005874, the entire disclosures of which are hereby incorporated by reference. In illustrative embodiments, 5F8 or an antigen-binding fragment thereof for use in the methods provided herein comprises a heavy chain variable region comprising the amino acid sequence of (SEQ ID NO: 57); and/or a light chain variable region comprising the amino acid sequence of (SEQ ID NO: 58).

In an embodiment, the targeting moiety comprises the anti-PD-L1 antibody 10H10, or fragments thereof, as disclosed in US 2013/0309250 and WO2007/005874, the entire disclosures of which are hereby incorporated by reference. In illustrative embodiments, 10H10 or an antigen-binding fragment thereof for use in the methods provided herein comprises a heavy chain variable region comprising the amino acid sequence of (SEQ ID NO: 59); and/or a light chain variable region comprising the amino acid sequence of (SEQ ID NO: 60).

In an embodiment, the targeting moiety comprises the anti-PD-L1 antibody 1B12, or fragments thereof, as disclosed in US 2013/0309250 and WO2007/005874, the entire disclosures of which are hereby incorporated by reference. In illustrative embodiments, 1B12 or an antigen-binding fragment thereof for use in the methods provided herein comprises a heavy chain variable region comprising the amino acid sequence of (SEQ ID NO: 61); and/or a light chain variable region comprising the amino acid sequence of (SEQ ID NO: 62).

In an embodiment, the targeting moiety comprises the anti-PD-L1 antibody 7H1, or fragments thereof, as disclosed in US 2013/0309250 and WO2007/005874, the entire disclosures of which are hereby incorporated by reference. In illustrative embodiments, 7H1 or an antigen-binding fragment thereof for use in the methods provided herein comprises a heavy chain variable region comprising the amino acid sequence of (SEQ ID NO: 63); and/or a light chain variable region comprising the amino acid sequence of (SEQ ID NO: 64).

In an embodiment, the targeting moiety comprises the anti-PD-L1 antibody 11E6, or fragments thereof, as disclosed in US 2013/0309250 and WO2007/005874, the entire disclosures of which are hereby incorporated by reference. In illustrative embodiments, 11E6 or an antigen-binding fragment thereof for use in the methods provided herein comprises a heavy chain variable region comprising the amino acid sequence of (SEQ ID NO: 65); and/or a light chain variable region comprising the amino acid sequence of (SEQ ID NO: 66).

In an embodiment, the targeting moiety comprises the anti-PD-L1 antibody 12B7, or fragments thereof, as disclosed in US 2013/0309250 and WO2007/005874, the entire disclosures of which are hereby incorporated by reference. In illustrative embodiments, 12B7 or an antigen-binding fragment thereof for use in the methods provided herein comprises a heavy chain variable region comprising the amino acid sequence of (SEQ ID NO: 67); and/or a light chain variable region comprising the amino acid sequence of (SEQ ID NO: 68).

In an embodiment, the targeting moiety comprises the anti-PD-L1 antibody 13G4, or fragments thereof, as disclosed in US 2013/0309250 and WO2007/005874, the entire disclosures of which are hereby incorporated by reference. In illustrative embodiments, 13G4 or an antigen-binding fragment thereof for use in the methods provided herein comprises a heavy chain variable region comprising the amino acid sequence of (SEQ ID NO: 69); and/or a light chain variable region comprising the amino acid sequence of (SEQ ID NO: 70).

In an embodiment, the targeting moiety comprises the anti-PD-L1 antibody 1E12, or fragments thereof, as disclosed in US 2014/0044738, the entire disclosures of which are hereby incorporated by reference.

In illustrative embodiments, 1E12 or an antigen-binding fragment thereof for use in the methods provided herein comprises a heavy chain variable region comprising the amino acid sequence of (SEQ ID NO: 71); and/or a light chain variable region comprising the amino acid sequence of (SEQ ID NO: 72).

In an embodiment, the targeting moiety comprises the anti-PD-L1 antibody 1F4, or fragments thereof, as disclosed in US 2014/0044738, the entire disclosures of which are hereby incorporated by reference. In illustrative embodiments, 1F4 or an antigen-binding fragment thereof for use in the methods provided herein comprises a heavy chain variable region comprising the amino acid sequence of (SEQ ID NO: 73); and/or a light chain variable region comprising the amino acid sequence of (SEQ ID NO: 74).

In an embodiment, the targeting moiety comprises the anti-PD-L1 antibody 2G11, or fragments thereof, as disclosed in US 2014/0044738, the entire disclosures of which are hereby incorporated by reference.

In illustrative embodiments, 2G11 or an antigen-binding fragment thereof for use in the methods provided herein comprises a heavy chain variable region comprising the amino acid sequence of (SEQ ID NO: 75); and/or a light chain variable region comprising the amino acid sequence of (SEQ ID NO: 76).

In an embodiment, the targeting moiety comprises the anti-PD-L1 antibody 3B6, or fragments thereof, as disclosed in US 2014/0044738, the entire disclosures of which are hereby incorporated by reference. In illustrative embodiments, 3B6 or an antigen-binding fragment thereof for use in the methods provided herein comprises a heavy chain variable region comprising the amino acid sequence of (SEQ ID NO: 77); and/or a light chain variable region comprising the amino acid sequence of (SEQ ID NO: 78).

In an embodiment, the targeting moiety comprises the anti-PD-L1 antibody 3D10, or fragments thereof, as disclosed in US 2014/0044738 and WO2012/145493, the entire disclosures of which are hereby incorporated by reference. In illustrative embodiments, 3D10 or an antigen-binding fragment thereof for use in the methods provided herein comprises a heavy chain variable region comprising the amino acid sequence of (SEQ ID NO: 79); and/or a light chain variable region comprising the amino acid sequence of (SEQ ID NO: 80).

In an embodiment, the targeting moiety comprises any one of the anti-PD-L1 antibodies disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 34-38 of US2011/0271358 (SEQ ID No: 34 of US2011/0271358 (SEQ ID NO: 81); SEQ ID No: 35 of US2011/0271358 (SEQ ID NO: 82); SEQ ID No: 36 of US2011/0271358 (SEQ ID NO: 83); SEQ ID No: 37 of US2011/0271358 (SEQ ID NO: 84); and SEQ ID No: 38 of US2011/0271358 (SEQ ID NO: 85)); and/or a light chain comprising an amino acid sequence selected from SEQ ID Nos: 39-42 of US2011/0271358 (SEQ ID No: 39 of US2011/0271358 (SEQ ID NO: 86); SEQ ID No: 40 of US2011/0271358 (SEQ ID NO: 87); SEQ ID No: 41 of US2011/0271358 (SEQ ID NO: 88); and SEQ ID No: 42 of US2011/0271358 (SEQ ID NO: 89)).

In an embodiment, the targeting moiety comprises the anti-PD-L1 antibody 2.7A4, or fragments thereof, as disclosed in WO 2011/066389, U.S. Pat. No. 8,779,108, and US2014/0356353, the entire disclosures of which are hereby incorporated by reference. In illustrative embodiments, 2.7A4 or an antigen-binding fragment thereof for use in the methods provided herein comprises a heavy chain variable region comprising the amino acid sequence of SEQ ID No: 2 of WO 2011/066389 (SEQ ID NO: 90); and/or a light chain variable region comprising the amino acid sequence of SEQ ID No: 7 of WO 2011/066389 (SEQ ID NO: 91).

In an embodiment, the targeting moiety comprises the anti-PD-L1 antibody 2.9D10, or fragments thereof, as disclosed in WO 2011/066389, U.S. Pat. No. 8,779,108, and US2014/0356353, the entire disclosures of which are hereby incorporated by reference. In illustrative embodiments, 2.9D10 or an antigen-binding fragment thereof for use in the methods provided herein comprises a heavy chain variable region comprising the amino acid sequence of SEQ ID No: 12 of WO 2011/066389 (SEQ ID NO: 92); and/or a light chain variable region comprising the amino acid sequence of SEQ ID No: 17 of WO 2011/066389 (SEQ ID NO: 93).

In an embodiment, the targeting moiety comprises the anti-PD-L1 antibody 2.14H9, or fragments thereof, as disclosed in WO 2011/066389, U.S. Pat. No. 8,779,108, and US2014/0356353, the entire disclosures of which are hereby incorporated by reference. In illustrative embodiments, 2.14H9 or an antigen-binding fragment thereof for use in the methods provided herein comprises a heavy chain variable region comprising the amino acid sequence of SEQ ID No: 22 of WO 2011/066389 (SEQ ID NO: 94); and/or a light chain variable region comprising the amino acid sequence of SEQ ID No: 27 of WO 2011/066389 (SEQ ID NO: 95).

In an embodiment, the targeting moiety comprises the anti-PD-L1 antibody 2.20A8, or fragments thereof, as disclosed in WO 2011/066389, U.S. Pat. No. 8,779,108, and US2014/0356353, the entire disclosures of which are hereby incorporated by reference. In illustrative embodiments, 2.20A8 or an antigen-binding fragment thereof for use in the methods provided herein comprises a heavy chain variable region comprising the amino acid sequence of SEQ ID No: 32 of WO 2011/066389 (SEQ ID NO: 96); and/or a light chain variable region comprising the amino acid sequence of SEQ ID No: 37 of WO 2011/066389 (SEQ ID NO: 97).

In an embodiment, the targeting moiety comprises the anti-PD-L1 antibody 3.15G8, or fragments thereof, as disclosed in WO 2011/066389, U.S. Pat. No. 8,779,108, and US2014/0356353, the entire disclosures of which are hereby incorporated by reference. In illustrative embodiments, 3.15G8 or an antigen-binding fragment thereof for use in the methods provided herein comprises a heavy chain variable region comprising the amino acid sequence of SEQ ID No: 42 of WO 2011/066389 (SEQ ID NO: 98); and/or a light chain variable region comprising the amino acid sequence of SEQ ID No: 47 of WO 2011/066389 (SEQ ID NO: 99).

In an embodiment, the targeting moiety comprises the anti-PD-L1 antibody 3.18G1, or fragments thereof, as disclosed in WO 2011/066389, U.S. Pat. No. 8,779,108, and US2014/0356353, the entire disclosures of which are hereby incorporated by reference. In illustrative embodiments, 3.18G1 or an antigen-binding fragment thereof for use in the methods provided herein comprises a heavy chain variable region comprising the amino acid sequence of SEQ ID No: 52 of WO 2011/066389 (SEQ ID NO:100); and/or a light chain variable region comprising the amino acid sequence of SEQ ID No: 57 of WO 2011/066389 (SEQ ID NO: 101).

In an embodiment, the targeting moiety comprises the anti-PD-L1 antibody 2.7A4OPT, or fragments thereof, as disclosed in WO 2011/066389, U.S. Pat. No. 8,779,108, and US2014/0356353, and US2014/0356353, the entire disclosures of which are hereby incorporated by reference. In illustrative embodiments, 2.7A4OPT or an antigen-binding fragment thereof for use in the methods provided herein comprises a heavy chain variable region comprising the amino acid sequence of SEQ ID No: 62 of WO 2011/066389 (SEQ ID NO:102); and/or a light chain variable region comprising the amino acid sequence of SEQ ID No: 67 of WO 2011/066389 (SEQ ID NO:103).

In an embodiment, the targeting moiety comprises the anti-PD-L1 antibody 2.14H9OPT, or fragments thereof, as disclosed in WO 2011/066389, U.S. Pat. No. 8,779,108, and US2014/0356353, the entire disclosures of which are hereby incorporated by reference. In illustrative embodiments, 2.14H9OPT or an antigen-binding fragment thereof for use in the methods provided herein comprises a heavy chain variable region comprising the amino acid sequence of SEQ ID No: 72 of WO 2011/066389 (SEQ ID NO:104); and/or a light chain variable region comprising the amino acid sequence of SEQ ID No: 77 of WO 2011/066389 (SEQ ID NO:105).

In an embodiment, the targeting moiety comprises any one of the anti-PD-L1 antibodies disclosed in WO2016/061142, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 18, 30, 38, 46, 50, 54, 62, 70, and 78 of WO2016/061142 (SEQ ID No: 18 of WO2016/061142 (SEQ ID NO:106); SEQ ID No: 30 of WO2016/061142 (SEQ ID NO:107); SEQ ID No: 38 of WO2016/061142 (SEQ ID NO:108); SEQ ID No: 46 of WO2016/061142 (SEQ ID NO:109); SEQ ID No: 50 of WO2016/061142 (SEQ ID NO: 110); SEQ ID No: 54 of WO2016/061142 (SEQ ID NO:111); SEQ ID No: 62 of WO2016/061142 (SEQ ID NO: 112); SEQ ID No: 70 of WO2016/061142 (SEQ ID NO:113); and SEQ ID No: 78 of WO2016/061142 (SEQ ID NO:114); and/or a light chain comprising an amino acid sequence selected from SEQ ID Nos: 22, 26, 34, 42, 58, 66, 74, 82, and 86 of WO2016/061142 (SEQ ID No: 22 of WO2016/061142 (SEQ ID NO:115); SEQ ID No: 26 of WO2016/061142 (SEQ ID NO:116); SEQ ID No: 34 of WO2016/061142 (SEQ ID NO:117); SEQ ID No: 42 of WO2016/061142 (SEQ ID NO:118); SEQ ID No: 58 of WO2016/061142 (SEQ ID NO:119); SEQ ID No: 66 of WO2016/061142 (SEQ ID NO:120); SEQ ID No: 74 of WO2016/061142 (SEQ ID NO:121); SEQ ID No: 82 of WO2016/061142 (SEQ ID NO:122); and SEQ ID No: 86 of WO2016/061142 (SEQ ID NO:123).

In an embodiment, the targeting moiety comprises any one of the anti-PD-L1 antibodies disclosed in WO2016/022630, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 2, 6, 10, 14, 18, 22, 26, 30, 34, 38, 42, and 46 of WO2016/022630 (SEQ ID No: 2 of WO2016/022630 (SEQ ID NO: 124); SEQ ID No: 6 of WO2016/022630 (SEQ ID NO:125); SEQ ID No: 10 of WO2016/022630 (SEQ ID NO: 126); SEQ ID No: 14 of WO2016/022630 (SEQ ID NO:127); SEQ ID No: 18 of WO2016/022630 (SEQ ID NO:128); SEQ ID No: 22 of WO2016/022630 (SEQ ID NO:129); SEQ ID No: 26 of WO2016/022630 (SEQ ID NO:130); SEQ ID No: 30 of WO2016/022630 (SEQ ID NO:131); SEQ ID No: 34 of WO2016/022630 (SEQ ID NO:132); SEQ ID No: 38 of WO2016/022630 (SEQ ID NO:133); SEQ ID No: 42 of WO2016/022630 (SEQ ID NO:134); and SEQ ID No: 46 of WO2016/022630 (SEQ ID NO:135)); and/or a light chain comprising an amino acid sequence selected from SEQ ID Nos: 4, 8, 12, 16, 20, 24, 28, 32, 36, 40, 44, and 48 of WO2016/022630 (SEQ ID No: 4 of WO2016/022630 (SEQ ID NO:136); SEQ ID No: 8 of WO2016/022630 (SEQ ID NO:137); SEQ ID No: 12 of WO2016/022630 (SEQ ID NO: 138); SEQ ID No: 16 of WO2016/022630 (SEQ ID NO:139); SEQ ID No: 20 of WO2016/022630 (SEQ ID NO: 140); SEQ ID No: 24 of WO2016/022630 (SEQ ID NO:141); SEQ ID No: 28 of WO2016/022630 (SEQ ID NO:142); SEQ ID No: 32 of WO2016/022630 (SEQ ID NO:143); SEQ ID No: 36 of WO2016/022630 (SEQ ID NO:144); SEQ ID No: 40 of WO2016/022630 (SEQ ID NO:145); SEQ ID No: 44 of WO2016/022630 (SEQ ID NO:146); and SEQ ID No: 48 of WO2016/022630 (SEQ ID NO:147).

In an embodiment, the targeting moiety comprises any one of the anti-PD-L1 antibodies disclosed in WO2015/112900, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 38, 50, 82, and 86 of WO 2015/112900 (SEQ ID No: 38 of WO2015/112900 (SEQ ID NO:148); SEQ ID No: 50 of WO 2015/112900 (SEQ ID NO:149); SEQ ID No: 82 of WO 2015/112900 (SEQ ID NO:150); and SEQ ID No: 86 of WO 2015/112900 (SEQ ID NO:151)); and/or a light chain comprising an amino acid sequence selected from SEQ ID Nos: 42, 46, 54, 58, 62, 66, 70, 74, and 78 of WO 2015/112900 (SEQ ID No: 42 of WO2015/112900 (SEQ ID NO:152); SEQ ID No: 46 of WO 2015/112900: (SEQ ID NO:153); SEQ ID No: 54 of WO 2015/112900 (SEQ ID NO:154); SEQ ID No: 58 of WO 2015/112900 (SEQ ID NO:155); SEQ ID No: 62 of WO 2015/112900 (SEQ ID NO:156); SEQ ID No: 66 of WO 2015/112900 (SEQ ID NO: 157); SEQ ID No: 70 of WO 2015/112900 (SEQ ID NO:158); SEQ ID No: 74 of WO 2015/112900 (SEQ ID NO:159); and SEQ ID No: 78 of WO 2015/112900 (SEQ ID NO:160)).

In an embodiment, the targeting moiety comprises any one of the anti-PD-L1 antibodies disclosed in WO 2010/077634 and U.S. Pat. No. 8,217,149, the entire disclosures of which are hereby incorporated by reference.

In illustrative embodiments, the anti-PD-L1 antibody or an antigen-binding fragment thereof for use in the methods provided herein comprises a heavy chain region comprising the amino acid sequence of SEQ ID No: 20 of WO 2010/077634 (SEQ ID NO: 161); and/or a light chain variable region comprising the amino acid sequence of SEQ ID No: 21 of WO 2010/077634 (SEQ ID NO: 162).

In an embodiment, the targeting moiety comprises any one of the anti-PD-L1 antibodies obtainable from the hybridoma accessible under CNCM deposit numbers CNCM I-4122, CNCM I-4080 and CNCM I-4081 as disclosed in US20120039906, the entire disclosures of which are hereby incorporated by reference.

In an embodiment, the targeting moiety comprises a VHH directed against PD-L1 as disclosed, for example, in U.S. Pat. No. 8,907,065 and WO 2008/071447, the entire disclosures of which are hereby incorporated by reference. In illustrative embodiments, the VHHs against PD-L1 comprise SEQ ID NOS: 394-399 of U.S. Pat. No. 8,907,065 (SEQ ID No: 394 of U.S. Pat. No. 8,907,065 (SEQ ID NO:163); SEQ ID No: 395 of U.S. Pat. No. 8,907,065 (SEQ ID NO:164); SEQ ID No: 396 of U.S. Pat. No. 8,907,065 (SEQ ID NO:165); SEQ ID No: 397 of U.S. Pat. No. 8,907,065 (SEQ ID NO:166); SEQ ID No: 398 of U.S. Pat. No. 8,907,065 (SEQ ID NO:167); and SEQ ID No: 399 of U.S. Pat. No. 8,907,065 (SEQ ID NO:168)).

In various embodiments, the present chimeric proteins or chimeric protein complexes such as Fc-based chimeric protein complexes have one or more targeting moieties directed against PD-L2. In some embodiments, the vaccine compositions, adjuvants, chimeric proteins or chimeric protein complexes have one or more targeting moieties which selectively bind a PD-L2 polypeptide. In some embodiments, the vaccine compositions, adjuvants, chimeric proteins or chimeric protein complexes comprise one or more antibodies, antibody derivatives or formats, peptides or polypeptides, or fusion proteins that selectively bind a PD-L2 polypeptide.

In an embodiment, the targeting moiety comprises a VHH directed against PD-L2 as disclosed, for example, in U.S. Pat. No. 8,907,065 and WO 2008/071447, the entire disclosures of which are hereby incorporated by reference. In illustrative embodiments, the VHHs against PD-L2 comprise SEQ ID Nos: 449-455 of U.S. Pat. No. 8,907,065 (SEQ ID No: 449 of U.S. Pat. No. 8,907,065 (SEQ ID NO:169); SEQ ID No: 450 of U.S. Pat. No. 8,907,065 (SEQ ID NO:170); SEQ ID No: 451 of U.S. Pat. No. 8,907,065 (SEQ ID NO:171); SEQ ID No: 452 of U.S. Pat. No. 8,907,065 (SEQ ID NO:172); SEQ ID No: 453 of U.S. Pat. No. 8,907,065 (SEQ ID NO:173); SEQ ID No: 454 of U.S. Pat. No. 8,907,065 (SEQ ID NO:174); and SEQ ID No: 455 of U.S. Pat. No. 8,907,065 (SEQ ID NO:175)).

In an embodiment, the targeting moiety comprises any one of the anti-PD-L2 antibodies disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 43-47 of US2011/0271358 (SEQ ID No: 43 of US2011/0271358 (SEQ ID NO:176); SEQ ID No: 44 of US2011/0271358 (SEQ ID NO:177); SEQ ID No: 45 of US2011/0271358 (SEQ ID NO:178); SEQ ID No: 46 of US2011/0271358 (SEQ ID NO:179); and SEQ ID No: 47 of US2011/0271358 (SEQ ID NO: 180)); and/or a light chain comprising an amino acid sequence selected from SEQ ID Nos: 48-51 of US2011/0271358 (SEQ ID No: 48 of US2011/0271358 (SEQ ID NO:181); SEQ ID No: 49 of US2011/0271358 (SEQ ID NO:182); SEQ ID No: 50 of US2011/0271358 (SEQ ID NO:183); and SEQ ID No: 51 of US2011/0271358 (SEQ ID NO:184)).

In various embodiments, the targeting moieties of the invention may comprise a sequence that targets PD-1, PD-L1, and/or PD-L2 which is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity with any of the sequences disclosed herein).

In various embodiments, the targeting moieties of the invention may comprise any combination of heavy chain, light chain, heavy chain variable region, light chain variable region, complementarity-determining region (CDR), and framework region sequences that target PD-1, PD-L1, and/or PD-L2 as disclosed herein.

Additional antibodies, antibody derivatives or formats, peptides or polypeptides, or fusion proteins that selectively bind or target PD-1, PD-L1 and/or PD-L2 are disclosed in WO 2011/066389, US 2008/0025980, US 2013/0034559, U.S. Pat. No. 8,779,108, US 2014/0356353, U.S. Pat. No. 8,609,089, US 2010/028330, US 2012/0114649, WO 2010/027827, WO 2011/066342, U.S. Pat. No. 8,907,065, WO 2016/062722, WO 2009/101611, WO2010/027827, WO 2011/066342, WO 2007/005874, WO 2001/014556, US2011/0271358, WO 2010/036959, WO 2010/077634, U.S. Pat. No. 8,217,149, US 2012/0039906, WO 2012/145493, US 2011/0318373, U.S. Pat. No. 8,779,108, US20140044738, WO 2009/089149, WO 2007/00587, WO 2016061142, WO 2016,02263, WO 2010/077634, and WO 2015/112900, the entire disclosures of which are hereby incorporated by reference.

In one embodiment, the present chimeric proteins, vaccines, adjuvants, or chimeric protein complexes have (i) a targeting moiety directed against a T cell, for example, mediated by targeting to CD8 and (ii) a targeting moiety is directed against a tumor cell, along with any of the signaling agents (e.g., IL-1α, pro-IL-1α, or a variant thereof) described herein. In an embodiment, the present chimeric proteins or chimeric protein complexes such as Fc-based chimeric protein complexes have a targeting moiety directed against CD8 on T cells and a second targeting moiety directed against PD-L1 or PD-L2 on tumor cells.

In one embodiment, the present chimeric proteins, vaccines, adjuvants, or chimeric protein complexes have (i) a targeting moiety directed against a T cell, for example, mediated by targeting to CD4 and (ii) a targeting moiety is directed against a tumor cell, along with any of the signaling agents (e.g., IL-1α or variant thereof) described herein. In an embodiment, the present chimeric proteins or chimeric protein complexes such as Fc-based chimeric protein complexes have a targeting moiety directed against CD4 on T cells and a second targeting moiety directed against PD-L1 or PD-L2 on tumor cells.

In one embodiment, the present chimeric proteins, vaccines, adjuvants, or chimeric protein complexes have (i) a targeting moiety directed against a T cell, for example, mediated by targeting to CD3, CXCR3, CCR4, CCR9, CD70, CD103, or one or more immune checkpoint markers and (ii) a targeting moiety is directed against a tumor cell, along with any of the signaling agents (e.g., IL-1α or a variant thereof) described herein. In an embodiment, the present chimeric proteins or chimeric protein complexes such as Fc-based chimeric protein complexes have a targeting moiety directed against CD3 on T cells and a second targeting moiety directed against PD-L1 or PD-L2 on tumor cells.

In some embodiments, the present chimeric proteins or chimeric protein complexes such as Fc-based chimeric protein complexes have one or more targeting moieties directed against CD3 expressed on T cells. In some embodiments, the vaccine compositions, adjuvants, chimeric proteins or chimeric protein complexes have one or more targeting moieties, which selectively bind a CD3 polypeptide. In some embodiments, the vaccine compositions, adjuvants, chimeric proteins or chimeric protein complexes comprise one or more antibodies, antibody derivatives or formats, peptides or polypeptides, or fusion proteins that selectively bind a CD3 polypeptide.

In an embodiment, the targeting moiety comprises the anti-CD3 antibody muromonab-CD3 (aka Orthoclone OKT3), or fragments thereof. Muromonab-CD3 is disclosed in U.S. Pat. No. 4,361,549 and Wilde et al. (1996) 51:865-894, the entire disclosures of which are hereby incorporated by reference. In illustrative embodiments, muromonab-CD3 or an antigen-binding fragment thereof for use in the methods provided herein comprises a heavy chain comprising the amino acid sequence of (SEQ ID NO:185); and/or a light chain comprising the amino acid sequence of (SEQ ID NO:186).

In an embodiment, the targeting moiety comprises the anti-CD3 antibody otelixizumab, or fragments thereof. Otelixizumab is disclosed in U.S. Patent Publication No. 20160000916 and Chatenoud et al. (2012) 9:372-381, the entire disclosures of which are hereby incorporated by reference. In illustrative embodiments, otelixizumab or an antigen-binding fragment thereof for use in the methods provided herein comprises a heavy chain comprising the amino acid sequence of: SEQ ID NO:187; and/or a light chain comprising the amino acid sequence of SEQ ID NO:188.

In an embodiment, the targeting moiety comprises the anti-CD3 antibody teplizumab (AKA MGA031 and hOKT3γ1 (Ala-Ala)), or fragments thereof. Teplizumab is disclosed in Chatenoud et al. (2012) 9:372-381, the entire disclosures of which are hereby incorporated by reference. In illustrative embodiments, teplizumab or an antigen-binding fragment thereof for use in the methods provided herein comprises a heavy chain comprising the amino acid sequence of SEQ ID NO:189; and/or a light chain comprising the amino acid sequence of SEQ ID NO: 190.

In an embodiment, the targeting moiety comprises the anti-CD3 antibody visilizumab (AKA Nuvion®; HuM291), or fragments thereof. Visilizumab is disclosed in U.S. Pat. No. 5,834,597 and WO2004052397, and Cole et al., Transplantation (1999) 68:563-571, the entire disclosures of which are hereby incorporated by reference. In illustrative embodiments, visilizumab or an antigen-binding fragment thereof for use in the methods provided herein comprises a heavy chain variable region comprising the amino acid sequence of SEQ ID NO:191; and/or a light chain variable region comprising the amino acid sequence of SEQ ID NO:192.

In an embodiment, the targeting moiety comprises the anti-CD3 antibody foralumab (aka NI-0401), or fragments thereof. In various embodiments, the targeting moiety comprises any one of the anti-CD3 antibodies disclosed in US20140193399, U.S. Pat. No. 7,728,114, US20100183554, and U.S. Pat. No. 8,551,478, the entire disclosures of which are hereby incorporated by reference.

In illustrative embodiments, the anti-CD3 antibody or an antigen-binding fragment thereof for use in the methods provided herein comprises a heavy chain variable region comprising the amino acid sequence of SEQ ID Nos: 2 and 6 of U.S. Pat. No. 7,728,114 (SEQ ID No: 2 of U.S. Pat. No. 7,728,114 (SEQ ID NO:193) and SEQ ID No: 6 of U.S. Pat. No. 7,728,114 (SEQ ID NO:194)); and/or a light chain variable region comprising the amino acid sequence of SEQ ID NOs 4 and 8 of U.S. Pat. No. 7,728,114 (SEQ ID No: 4 of U.S. Pat. No. 7,728,114 (SEQ ID NO:195) and SEQ ID No: 8 of U.S. Pat. No. 7,728,114 (SEQ ID NO:196)).

In an embodiment, the targeting moiety comprises a heavy chain variable region comprising the amino acid sequence of SEQ ID NO:2 of U.S. Pat. No. 7,728,114 and a light chain variable region comprising the amino acid sequence of SEQ ID NO:4 of U.S. Pat. No. 7,728,114. In an embodiment, the targeting moiety comprises any one of the anti-CD3 antibodies disclosed in US2016/0168247, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6-9 of US2016/0168247 (SEQ ID No: 6 of US2016/0168247 (SEQ ID NO: 197); SEQ ID No: 7 of US2016/0168247 (SEQ ID NO:198); SEQ ID No: 8 of US2016/0168247 (SEQ ID NO:199); and SEQ ID No: 9 of US2016/0168247 (SEQ ID NO:200)); and/or a light chain comprising an amino acid sequence selected from SEQ ID Nos: 10-12 of US2016/0168247 (SEQ ID No: 10 of US2016/0168247 (SEQ ID NO:201); SEQ ID No: 11 of US2016/0168247 (SEQ ID NO:202); and SEQ ID No: 12 of US2016/0168247 (SEQ ID NO:203).

In an embodiment, the targeting moiety comprises any one of the anti-CD3 antibodies disclosed in US2015/0175699, the entire contents of which are hereby incorporated by reference. In illustrative embodiments, the antibody or an antigen-binding fragment thereof for use in the methods provided herein comprises a heavy chain comprising an amino acid sequence selected from SEQ ID No: 9 of US2015/0175699 (SEQ ID NO:204); and/or a light chain comprising an amino acid sequence selected from SEQ ID No: 10 of US2015/0175699 (SEQ ID NO:205).

In an embodiment, the targeting moiety comprises any one of the anti-CD3 antibodies disclosed in U.S. Pat. No. 8,784,821, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 2, 18, 34, 50, 66, 82, 98 and 114 of U.S. Pat. No. 8,784,821 (SEQ ID No: 2 of U.S. Pat. No. 8,784,821 (SEQ ID NO:206); SEQ ID No: 18 of U.S. Pat. No. 8,784,821 (SEQ ID NO:207); SEQ ID No: 34 of U.S. Pat. No. 8,784,821 (SEQ ID NO:208); SEQ ID No: 50 of U.S. Pat. No. 8,784,821 (SEQ ID NO:209); SEQ ID No: 66 of U.S. Pat. No. 8,784,821 (SEQ ID NO:210); SEQ ID No: 82 of U.S. Pat. No. 8,784,821 (SEQ ID NO:211); SEQ ID No: 98 of U.S. Pat. No. 8,784,821 (SEQ ID NO:212); and SEQ ID No: 114 of U.S. Pat. No. 8,784,821 (SEQ ID NO:213)); and/or a light chain comprising an amino acid sequence selected from SEQ ID Nos: 10, 26, 42, 58, 74, 90, 106 and 122 of U.S. Pat. No. 8,784,821 (SEQ ID No: 10 of U.S. Pat. No. 8,784,821 (SEQ ID NO: 214); SEQ ID No: 26 of U.S. Pat. No. 8,784,821 (SEQ ID NO:215); SEQ ID No: 42 of U.S. Pat. No. 8,784,821 (SEQ ID NO: 216); SEQ ID No: 58 of U.S. Pat. No. 8,784,821 (SEQ ID NO:217); SEQ ID No: 74 of U.S. Pat. No. 8,784,821 (SEQ ID NO: 218); SEQ ID No: 90 of U.S. Pat. No. 8,784,821 (SEQ ID NO:219); SEQ ID No: 106 of U.S. Pat. No. 8,784,821 (SEQ ID NO: 220); and SEQ ID No: 122 of U.S. Pat. No. 8,784,821 (SEQ ID NO:221).

In an embodiment, the targeting moiety comprises any one of the anti-CD3 binding constructs disclosed in US20150118252, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6 and 86 of US20150118252 (SEQ ID No: 6 of US20150118252 (SEQ ID NO:222) and SEQ ID No: 86 of US20150118252 (SEQ ID NO:223) and/or a light chain comprising an amino acid sequence selected from SEQ ID No: 3 of US2015/0175699 (SEQ ID No: 3 of US20150118252 (SEQ ID NO:224).

In an embodiment, the targeting moiety comprises any one of the anti-CD3 binding proteins disclosed in US2016/0039934, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6-9 of US2016/0039934 (SEQ ID No: 6 of US2016/0039934 (SEQ ID NO:225); SEQ ID No: 7 of US2016/0039934 (SEQ ID NO:226); SEQ ID No: 8 of US2016/0039934 (SEQ ID NO:227); and SEQ ID No: 9 of US2016/0039934 (SEQ ID NO:228)); and/or a light chain comprising an amino acid sequence selected from SEQ ID Nos: 1-4 of US2016/0039934 (SEQ ID No: 1 of US2016/0039934 (SEQ ID NO: 229); SEQ ID No: 2 of US2016/0039934 (SEQ ID NO:230); SEQ ID No: 3 of US2016/0039934 (SEQ ID NO: 231); and SEQ ID No: 4 of US2016/0039934 (SEQ ID NO:232)).

In various embodiments, the targeting moieties of the invention may comprise a sequence that targets CD3 which is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity with any of the sequences disclosed herein).

In various embodiments, the targeting moieties of the invention may comprise any combination of heavy chain, light chain, heavy chain variable region, light chain variable region, complementarity determining region (CDR), and framework region sequences that target CD3 as disclosed herein. In various embodiments, the targeting moieties of the invention may comprise any heavy chain, light chain, heavy chain variable region, light chain variable region, complementarity determining region (CDR), and framework region sequences of the CD3-specific antibodies including, but not limited to, X35-3, VIT3, BMA030 (BW264/56), CLB-T3/3, CRIS7, YTH12.5, FI 11-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, WT31 and F101.01. These CD3-specific antibodies are well known in the art and, inter alia, described in Tunnacliffe (1989), Int. Immunol. 1, 546-550, the entire disclosures of which are hereby incorporated by reference.

Additional antibodies, antibody derivatives or formats, peptides or polypeptides, or fusion proteins that selectively bind or target CD3 are disclosed in US Patent Publication No. 2016/0000916, U.S. Pat. Nos. 4,361,549, 5,834,597, 6,491,916, 6,406,696, 6,143,297, 6,750,325 and International Publication No. WO 2004/052397, the entire disclosures of which are hereby incorporated by reference.

In one embodiment, the vaccine compositions, adjuvants, chimeric proteins or chimeric protein complexes have (i) a targeting moiety directed against a T cell, for example, mediated by targeting to PD-1 and (ii) a targeting moiety is directed against a tumor cell, along with any of the signaling agents (e.g., IL-1α or variant thereof) described herein.

By way of non-limiting example, in various embodiments, the vaccine compositions, adjuvants, chimeric proteins or chimeric protein complexes have (i) a targeting moiety directed against a B cell, for example, mediated by targeting to CD10, CD19, CD20, CD21, CD22, CD23, CD24, CD37, CD38, CD39, CD40, CD70, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138, or CDw150; and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against CD20.

In one embodiment, the vaccine compositions, adjuvants, chimeric proteins or chimeric protein complexes have (i) a targeting moiety directed against a B cell, for example, mediated by targeting to CD19, CD20 or CD70 and (ii) a targeting moiety is directed against a tumor cell, along with any of the signaling agents (e.g., IL-1α or variant thereof) described herein.

In one embodiment, the vaccine compositions, adjuvants, chimeric proteins or chimeric protein complexes have (i) a targeting moiety directed against a B cell, for example, mediated by targeting to CD20 and (ii) a targeting moiety is directed against a tumor cell, along with any of the signaling agents (e.g., IL-1α or a variant thereof) described herein. In an embodiment, the vaccine compositions, adjuvants, chimeric proteins or chimeric protein complexes have a targeting moiety directed against CD20 on B cells and a second targeting moiety directed against PD-L1 or PD-L2 on tumor cells. By way of example, in some embodiments, the CD20 targeting moiety is a recombinant heavy-chain-only antibody (VHH) having the sequence of:

(SEQ ID NO: 288)
QVQLQESGGGLAQAGGSLRLSCAASGRTFSMGWFRQAPGKEREFVAAITY
SGGSPYYASSVRGRFTISRDNAKNTVYLQMNSLKPEDTAVYYCAANPTYG
SDWNAENWGQGTQVTVSS.

By way of non-limiting example, in various embodiments, the vaccine compositions, adjuvants, chimeric proteins or chimeric protein complexes have (i) a targeting moiety directed against a NK cell, for example, mediated by targeting to 2B4/SLAMF4, KIR2DS4, CD155/PVR, KIR3DL1, CD94, LMIR1/CD300A, CD69, LMIR2/CD300c, CRACC/SLAMF7, LMIR3/CD300LF, DNAM-1, LMIR5/CD300LB, Fc-epsilon RII, LMIR6/CD300LE, Fc-γ RI/CD64, MICA, Fc-γ RIIB/CD32b, MICB, Fc-γ RIIC/CD32c, MULT-1, Fc-γ RIIA/CD32a, Nectin-2/CD112, Fc-γ RIII/CD16, NKG2A, FcRH1/IRTA5, NKG2C, FcRH2/IRTA4, NKG2D, FcRH4/IRTA1, NKp30, FcRH5/IRTA2, NKp44, Fc-Receptor-like 3/CD16-2, NKp46/NCR1, NKp80/KLRF1, NTB-A/SLAMF6, Rae-1, Rae-1 α, Rae-1 β, Rae-1 delta, H60, Rae-1 epsilon, ILT2/CD85j, Rae-1 γ, ILT3/CD85k, TREM-1, ILT4/CD85d, TREM-2, ILT5/CD85a, TREM-3, KIR/CD158, TREML1/TLT-1, KIR2DL1, ULBP-1, KIR2DL3, ULBP-2, KIR2DL4/CD158d, or ULBP-3; and (ii) a targeting moiety is directed against a tumor cell, along with any of the signaling agents (e.g., IL-1α or variant thereof) described herein.

In one embodiment, the vaccine compositions, adjuvants, chimeric proteins or chimeric protein complexes have (i) a targeting moiety directed against a NK cell, for example, mediated by targeting to Kir1alpha, DNAM-1 or CD64 and (ii) a targeting moiety is directed against a tumor cell, along with any of the signaling agents (e.g., IL-1α or variant thereof) described herein.

In one embodiment, the vaccine compositions, adjuvants, chimeric proteins or chimeric protein complexes have (i) a targeting moiety directed against a NK cell, for example, mediated by targeting to KIR1 and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against KIR1 on NK cells and a second targeting moiety directed against PD-L1 or PD-L2 on tumor cells.

In one embodiment, the vaccine compositions, adjuvants, chimeric proteins or chimeric protein complexes have (i) a targeting moiety directed against a NK cell, for example, mediated by targeting to TIGIT or KIR1 and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against TIGIT on NK cells and a second targeting moiety directed against PD-L1 or PD-L2 on tumor cells.

By way of non-limiting example, in various embodiments, the vaccine compositions, adjuvants, chimeric proteins or chimeric protein complexes have (i) a targeting moiety directed against a dendritic cell, for example, mediated by targeting to CLEC-9A, XCR1, RANK, CD36/SRB3, LOX-1/SR-E1, CD68, MARCO, CD163, SR-A1/MSR, CD5L, SREC-1, CL-PI/COLEC12, SREC-II, LIMPIIISRB2, RP105, TLR4, TLR1, TLR5, TLR2, TLR6, TLR3, TLR9, 4-IBB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, lutegrin α 4/CD49d, Aag, Integrin β 2/CD18, AMICA, Langerin, B7-2/CD86, Leukotriene B4 RI, B7-H3, LMIR1/CD300A, BLAME/SLAMF8, LMIR2/CD300c, Clq R1/CD93, LMIR3/CD300LF, CCR6, LMIR5/CD300LB CCR7, LMIR6/CD300LE, CD40/TNFRSF5, MAG/Siglec-4-a, CD43, MCAM, CD45, MD-1, CD68, MD-2, CD83, MDL-1/CLEC5A, CD84/SLAMF5, MMR, CD97, NCAMLI, CD2F-10/SLAMF9, Osteoactivin GPNMB, Chern 23, PD-L2, CLEC-1, RP105, CLEC-2, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC, SLAM, EMMPRIN/CD147, TCCR/WSX-1, Fc-γ R1/CD64, TLR3, Fc-Y RIIB/CD32b, TREM-1, Fc-γ RIIC/CD32c, TREM-2, Fc-γ RIIA/CD32a, TREM-3, Fc-γ RIII/CD16, TREML1/TLT-1, ICAM-2/CD102, or Vanilloid R1; and (ii) a targeting moiety is directed against a tumor cell, along with any of the signaling agents (e.g., IL-1α or variant thereof) described herein.

In one embodiment, the vaccine compositions, adjuvants, chimeric proteins or chimeric protein complexes have (i) a targeting moiety directed against a dendritic cell, for example, mediated by targeting to CLEC-9A, DC-SIGN, CD64, CLEC4A, or DEC205 and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against CLEC9A on dendritic cells and a second targeting moiety directed against PD-L1 or PD-L2 on tumor cells.

In one embodiment, the vaccine compositions, adjuvants, chimeric proteins or chimeric protein complexes have (i) a targeting moiety directed against a dendritic cell, for example, mediated by targeting to CLEC9A and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against CLEC9A on dendritic cells and a second targeting moiety directed against PD-L1 or PD-L2 on tumor cells.

In one embodiment, the vaccine compositions, adjuvants, chimeric proteins or chimeric protein complexes have (i) a targeting moiety directed against a dendritic cell, for example, mediated by targeting to XCR1 and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against XCR1 on dendritic cells and a second targeting moiety directed against PD-L1 or PD-L2 on tumor cells.

In one embodiment, the vaccine compositions, adjuvants, chimeric proteins or chimeric protein complexes have (i) a targeting moiety directed against a dendritic cell, for example, mediated by targeting to RANK and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against RANK on dendritic cells and a second targeting moiety directed against PD-L1 or PD-L2 on tumor cells.

By way of non-limiting example, in various embodiments, the vaccine compositions, adjuvants, chimeric proteins or chimeric protein complexes have (i) a targeting moiety directed against a monocyte/macrophage, for example, mediated by targeting to SIRP1a, B7-1/CD80, ILT4/CD85d, B7-H1, ILT5/CD85a, Common β Chain, Integrin α 4/CD49d, BLAME/SLAMF8, Integrin α X/CDIIc, CCL6/C10, Integrin β 2/CD18, CD155/PVR, Integrin β 3/CD61, CD31/PECAM-1, Latexin, CD36/SR-B3, Leukotriene B4 R1, CD40/TNFRSF5, LIMPIIISR-B2, CD43, LMIR1/CD300A, CD45, LMIR2/CD300c, CD68, LMIR3/CD300LF, CD84/SLAMF5, LMIR5/CD300LB, CD97, LMIR6/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc-γ RI/CD64, Osteopontin, Fc-γ RIIB/CD32b, PD-L2, Fc-γ RIIC/CD32c, Siglec-3/CD33, Fc-γ RIIA/CD32a, SIGNR1/CD209, Fc-γ RIII/CD16, SLAM, GM-CSF R α, TCCR/WSX-1, ICAM-2/CD102, TLR3, IFN-γ RI, TLR4, IFN-γ R2, TREM-I, IL-I RII, TREM-2, ILT2/CD85j, TREM-3, ILT3/CD85k, TREML1/TLT-1, 2B4/SLAMF 4, IL-10 R α, ALCAM, IL-10 R β, AminopeptidaseN/ANPEP, ILT2/CD85j, Common β Chain, ILT3/CD85k, Clq R1/CD93, ILT4/CD85d, CCR1, ILT5/CD85a, CCR2, CD206, Integrin α 4/CD49d, CCR5, Integrin α M/CDII b, CCR8, Integrin α X/CDIIc, CD155/PVR, Integrin β 2/CD18, CD14, Integrin β 3/CD61, CD36/SR-B3, LAIR1, CD43, LAIR2, CD45, Leukotriene B4-R1, CD68, LIMPIIISR-B2, CD84/SLAMF5, LMIR1/CD300A, CD97, LMIR2/CD300c, CD163, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc-γ RI/CD64, PSGL-1, Fc-γ RIIIICD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-I, IL-6 R, TREM-2, CXCRI/IL-8 RA, TREM-3, or TREMLI/TLT-1; and (ii) a targeting moiety is directed against a tumor cell, along with any of the signaling agents (e.g., IL-1α or variant thereof) described herein.

In one embodiment, the vaccine compositions, adjuvants, chimeric proteins or chimeric protein complexes have (i) a targeting moiety directed against a monocyte/macrophage, for example, mediated by targeting to B7-H1, CD31/PECAM-1, CD163, CCR2, or Macrophage Mannose Receptor CD206 and (ii) a targeting moiety is directed against a tumor cell, along with any of the signaling agents (e.g., IL-1α or variant thereof) described herein.

In one embodiment, the vaccine compositions, adjuvants, chimeric proteins or chimeric protein complexes have (i) a targeting moiety directed against a monocyte/macrophage, for example, mediated by targeting to SIRP1a and (ii) a targeting moiety is directed against a tumor cell, along with any of the signaling agents (e.g., IL-1α or variant thereof) described herein. In an embodiment, the vaccine compositions, adjuvants, chimeric proteins or chimeric protein complexes have a targeting moiety directed against SIRP1a on macrophage cells and a second targeting moiety directed against PD-L1 or PD-L2 on tumor cells.

In various embodiments, the vaccine compositions, adjuvants, chimeric proteins or chimeric protein complexes have one or more targeting moieties directed against a checkpoint marker, e.g. one or more of PD-1/PD-L1 or PD-L2, CD28/CD80 or CD86, CTLA4/CD80 or CD86, ICOS/ICOSL or B7RP1, BTLA/HVEM, KIR, LAG3, CD137/CD137L, OX40/OX40L, CD27, CD40L, TIM3/Gal9, CD47, CD70, and A2aR. In one embodiment, the vaccine compositions, adjuvants, chimeric proteins or chimeric protein complexes have (i) a targeting moiety directed against a checkpoint marker on a T cell, for example, PD-1 and (ii) a targeting moiety directed against a tumor cell, for example, PD-L1 or PD-L2, along with any of the signaling agents (e.g., IL-1α or variant thereof) described herein. In an embodiment, the vaccine compositions, adjuvants, chimeric proteins or chimeric protein complexes have a targeting moiety directed against PD-1 on T cells and a second targeting moiety directed against PD-L1 on tumor cells. In another embodiment, the vaccine compositions, adjuvants, chimeric proteins or chimeric protein complexes have a targeting moiety directed against PD-1 on T cells and a second targeting moiety directed against PD-L2 on tumor cells.

In some embodiments, the vaccine compositions, adjuvants, chimeric proteins or chimeric protein complexes comprises two or more targeting moieties directed to the same or different immune cells. In some embodiments, the vaccine compositions, adjuvants, chimeric proteins or chimeric protein complexes have (i) one or more targeting moieties directed against an immune cell selected from a T cell, a B cell, a dendritic cell, a macrophage, a NK cell, or subsets thereof and (ii) one or more targeting moieties directed against either the same or another immune cell selected from a T cell, a B cell, a dendritic cell, a macrophage, a NK cell, or subsets thereof, along with any of the signaling agents (e.g., IL-1α or variant thereof) described herein.

In one embodiment, the vaccine compositions, adjuvants, chimeric proteins or chimeric protein complexes comprises one or more targeting moieties directed against a T cell and one or more targeting moieties directed against the same or another T cell. In one embodiment, the vaccine compositions, adjuvants, chimeric proteins or chimeric protein complexes comprises one or more targeting moieties directed against a T cell and one or more targeting moieties directed against a B cell. In one embodiment, the vaccine compositions, adjuvants, chimeric proteins or chimeric protein complexes comprises one or more targeting moieties directed against a T cell and one or more targeting moieties directed against a dendritic cell. In one embodiment, the vaccine compositions, adjuvants, chimeric proteins or chimeric protein complexes comprises one or more targeting moieties against a T cell and one or more targeting moieties directed against a macrophage. In one embodiment, the vaccine compositions, adjuvants, chimeric proteins or chimeric protein complexes comprises one or more targeting moieties against a T cell and one or more targeting moieties directed against a NK cell. For example, in an illustrative embodiment, the vaccine compositions, adjuvants, chimeric proteins or chimeric protein complexes may include a targeting moiety against CD8 and a targeting moiety against Clec9A. In another illustrative embodiment, the vaccine compositions, adjuvants, chimeric proteins or chimeric protein complexes may include a targeting moiety against CD8 and a targeting moiety against CD3. In another illustrative embodiment, the vaccine compositions, adjuvants, chimeric proteins or chimeric protein complexes may include a targeting moiety against CD8 and a targeting moiety against PD-1.

In various embodiments, the chimeric proteins or the chimeric protein complexes disclosed herein include a CD8 binding agent that is a protein-based agent capable of specific binding to CD8. In various embodiments, the present CD8 binding agent is a protein-based agent capable of specific binding to CD8 without functionally modulating (e.g. partial or complete neutralization) CD8.

In various embodiments, the present CD8 binding agent comprises a targeting moiety capable of specific binding. In various embodiments, the CD8 binding agent comprises a targeting moiety having an antigen recognition domain such as an antibody or derivatives thereof. In an embodiment, the CD8 binding agent comprises a targeting moiety, which is an antibody. In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity-determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the CD8 binding agent comprises a targeting moiety, which is an antibody derivative or format. In some embodiments, the present CD8 binding agent comprises a targeting moiety which is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; 25 an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. No. 7,250,297, U.S. Pat. No. 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3 (3): 310-317.

In some embodiments, the CD8 binding agent comprises a targeting moiety, which is a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3).

In an embodiment, the CD8 binding agent comprises a VHH. In some embodiments, the VHH is a humanized VHH or camelized VHH.

In some embodiments, the VHH comprises a fully human V_{H} domain, e.g. a HUMABODY (Crescendo Biologics, Cambridge, UK). In some embodiments, fully human V_{H} domain, e.g. a HUMABODY is monovalent, bivalent, or trivalent. In some embodiments, the fully human V_{H} domain, e.g. a HUMABODY is mono- or multi-specific such as monospecific, bispecific, or trispecific. Illustrative fully human V_{H} domains, e.g. a HUMABODIES are described in, for example, WO2016/113555 and WO2016/113557, the entire disclosure of which is incorporated by reference.

In some embodiments, the CD8 binding agent comprises a targeting moiety which is a VHH comprising a single amino acid chain having four “framework regions” or FRs and three “complementary determining regions” or CDRs. As used herein, “framework region” or “FR” refers to a region in the variable domain, which is located between the CDRs. As used herein, “complementary determining region” or “CDR” refers to variable regions in VHHs that contains the amino acid sequences capable of specifically binding to antigenic targets. In various embodiments, the CD8 binding agent comprises a VHH having a variable domain comprising at least one CDR1, CDR2, and/or CDR3 sequences.

In some embodiments, the targeting moiety comprises anti-CD8 antibody as described in WO 2019033043, the entire disclosures of which are hereby incorporated by reference. In some embodiments, the anti-CD8 antibody comprises at least one heavy chain variable region comprising the amino acid sequence of CDR H1: GFNIKDTYIH (SEQ ID NO: 293); CDR H2: RIDPANDNTLYASKFQG (SEQ ID NO: 294); CDR H2: RIDPANDNTLYARKFQG (SEQ ID NO: 295); CDR H3: GRGYGYYVFDH (SEQ ID NO: 296); or CDR H3: TRGYGYYVFDT (SEQ ID NO: 297).

In some embodiments, the anti-CD8 antibody comprises at least one light chain variable region comprising the amino acid sequence of CDR L1: SISQY (SEQ ID NO: 298); CDR L1: SISKY (SEQ ID NO: 299); CDR L2: SGSTLQ (SEQ ID NO: 300); CDR L3: HNENPL (SEQ ID NO: 301); CDR L3: HNEFPV (SEQ ID NO: 302); CDR L3: HNEFPP (SEQ ID NO: 303); CDR L3: VNEFPP (SEQ ID NO: 304); CDR L3: VNEFPV (SEQ ID NO: 305).

In some embodiments, the targeting moiety comprises anti-CD8 antibody as described in WO2019023148, the entire disclosures of which are hereby incorporated by reference. In some embodiments, the anti-CD8 antibody comprises at least one heavy chain variable region comprising the amino acid sequence of CDR H1: GFIFSNYG (SEQ ID NO: 306); CDR H2: IWYDGSNK (SEQ ID NO: 307); CDR H3: ARSYDMLTGSGDYYGL (SEQ ID NO: 308). In some embodiments, the anti-CD8 antibody comprises at least one light chain variable region comprising the amino acid sequence of CDR L1: QDITNY (SEQ ID NO: 309); CDR L2: GAS; CDR L3: QQYNNYPLT (SEQ ID NO: 310).

In some embodiments, the targeting moiety comprises anti-CD8 antibody as described in WO2015184203, the entire disclosures of which are hereby incorporated by reference. In some embodiments, the anti-CD8 antibody comprises at least one heavy chain variable region comprising the amino acid sequence of CDR H1: SGYTGTDYNMH (SEQ ID NO: 311); CDR H2: YIYPYTGGTGYNQKFKN (SEQ ID NO: 312); CDR H1: DFGMN (SEQ ID NO: 313); CDR H2: LIYYDGSNKFY (SEQ ID NO: 314); CDR H3: PHYDGYYHFFDS (SEQ ID NO: 315). In some embodiments, the anti-CD8 antibody comprises at least one light chain variable region comprising the amino acid sequence of CDR L1: RASESVDSYDNSLMH (SEQ ID NO: 316); CDR L2: LASNLES (SEQ ID NO: 317); CDR L3: QQNNEDPYT (SEQ ID NO: 318); CDR L1: KGSQDINNYLA (SEQ ID NO: 319); CDR L2: NTDILHT (SEQ ID NO: 320); CDR L3: YQYNNGYT (SEQ ID NO: 321).

In some embodiments, the targeting moiety comprises anti-CD8 antibody as described in WO2018170096, the entire disclosures of which are hereby incorporated by reference. In some embodiments, the anti-CD8 antibody comprises at least one heavy chain variable region comprising the amino acid sequence of CDR H1: GYTFTSY (SEQ ID NO: 322); CDR H2: DPSDNY (SEQ ID NO: 333); CDR H3: PKSAYAFDVGGYAMDY (SEQ ID NO: 334). In some embodiments, the anti-CD8 antibody comprises at least one light chain variable region comprising the amino acid sequence of CDR L1: RTSENIDSYLT (SEQ ID NO: 335); CDR L2: AATLLAD (SEQ ID NO: 336); CDR L3: QHYYSTPWT (SEQ ID NO: 337).

In some embodiments, the targeting moiety comprises anti-CD8 antibody as described in WO2014164553, the entire disclosures of which are hereby incorporated by reference. In some embodiments, the anti-CD8 antibody comprises at least one heavy chain variable region comprising the amino acid sequence of CDR H1: GFNIKD (SEQ ID NO: 338); CDR H2: RIDPANDNT (SEQ ID NO: 339); CDR H3: GYGYYVFDH (SEQ ID NO: 340). In some embodiments, the anti-CD8 antibody comprises at least one light chain variable region comprising the amino acid sequence of CDR L1: RTSRSISQYLA (SEQ ID NO: 341); CDR L2: SGSTLQS (SEQ ID NO: 342); CDR L3: QQHNENPLT (SEQ ID NO: 343).

In various embodiments, the chimeric proteins or the chimeric protein complexes disclosed herein include a CD4 binding agent that is a protein-based agent capable of specific binding to CD4. In various embodiments, the present CD4 binding agent is a protein-based agent capable of specific binding to CD4 without functionally modulating (e.g. partial or complete neutralization) CD4.

In various embodiments, the present CD4 binding agent comprises a targeting moiety capable of specific binding. In various embodiments, the CD4 binding agent comprises a targeting moiety having an antigen recognition domain such as an antibody or derivatives thereof. In an embodiment, the CD4 binding agent comprises a targeting moiety, which is an antibody. In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity-determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the CD4 binding agent comprises a targeting moiety, which is an antibody derivative or format. In some embodiments, the present CD4 binding agent comprises a targeting moiety which is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3 (3): 310-317.

In some embodiments, the CD4 binding agent comprises a targeting moiety, which is a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3).

In an embodiment, the CD4 binding agent comprises a VHH. In some embodiments, the VHH is a humanized VHH or camelized VHH. In some embodiments, the VHH comprises a fully human V_{H} domain, e.g. a HUMABODY (Crescendo Biologics, Cambridge, UK). In some embodiments, fully human V_{H} domain, e.g. a HUMABODY is monovalent, bivalent, or trivalent. In some embodiments, the fully human V_{H} domain, e.g. a HUMABODY is mono- or multi-specific such as monospecific, bispecific, or trispecific. Illustrative fully human V_{H} domains, e.g. a HUMABODIES are described in, for example, WO2016/113555 and WO2016/113557, the entire disclosure of which is incorporated by reference.

In some embodiments, the CD4 binding agent comprises a targeting moiety which is a VHH comprising a single amino acid chain having four “framework regions” or FRs and three “complementary determining regions” or CDRs. As used herein, “framework region” or “FR” refers to a region in the variable domain, which is located between the CDRs. As used herein, “complementary determining region” or “CDR” refers to variable regions in VHHs that contains the amino acid sequences capable of specifically binding to antigenic targets. In various embodiments, the CD4 binding agent comprises a VHH having a variable domain comprising at least one CDR1, CDR2, and/or CDR3 sequences. In some embodiments, the targeting moiety comprises anti-CD4 antibody as described in WO2020082045, the entire disclosures of which are hereby incorporated by reference. In some embodiments, the anti-CD4 antibody comprises at least one heavy chain variable region comprising the amino acid sequence of CDRH1: GYTFTAHI (SEQ ID NO: 344); CDRH2: IKPQYGAV (SEQ ID NO: 345); or CDRH3: AR. In some embodiments, the anti-CD4 antibody comprises at least one light chain variable region comprising the amino acid sequence of CDRL1: QGVGSD (SEQ ID NO: 346); CDRL2: HTS; or CDRL3: QVLQF (SEQ ID NO: 347).

In some embodiments, the targeting moiety comprises anti-CD4 antibody as described in WO2018170096, the entire disclosures of which are hereby incorporated by reference. In some embodiments, the anti-CD4 antibody comprises at least one heavy chain variable region comprising the amino acid sequence of CDRH1: GYTFTSN (SEQ ID NO: 348); CDRH2: YPRSGN (SEQ ID NO: 349); or CDRH3: RVPYFDH (SEQ ID NO: 350). In some embodiments, the anti-CD4 antibody comprises at least one light chain variable region comprising the amino acid sequence of CDRL1: KASQSVGNNVA (SEQ ID NO: 351); CDRL2: YASNRYT (SEQ ID NO: 352); or CDRL3: QQHYSSPFT (SEQ ID NO: 353).

In some embodiments, the targeting moiety comprises anti-CD4 antibody as described in WO2016156570, the entire disclosures of which are hereby incorporated by reference. In some embodiments, the anti-CD4 antibody comprises at least one CDR1 comprising the amino acid sequence of: GYWMY (SEQ ID NO: 354); CDR1: SYSMG (SEQ ID NO: 355); CDR1: FNAMG (SEQ ID NO: 356); or CDR1: VMG. In some embodiments, the anti-CD4 antibody comprises at least one CDR2 comprising the amino acid sequence of CDR2: AISPGGGSTYYPDSVK (SEQ ID NO: 357); CDR2: AISWSGDETSYADSVK (SEQ ID NO: 358); CDR2: TIARAGATKYADSVKG (SEQ ID NO: 359); or CDR2: AVRWSSTGIYYTQYAD (SEQ ID NO: 360). In some embodiments, the anti-CD4 antibody comprises at least one CDR3 comprising the amino acid sequence of CDR3: SLTATHTYEYDY (SEQ ID NO: 361); CDR3: DRWWRPAGLQWDY (SEQ ID NO: 362); CDR3: RVFDLPNDY (SEQ ID NO: 363); or CDR3: DTYNSNPARWDGYDF (SEQ ID NO: 364).

In some embodiments, the targeting moiety comprises anti-CD4 antibody as described in WO2012145238, the entire disclosures of which are hereby incorporated by reference. In some embodiments, the anti-CD4 antibody comprises at least one heavy chain variable region comprising the amino acid sequence of CDRH1: AYVIS (SEQ ID NO: 365); CDRH2: EIYPGSGSSYYNEKFKG (SEQ ID NO: 366); or CDRH3: SGDGSKFVY (SEQ ID NO: 367). In some embodiments, the anti-CD4 antibody comprises at least one light chain variable region comprising the amino acid sequence of CDRL1: KASQSVDYCGDSYMN (SEQ ID NO: 368); CDRL2: VASNLES (SEQ ID NO: 369); or CDRL3: QQSLQDPPT (SEQ ID NO: 370).

In some embodiments, the targeting moiety comprises anti-CD4 antibody as described in WO2008134046, the entire disclosures of which are hereby incorporated by reference. In some embodiments, the anti-CD4 antibody comprises at least one heavy chain variable region comprising the amino acid sequence of CDRH1: GYTFTSYVIH (SEQ ID NO: 371); CDRH2: YINPYNDGTDYDEKFK (SEQ ID NO: 372); or CDRH3: EKDNYATGAWFAY (SEQ ID NO: 373). In some embodiments, the anti-CD4 antibody comprises at least one light chain variable region comprising the amino acid sequence of CDRL1: KSSQSLLYSTNQKNY (SEQ ID NO: 374); CDRL2: WASTRES (SEQ ID NO: 375); or CDRL3: QQYYSYRT (SEQ ID NO: 376).

In some embodiments, the targeting moiety comprises anti-CD4 antibody as described in WO2009012944, the entire disclosures of which are hereby incorporated by reference. In some embodiments, the anti-CD4 antibody comprises at least one heavy chain variable region comprising the amino acid sequence of CDRH1: SYVIH (SEQ ID NO: 377); CDRH1: GFTFSNYAMS (SEQ ID NO: 378); or CDRH2: AISDHSTNTYYP (SEQ ID NO: 379); CDRH3: EKDNYATGAWFAY (SEQ ID NO: 380); or CDRH3: ARKYGGDYDPF (SEQ ID NO: 381). In some embodiments, the anti-CD4 antibody comprises at least one light chain variable region comprising the amino acid sequence of CDRL1: KSSQSLLYSTNQKNYL (SEQ ID NO: 382); CDRL1: KSSGSLLYSTNQKNYL (SEQ ID NO: 383); CDRL1: KASQDINNY (SEQ ID NO: 384); CDRL2: WASTRES (SEQ ID NO: 385); CDRL2: YTSTLQPGVPS (SEQ ID NO: 386); CDRL3: QQYYSYRT (SEQ ID NO: 387); or CDRL3: YDNLLF (SEQ ID NO: 388).

In some embodiments, the targeting moiety comprises anti-CD4 antibody as described in WO2004005350, the entire disclosures of which are hereby incorporated by reference. In some embodiments, the anti-CD4 antibody comprises at least one heavy chain variable region comprising the amino acid sequence of CDRH1: TFGVH (SEQ ID NO: 389); CDRH1: TAGVH (SEQ ID NO: 390); or CDRH1: TFGVA (SEQ ID NO: 391); CDRH2: VIWRSGITDYNVPFMS (SEQ ID NO: 392); CDRH2: VIARSGITDYNVPFMS (SEQ ID NO: 393); CDRH2: VIWASGITDYNVPFMS (SEQ ID NO: 394); CDRH3: NDPGTGFAY (SEQ ID NO: 395); CDRH3: NDPGTGAAY (SEQ ID NO: 396); or CDRH3: NDPGTGFAA (SEQ ID NO: 397). In some embodiments, the anti-CD4 antibody comprises at least one light chain variable region comprising the amino acid sequence of CDRL1: RASENIYSYLA (SEQ ID NO: 398); CDRL1: RASENIYSALA (SEQ ID NO: 399); CDRL2: DAKTLAE (SEQ ID NO: 400); CDRL3: QHHYGNPPT (SEQ ID NO: 401); CDRL3: QHAYGNPPT (SEQ ID NO: 402); or CDRL3: QHHAGNPPT (SEQ ID NO: 403).

In some embodiments, the targeting moiety comprises anti-CD4 antibody as described in WO2004083247, the entire disclosures of which are hereby incorporated by reference. In some embodiments, the anti-CD4 antibody comprises at least one heavy chain variable region comprising the amino acid sequence of CDRH1: DYVIN (SEQ ID NO: 404); CDRH2: EIYPGSGSDYYNENLKD (SEQ ID NO: 405); or CDRH3: KGENGNSLAFAY (SEQ ID NO: 406). In some embodiments, the anti-CD4 antibody comprises at least one light chain variable region comprising the amino acid sequence of CDRL1: QSVDYDGDSYMN (SEQ ID NO: 407); CDRL2: AASNLES (SEQ ID NO: 408); or CDRL3: QQSIQDPCT (SEQ ID NO: 409).

In some embodiments, the targeting moiety comprises anti-CD4 antibody as described in WO2014100139, the entire disclosures of which are hereby incorporated by reference. In some embodiments, the anti-CD4 antibody comprises at least one heavy chain comprising the following amino acid sequence:

Anti-CD4 antibody MV1, Heavy Chain
(SEQ ID NO: 410)
MEWSGVFMFLLSVTAGVHSQVQLQQSGPEVVKPGASVKMSCKASGYTFTS
YVIHWVRQKPGQGLDWIGYINPYNDGTDYDEKFKGKATLTSDTSTSTAYM
ELSSLRSEDTAVYYCAREKDNYATGAWFAYWGQGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTC
PPCPAPEFEGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPASIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCS
VMHEALHNHYTQKSLSLSPGK

In some embodiments, the anti-CD4 antibody comprises at least one light chain comprising the following amino acid sequence:

Anti-CD4 antibody MV1, Light Chain
(SEQ ID NO: 411)
MEWSGVFIFLLSVTAGVHSDIVMTQSPDSLAVSLGERVTMNCKSSQSLLY
STNQKNYLAWYQQKPGQSPKLLIYWASTRESGVPDRFSGSGSGTDFTLTI
SSVQAEDVAVYYCQQYYYRTFGGGTKLEIKRTVAAPSVFIFPPSDEQLKS
GTASWVCLLNNFYPREAKVQWKVDNALQSGNSQESVTEQDSKDSTYSLSS
TLTLSKADYEKHKVYACEVTHQGLSSPVTKSFNRGEC

In some embodiments, the targeting moiety comprises anti-CD4 antibody as described in WO2004083247, the entire disclosures of which are hereby incorporated by reference. In some embodiments, the anti-CD4 antibody comprises at least one heavy chain comprising the following amino acid sequence:

(SEQ ID NO: 412)
EEQLVESGGGLVKPGGSLRLSCAASGFSFSDCRMYWLRQAPGKGLEWIGV
ISVKSENYGANYAESVRGRFTISRDDSKNTVYLQMNSLKTEDTAVYYCSA
SYYRYDVGAFAYGQGTLVTVSS

In some embodiments, the anti-CD4 antibody comprises at least one light chain comprising the following amino acid sequence:

(SEQ ID NO: 413)
DIVMTQSPDSLAVSLGERATINCRASKSVSTSGYSYIYWYQQKPGQPPKL
LIYLASILESGVPDRFSGSGSGTDFTLTISSLQAEDVAVYYCQHSRELPT
FGQGTKVEIK

In one embodiment, the vaccine compositions, adjuvants, chimeric proteins or chimeric protein complexes comprises one or more targeting moieties directed against a B cell and one or more targeting moieties directed against the same or another B cell. In one embodiment, the vaccine compositions, adjuvants, chimeric proteins or chimeric protein complexes comprises one or more targeting moieties directed against a B cell and one or more targeting moieties directed against a T cell. In one embodiment, the vaccine compositions, adjuvants, chimeric proteins or chimeric protein complexes comprises one or more targeting moieties directed against a B cell and one or more targeting moieties directed against a dendritic cell. In one embodiment, the vaccine compositions, adjuvants, chimeric proteins or chimeric protein complexes comprises one or more targeting moieties against a B cell and one or more targeting moieties directed against a macrophage. In one embodiment, the vaccine compositions, adjuvants, chimeric proteins or chimeric protein complexes comprises one or more targeting moieties against a B cell and one or more targeting moieties directed against a NK cell.

In one embodiment, the vaccine compositions, adjuvants, chimeric proteins or chimeric protein complexes comprises one or more targeting moieties directed against a dendritic cell and one or more targeting moieties directed against the same or another dendritic cell. In one embodiment, the vaccine compositions, adjuvants, chimeric proteins or chimeric protein complexes comprises one or more targeting moieties directed against a dendritic cell and one or more targeting moieties directed against a T cell. In one embodiment, the vaccine compositions, adjuvants, chimeric proteins or chimeric protein complexes comprises one or more targeting moieties directed against a dendritic cell and one or more targeting moieties directed against a B cell. In one embodiment, the vaccine compositions, adjuvants, chimeric proteins or chimeric protein complexes comprises one or more targeting moieties against a dendritic cell and one or more targeting moieties directed against a macrophage. In one embodiment, the vaccine compositions, adjuvants, chimeric proteins or chimeric protein complexes comprises one or more targeting moieties against a dendritic cell and one or more targeting moieties directed against a NK cell.

In one embodiment, the vaccine compositions, adjuvants, chimeric proteins or chimeric protein complexes comprises one or more targeting moieties directed against a macrophage and one or more targeting moieties directed against the same or another macrophage. In one embodiment, the vaccine compositions, adjuvants, chimeric proteins or chimeric protein complexes comprises one or more targeting moieties directed against a macrophage and one or more targeting moieties directed against a T cell. In one embodiment, the vaccine compositions, adjuvants, chimeric proteins or chimeric protein complexes comprises one or more targeting moieties directed against a macrophage and one or more targeting moieties directed against a B cell. In one embodiment, the vaccine compositions, adjuvants, chimeric proteins or chimeric protein complexes comprises one or more targeting moieties against a macrophage and one or more targeting moieties directed against a dendritic cell. In one embodiment, the vaccine compositions, adjuvants, chimeric proteins or chimeric protein complexes comprises one or more targeting moieties against a macrophage and one or more targeting moieties directed against a NK cell.

In one embodiment, the vaccine compositions, adjuvants, chimeric proteins or chimeric protein complexes comprises one or more targeting moieties directed against an NK cell and one or more targeting moieties directed against the same or another NK cell. In one embodiment, the vaccine compositions, adjuvants, chimeric proteins or chimeric protein complexes comprises one or more targeting moieties directed against an NK cell and one or more targeting moieties directed against a T cell. In one embodiment, the vaccine compositions, adjuvants, chimeric proteins or chimeric protein complexes comprises one or more targeting moieties directed against an NK cell and one or more targeting moieties directed against a B cell. In one embodiment, the vaccine compositions, adjuvants, chimeric proteins or chimeric protein complexes comprises one or more targeting moieties against an NK cell and one or more targeting moieties directed against a macrophage. In one embodiment, the vaccine compositions, adjuvants, chimeric proteins or chimeric protein complexes comprises one or more targeting moieties against an NK cell and one or more targeting moieties directed against a dendritic cell.

In one embodiment, the vaccine compositions, adjuvants, chimeric proteins or chimeric protein complexes comprises a targeting moiety directed against a tumor cell and a second targeting moiety directed against the same or a different tumor cell. In such embodiments, the targeting moieties may bind to any of the tumor antigens described herein.

In some embodiments, the vaccine compositions, adjuvants, chimeric proteins or chimeric protein complexes of the invention comprises one or more targeting moieties having recognition domains that bind to a target (e.g. antigen, receptor) of interest including those found on one or more cells selected from adipocytes (e.g., white fat cell, brown fat cell), liver lipocytes, hepatic cells, kidney cells (e.g., kidney parietal cell, kidney salivary gland, mammary gland, etc.), duct cells (of seminal vesicle, prostate gland, etc.), intestinal brush border cells (with microvilli), exocrine gland striated duct cells, gall bladder epithelial cells, ductulus efferens nonciliated cells, epididymal principal cells, epididymal basal cells, endothelial cells, ameloblast epithelial cells (tooth enamel secretion), planum semilunatum epithelial cells of vestibular system of ear (proteoglycan secretion), organ of Corti interdental epithelial cells (secreting tectorial membrane covering hair cells), loose connective tissue fibroblasts, corneal fibroblasts (corneal keratocytes), tendon fibroblasts, bone marrow reticular tissue fibroblasts, nonepithelial fibroblasts, pericytes, nucleus pulposus cells of intervertebral disc, cementoblasts/cementocytes (tooth root bonelike ewan cell secretion), odontoblasts/odontocytes (tooth dentin secretion), hyaline cartilage chondrocytes, fibrocartilage chondrocytes, elastic cartilage chondrocytes, osteoblasts/osteocytes, osteoprogenitor cells (stem cell of osteoblasts), hyalocytes of vitreous body of eye, stellate cells of perilymphatic space of ear, hepatic stellate cells (Ito cell), pancreatic stelle cells, skeletal muscle cells, satellite cells, heart muscle cells, smooth muscle cells, myoepithelial cells of iris, myoepithelial cells of exocrine glands, exocrine secretory epithelial cells (e.g., salivary gland cells, mammary gland cells, lacrimal gland cells, sweat gland cells, sebaceious gland cells, prostate gland cells, gastric glad cells, pancreatic acinar cells, pneumocytes), a hormone secreting cells (e.g., pituitary cells, neurosecretory cells, gut and respiratory tract cells, thyroid gland cells, parathyroid glad cells, adrenal gland cells, Leydig cells of testes, pancreatic islet cells), keratinizing epithelial cells, wet stratified barrier epithelial cells, neuronal cells (e.g., sensory transducer cells, autonomic neuron cells, sense organ and peripheral neuron supporting cells, and central nervous system neurons and glial cells such as interneurons, principal cells, astrocytes, oligodendrocytes, and ependymal cells).

### Targeting Moiety Formats

In various embodiments, the targeting moiety of the vaccine composition, adjuvant, chimeric protein, or chimeric protein complex is a protein-based agent capable of specific binding, such as an antibody or derivatives thereof. In an embodiment, the targeting moiety comprises an antibody. In various embodiments, the antibody is a full-length multimeric protein that includes two heavy chains and two light chains. Each heavy chain includes one variable region (e.g., V_{H}) and at least three constant regions (e.g., CH1, CH2 and CH3), and each light chain includes one variable region (V_{L}) and one constant region (C_{L}). The variable regions determine the specificity of the antibody. Each variable region comprises three hypervariable regions also known as complementarity-determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the targeting moiety comprises antibody derivatives or formats. In some embodiments, the targeting moiety of the vaccine composition, adjuvant, chimeric protein, or chimeric protein complex is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterases; a plastic antibodies; a phylomer; a stradobodies; a maxibodies; an evibody; a fynomer, an armadillo repeat protein, a Kunitz domain, an avimer, an atrimer, a probody, an immunobody, a triomab, a troybody; a pepbody; a vaccibody, a UniBody; affimers, a DuoBody, a Fv, a Fab, a Fab′, a F(ab′)₂, a peptide mimetic molecule, or a synthetic molecule, as described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3 (3): 310-317.

In one embodiment, the targeting moiety comprises a single-domain antibody, such as VHH from, for example, an organism that produces VHH antibody such as a camelid, a shark, or a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3). VHHs are commercially available under the trademark of NANOBODY or NANOBODIES.

In an embodiment, the targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. a HUMABODIES are described in, for example, WO 2016/113555 and WO2016/113557, the entire disclosure of which is incorporated by reference.

In various embodiments, the targeting moiety of the vaccine composition, adjuvant, chimeric protein, or chimeric protein complex is a protein-based agent capable of specific binding to a cell receptor, such as a natural ligand for the cell receptor. In various embodiments, the cell receptor is found on one or more immune cells, which can include, without limitation, T cells, cytotoxic T lymphocytes, T helper cells, natural killer (NK) cells, natural killer T (NKT) cells, anti-tumor macrophages (e.g. M1 macrophages), B cells, dendritic cells, or subsets thereof. In some embodiments, the cell receptor is found on megakaryocytes, thrombocytes, erythrocytes, mast cells, basophils, neutrophils, eosinophils, or subsets thereof.

In some embodiments, the targeting moiety is a natural ligand such as a chemokine. Illustrative chemokines that may be included in the chimeric proteins or chimeric protein complexes such as Fc-based chimeric protein complex of the invention include, but are not limited to, CCL1, CCL2, CCL4, CCL5, CCL6, CCL7, CCL8, CCL9, CCL10, CCL11, CCL12, CCL13, CCL14, CCL15, CCL16, C_{L}17, CCL18, CCL19, CCL20, CCL21, CCL22, CCL23, CCL24, CLL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, HCC-4, and LDGF-PBP. In an illustrative embodiment, the targeting moiety may be XCL1, which is a chemokine that recognizes and binds to the dendritic cell receptor XCR1. In another illustrative embodiment, the targeting moiety is CCL1, which is a chemokine that recognizes and binds to CCR8. In another illustrative embodiment, the targeting moiety is CCL2, which is a chemokine that recognizes and binds to CCR2 or CCR9. In another illustrative embodiment, the targeting moiety is CCL3, which is a chemokine that recognizes and binds to CCR1, CCR5, or CCR9. In another illustrative embodiment, the targeting moiety is CCL4, which is a chemokine that recognizes and binds to CCR1 or CCR5 or CCR9. In another illustrative embodiment, the targeting moiety is CCL5, which is a chemokine that recognizes and binds to CCR1 or CCR3 or CCR4 or CCR5.

In another illustrative embodiment, the targeting moiety is CCL6, which is a chemokine that recognizes and binds to CCR1. In another illustrative embodiment, the targeting moiety is CCL7, which is a chemokine that recognizes and binds to CCR2 or CCR9. In another illustrative embodiment, the targeting moiety is CCL8, which is a chemokine that recognizes and binds to CCR1 or CCR2 or CCR2B or CCR5 or CCR9. In another illustrative embodiment, the targeting moiety is CCL9, which is a chemokine that recognizes and binds to CCR1. In another illustrative embodiment, the targeting moiety is CCL 10, which is a chemokine that recognizes and binds to CCR1. In another illustrative embodiment, the targeting moiety is CCL11, which is a chemokine that recognizes and binds to CCR2 or CCR3 or CCR5 or CCR9. In another illustrative embodiment, the targeting moiety is CCL13, which is a chemokine that recognizes and binds to CCR2 or CCR3 or CCR5 or CCR9. In another illustrative embodiment, the targeting moiety is CCL14, which is a chemokine that recognizes and binds to CCR1 or CCR9. In another illustrative embodiment, the targeting moiety is CCL15, which is a chemokine that recognizes and binds to CCR1 or CCR3. In another illustrative embodiment, the targeting moiety is CCL16, which is a chemokine that recognizes and binds to CCR1, CCR2, CCR5, or CCR8. In another illustrative embodiment, the targeting moiety is CCL17, which is a chemokine that recognizes and binds to CCR4. In another illustrative embodiment, the targeting moiety is CCL19, which is a chemokine that recognizes and binds to CCR7. In another illustrative embodiment, the targeting moiety is CCL20, which is a chemokine that recognizes and binds to CCR6. In another illustrative embodiment, the targeting moiety is CCL21, which is a chemokine that recognizes and binds to CCR7. In another illustrative embodiment, the targeting moiety is CCL22, which is a chemokine that recognizes and binds to CCR4. In another illustrative embodiment, the targeting moiety is CCL23, which is a chemokine that recognizes and binds to CCR1. In another illustrative embodiment, the targeting moiety is CCL24, which is a chemokine that recognizes and binds to CCR3. In another illustrative embodiment, the targeting moiety is CCL25, which is a chemokine that recognizes and binds to CCR9. In another illustrative embodiment, the targeting moiety is CCL26, which is a chemokine that recognizes and binds to CCR3. In another illustrative embodiment, the targeting moiety is CCL27, which is a chemokine that recognizes and binds to CCR10. In another illustrative embodiment, the targeting moiety is CCL28, which is a chemokine that recognizes and binds to CCR3 or CCR10. In another illustrative embodiment, the targeting moiety is CXCL1, which is a chemokine that recognizes and binds to CXCR1 or CXCR2. In another illustrative embodiment, the targeting moiety is CXCL2, which is a chemokine that recognizes and binds to CXCR2. In another illustrative embodiment, the targeting moiety is CXCL3, which is a chemokine that recognizes and binds to CXCR2. In another illustrative embodiment, the targeting moiety is CXCL4, which is a chemokine that recognizes and binds to CXCR3B. In another illustrative embodiment, the targeting moiety is CXCL5, which is a chemokine that recognizes and binds to CXCR2. In another illustrative embodiment, the targeting moiety is CXCL6, which is a chemokine that recognizes and binds to CXCR1 or CXCR2. In another illustrative embodiment, the targeting moiety is CXCL8, which is a chemokine that recognizes and binds to CXCR1 or CXCR2. In another illustrative embodiment, the targeting moiety is CXCL9, which is a chemokine that recognizes and binds to CXCR3. In another illustrative embodiment, the targeting moiety is CXCL10, which is a chemokine that recognizes and binds to CXCR3. In another illustrative embodiment, the targeting moiety is CXCL11, which is a chemokine that recognizes and binds to CXCR3 or CXCR7. In another illustrative embodiment, the targeting moiety is CXCL12, which is a chemokine that recognizes and binds to CXCR4 or CXCR7. In another illustrative embodiment, the targeting moiety is CXCL13, which is a chemokine that recognizes and binds to CXCR5. In another illustrative embodiment, the targeting moiety is CXCL16, which is a chemokine that recognizes and binds to CXCR6. In another illustrative embodiment, the targeting moiety is LDGF-PBP, which is a chemokine that recognizes and binds to CXCR2. In another illustrative embodiment, the targeting moiety is XCL2, which is a chemokine that recognizes and binds to XCR1. In another illustrative embodiment, the targeting moiety is CX3C_{L}1, which is a chemokine that recognizes and binds to CX3CR1.

In some embodiments, the targeting moiety is a natural ligand such as FMS-like tyrosine kinase 3 ligand (Flt3L) or a truncated region thereof (e.g., which is able to bind Flt3). In some embodiments, the targeting moiety is an extracellular domain of Flt3L. In some embodiments, the targeting moiety comprising a Flt3L domain, wherein the FIt3L domain is a single chain dimer, optionally where one FIt3L domain is connected to the other FIt3L domain via one or more linkers, wherein the linker is a flexible linker. In some embodiments, the targeting moiety of the present invention comprises Flt3L domain, wherein the FIt3L domain is a single chain dimer and an Fc domain, the Fc domain optionally having one or more mutations that reduces or eliminates one or more effector functions of the Fc domain, promotes Fc chain pairing in the Fc domain, and/or stabilizes a hinge region in the Fc domain. In some embodiments, the targeting moiety recognizes CD20. In some embodiments, the targeting moiety recognizes PD-L1. In some embodiments, the targeting moiety recognizes Clec9A.

In various embodiments, the vaccine composition, adjuvant, chimeric protein, or chimeric protein complex comprises targeting moieties in various combinations. In an illustrative embodiment, the vaccine composition, adjuvant, chimeric protein, or chimeric protein complex may comprise two targeting moieties, wherein both targeting moieties are antibodies or derivatives thereof. In another illustrative embodiment, the vaccine composition, adjuvant, chimeric protein, or chimeric protein complex may comprise two targeting moieties, wherein both targeting moieties are natural ligands for cell receptors. In a further illustrative embodiment, the vaccine composition, adjuvant, chimeric protein, or chimeric protein complex may comprise two targeting moieties, wherein one of the targeting moieties is an antibody or derivative thereof, and the other targeting moiety is a natural ligand for a cell receptor.

In various embodiments, the recognition domain of the vaccine composition, adjuvant, chimeric protein, or chimeric protein complex functionally modulates (by way of non-limitation, partially or completely neutralizes) the target (e.g. antigen, receptor) of interest, e.g. substantially inhibiting, reducing, or neutralizing a biological effect that the antigen has. For example, various recognition domains may be directed against one or more tumor antigens that are actively suppressing, or have the capacity to suppress, the immune system of, for example, a patient bearing a tumor. For example, in some embodiments, the vaccine composition, adjuvant, chimeric protein, or chimeric protein complex functionally modulates immune inhibitory signals (e.g. checkpoint inhibitors), for example, one or more of TIM-3, BTLA, PD-1, CTLA-4, B7-H4, GITR, galectin-9, HVEM, PD-L1, PD-L2, B7-H3, CD244, CD160, TIGIT, SIRPα, ICOS, CD172a, and TMIGD2. For example, in some embodiments, the vaccine composition, adjuvant, chimeric protein, or chimeric protein complex is engineered to disrupt, block, reduce, and/or inhibit the transmission of an immune inhibitory signal, by way of non-limiting example, the binding of PD-1 with PD-L1 or PD-L2 and/or the binding of CTLA-4 with one or more of AP2M1, CD80, CD86, SHP-2, and PPP2R5A.

In various embodiments, the recognition domain of the vaccine composition, adjuvant, chimeric protein, or chimeric protein complex binds but does not functionally modulate the target (e.g. antigen, receptor) of interest, e.g. the recognition domain is, or is akin to, a binding antibody. For instance, in various embodiments, the recognition domain simply targets the antigen or receptor but does not substantially inhibit, reduce or functionally modulate a biological effect that the antigen or receptor has. For example, some of the smaller antibody formats described above (e.g. as compared to, for example, full antibodies) have the ability to target hard to access epitopes and provide a larger spectrum of specific binding locales. In various embodiments, the recognition domain binds an epitope that is physically separate from an antigen or receptor site that is important for its biological activity (e.g. the antigen's active site).

Such non-neutralizing binding finds use in various embodiments of the present invention, including methods in which the vaccine composition, adjuvant, chimeric protein, or chimeric protein complex is used to directly or indirectly recruit active immune cells to a site of need via an effector antigen, such as any of those described herein. For example, in various embodiments, the vaccine composition, adjuvant, chimeric protein, or chimeric protein complex such as Fc-based chimeric protein complex may be used to directly or indirectly recruit cytotoxic T cells via CD8 to a tumor cell in a method of reducing or eliminating a tumor (e.g. the chimeric proteins or chimeric protein complexes such as Fc-based chimeric protein complex may comprise an anti-CD8 recognition domain and a recognition domain directed against a tumor antigen). In such embodiments, it is desirable to directly or indirectly recruit CD8-expressing cytotoxic T cells but not to functionally modulate the CD8 activity. On the contrary, in these embodiments, CD8 signaling is an important piece of the tumor reducing or eliminating effect. By way of further example, in various methods of reducing or eliminating tumors, the vaccine composition, adjuvant, chimeric protein, or chimeric protein complex is used to directly or indirectly recruit dendritic cells (DCs) via CLEC9A (e.g. the chimeric proteins or chimeric protein complexes such as Fc-based chimeric protein complex may comprise an anti-CLEC9A recognition domain and a recognition domain directed against a tumor antigen). In such embodiments, it is desirable to directly or indirectly recruit CLEC9A-expressing DCs but not to functionally modulate the CLEC9A activity. On the contrary, in these embodiments, CLEC9A signaling is an important piece of the tumor reducing or eliminating effect.

In various embodiments, the recognition domain of the vaccine composition, adjuvant, chimeric protein, or chimeric protein complex binds to XCR1 e.g. on dendritic cells. For instance, the recognition domain, in some embodiments comprises all or part of XCL1 or a non-neutralizing anti-XCR1 agent.

In various embodiments, the recognition domain of the vaccine composition, adjuvant, chimeric protein, or chimeric protein complex binds to an immune modulatory antigen (e.g. immune stimulatory or immune inhibitory). In various embodiments, the immune modulatory antigen is one or more of 4-1BB, OX-40, HVEM, GITR, CD27, CD28, CD30, CD40, ICOS ligand; OX-40 ligand, LIGHT (CD258), GITR ligand, CD70, B7-1, B7-2, CD30 ligand, CD40 ligand, ICOS, ICOS ligand, CD137 ligand and TL1A. In various embodiments, such immune stimulatory antigens are expressed on a tumor cell. In various embodiments, the recognition domain of the vaccine composition, adjuvant, chimeric protein, or chimeric protein complex binds but does not functionally modulate such immune stimulatory antigens and therefore allows recruitment of cells expressing these antigens without the reduction or loss of their potential tumor reducing or eliminating capacity.

In various embodiments, the recognition domain of the vaccine composition, adjuvant, chimeric protein, or chimeric protein complex may be in the context of chimeric proteins or chimeric protein complexes such as Fc-based chimeric protein complex that comprises two recognition domains that have neutralizing activity, or comprises two recognition domains that have non-neutralizing (e.g. binding) activity, or comprises one recognition domain that has neutralizing activity and one recognition domain that has non-neutralizing (e.g. binding) activity.

### Fc Domains

The fragment crystallizable domain (Fc domain) is the tail region of an antibody that interacts with Fc receptors located on the cell surface of cells that are involved in the immune system, e.g., B lymphocytes, dendritic cells, natural killer cells, macrophages, neutrophils, eosinophils, basophils, and mast cells. In IgG, IgA and IgD antibody isotypes, the Fc domain is composed of two identical protein fragments, derived from the second and third constant domains of the antibody's two heavy chains. In IgM and IgE antibody isotypes, the Fc domain contains three heavy chain constant domains (CH domains 2-4) in each polypeptide chain.

In some embodiments, the chimeric protein complex (e.g., a Fc-based chimeric protein complex) includes a Fc domain. In some embodiments, the Fc domains are from selected from IgG, IgA, IgD, IgM or IgE. In some embodiments, the Fc domains are from selected from IgG1, IgG2, IgG3, or IgG4.

In some embodiments, the Fc domains are from selected from human IgG, IgA, IgD, IgM or IgE. In some embodiments, the Fc domains are from selected from human IgG1, IgG2, IgG3, or IgG4.

In some embodiments, the Fc domains of the chimeric protein complex (e.g., a Fc-based chimeric protein complex) comprise the CH2 and CH3 regions of IgG. In some embodiments, the IgG is human IgG. In some embodiments, the human IgG is selected from IgG1, IgG2, IgG3, or IgG4.

In some embodiments, the Fc domains comprise one or more mutations. In some embodiments, the mutation(s) to the Fc domains reduces or eliminates the effector function the Fc domains. In some embodiments, the mutated Fc domain has reduced affinity or binding to a target receptor. By way of example, in some embodiments, the mutation to the Fc domains reduces or eliminates the binding of the Fc domains to FcγR. In some embodiments, the FcγR is selected from FcγRI; FcγRIIa, 131 R/R; FcγRIIa, 131 H/H, FcγRIIb; and FcγRIII. In some embodiments, the mutation to the Fc domains reduces or eliminated binding to complement proteins, such as, e.g., Clq. In some embodiments, the mutation to the Fc domains reduces or eliminated binding to both FcγR and complement proteins, such as, e.g., Clq.

In some embodiments, the Fc domains comprise the LALA mutation to reduce or eliminate the effector function of the Fc domains. By way of example, in some embodiments, the LALA mutation comprises L234A and L235A substitutions in human IgG (e.g., IgG1) (wherein the numbering is based on the commonly used numbering of the CH2 residues for human IgG1 according to EU convention (PNAS, Edelman et al., 1969; 63 (1) 78-85)).

In some embodiments, the Fc domains of human IgG comprise a mutation to reduce or eliminate the effector function of the Fc domains. By way of example, in some embodiments, the mutations are selected from L234A, L234F, L235A, L235E, L235Q, K322A, K322Q, D265A, P329G, P329A, P331G, and P331S.

In some embodiments, the Fc domains comprise the FALA mutation to reduce or eliminate the effector function of the Fc domains. By way of example, in some embodiments, the FALA mutation comprises F234A and L235A substitutions in human IgG4.

In some embodiments, the Fc domains of human IgG4 comprise a mutation at one or more of F234, L235, K322, D265, and P329 to reduce or eliminate the effector function of the Fc domains. By way of example, in some embodiments, the mutations are selected from F234A, L235A, L235E, L235Q, K322A, K322Q, D265A, P329G, and P329A.

In some embodiments, the mutation(s) to the Fc domain stabilize a hinge region in the Fc domain. By way of example, in some embodiments, the Fc domain comprises a mutation at S228 of human IgG to stabilize a hinge region. In some embodiments, the mutation is S228P.

In some embodiments, the mutation(s) to the Fc domain promote chain pairing in the Fc domain. In some embodiments, chain pairing is promoted by ionic pairing (a/k/a charged pairs, ionic bond, or charged residue pair).

In some embodiments, the Fc domain comprises a mutation at one more of the following amino acid residues of IgG to promote of ionic pairing: D356, E357, L368, K370, K392, D399, and K409.

By way of example, in some embodiments, the human IgG Fc domain comprise one of the mutation combinations in Table 1 to promote of ionic pairing.

TABLE 1

| Substitution(s) on one Fc Chain | Substitution(s) on other Fc Chain |
|---|---|
| D356K D399K | K392D K409D |
| E357R L368R | K370D K409D |
| E357R L368K | K370D K409D |
| E357R D399K | K370D K409D |
| E357R | K370D |
| L368R D399K | K392D K409D |
| L368K D399K | K392D K409D |
| L368R D399K | K409D |
| L368K D399K | K409D |
| L368R | K409D |
| L368K | K409D |
| K370D K409D | E357R D399K |
| K370D K409D | E357R L368R |
| K370D K409D | E357R L368K |
| K370D K409D | E357R D399K |
| K370D K409D | E357R L368R |
| K370D K409D | E357R L368K |
| K370D | E357R |
| K370D | E357R |
| K392D K409D | D356K D399K |
| K392D K409D | L368R D399K |
| K392D K409D | L368K D399K |
| K392D K409D | D399K |
| D399K | K392D K409D |
| D399K | K409D |
| K409D | L368R |
| K409D | L368K |
| K409D | L368R D399K |
| K409D | L368K D399K |
| K409D | L368R |
| K409D | L368K |
| K409D | L368R D399K |
| K409D | L368K D399K |
| K409D | D399K |

In some embodiments, chain pairing is promoted by a knob-in-hole mutations. In some embodiments, the Fc domain comprises one or more mutations to allow for a knob-in-hole interaction in the Fc domain. In some embodiments, a first Fc chain is engineered to express the “knob” and a second Fc chain is engineered to express the complementary “hole.” By way of example, in some embodiments, human IgG Fc domain comprises the mutations of Table 2 to allow for a knob-in-hole interaction.

TABLE 2

| Substitution(s) on one Fc Chain | Substitution(s) on other Fc Chain |
|---|---|
| T366Y | Y407T |
| T366Y/F405A | T394W/Y407T |
| T366W | Y407A |
| T366W | Y407V |
| T366Y | Y407A |
| T366Y | Y407V |
| T366Y | Y407T |

In some embodiments, the Fc domains in the chimeric protein complexes (e.g., Fc-based chimeric protein complexes) of the present technology comprise any combination of the above-disclosed mutations. By way of example, in some embodiments, the Fc domain comprises mutations that promote ionic pairing and/or a knob-in-hole interaction. By way of example, in some embodiments, the Fc domain comprises mutations that have one or more of the following properties: promote ionic pairing, induce a knob-in-hole interaction, reduce or eliminate the effector function of the Fc domain, and cause Fc stabilization (e.g. at hinge).

By way of example, in some embodiments, a human IgG Fc domains comprise mutations disclosed in Table 3, which promote ionic pairing and/or promote a knob-in-hole interaction in the Fc domain.

TABLE 3

| Substitution(s) on one Fc Chain | Substitution(s) on other Fc Chain |
|---|---|
| T366W K370D | E357R Y407A |
| T366W K370D | E357R Y407V |
| T366W K409D | L368R Y407A |
| T366W K409D | L368R Y407V |
| T366W K409D | L368K Y407A |
| T366W K409D | L368K Y407V |
| T366W K409D | L368R D399K Y407A |
| T366W K409D | L368R D399K Y407V |
| T366W K409D | L368K D399K Y407A |
| T366W K409D | L368K D399K Y407V |
| T366W K409D | D399K Y407A |
| T366W K409D | D399K Y407V |
| T366W K392D K409D | D399K Y407A |
| T366W K392D K409D | D399K Y407V |
| T366W K392D K409D | D356K D399K Y407A |
| T366W K392D K409D | D356K D399K Y407V |
| T366W K370D K409D | E357R D399K Y407A |
| T366W K370D K409D | E357R D399K Y407V |
| T366W K370D K409D | E357R L368R Y407A |
| T366W K370D K409D | E357R L368R Y407V |
| T366W K370D K409D | E357R L368K Y407A |
| T366W K370D K409D | E357R L368K Y407V |
| T366W K392D K409D | L368R D399K Y407A |
| T366W K392D K409D | L368R D399K Y407V |
| T366W K392D K409D | L368K D399K Y407A |
| T366W K392D K409D | L368K D399K Y407V |
| E357R T366W | K370D Y407A |
| E357R T366W | K370D Y407V |
| T366W L368R | Y407A K409D |
| T366W L368R | Y407V K409D |
| T366W L368K | Y407A K409D |
| T366W L368K | Y407V K409D |
| T366W L368R D399K | Y407A K409D |
| T366W L368R D399K | Y407V K409D |
| T366W L368K D399K | Y407A K409D |
| T366W L368K D399K | Y407V K409D |
| T366W D399K | Y407A K409D |
| T366W D399K | Y407V K409D |
| 1366W D399K | K392D Y407A K409D |
| T366W D399K | K392D Y407V K409D |
| T366W D356K D399K | K392D Y407A K409D |
| T366W D356K D399K | K392D Y407V K409D |
| E357R T366W D399K | K370D Y407A K409D |
| E357R T366W D399K | K370D Y407V K409D |
| E357R T366W L368R | K370D Y407A K409D |
| E357R T366W L368R | K370D Y407V K409D |
| E357R T366W L368K | K370D Y407A K409D |
| E357R T366W L368K | K370D Y407V K409D |
| T366W L368R D399K | K392D Y407A K409D |
| T366W L368R D399K | K392D Y407V K409D |
| T366W L368K D399K | K392D Y407A K409D |

By way of example, in some embodiments, a human IgG Fc domains comprise mutations disclosed in Table 4, which promote ionic pairing, promote a knob-in-hole interaction, or a combination thereof in the Fc domain. In embodiments, the “Chain 1” and “Chain 2” of Table 4 can be interchanged (e.g. Chain 1 can have Y407T and Chain 2 can have T366Y).

TABLE 4

| Chain 1 mutation | Chain 2 mutation | Reference | IgG |
|---|---|---|---|
| T366Y | Y407T | Ridgway et al., 1996 Protein | IgG1 |
| | | Engineering, Design and | |
| | | Selection, Volume 9, Issue 7, 1 | |
| | | Jul. 1996, Pages 617-62 | |
| T366Y/F405A | T394W/Y407T | Ridgway et al., 1996 Protein | IgG1 |
| | | Engineering, Design and | |
| | | Selection, Volume 9, Issue 7, 1 | |
| | | Jul. 1996, Pages 617-62 | |
| T366W | Y407A | Atwell et al., 1997 JMB | IgG1 |
| | | Volume 270, Issue 1, 4 Jul. | |
| | | 1997, Pages 26-35 | |
| T366W | T366S/L368V/Y407A | Atwell et al., 1997 JMB | IgG1 |
| | | Volume 270, Issue 1, 4 Jul. | |
| | | 1997, Pages 26-35 | |
| T366W | L368A/Y407A | Atwell et al., 1997 JMB | IgG1 |
| | | Volume 270, Issue 1, 4 Jul. | |
| | | 1997, Pages 26-35 | |
| T366W | T366S/L368A/Y407A | Atwell et al., 1997 JMB | IgG1 |
| | | Volume 270, Issue 1, 4 Jul. | |
| | | 1997, Pages 26-35 | |
| T366W | T366S/L368G/Y407V | Atwell et al., 1997 JMB | IgG1 |
| | | Volume 270, Issue 1, 4 Jul. | |
| | | 1997, Pages 26-35 | |
| T366W/D399C | T366S/L368A/K392C/Y407V | Merchant et al., 1998 Nature | IgG1 |
| | | Biotechnology volume 16, pages | |
| | | 677-681 (1998) | |
| T366W/K392C | T366S/L368A/D399C/Y407V | Merchant et al., 1998 Nature | IgG1 |
| | | Biotechnology volume 16, pages | |
| | | 677-681 (1998) | |
| S354C/T366W | Y349C/T366S/L368A/Y407V | Merchant et al., 1998 Nature | IgG1 |
| | | Biotechnology volume 16, pages | |
| | | 677-681 (1998) | |
| Y349C/T366W | S354C/T366S/L368A/Y407V | Merchant et al., 1998 Nature | IgG1 |
| | | Biotechnology volume 16, pages | |
| | | 677-681 (1998) | |
| E356C/T366W | Y349C/T366S/L368A/Y407V | Merchant et al., 1998 Nature | IgG1 |
| | | Biotechnology volume 16, pages | |
| | | 677-681 (1998) | |
| Y349C/T366W | E356C/T366S/L368A/Y407V | Merchant et al., 1998 Nature | IgG1 |
| | | Biotechnology volume 16, pages | |
| | | 677-681 (1998) | |
| E357C/T366W | Y349C/T366S/L368A/Y407V | Merchant et al., 1998 Nature | IgG1 |
| | | Biotechnology volume 16, pages | |
| | | 677-681 (1998) | |
| Y349C/T366W | E357C/T366S/L368A/Y407V | Merchant et al., 1998 Nature | IgG1 |
| | | Biotechnology volume 16, pages | |
| | | 677-681 (1998) | |
| D339R | K409E | Gunasekaran et al., 2010 The | IgG1 |
| | | Journal of Biological Chemistry | |
| | | 285, 19637-19646. | |
| D339K | K409E | Gunasekaran et al., 2010 The | IgG1 |
| | | Journal of Biological Chemistry | |
| | | 285, 19637-19646. | |
| D339R | K409D | Gunasekaran et al., 2010 The | IgG1 |
| | | Journal of Biological Chemistry | |
| | | 285, 19637-19646. | |
| D339K | K409D | Gunasekaran et al., 2010 The | IgG1 |
| | | Journal of Biological Chemistry | |
| | | 285, 19637-19646. | |
| D339K | K360D/K409E | Gunasekaran et al., 2010 The | IgG1 |
| | | Journal of Biological Chemistry | |
| | | 285, 19637-19646. | |
| D339K | K392D/K409E | Gunasekaran et al., 2010 The | IgG1 |
| | | Journal of Biological Chemistry | |
| | | 285, 19637-19646. | |
| D339K/E356K | K392D/K409E | Gunasekaran et al., 2010 The | IgG1 |
| | | Journal of Biological Chemistry | |
| | | 285, 19637-19646. | |
| D339K/E357K | K392D/K409E | Gunasekaran et al., 2010 The | IgG1 |
| | | Journal of Biological Chemistry | |
| | | 285, 19637-19646. | |
| D339K/E356K | K409E/K439D | Gunasekaran et al., 2010 The | IgG1 |
| | | Journal of Biological Chemistry | |
| | | 285, 19637-19646. | |
| D339K/E357K | K370D/K409E | Gunasekaran et al., 2010 The | IgG1 |
| | | Journal of Biological Chemistry | |
| | | 285, 19637-19646. | |
| D339K/E356K/E357K | K370D/K392D/K409E | Gunasekaran et al., 2010 The | IgG1 |
| | | Journal of Biological Chemistry | |
| | | 285, 19637-19646. | |
| S364H/F405A | Y349T/T394F | Moore et al., 2011 mAbs, 3: 6, | IgG1 |
| | | 546-557 | |
| S364H/T394F | Y349T/F405A | Moore et al., 2011 mAbs, 3: 6, | IgG1 |
| | | 546-557 | |
| D221R/P228R/K409R | D221E/P228E/L368E | Strop et al., 2012 JMB Volume | IgG1 |
| | | 420, Issue 3, 13 Jul. 2012, | |
| | | Pages 204-219 | |
| C223R/E225R/P228R/K409R | C223E/P228E/L368E | Strop et al., 2012 JMB Volume | IgG2 |
| | | 420, Issue 3, 13 Jul. 2012, | |
| | | Pages 204-219 | |
| F405L | K409R | Labrijn et al., 2013 PNAS Mar. | IgG1 |
| | | 26, 2013. 110 (13) 5145-5150 | |
| F405A/Y407V | T394W | Von Kreudenstein et al., 2013 | IgG1 |
| | | mAbs | |
| | | Volume 5, 2013 - Issue 5, | |
| | | pp. 644-654 | |
| F405A/Y407V | T366I/T394W | Von Kreudenstein et al., 2013 | IgG1 |
| | | mAbs | |
| | | Volume 5, 2013 - Issue 5, | |
| | | pp. 644-654 | |
| F405A/Y407V | T366L/T394W | Von Kreudenstein et al., 2013 | IgG1 |
| | | mAbs | |
| | | Volume 5, 2013 - Issue 5, | |
| | | pp. 644-654 | |
| F405A/Y407V | T366L/K392M/T394W | Von Kreudenstein et al., 2013 | IgG1 |
| | | mAbs | |
| | | Volume 5, 2013 - Issue 5, | |
| | | pp. 644-654 | |
| L351Y/F405A/Y407V | T366L/K392M/T394W | Von Kreudenstein et al., 2013 | IgG1 |
| | | mAbs | |
| | | Volume 5, 2013 - Issue 5, | |
| | | pp. 644-654 | |
| T350V/L351Y/F405A/Y407V | T350V/T366L/K392M/T394W | Von Kreudenstein et al., 2013 | IgG1 |
| | | mAbs | |
| | | Volume 5, 2013 - Issue 5, | |
| | | pp. 644-654 | |
| T350V/L351Y/F405A/Y407V | T350V/T366L/K392L/T394W | Von Kreudenstein et al., 2013 | IgG1 |
| | | mAbs | |
| | | Volume 5, 2013 - Issue 5, | |
| | | pp. 644-654 | |
| K409W | D339V/F405T | Choi et al., 2013 PNAS Jan. | IgG1 |
| | | 2, 2013. 110 (1) 270-275 | |
| K360E | Q347R | Choi et al., 2013 PNAS Jan. | IgG1 |
| | | 2, 2013. 110 (1) 270-275 | |
| K360E/K409W | D339V/Q347R/F405T | Choi et al., 2013 PNAS Jan. | IgG1 |
| | | 2, 2013. 110 (1) 270-275 | |
| Y349C/K360E/K409W | D339V/Q347R/S354C/F405T | Choi et al., 2013 PNAS Jan. | IgG1 |
| | | 2, 2013. 110 (1) 270-275 | |
| K392A/K409D | E356K/D399K | Leaver-Fey et al., 2016 | IgG1 |
| | | Structure Volume 24, Issue 4, 5 | |
| | | Apr. 2016, Pages 641-651 | |
| T366W | T366S/L358A/Y407A | Leaver-Fey et al., 2016 | IgG1 |
| | | Structure Volume 24, Issue 4, 5 | |
| | | Apr. 2016, Pages 641-651 | |
| D339M/Y407A | T336V/K409V | Leaver-Fey et al., 2016 | IgG1 |
| | | Structure Volume 24, Issue 4, 5 | |
| | | Apr. 2016, Pages 641-651 | |
| D339M/K360D/Y407A | T336V/E345R/Q347R/K409V | Leaver-Fey et al., 2016 | IgG1 |
| | | Structure Volume 24, Issue 4, 5 | |
| | | Apr. 2016, Pages 641-651 | |
| Y349S/T366V/K370Y/K409V | E357D/S364Q/Y407A | Leaver-Fey et al., 2016 | IgG1 |
| | | Structure Volume 24, Issue 4, 5 | |
| | | Apr. 2016, Pages 641-651 | |
| Y349S/T366M/K370Y/K409V | E356G/E357D/S364Q/Y407A | Leaver-Fey et al., 2016 | IgG1 |
| | | Structure Volume 24, Issue 4, 5 | |
| | | Apr. 2016, Pages 641-651 | |
| Y349S/T366M/K370Y/K409V | E357D/S364R/Y407A | Leaver-Fey et al., 2016 | IgG1 |
| | | Structure Volume 24, Issue 4, 5 | |
| | | Apr. 2016, Pages 641-651 | |

And any combination as described in Tables 1-3 of US20150284475A1

By way of example, in some embodiments, a human IgG Fc domains comprise mutations disclosed in Table 5, which reduce or eliminate FcγR and/or complement binding in the Fc domain. In embodiments, the Table 5 mutations are in both chains.

TABLE 5

| Chain 1 mutation | Reference | IgG |
|---|---|---|
| L234A/L235A | Alegre et al., 1994 | IgG1 |
| | Transplantation 57: 1537- | |
| | 1543 | |
| F234A/L235A | Alegre et al., 1994 | IgG4 |
| | Transplantation 57: 1537- | |
| | 1543 | |
| L235E | Morgan et al., 1995 | IgG1 |
| | Immunology. 1995 October; | |
| | 86(2): 319-324. | |
| L235E | Morgan et al., 1995 | IgG4 |
| | Immunology. 1995 October; | |
| | 86(2): 319-324. | |
| L235A | Morgan et al., 1995 | IgG1 |
| | Immunology. 1995 October; | |
| | 86(2): 319-324. | |
| G237A | Morgan et al., 1995 | IgG1 |
| | Immunology. 1995 October; | |
| | 86(2): 319-324. | |
| N297H | Tao and Morrison, | IgG1 |
| | J. Immunol. 1989; | |
| | 143: 2595-2601 | |
| N297Q | Tao and Morrison, | IgG1 |
| | J. Immunol. 1989; | |
| | 143: 2595-2601 | |
| N297K | Tao and Morrison, | IgG3 |
| | J. Immunol. 1989; | |
| | 143: 2595-2601 | |
| N297Q | Tao and Morrison, | IgG3 |
| | J. Immunol. 1989; | |
| | 143: 2595-2601 | |
| D265A | Idusogie et al., 2000 J | IgG1 |
| | Immunol Apr. 15, 2000, | |
| | 164 (8) 4178-4184 | |
| D270A, V, K | Idusogie et al., 2000 J | IgG1 |
| | Immunol Apr. 15, 2000, | |
| | 164 (8) 4178-4184 | |
| K322A, L, M, D, E | Idusogie et al., 2000 J | IgG1 |
| | Immunol Apr. 15, 2000, | |
| | 164 (8) 4178-4184 | |
| P329A, X | Idusogie et al., 2000 J | IgG1 |
| | Immunol Apr. 15, 2000, | |
| | 164 (8) 4178-4184 | |
| P331A, S, G, X | Idusogie et al., 2000 J | IgG1 |
| | Immunol Apr. 15, 2000, | |
| | 164 (8) 4178-4184 | |
| D265A | Idusogie et al., 2000 J | IgG1 |
| | Immunol Apr. 15, 2000, | |
| | 164 (8) 4178-4184 | |
| L234A | Hezareh et al., 2001 J. | IgG1 |
| | Virol. December 2001 vol. | |
| | 75 no. 24 12161-12168 | |
| L234A/L235A | Hezareh et al., 2001 J. | IgG1 |
| | Virol. December 2001 vol. | |
| | 75 no. 24 12161-12168 | |
| L234F/L235E/P331S | Oganesyan et al., 2008 | IgG1 |
| | Acta Cryst. (2008). D64, | |
| | 700-704 | |
| H268Q/V309L/A330S/P331S | An et al., 2009 mAbs | IgG1 |
| | Volume 1, 2009 - Issue 6, | |
| | pp. 572-579 | |
| G236R/L328R | Moore et al., 2011 mAbs | IgG1 |
| | Volume 3, 2011 - Issue 6, | |
| | pp. 546-557 | |
| N297G | Couch et al., 2013 Sci. | IgG1 |
| | Transl. Med., 5 (2013) | |
| | 183ra57, 1-12 | |
| N297G/D265A | Couch et al., 2013 Sci. | IgG1 |
| | Transl. Med., 5 (2013) | |
| | 183ra57, 1-12 | |
| V234A/G237A/P328S/H268A/ | Vafa et al., 2014 Methods | IgG2 |
| V309L/A330S/P331S | Volume 65, Issue 1, 1 | |
| | Jan. 2014, Pages 114- | |
| | 126 | |
| L234A/L235A/P329G | Lo et al., 2016 The | IgG1 |
| | Journal of Biological | |
| | Chemistry | |
| | 292, 3900-3908 | |
| N297D | Schlothauer et al., 2016 | IgG1 |
| | Protein Engineering, | |
| | Design and Selection, | |
| | Volume 29, Issue 10, 1 | |
| | Oct. 2016, Pages | |
| | 457-466 | |
| S228P/L235E | Schlothauer et al., 2016 | IgG4 |
| | Protein Engineering, | |
| | Design and Selection, | |
| | Volume 29, Issue 10, 1 | |
| | Oct. 2016, Pages | |
| | 457-466 | |
| S228P/L235E/P329G | Schlothauer et al., 2016 | IgG4 |
| | Protein Engineering, | |
| | Design and Selection, | |
| | Volume 29, Issue 10, 1 | |
| | Oct. 2016, Pages | |
| | 457-466 | |
| L234F/L235A/K322Q | Borrok et al., 2017 J | IgG1 |
| | Pharm Sci April 2017 | |
| | Volume 106, Issue 4, | |
| | Pages 1008-1017 | |
| L234F/L235Q/P331G | Borrok et al., 2017 J | IgG1 |
| | Pharm Sci April 2017 | |
| | Volume 106, Issue 4, | |
| | Pages 1008-1017 | |
| L234F/L235Q/K322Q | Borrok et al., 2017 J | IgG1 |
| | Pharm Sci April 2017 | |
| | Volume 106, Issue 4, | |
| | Pages 1008-1017 | |
| L234A/L235A/G237A/P328S/ | Tam et al., 2017 Open | IgG1 |
| H268A/A330S/P331S | Access | |
| | Antibodies 2017, 6(3), 12; | |
| | doi: 10.3390/antib6030012 | |
| S228P/F234A/L235A | Tam et al., 2017 Open | IgG4 |
| | Access | |
| | Antibodies 2017, 6(3), 12; | |
| | doi: 10.3390/antib6030012 | |
| S228P/F234A/L235A/ | Tam et al., 2017 Open | IgG4 |
| G237A/P238S | Access | |
| | Antibodies 2017, 6(3), 12; | |
| | doi: 10.3390/antib6030012 | |
| S228P/F234A/L235A/G236/ | Tam et al., 2017 Open | IgG4 |
| G237A/P238S | Access | |
| | Antibodies 2017, 6(3), 12; | |
| | doi: 10.3390/antib6030012 | |

In some embodiments, the Fc domains in the chimeric protein complexes (e.g., Fc-based chimeric protein complexes) of the present technology are homodimeric, i.e., the Fc region in the chimeric protein complex comprises two identical protein fragments.

In some embodiments, the Fc domains in the chimeric protein complexes (e.g., Fc-based chimeric protein complexes) of the present technology are heterodimeric, i.e., the Fc domain comprises two non-identical protein fragments.

In some embodiments, heterodimeric Fc domains are engineered using ionic pairing and/or knob-in-hole mutations described herein. In some embodiments, the heterodimeric chimeric protein complexes (e.g., Fc-based chimeric protein complexes) have a trans orientation/configuration. In a trans orientation/configuration, the targeting moiety and signaling agent, in embodiments, not found on the same polypeptide chain in the present chimeric protein complexes (e.g., Fc-based chimeric protein complexes).

In some embodiments, the Fc domains includes or starts with the core hinge region of wild-type human IgG1, which contains the sequence Cys-Pro-Pro-Cys. In some embodiments, the Fc domains also include the upper hinge, or parts thereof (e.g., DKTHTCPPC; see WO 2009053368), EPKSCDKTHTCPPC, or EPKSSDKTHTCPPC; see Lo et al., Protein Engineering vol. 11 no.6 pp. 495-500, 1998).

### Chimeric Protein Complexes

The chimeric protein complexes (e.g., Fc-based chimeric protein complexes) of the present technology comprise at least one Fc domain disclosed herein, at least one signaling agent, e.g. IL-1α or pro-IL-1α (SA) disclosed herein, e.g. IL-1α, and at least one targeting moiety (TM) disclosed herein.

It is understood that the present chimeric protein complexes (e.g., Fc-based chimeric protein complexes) may encompass a complex of two fusion proteins, each comprising an Fc domain. In some embodiments, the chimeric protein complex (e.g., Fc-based chimeric protein complex) is heterodimeric. In some embodiments, the heterodimeric chimeric protein complex (e.g., Fc-based chimeric protein complex) has a trans orientation/configuration. In some embodiments, the heterodimeric chimeric protein complex (e.g., Fc-based chimeric protein complex) has a cis orientation/configuration.

In some embodiments, heterodimeric Fc domains are engineered using ionic pairing and/or knob-in-hole mutations described herein. In some embodiments, the heterodimeric chimeric protein complexes (e.g., Fc-based chimeric protein complexes) have a trans orientation. In a trans orientation, the targeting moiety and signaling agent are, in embodiments, not found on the same polypeptide chain in the present chimeric protein complexes (e.g., Fc-based chimeric protein complexes). In a trans orientation, the targeting moiety and signaling agent are, in embodiments, found on separate polypeptide chains in the chimeric protein complexes (e.g., Fc-based chimeric protein complexes). In a cis orientation, the targeting moiety and signaling agent are, in embodiments, found on the same polypeptide chain in the chimeric protein complexes (e.g., Fc-based chimeric protein complexes).

In some embodiments, where more than one targeting moiety is present in the heterodimeric protein complexes described herein, one targeting moiety may be in trans orientation (relative to the signaling agent), whereas another targeting moiety may be in cis orientation (relative to the signaling agent). In some embodiments, the signaling agent and target moiety are on the same ends/sides (N-terminal or C-terminal ends) of an Fc domain. In some embodiments, the signaling agent and targeting moiety are on different sides/ends of a Fc domain (N-terminal and C-terminal ends).

In some embodiments, where more than one targeting moiety is present in the heterodimeric protein complexes described herein, the targeting moieties may be found on the same Fc chain or on two different Fc chains in the heterodimeric protein complex (in the latter case the targeting moieties would be in trans relative to each other, as they are on different Fc chains). In some embodiments, where more than one targeting moiety is present on the same Fc chain, the targeting moieties may be on the same or different sides/ends of a Fc chain (N-terminal or/and C-terminal ends).

In some embodiments, where more than one signaling agent is present in the heterodimeric protein complexes described herein, the signaling agents may be found on the same Fc chain or on two different Fc chains in the heterodimeric protein complex (in the latter case the signaling agents would be in trans relative to each other, as they are on different Fc chains). In some embodiments, where more than one signaling agent is present on the same Fc chain, the signaling agents may be on the same or different sides/ends of a Fc chain (N-terminal or/and C-terminal ends).

In some embodiments, where more than one signaling agent is present in the heterodimeric protein complexes described herein, one signaling agent may be in trans orientation (as relates to the targeting moiety), whereas another signaling agent may be in cis orientation (as relates to the targeting moiety).

In some embodiments, the heterodimeric chimeric protein complex (e.g., Fc-based chimeric protein complex) does not comprise the signaling agent, e.g. IL-1α and targeting moiety on a single polypeptide.

In some embodiments, the chimeric protein complex (e.g., Fc-based chimeric protein complex) has an improved in vivo half-life relative to a chimeric protein lacking an Fc or a chimeric protein, which is not a heterodimeric complex. In some embodiments, the chimeric protein complex (e.g., Fc-based chimeric protein complex) has an improved solubility, stability and other pharmacological properties relative to a chimeric protein lacking an Fc or a chimeric protein, which is not a heterodimeric complex.

Heterodimeric chimeric protein complexes (e.g., Fc-based chimeric protein complexes) are composed of two different polypeptides. In embodiments described herein, the targeting domain is on a different polypeptide than the signaling agent, e.g. IL-1, and accordingly, proteins that contain only one targeting domain copy, and also only one signaling agent, e.g. IL-1α copy can be made (this provides a configuration in which potential interference with desired properties can be controlled). Further, in embodiments, one targeting domain (e.g. VHH) only can avoid cross-linking of the antigen on the cell surface (which could elicit undesired effects in some cases). Further, in embodiments, one signaling agent, e.g. IL-1α may alleviate molecular “crowding” and potential interference with avidity mediated induction and/or restoration of effector function in dependence of the targeting domain. Further, in embodiments, heterodimeric chimeric protein complexes (e.g., Fc-based chimeric protein complexes) can have two targeting moieties and these can be placed on the two different polypeptides. For instance, in embodiments, the C-terminus of both targeting moieties (e.g. VHHs) can be masked to avoid potential autoantibodies or pre-existing antibodies (e.g. VHH autoantibodies or pre-existing antibodies). Further, in embodiments, heterodimeric chimeric protein complexes (e.g., Fc-based chimeric protein complexes), e.g. with the targeting domain on a different polypeptide than the signaling agent, e.g. IL-1α (e.g. wild type signaling agent, e.g. wild type IL-1α), may favor “cross-linking” of two cell types (e.g. a tumor cell and an immune cell). Further, in embodiments, heterodimeric chimeric protein complexes (e.g., Fc-based chimeric protein complexes) can have two signaling agent, each on different polypeptides to allow more complex effector responses.

Further, in embodiments, heterodimeric chimeric protein complexes (e.g., Fc-based chimeric protein complexes), e.g. with the targeting domain on a different polypeptide than the signaling agent, e.g. IL-1α, combinatorial diversity of targeting moiety and signaling agent, e.g. IL-1α is provided in a practical manner. For instance, in embodiments, polypeptides with any of the targeting moieties described herein can be combined “off the shelf” with polypeptides with any of the signaling agents described herein to allow rapid generation of various combinations of targeting moieties and signaling agents in single chimeric protein complexes (e.g., Fc-based chimeric protein complexes).

In some embodiments, the chimeric protein complex (e.g., Fc-based chimeric protein complex) comprises one or more linkers. In some embodiments, the chimeric protein complex (e.g., Fc-based chimeric protein complex) includes a linker that connects the Fc domain, signaling agent, e.g. IL-1α(s) and targeting moiety(ies). In some embodiments, the chimeric protein complex (e.g., Fc-based chimeric protein complex) includes a linker that connects each signaling agent, e.g. IL-1α and targeting moiety (or, if more than one targeting moiety, a signaling agent, e.g. IL-1α to one of the targeting moieties). In some embodiments, the chimeric protein complex (e.g., Fc-based chimeric protein complex) includes a linker that connects each signaling agent, e.g. IL-1α to the Fc domain. In some embodiments, the Fc-based chimeric protein complex includes a linker that connects each targeting moiety to the Fc domain. In some embodiments, the chimeric protein complex (e.g., Fc-based chimeric protein complex) includes a linker that connects a targeting moiety to another targeting moiety. In some embodiments, chimeric protein complex (e.g., Fc-based chimeric protein complex) includes a linker that connects a signaling agent, e.g. IL-1α to another signaling agent.

In some embodiments, a chimeric protein complex (e.g., Fc-based chimeric protein complex) comprises two or more targeting moieties. In such embodiments, the targeting moieties can be the same targeting moiety or they can be different targeting moieties.

In some embodiments, a chimeric protein complex (e.g., Fc-based chimeric protein complex) comprises two or more signaling agents. In such embodiments, the signaling agents can be the same targeting moiety or they can be different targeting moieties.

By way of example, in some embodiments, the chimeric protein complex (e.g., Fc-based chimeric protein complex) comprise a Fc domain, at least two signaling agents (SA), and at least two targeting moieties (TM), wherein the Fc domain, signaling agents, and targeting moieties are selected from any of the Fc domains, signaling agents, and targeting moieties disclosed herein. In some embodiments, the Fc domain is homodimeric.

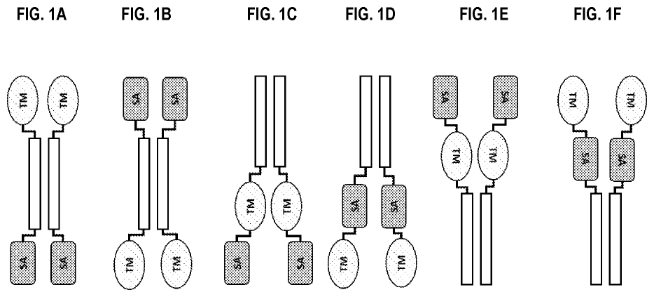

In various embodiments, the chimeric protein complex (e.g., Fc-based chimeric protein complex) takes the form of any of the schematics of FIGS. 1A-F.

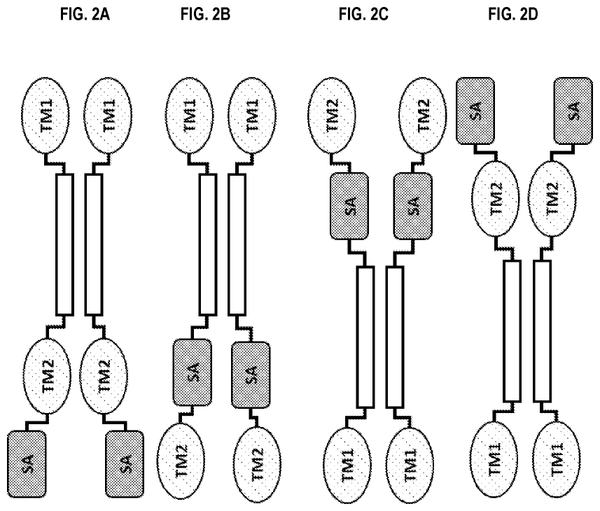

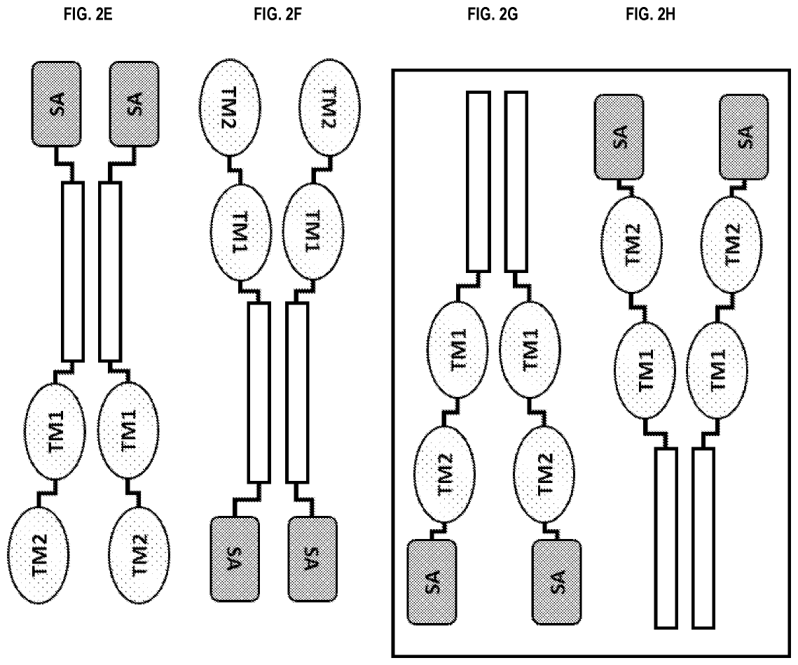

In various embodiments, the chimeric protein complex (e.g., Fc-based chimeric protein complex) takes the form of any of the schematics of FIGS. 2A-H.

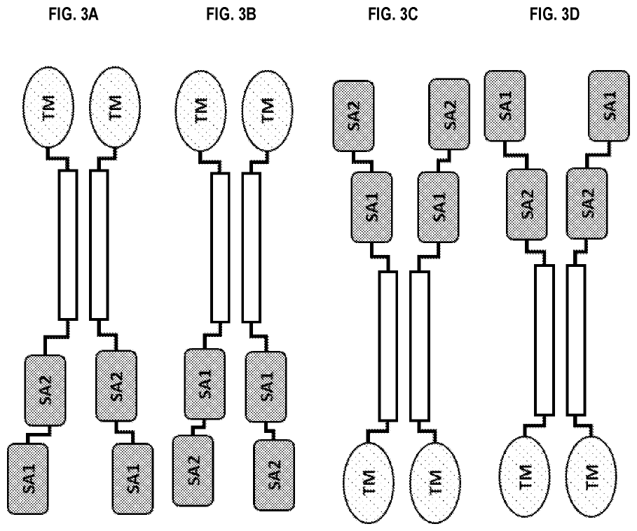

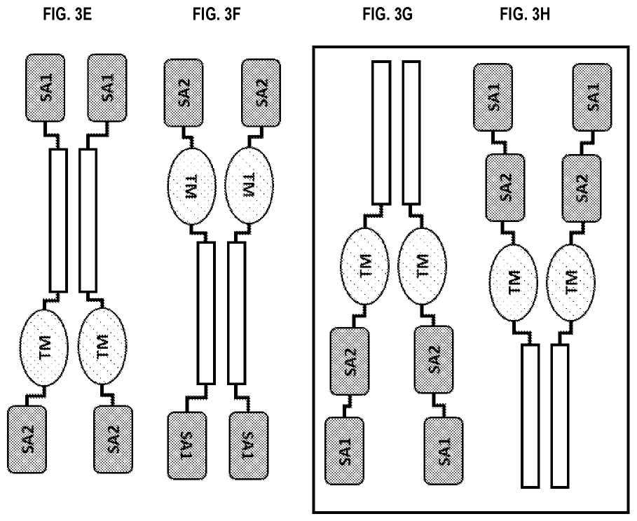

In various embodiments, the chimeric protein complex (e.g., Fc-based chimeric protein complex) takes the form of any of the schematics of FIGS. 3A-H.

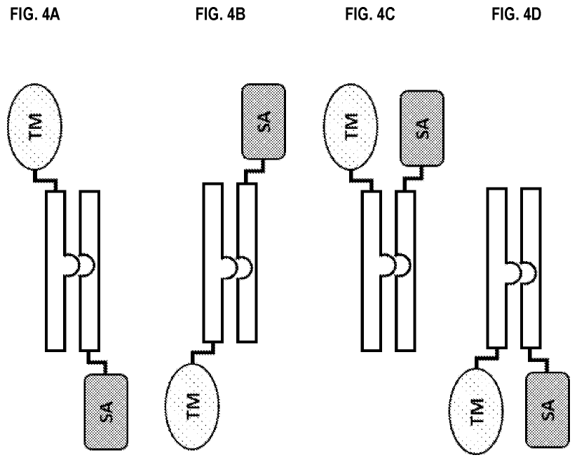

In various embodiments, the chimeric protein complex (e.g., Fc-based chimeric protein complex) takes the form of any of the schematics of FIGS. 4A-D.

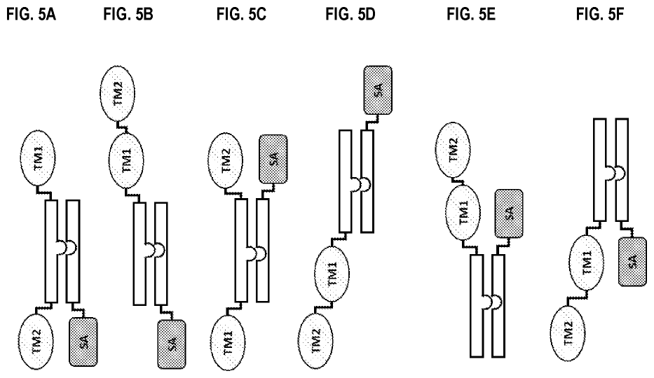

In various embodiments, the chimeric protein complex (e.g., Fc-based chimeric protein complex) takes the form of any of the schematics of FIGS. 5A-F.

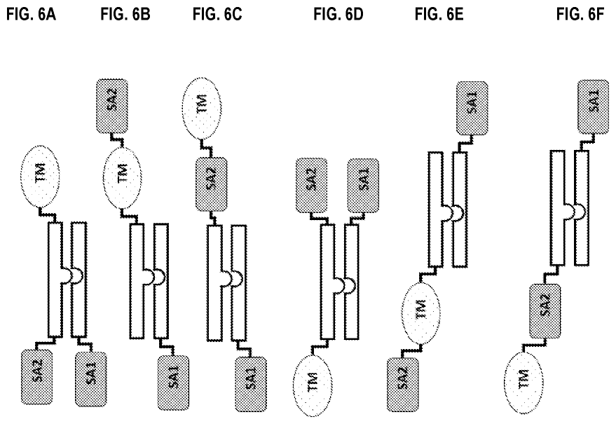

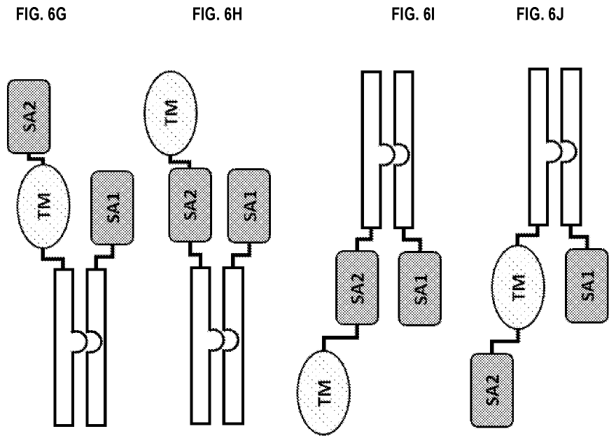

In various embodiments, the chimeric protein complex (e.g., Fc-based chimeric protein complex) takes the form of any of the schematics of FIGS. 6A-J.

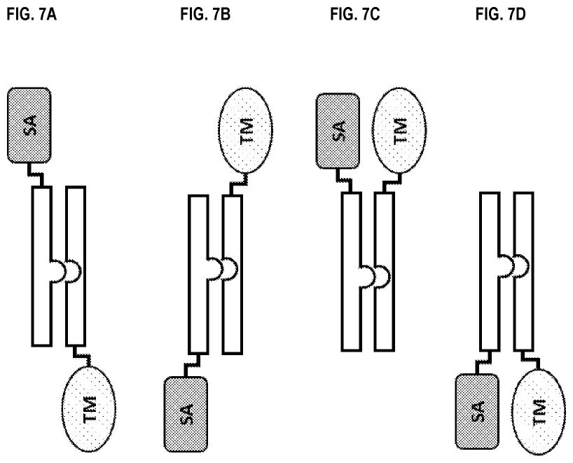

In various embodiments, the chimeric protein complex (e.g., Fc-based chimeric protein complex) takes the form of any of the schematics of FIGS. 7A-D.

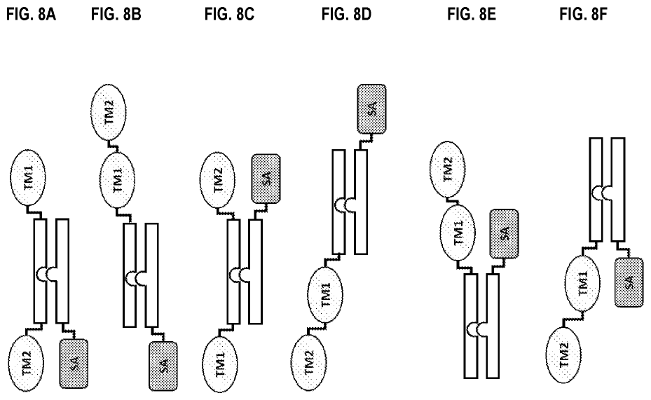

In various embodiments, the chimeric protein complex (e.g., Fc-based chimeric protein complex) takes the form of any of the schematics of FIGS. 8A-F.

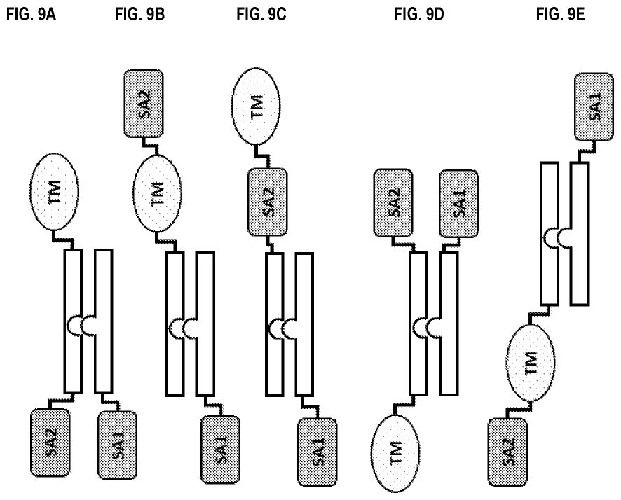

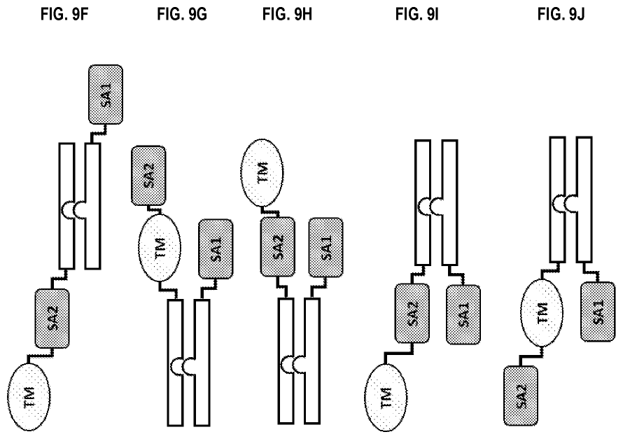

In various embodiments, the chimeric protein complex (e.g., Fc-based chimeric protein complex) takes the form of any of the schematics of FIGS. 9A-J.

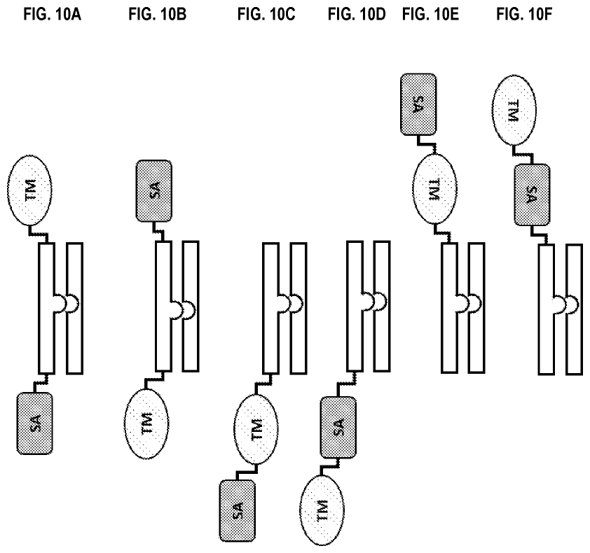

In various embodiments, the chimeric protein complex (e.g., Fc-based chimeric protein complex) takes the form of any of the schematics of FIGS. 10A-F.

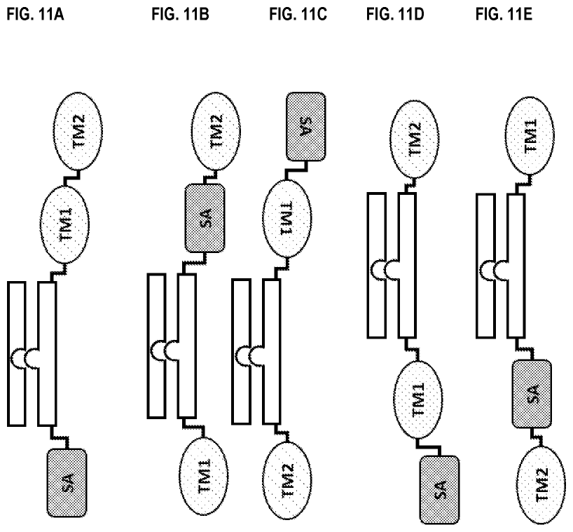

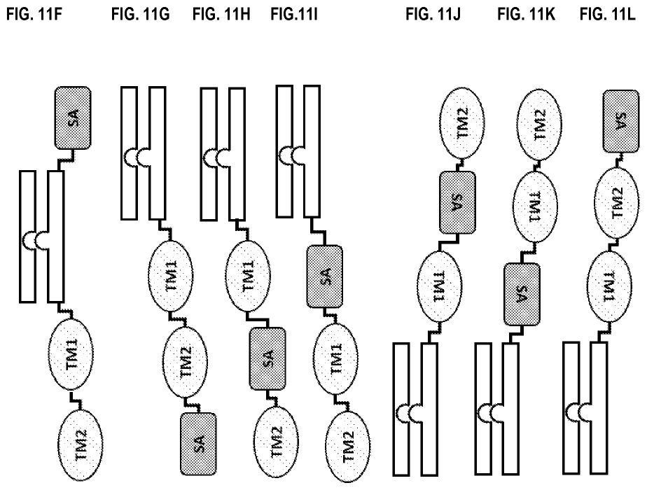

In various embodiments, the chimeric protein complex (e.g., Fc-based chimeric protein complex) takes the form of any of the schematics of FIGS. 11A-L.

In various embodiments, the chimeric protein complex (e.g., Fc-based chimeric protein complex) takes the form of any of the schematics of FIGS. 12A-L.

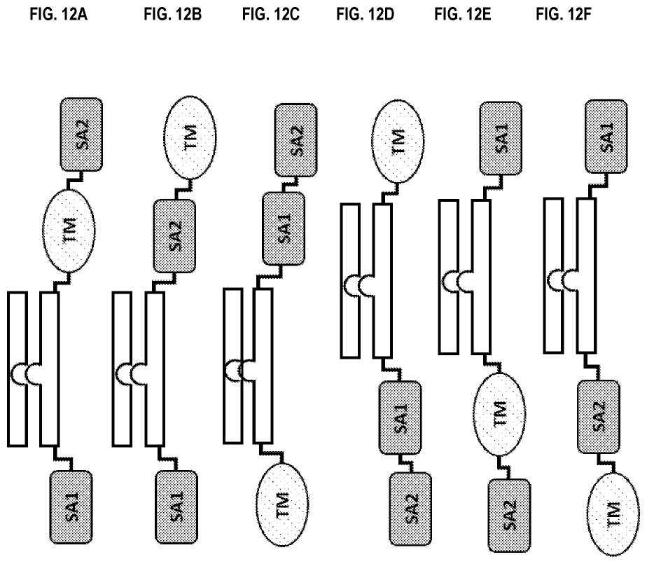

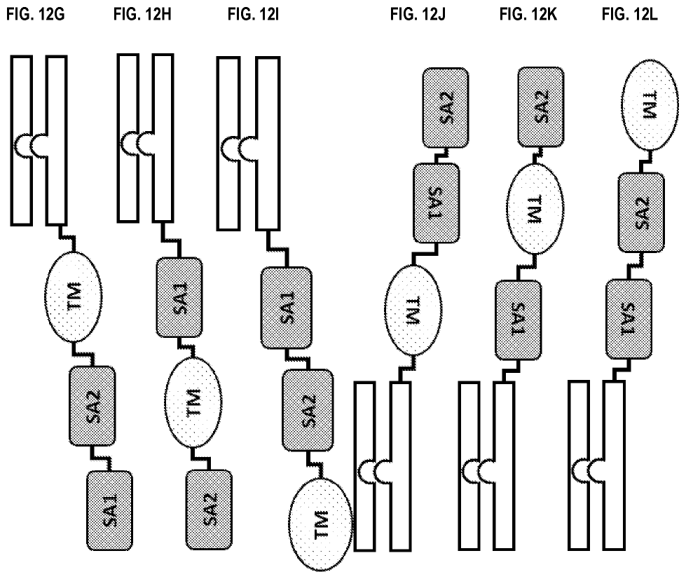

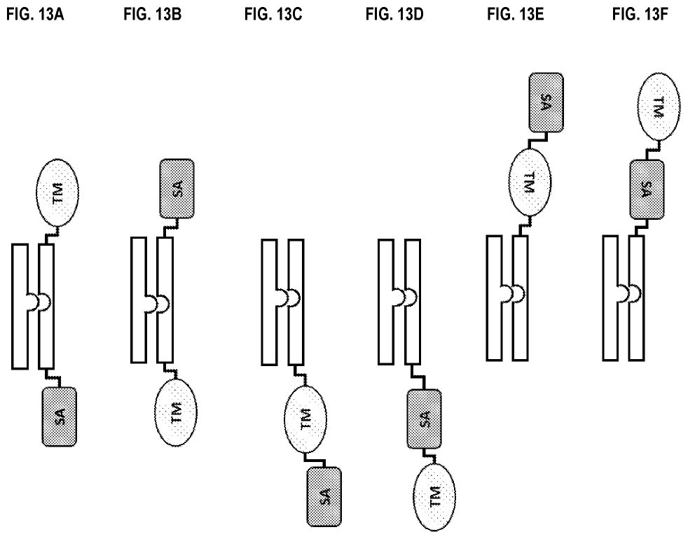

In various embodiments, the chimeric protein complex (e.g., Fc-based chimeric protein complex) takes the form of any of the schematics of FIGS. 13A-F.

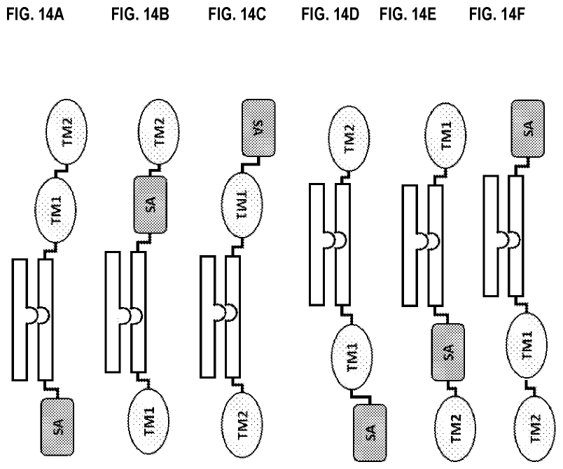

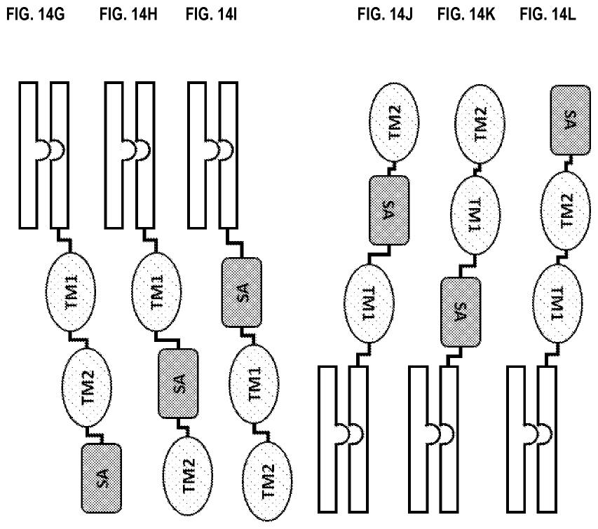

In various embodiments, the chimeric protein complex (e.g., Fc-based chimeric protein complex) takes the form of any of the schematics of FIGS. 14A-L.

In various embodiments, the chimeric protein complex (e.g., Fc-based chimeric protein complex) takes the form of any of the schematics of FIGS. 15A-L.

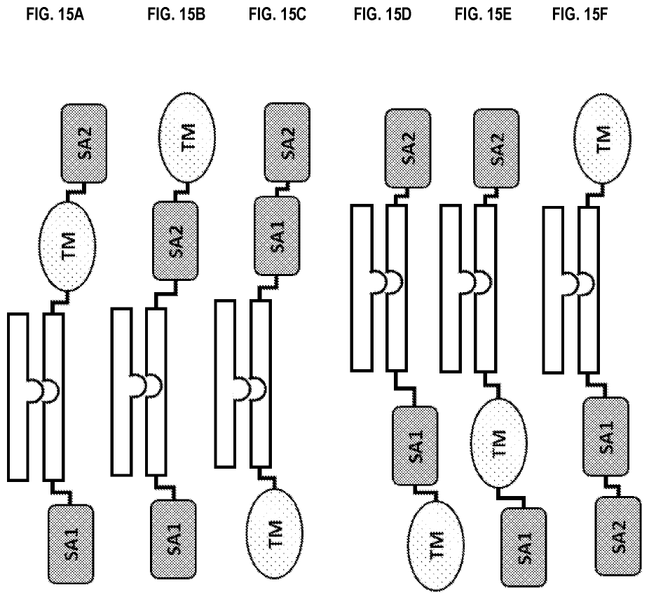

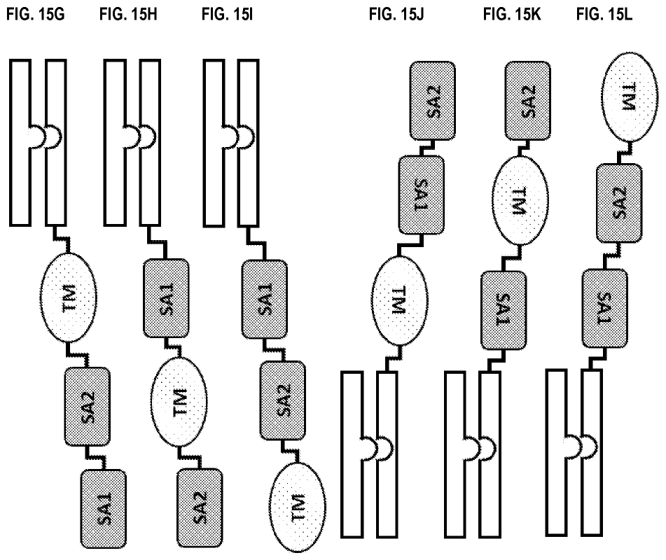

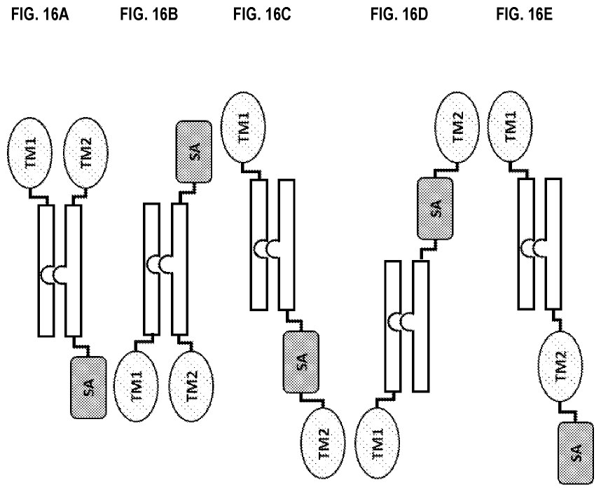

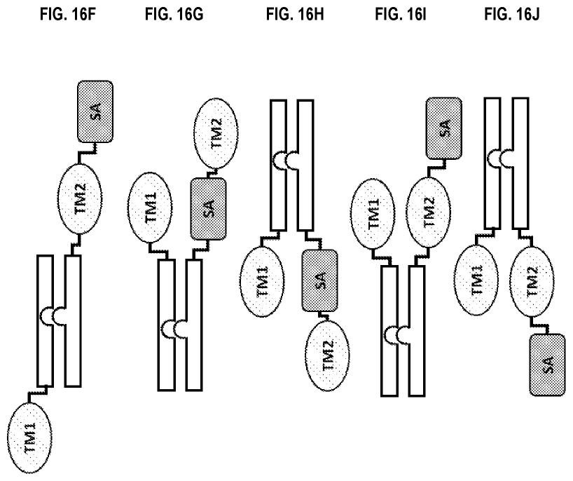

In various embodiments, the chimeric protein complex (e.g., Fc-based chimeric protein complex) takes the form of any of the schematics of FIGS. 16A-J.

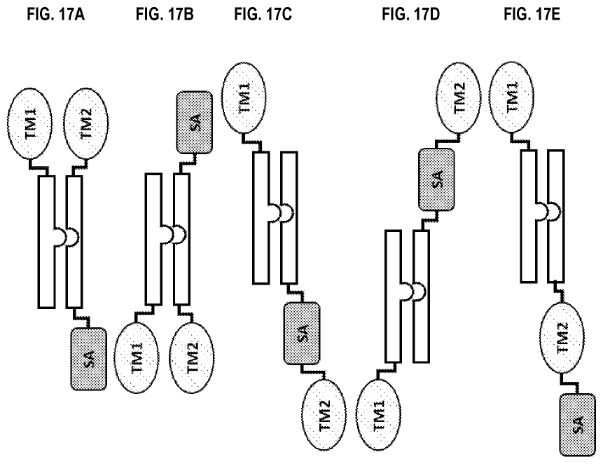

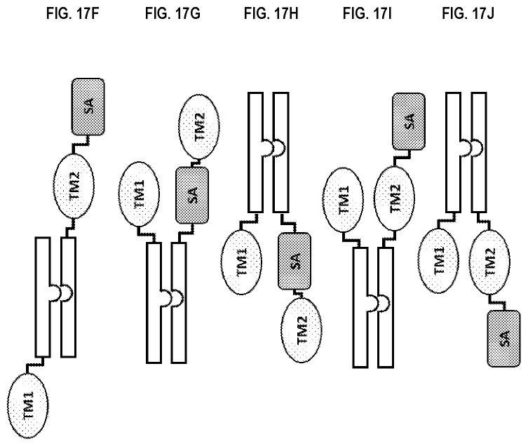

In various embodiments, the chimeric protein complex (e.g., Fc-based chimeric protein complex) takes the form of any of the schematics of FIGS. 17A-J.

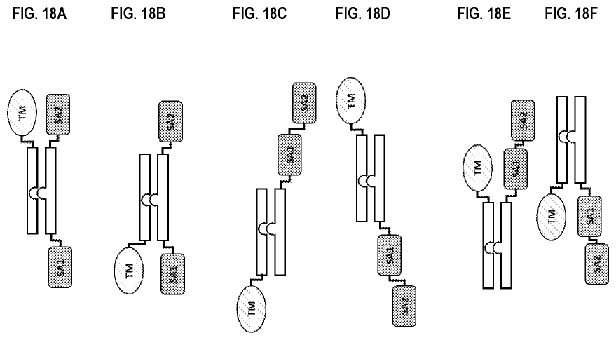

In various embodiments, the chimeric protein complex (e.g., Fc-based chimeric protein complex) takes the form of any of the schematics of FIGS. 18A-F.

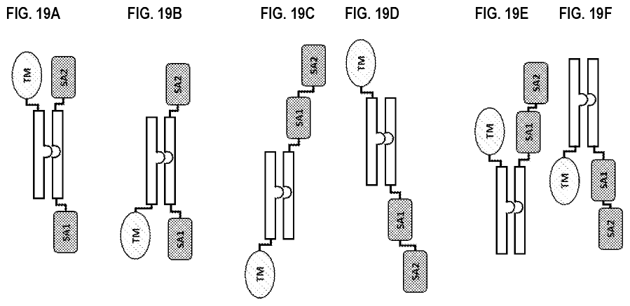

In various embodiments, the chimeric protein complex (e.g., Fc-based chimeric protein complex) takes the form of any of the schematics of FIGS. 19A-F.

In some embodiments, the signaling agents are linked to the targeting moieties and the targeting moieties are linked to the Fc domain on the same terminus (see FIGS. 1A-F). In some embodiments, the Fc domain is homodimeric.

In some embodiments, the signaling agents and targeting moieties are linked to the Fc domain, wherein the targeting moieties and signaling agents are linked on the same terminus (see FIGS. 1A-F). In some embodiments, the Fc domain is homodimeric.

In some embodiments, the targeting moieties are linked to signaling agents and the signaling agents are linked to the Fc domain on the same terminus (see FIGS. 1A-F). In some embodiments, the Fc domain is homodimeric.

In some embodiments, the homodimeric chimeric protein complex (e.g., Fc-based chimeric protein complex) has two or more targeting moieties. In some embodiments, there are four targeting moieties and two signaling agents, the targeting moieties are linked to the Fc domain and the signaling agents are linked to targeting moieties on the same terminus (see FIGS. 2A-H). In some embodiments, the Fc domain is homodimeric. In some embodiments, where there are four targeting moieties and two signaling agents, two targeting moieties are linked to the Fc domain and two targeting moieties are linked to the signaling agents, which are linked to the Fc domain on the same terminus (see FIGS. 2A-H). In some embodiments, the Fc domain is homodimeric. In some embodiments, where there are four targeting moieties and two signaling agents, two targeting moieties are linked to each other and one of the targeting moieties of from each pair is linked to the Fc domain on the same terminus and the signaling agents are linked to the Fc domain on the same terminus (see FIGS. 2A-H). In some embodiments, the Fc domain is homodimeric. In some embodiments, where there are four targeting moieties and two signaling agents, two targeting moieties are linked to each other, wherein one of the targeting moieties of from each pair is linked to a signaling agent, e.g. IL-1α and the other targeting moiety of the pair is linked the Fc domain, wherein the targeting moieties linked to the Fc domain are linked on the same terminus (see FIGS. 2A-H). In some embodiments, the Fc domain is homodimeric.

In some embodiments, the homodimeric chimeric protein complex (e.g., Fc-based chimeric protein complex) has two or more signaling agents. In some embodiments, where there are four signaling agents and two targeting moieties, two signaling agents are linked to each other and one of the signaling agents of from pair is linked to the Fc domain on the same terminus and the targeting moieties are linked to the Fc domain on the same terminus (see FIGS. 3A-H). In some embodiments, the Fc domain is homodimeric.

In some embodiments, where there are four signaling agents and two targeting moieties, two signaling agents are linked to the Fc domain one the same terminus and two of the signaling agents are each linked to a targeting moiety, wherein the targeting moieties are linked to the Fc domain at the same terminus (see FIGS. 3A-H). In some embodiments, the Fc domain is homodimeric. In some embodiments, where there are four signaling agents and two targeting moieties, two signaling agents are linked to each other and one of the signaling agents of from pair is linked to a targeting moiety and the targeting moieties are linked to the Fc domain on the same terminus (see FIGS. 3A-H). In some embodiments, the Fc domain is homodimeric.

By way of example, in some embodiments, the chimeric protein complex (e.g., Fc-based chimeric protein complex) comprise a Fc domain, wherein the Fc domain comprises ionic pairing mutation(s) and/or knob-in-hole mutation(s), at least one signaling agent, e.g. IL-1α, and at least one targeting moiety, wherein the ionic pairing motif and/or a knob-in-hole motif, signaling agent, e.g. IL-1α, and targeting moiety are selected from any of the ionic pairing motif and/or a knob-in-hole motif, signaling agents, and targeting moieties disclosed herein. In some embodiments, the Fc domain is heterodimeric. In some embodiments, the Fc domain comprises a mutation that reduces or eliminates its effector function.

In some embodiments, the signaling agent, e.g. IL-1α is linked to the targeting moiety, which is linked to the Fc domain (see FIGS. 10A-F and 13A-F). In some embodiments, the targeting moiety is linked to the signaling agent, e.g. IL-1α, which is linked to the Fc domain (see FIGS. 10A-F and 13A-F). In some embodiments, the Fc domain is heterodimeric. In some embodiments, the Fc domain comprises a mutation that reduces or eliminates its effector function.

In some embodiments, the signaling agent, e.g. IL-1α and targeting moiety are linked to the Fc domain (see FIGS. 4A-D, 7A-D, 10A-F, and 13A-F). In some embodiments, the targeting moiety and the signaling agent, e.g. IL-1α are linked to different Fc chains on the same terminus (see FIGS. 4A-D and 7A-D). In some embodiments, the targeting moiety and the signaling agent, e.g. IL-1α are linked to different Fc chains on different termini (see FIGS. 4A-D and 7A-D). In some embodiments, the targeting moiety and the signaling agent, e.g. IL-1α are linked to the same Fc chain (see FIGS. 10A-F and 13A-F). In some embodiments, the Fc domain is heterodimeric. In some embodiments, the Fc domain comprises a mutation that reduces or eliminates its effector function.

In some embodiments, where there are one signaling agent, e.g. IL-1α and two targeting moieties, the signaling agent, e.g. IL-1α is linked to the Fc domain and two targeting moieties can be: 1) linked to each other with one of the targeting moieties linked to the Fc domain; or 2) each linked to the Fc domain (see FIGS. 5A-F, 8A-F, 11A-L, 14A-L, 16A-J, and 17A-J). In some embodiments, the targeting moieties are linked on one Fc chain and the signaling agent, e.g. IL-1α is on the other Fc chain (see FIGS. 5A-F and 8A-F). In some embodiments, the paired targeting moieties and the signaling agent, e.g. IL-1α are linked to the same Fc chain (see FIGS. 11A-L and 14A-L). In some embodiments, a targeting moiety is linked to the Fc domain and the other targeting moiety is linked to the signaling agent, e.g. IL-1α, and the paired targeting moiety is linked to the Fc domain (see FIGS. 11A-L, 14A-L, 16A-J, and 17A-J). In some embodiments, the unpaired targeting moiety and paired targeting moiety are linked to the same Fc chain (see FIGS. 11A-L and 14A-L). In some embodiments, the unpaired targeting moiety and paired targeting moiety are linked to different Fc chains (see FIGS. 16A-J and 17A-J). In some embodiments, the unpaired targeting moiety and paired targeting moiety are linked on the same terminus (see FIGS. 16A-J and 17A-J). In some embodiments, the Fc domain is heterodimeric. In some embodiments, the Fc domain comprises a mutation that reduces or eliminates its effector function.

In some embodiments, where there are one signaling agent, e.g. IL-1α and two targeting moieties, a targeting moiety is linked to the signaling agent, e.g. IL-1α, which is linked to the Fc domain, and the unpaired targeting moiety is linked the Fc domain (see FIGS. 11A-L, 14A-L, 16A-J, and 17A-J). In some embodiments, the paired signaling agent, e.g., IL-1α and unpaired targeting moiety are linked to the same Fc chain (see FIGS. 11A-L and 14A-L). In some embodiments, the paired signaling agent, e.g. IL-1α and unpaired targeting moiety are linked to different Fc chains (see FIGS. 16A-J and 17A-J). In some embodiments, the paired signaling agent, e.g. IL-1α and unpaired targeting moiety are linked on the same terminus (see FIGS. 16A-J and 17A-J). In some embodiments, the Fc domain is heterodimeric. In some embodiments, the Fc domain comprises a mutation that reduces or eliminates its effector function.

In some embodiments, where there are one signaling agent, e.g. IL-1α and two targeting moieties, the targeting moieties are linked together and the signaling agent, e.g. IL-1α is linked to one of the paired targeting moieties, wherein the targeting moiety not linked to the signaling agent, e.g. IL-1α is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent, e.g. IL-1α and two targeting moieties, the targeting moieties are linked together and the signaling agent, e.g. IL-1α is linked to one of the paired targeting moieties, wherein the signaling agent, e.g. IL-1α is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent, e.g. IL-1α and two targeting moieties, the targeting moieties are both linked to the signaling agent, e.g. IL-1α, wherein one of the targeting moieties is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent, e.g. IL-1α and two targeting moieties, the targeting moieties and the signaling agent, e.g. IL-1α are linked to the Fc domain (see FIGS. 16A-J and 17A-J). In some embodiments, the targeting moieties are linked on the terminus (see FIGS. 16A-J and 17A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked to the Fc domain on the same terminus and the targeting moiety is linked to the Fc domain (see FIGS. 6A-J and 9A-J). In some embodiments, the signaling agents are linked to the Fc domain on the same Fc chain and the targeting moiety is linked on the other Fc chain (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, a signaling agent, e.g. IL-1α is linked to the targeting moiety, which is linked to the Fc domain and the other signaling agent, e.g. IL-1α is linked to the Fc domain (see FIGS. 6A-J, 9A-J, 12A-L, and 15A-L). In some embodiments, the targeting moiety and the unpaired signaling agent, e.g. IL-1α are linked to different Fc chains (see FIGS. 6A-J and 9A-J). In some embodiments, the targeting moiety and the unpaired signaling agent, e.g. IL-1α are linked to different Fc chains on the same terminus (see FIGS. 6A-J and 9A-J). In some embodiments, the targeting moiety and the unpaired signaling agent, e.g. IL-1α are linked to different Fc chains on different termini (see FIGS. 6A-J and 9A-J). In some embodiments, the targeting moiety and the unpaired signaling agent, e.g. IL-1α are linked to the same Fc chains (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the targeting moiety is linked to a signaling agent, e.g. IL-1α, which is linked to the Fc domain and the other signaling agent, e.g. IL-1α is linked to the Fc domain (see FIGS. 6A-J and 9A-J). In some embodiments, the paired signaling agent, e.g. IL-1α and the unpaired signaling agent, e.g. IL-1α are linked to different Fc chains (see FIGS. 6A-J and 9A-J). In some embodiments, the paired signaling agent, e.g. IL-1α and the unpaired signaling agent, e.g. IL-1α are linked to different Fc chains on the same terminus (see FIGS. 6A-J and 9A-J). In some embodiments, the paired signaling agent, e.g. IL-1 and the unpaired signaling agent, e.g. IL-1α are linked to different Fc chains on different termini (see FIGS. 6A-J and 9A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and the targeting moiety is linked to one of the paired signaling agents, wherein the targeting moiety is linked to the Fc domain (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the Fc domain and the targeting moiety is linked to the Fc domain (see FIGS. 12A-L, 15A-L, 18A-F, and 19A-F). In some embodiments, the paired signaling agents and targeting moiety are linked to the same Fc chain (see FIGS. 12A-L and 15A-L). In some embodiments, the paired signaling agents and targeting moiety are linked to different Fc chains (see FIGS. 18A-F and 19A-F). In some embodiments, the paired signaling agents and targeting moiety are linked to different Fc chains on the same terminus (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, the signaling agents are both linked to the targeting moiety, wherein one of the signaling agents is linked to the Fc domain (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the targeting moiety and the other signaling agent, e.g. IL-1α is linked to the Fc domain (see FIGS. 12A-L and 15A-L).

In some embodiments, where there are two signaling agents and one targeting moiety, each signaling agent, e.g. IL-1α is linked to the Fc domain and the targeting moiety is linked to one of the signaling agents (see FIGS. 12A-L and 15A-L). In some embodiments, the signaling agents are linked to the same Fc chain (see FIGS. 12A-L and 15A-L).

In some embodiments, a targeting moiety or signaling agent, e.g. IL-1α is linked to the Fc domain, comprising one or both of C_{H}2 and C_{H}3 domains, and optionally a hinge region. For example, vectors encoding the targeting moiety, signaling agent, e.g. IL-1α, or combination thereof, linked as a single nucleotide sequence to an Fc domain can be used to prepare such polypeptides.

### Additional Signaling Agents

In one aspect, the present invention provides a chimeric proteins or chimeric protein complexes such as Fc-based chimeric protein complex comprising one or more signaling agents (for instance, an immune-modulating agent) in addition to the IL-1α or a variant thereof described herein. In illustrative embodiments, the chimeric proteins or chimeric protein complexes such as Fc-based chimeric protein complex may comprise two, three, four, five, six, seven, eight, nine, ten or more signaling agents in addition to the IL-1α or a variant thereof described herein. In various embodiments, the additional signaling agent is modified to have reduced affinity or activity for one or more of its receptors, which allows for attenuation of activity (inclusive of agonism or antagonism) and/or prevents non-specific signaling or undesirable sequestration of the chimeric proteins or chimeric protein complexes such as Fc-based chimeric protein complex.

In various embodiments, the additional signaling agent is antagonistic in its wild type form and bears one or more mutations that attenuate its antagonistic activity. In various embodiments, the additional signaling agent is antagonistic due to one or more mutations, e.g. an agonistic signaling agent is converted to an antagonistic signaling agent and, such a converted signaling agent, optionally, also bears one or more mutations that attenuate its antagonistic activity (e.g. as described in WO 2015/007520, the entire contents of which are hereby incorporated by reference).

In various embodiments, the additional signaling agent is selected from modified versions of cytokines, growth factors, and hormones. Illustrative examples of such cytokines, growth factors, and hormones include, but are not limited to, lymphokines, monokines, traditional polypeptide hormones, such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and tumor necrosis factor-β; mullerian-inhibiting substance; mouse gonadotropin-associated peptide; inhibin; activin; vascular endothelial growth factor; integrin; thrombopoietin (TPO); nerve growth factors such as NGF-α; platelet-growth factor; transforming growth factors (TGFs) such as TGF-α and TGF-β; insulin-like growth factor-I and -II; osteo inductive factors; interferons such as, for example, interferon-α, interferon-β and interferon-γ (and interferon type I, II, and III), colony stimulating factors (CSFs) such as macrophage-CSF (M-CSF); granulocyte-macrophage-CSF (GM-CSF); and granulocyte-CSF (G-CSF); interleukins (ILs) such as, for example, IL-1β, IL-1α, IL-2, IL-3, IL-4, IL-5, IL-6, IL-7, IL-8, IL-9, IL-10, IL-11, IL-12, IL-13, and IL-18; a tumor necrosis factor such as, for example, TNF-α or TNF-β; and other polypeptide factors including, for example, LIF and kit ligand (KL). As used herein, cytokines, growth factors, and hormones include proteins obtained from natural sources or produced from recombinant bacterial, eukaryotic or mammalian cell culture systems and biologically active equivalents of the native sequence cytokines.

In some embodiments, the additional signaling agent is a modified version of a growth factor selected from, but not limited to, transforming growth factors (TGFs) such as TGF-α and TGF-β, epidermal growth factor (EGF), insulin-like growth factor such as insulin-like growth factor-I and -II, fibroblast growth factor (FGF), heregulin, platelet-derived growth factor (PDGF), vascular endothelial growth factor (VEGF).

In an embodiment, the growth factor is a modified version of a fibroblast growth factor (FGF). Illustrative FGFs include, but are not limited to, FGF1, FGF2, FGF3, FGF4, FGF5, FGF6, FGF7, FGF8, FGF9, FGF10, FGF11, FGF12, FGF13, FGF14, murine FGF15, FGF16, FGF17, FGF18, FGF19, FGF20, FGF21, FGF22, and FGF23.

In an embodiment, the growth factor is a modified version of a vascular endothelial growth factor (VEGF). Illustrative VEGFs include, but are not limited to, VEGF-A, VEGF-B, VEGF-C, VEGF-D, and PGF and isoforms thereof including the various isoforms of VEGF-A such as VEGF₁₂₁, VEGF₁₂₁b, VEGF₁₄₅, VEGF₁₆₅, VEGF₁₆₅b, VEGF₁₈₉, and VEGF₂₀₆.

In an embodiment, the growth factor is a modified version of a transforming growth factor (TGF). Illustrative TGFs include, but are not limited to, TGF-α and TGF-β and subtypes thereof including the various subtypes of TGF-β including TGFß1, TGFß2, and TGFß3.

In some embodiments, the additional signaling agent is a modified version of a hormone selected from, but not limited to, human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, calcitriol, calciferol, atrial-natriuretic peptide, gastrin, secretin, cholecystokinin, neuropeptide Y, ghrelin, PYY3-36, insulin-like growth factor (IGF), leptin, thrombopoietin, erythropoietin (EPO), and angiotensinogen.

In some embodiments, the additional signaling agent is an immune-modulating agent, e.g. one or more of an interleukin, interferon, and tumor necrosis factor.

In some embodiments, the additional signaling agent is an interleukin, including for example IL-1β; IL-2; IL-3; IL-4; IL-5; IL-6; IL-7; IL-8; IL-9; IL-10; IL-11; IL-12; IL-13; IL-14; IL-15; IL-16; IL-17; IL-18; IL-19; IL-20; IL-21; IL-22; IL-23; IL-24; IL-25; IL-26; IL-27; IL-28; IL-29; IL-30; IL-31; IL-32; IL-33; IL-35; IL-36 or a fragment, variant, analogue, or family-member thereof. Interleukins are a group of multi-functional cytokines synthesized by lymphocytes, monocytes, and macrophages. Known functions include stimulating proliferation of immune cells (e.g., T helper cells, B cells, eosinophils, and lymphocytes), chemotaxis of neutrophils and T lymphocytes, and/or inhibition of interferons. Interleukin activity can be determined using assays known in the art: Matthews et al., in Lymphokines and Interferens: A Practical Approach, Clemens et al., eds, IRL Press, Washington, D.C. 1987, pp. 221-225; and Orencole & Dinarello (1989) Cytokine 1, 14-20.

In some embodiments, the signaling agent is a modified version of an interferon such as interferon types I, II, and III. Illustrative interferons, including for example, interferon-α-1, 2, 4, 5, 6, 7, 8, 10, 13, 14, 16, 17, and 21, interferon-β and interferon-γ, interferon κ, interferon ε, interferon τ, and interferon ω̅.

In embodiments, the additional signaling agent is a type I interferon. In embodiments, the type I interferon is selected from IFN-α2, IFNα1, IFN-β, IFN-γ, Consensus IFN, IFN-ε, IFN-κ, IFN-τ, IFN-δ, and IFN-v. In some embodiments, the additional signaling agent is a modified version of a tumor necrosis factor (TNF) or a protein in the TNF family, including but not limited to, TNF-α, TNF-β, LT-β, CD40L, CD27L, CD30L, FASL, 4-1BBL, OX40L, and TRAIL.

In various embodiments, the additional signaling agent is a modified (e.g. mutant) form of the signaling agent having one or more mutations. In various embodiments, the mutations allow for the modified signaling agent to have one or more of attenuated activity such as one or more of reduced binding affinity, reduced endogenous activity, and reduced specific bioactivity relative to an unmodified or unmutated, i.e. the wild type form of the signaling agent (e.g. comparing the same signaling agent in a wild type form versus a modified (e.g. mutant) form). In various embodiments, the mutations allow for the modified signaling agent to have one or more of attenuated activity such as one or more of reduced binding affinity, reduced endogenous activity, and reduced specific bioactivity relative to unmodified or unmutated signaling agent. In some embodiments, the mutations, which attenuate or reduce binding or affinity, include those mutations that substantially reduce or ablate binding or activity. In some embodiments, the mutations, which attenuate or reduce binding or affinity, are different than those mutations which substantially reduce or ablate binding or activity. Consequentially, in various embodiments, the mutations allow for the signaling agent to be more safe, e.g. have reduced systemic toxicity, reduced side effects, and reduced off-target effects relative to unmutated, i.e. wild type, signaling agent (e.g. comparing the same signaling agent in a wild type form versus a modified (e.g. mutant) form). In various embodiments, the mutations allow for signaling agent to be safer, e.g. have reduced systemic toxicity, reduced side effects, and reduced off-target effects relative to an unmutated signaling agent.

In various embodiments, the additional signaling agent is modified to have one or more mutations that reduce its binding affinity or activity for one or more of its receptors. In some embodiments, the signaling agent is modified to have one or more mutations that substantially reduce or ablate binding affinity or activity for the receptors. In some embodiments, the activity provided by the wild type signaling agent is agonism at the receptor (e.g. activation of a cellular effect at a site of therapy). For example, the wild type signaling agent may activate its receptor. In such embodiments, the mutations result in the modified signaling agent to have reduced or ablated activating activity at the receptor. For example, the mutations may result in the modified signaling agent to deliver a reduced activating signal to a target cell or the activating signal could be ablated. In some embodiments, the activity provided by the wild type signaling agent is antagonism at the receptor (e.g. blocking or dampening of a cellular effect at a site of therapy). For example, the wild type signaling agent may antagonize or inhibit the receptor. In these embodiments, the mutations result in the modified signaling agent to have a reduced or ablated antagonizing activity at the receptor. For example, the mutations may result in the modified signaling agent to deliver a reduced inhibitory signal to a target cell or the inhibitory signal could be ablated. In various embodiments, the signaling agent is antagonistic due to one or more mutations, e.g. an agonistic signaling agent is converted to an antagonistic signaling agent (e.g. as described in WO 2015/007520, the entire contents of which are hereby incorporated by reference) and, such a converted signaling agent, optionally, also bears one or more mutations that reduce its binding affinity or activity for one or more of its receptors or that substantially reduce or ablate binding affinity or activity for one or more of its receptors.

In some embodiments, the reduced affinity or activity at the receptor is inducible and/or restorable by attachment with one or more of the targeting moieties or upon inclusion in the chimeric protein complex (e.g., Fc-based chimeric protein complex) disclosed herein. In other embodiments, the reduced affinity or activity at the receptor is not substantially inducible and/or restorable by the activity of one or more of the targeting moieties or upon inclusion in the chimeric protein complex (e.g., Fc-based chimeric protein complex) disclosed herein.

In various embodiments, the additional signaling agent is active on target cells because the targeting moiety(ies) compensates for the missing/insufficient binding (e.g., without limitation and/or avidity) required for substantial activation. In various embodiments, the modified signaling agent is substantially inactive en route to the site of therapeutic activity and has its effect substantially on specifically targeted cell types, which greatly reduces undesired side effects.

In some embodiments, the additional signaling agent may include one or more mutations that attenuate or reduce binding or affinity for one receptor (i.e., a therapeutic receptor) and one or more mutations that substantially reduce or ablate binding or activity at a second receptor. In such embodiments, these mutations may be at the same or at different positions (i.e., the same mutation or multiple mutations). In some embodiments, the mutation(s) that reduce binding and/or activity at one receptor is different than the mutation(s) that substantially reduce or ablate at another receptor. In some embodiments, the mutation(s) that reduce binding and/or activity at one receptor is the same as the mutation(s) that substantially reduce or ablate at another receptor. In some embodiments, the present chimeric proteins or chimeric protein complexes such as Fc-based chimeric protein complexes have a modified signaling agent that has both mutations that attenuate binding and/or activity at a therapeutic receptor and therefore allow for a more controlled, on-target therapeutic effect (e.g. relative wild type signaling agent) and mutations that substantially reduce or ablate binding and/or activity at another receptor and therefore reduce side effects (e.g. relative to wild type signaling agent).

In some embodiments, the substantial reduction or ablation of binding or activity is not substantially inducible and/or restorable with a targeting moiety or upon inclusion in the chimeric protein complex (e.g., Fc-based chimeric protein complex) disclosed herein. In some embodiments, the substantial reduction or ablation of binding or activity is inducible and/or restorable with a targeting moiety or upon inclusion in the chimeric protein complex (e.g., Fc-based chimeric protein complex) disclosed herein. In various embodiments, substantially reducing or ablating binding or activity at a second receptor also may prevent deleterious effects that are mediated by the other receptor. Alternatively, or in addition, substantially reducing or ablating binding or activity at the other receptor causes the therapeutic effect to improve as there is a reduced or eliminated sequestration of the therapeutic chimeric proteins or chimeric protein complexes such as Fc-based chimeric protein complexes away from the site of therapeutic action. For instance, in some embodiments, this obviates the need of high doses of the chimeric proteins or chimeric protein complexes such as Fc-based chimeric protein complexes that compensate for loss at the other receptor. Such ability to reduce dose further provides a lower likelihood of side effects.

In various embodiments, the additional modified signaling agent comprises one or more mutations that cause the signaling agent to have reduced, substantially reduced, or ablated affinity, e.g. binding (e.g. K_{D}) and/or activation (for instance, when the modified signaling agent is an agonist of its receptor, measurable as, for example, K_{A} and/or EC₅₀) and/or inhibition (for instance, when the modified signaling agent is an antagonist of its receptor, measurable as, for example, K_{I} and/or IC₅₀), for one or more of its receptors. In various embodiments, the reduced affinity at the signaling agent's receptor allows for attenuation of activity (inclusive of agonism or antagonism). In such embodiments, the modified signaling agent has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor relative to the wild type signaling agent. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type signaling agent.

In embodiments wherein the chimeric proteins or chimeric protein complexes such as Fc-based chimeric protein complex has mutations that reduce binding at one receptor and substantially reduce or ablate binding at a second receptor, the attenuation or reduction in binding affinity of a modified signaling agent for one receptor is less than the substantial reduction or ablation in affinity for the other receptor. In some embodiments, the attenuation or reduction in binding affinity of a modified signaling agent for one receptor is less than the substantial reduction or ablation in affinity for the other receptor by about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, or about 95%. In various embodiments, substantial reduction or ablation refers to a greater reduction in binding affinity and/or activity than attenuation or reduction.

In various embodiments, the additional modified signaling agent comprises one or more mutations that reduce the endogenous activity of the signaling agent to about 75%, or about 70%, or about 60%, or about 50%, or about 40%, or about 30%, or about 25%, or about 20%, or about 10%, or about 5%, or about 3%, or about 1%, e.g., relative to the wild type signaling agent.

In various embodiments, the additional modified signaling agent comprises one or more mutations that cause the signaling agent to have reduced affinity and/or activity for a receptor of any one of the cytokines, growth factors, and hormones as described herein.

In some embodiments, the additional modified signaling agent comprises one or more mutations that cause the signaling agent to have reduced affinity for its receptor that is lower than the binding affinity of the targeting moiety(ies) for its (their) receptor(s). In some embodiments, this binding affinity differential is between signaling agent/receptor and targeting moiety/receptor on the same cell. In some embodiments, this binding affinity differential allows for the signaling agent, e.g. mutated signaling agent, to have localized, on-target effects and to minimize off-target effects that underlie side effects that are observed with wild type signaling agent. In some embodiments, this binding affinity is at least about 2-fold, or at least about 5-fold, or at least about 10-fold, or at least about 15-fold lower, or at least about 25-fold, or at least about 50-fold lower, or at least about 100-fold, or at least about 150-fold.

Receptor binding activity may be measured using methods known in the art. For example, affinity and/or binding activity may be assessed by Scatchard plot analysis and computer-fitting of binding data (e.g. Scatchard, 1949) or by reflectometric interference spectroscopy under flow through conditions, as described by Brecht et al. (1993), the entire contents of all of which are hereby incorporated by reference.

The amino acid sequences of the wild type signaling agents described herein are well known in the art. Accordingly, in various embodiments the additional modified signaling agent comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known wild type amino acid sequences of the signaling agents described herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments the additional modified signaling agent comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments, the additional modified signaling agent comprises an amino acid sequence having one or more amino acid mutations. In some embodiments, the one or more amino acid mutations may be independently selected from substitutions, insertions, deletions, and truncations.

In some embodiments, the amino acid mutations are amino acid substitutions, and may include conservative and/or non-conservative substitutions as described herein.

As described herein, the additional modified signaling agents bear mutations that affect affinity and/or activity at one or more receptors. In various embodiments, there is reduced affinity and/or activity at a therapeutic receptor, e.g. a receptor through which a desired therapeutic effect is mediated (e.g. agonism or antagonism). In various embodiments, the modified signaling agents bear mutations that substantially reduce or ablate affinity and/or activity at a receptor, e.g. a receptor through which a desired therapeutic effect is not mediated (e.g. as the result of promiscuity of binding). The receptors of any modified signaling agents, e.g. one of the cytokines, growth factors, and hormones as described herein, are known in the art.

Illustrative mutations which provide reduced affinity and/or activity (e.g. agonistic) at a receptor are found in WO 2013/107791 (e.g. with regard to interferons), WO 2015/007542 (e.g. with regard to interleukins), and WO 2015/007903 (e.g. with regard to TNF), the entire contents of each of which are hereby incorporated by reference. Illustrative mutations which provide reduced affinity and/or activity (e.g. antagonistic) at a therapeutic receptor are found in WO 2015/007520, the entire contents of which are hereby incorporated by reference.

In some embodiments, the additional modified signaling agent comprises one or more mutations that cause the signaling agent to have reduced affinity and/or activity for a type I cytokine receptor, a type II cytokine receptor, a chemokine receptor, a receptor in the Tumor Necrosis Factor Receptor (TNFR) superfamily, TGF-beta Receptors, a receptor in the immunoglobulin (lg) superfamily, and/or a receptor in the tyrosine kinase superfamily.

In various embodiments, the receptor for the additional signaling agent is a Type I cytokine receptor. Type I cytokine receptors are known in the art and include, but are not limited to receptors for IL2 (beta-subunit), IL3, IL4, IL5, IL6, IL7, IL9, IL11, IL12, GM-CSF, G-CSF, LIF, CNTF, and also the receptors for Thrombopoietin (TPO), Prolactin, and Growth hormone. Illustrative type I cytokine receptors include, but are not limited to, GM-CSF receptor, G-CSF receptor, LIF receptor, CNTF receptor, TPO receptor, and type I IL receptors.

In various embodiments, the receptor for the additional signaling agent is a Type II cytokine receptor. Type II cytokine receptors are multimeric receptors composed of heterologous subunits, and are receptors mainly for interferons. This family of receptors includes, but is not limited to, receptors for interferon-α, interferon-β and interferon-γ, IL10, IL22, and tissue factor. Illustrative type II cytokine receptors include, but are not limited to, IFN-α receptor (e.g. IFNAR1 and IFNAR2), IFN-β receptor, IFN-γ receptor (e.g. IFNGR1 and IFNGR2), and type II IL receptors.

In various embodiments, the receptor for the additional signaling agent is a G protein-coupled receptor. Chemokine receptors are G protein-coupled receptors with seven transmembrane structure and coupled to G-protein for signal transduction. Chemokine receptors include, but are not limited to, CC chemokine receptors, CXC chemokine receptors, CX3C chemokine receptors, and XC chemokine receptor (XCR1). Illustrative chemokine receptors include, but are not limited to, CCR1, CCR2, CCR3, CCR4, CCR5, CCR6, CCR7, CCR8, CCR9, CCR10, CXCR1, CXCR2, CXCR3, CXCR3B, CXCR4, CXCR5, CSCR6, CXCR7, XCR1, and CX3CR1.

In various embodiments, the receptor for the additional signaling agent is a TNFR family member. Tumor necrosis factor receptor (TNFR) family members share a cysteine-rich domain (CRD) formed of three disulfide bonds surrounding a core motif of CXXCXXC creating an elongated molecule. Illustrative tumor necrosis factor receptor family members include: CDI 20a (TNFRSFIA), CD 120b (TNFRSFIB), Lymphotoxin beta receptor (LTBR, TNFRSF3), CD 134 (TNFRSF4), CD40 (CD40, TNFRSF5), FAS (FAS, TNFRSF6), TNFRSF6B (TNFRSF6B), CD27 (CD27, TNFRSF7), CD30 (TNFRSF8), CD137 (TNFRSF9), TNFRSFIOA (TNFRSFIOA), TNFRSFIOB, (TNFRSFIOB), TNFRSFIOC (TNFRSFIOC), TNFRSFIOD (TNFRSFIOD), RANK (TNFRSFI IA), Osteoprotegerin (TNFRSFI IB), TNFRSF12A (TNFRSF12A), TNFRSF13B (TNFRSF13B), TNFRSF13C (TNFRSF13C), TNFRSF14 (TNFRSF14), Nerve growth factor receptor (NGFR, TNFRSF16), TNFRSF17 (TNFRSF17), TNFRSF18 (TNFRSF18), TNFRSF19 (TNFRSF19), TNFRSF21 (TNFRSF21), and TNFRSF25 (TNFRSF25).

In various embodiments, the receptor for the additional signaling agent is a TGF-beta receptor. TGF-beta receptors are single pass serine/threonine kinase receptors. TGF-beta receptors include, but are not limited to, TGFBR1, TGFBR2, and TGFBR3.

In various embodiments, the receptor for the additional signaling agent is an Ig superfamily receptor. Receptors in the immunoglobulin (Ig) superfamily share structural homology with immunoglobulins. Receptors in the Ig superfamily include, but are not limited to, interleukin-1 receptors, CSF-1R, PDGFR (e.g. PDGFRA and PDGFRB), and SCFR.

In various embodiments, the receptor for the additional signaling agent is a tyrosine kinase superfamily receptor. Receptors in the tyrosine kinase superfamily are well known in the art. There are about 58 known receptor tyrosine kinases (RTKs), grouped into 20 subfamilies. Receptors in the tyrosine kinase superfamily include, but are not limited to, FGF receptors and their various isoforms such as FGFR1, FGFR2, FGFR3, FGFR4, and FGFR5.

In an embodiment, the additional modified signaling agent is interferon α. In such embodiments, the modified IFN-α agent has reduced affinity and/or activity for the IFN-α/B receptor (IFNAR), i.e., IFNAR1 and/or IFNAR2 chains. In some embodiments, the modified IFN-α agent has substantially reduced or ablated affinity and/or activity for the IFN-α/B receptor (IFNAR), i.e., IFNAR1 and/or IFNAR2 chains. Mutant forms of interferon α are known to the person skilled in the art. In an illustrative embodiment, the modified signaling agent is the allelic form IFN-α2a having the amino acid sequence of SEQ ID NO:233.

In an illustrative embodiment, the modified signaling agent is the allelic form IFN-α2b having the amino acid sequence of SEQ ID NO:234 (which differs from IFN-α2a at amino acid position 23).

In some embodiments, said IFN-α2 mutant (IFN-α2a or IFN-α2b) is mutated at one or more amino acids at positions 144-154, such as amino acid positions 148, 149 and/or 153. In some embodiments, the IFN-α2 mutant comprises one or more mutations selected from L153A, R149A, and M148A. Such mutants are described, for example, in WO2013/107791 and Piehler et al., (2000) J. Biol. Chem, 275:40425-33, the entire contents of all of which are hereby incorporated by reference.

In some embodiments, the IFN-α2 mutants have reduced affinity and/or activity for IFNAR1. In some embodiments, the IFN-α2 mutant comprises one or more mutations selected from F64A, N65A, T69A, L80A, Y85A, and Y89A, as described in WO2010/030671, the entire contents of which is hereby incorporated by reference.

In some embodiments, the IFN-α2 mutant comprises one or more mutations selected from K133A, R144A, R149A, and L153A as described in WO2008/124086, the entire contents of which is hereby incorporated by reference.

In some embodiments, the IFN-α2 mutant comprises one or more mutations selected from R120E and R120E/K121E, as described in WO2015/007520 and WO2010/030671, the entire contents of which are hereby incorporated by reference. In such embodiments, said IFN-α2 mutant antagonizes wildtype IFN-α2 activity. In such embodiments, said mutant IFN-α2 has reduced affinity and/or activity for IFNAR1 while affinity and/or activity of IFNR2 is retained.

In some embodiments, the human IFN-α2 mutant comprises (1) one or more mutations selected from R120E and R120E/K121E, which, without wishing to be bound by theory, create an antagonistic effect and (2) one or more mutations selected from K133A, R144A, R149A, and L153A, which, without wishing to be bound by theory, allow for an attenuated effect at, for example, IFNAR2. In an embodiment, the human IFN-α2 mutant comprises R120E and L153A.

In some embodiments, the human IFN-α2 mutant comprises one or more mutations selected from, L15A, A19W, R22A, R23A, L26A, F27A, L30A, L30V, K31A, D32A, R33K, R33A, R33Q, H34A, D35A, Q40A, D114R, L117A, R120A, R125A, K134A, R144A, A145G, A145M, M148A, R149A, S152A, L153A, and N156A as disclosed in WO 2013/059885, the entire disclosures of which are hereby incorporated by reference. In some embodiments, the human IFN-α2 mutant comprises the mutations H57Y, E58N, Q61S, and/or L30A as disclosed in WO 2013/059885. In some embodiments, the human IFN-α2 mutant comprises the mutations H57Y, E58N, Q61S, and/or R33A as disclosed in WO 2013/059885. In some embodiments, the human IFN-α2 mutant comprises the mutations H57Y, E58N, Q61S, and/or M148A as disclosed in WO 2013/059885. In some embodiments, the human IFN-α2 mutant comprises the mutations H57Y, E58N, Q61S, and/or L153A as disclosed in WO 2013/059885. In some embodiments, the human IFN-α2 mutant comprises the mutations N65A, L80A, Y85A, and/or Y89A as disclosed in WO 2013/059885. In some embodiments, the human IFN-α2 mutant comprises the mutations N65A, L80A, Y85A, Y89A, and/or D114A as disclosed in WO 2013/059885. In some embodiments, the human IFN-α2 mutant comprises one or more mutations selected from R144X₁, A145X₂, and R33A, wherein X₁ is selected from A, S, T, Y, L, and I, and wherein X₂ is selected from G, H, Y, K, and D. In some embodiments, the human IFN-α2 mutant comprises one or more mutations selected from R33A, T106X₃, R120E, R144X₁ A145X₂, M148A, R149A, and L153A with respect to amino acid sequence of SEQ ID NO: 233 or 234, wherein X₁ is selected from A, S, T, Y, L, and I, wherein X₂ is selected from G, H, Y, K, and D, and wherein X₃ is selected from A and E.

In an embodiment, the additional modified signaling agent is interferon α1 (IFNα1). In such embodiments, the modified IFN-α1 agent has reduced affinity and/or activity for the IFN-α/β receptor (IFNAR), i.e., IFNAR1 and/or IFNAR2 chains. In some embodiments, the present invention provides a chimeric protein or chimeric protein complexes, such as Fc-based chimeric protein complexes that include a wild type IFNα1. In various embodiments, the wild-type IFNα1 comprises the following amino acid sequence:

(SEQ ID NO: 5)
CDLPETHSLDNRRTLMLLAQMSRISPSSCLMDRHDFGFPQEEFDGNQFQK
APAISVLHELIQQIFNLFTTKDSSAAWDEDLLDKFCTELYQQLNDLEACV
MQEERVGETPLMNADSILAVKKYFRRITLYLTEKKYSPCAWEVVRAEIMR
SLSLSTNLQERLRRKE

In various embodiments, the IFNα1 variant has one or more mutations that reduce its binding to or its affinity for the IFNAR1 subunit of IFNAR. In one embodiment, the IFNα1 variant has reduced affinity and/or activity at IFNAR1. In some embodiments, the IFNα1 variant has one or more mutations that reduce its binding to or its affinity for the IFNAR2 subunit of IFNAR. In some embodiments, the IFNα1 variant has one or more mutations that reduce its binding to or its affinity for both IFNAR1 and IFNAR2 subunits.

In some embodiments, the IFNα1 variant has one or more mutations that reduce its binding to or its affinity for IFNAR1 and one or more mutations that substantially reduce or ablate binding to or its affinity for IFNAR2. In some embodiments, chimeric proteins and chimeric protein complexes, such as Fc-based chimeric protein complexes, with such IFNα1 variant can provide target-selective IFNAR1 activity (e.g. IFNAR1 activity is inducible and/or restorable via targeting through the targeting moiety or upon inclusion in the chimeric protein complex (e.g., Fc-based chimeric protein complex) disclosed herein).

In some embodiments, the IFNα1 variant has one or more mutations that reduce its binding to or its affinity for IFNAR2 and one or more mutations that substantially reduce or ablate binding to or its affinity for IFNAR1. In some embodiments, chimeric proteins or chimeric protein complexes, such as Fc-based chimeric protein complexes with such IFNα1 variant can provide target-selective IFNAR2 activity (e.g. IFNAR2 activity is inducible and/or restorable via targeting through the targeting moiety or upon inclusion in the chimeric protein complex (e.g., Fc-based chimeric protein complex) disclosed herein).

In some embodiments, the IFNα1 variant has one or more mutations that reduce its binding to or its affinity for IFNAR1 and one or more mutations that reduce its binding to or its affinity for IFNAR2. In some embodiments, chimeric proteins or chimeric protein complexes, such as Fc-based chimeric protein complexes with such IFNα1 variant can provide target-selective IFNAR1 and/or IFNAR2 activity (e.g. IFNAR1 and/IFNAR2 activity is inducible and/or restorable via targeting through the targeting moiety or upon inclusion in the chimeric protein complex (e.g., Fc-based chimeric protein complex) disclosed herein).

In some embodiments, the IFNα1 variant comprises one or more mutations which reduce undesired disulphide pairings to improve product homogeneity and pharmaceutical properties of the chimeric protein or chimeric protein complexes, while simultaneously maintaining or avoiding substantial loss of IFNAR-activation of the modified IFNα1 compared to wild type IFNα1 in the context of chimeric proteins or chimeric protein complexes, including maintaining or avoiding substantial loss of restoration and/or induction of IFNAR-activation by the modified IFNα1 when directed or targeted to a target cell through a targeting moiety. In some embodiments, the IFNα1 is a variant that comprises one or more mutations which reduce undesired disulphide pairings wherein the one or more mutations are, e.g., at amino acid positions C1, C29, C86, C99, or C139 with reference to SEQ ID NO: 5. In some embodiments, the mutation at position C86 can be, e.g., C86S or C86A or C86Y. These C86 mutants of IFNα1 are called reduced cysteine-based aggregation mutants. In some embodiment, the IFNα1 variant includes mutations at positions C1, C86 and C99 with reference to SEQ ID NO: 5. In embodiments, any of C1, C86 and C99 made be deleted or substituted.

In various embodiments, the chimeric protein or chimeric protein complexes, such as Fc-based chimeric protein complexes of the invention comprises a modified version of IFNα1, i.e., a IFNα1 variant including a IFNα1 mutant, as a signaling agent. In various embodiments, the IFNα1 variant encompasses mutants, functional derivatives, analogs, precursors, isoforms, splice variants, or fragments of the interferon.

In some embodiments, the IFNα1 variant comprises one or more mutations that cause the IFNα1 variant to have reduced affinity for a receptor. In some embodiments, the IFNα1 variant's binding affinity for a receptor is lower than the binding affinity of the targeting moiety for its receptor. In some embodiments, this binding affinity differential is between the IFNα1 variant/receptor and targeting moiety/receptor on the same cell. In some embodiments, this binding affinity, differential allows for the IFNα1 variant to have localized, on-target effects and to minimize off-target effects that underlie side effects that are observed with wild type IFNα1. In some embodiments, this binding affinity is at least about 2-fold, or at least about 5-fold, or at least about 10-fold, or at least about 15-fold lower, or at least about 25-fold, or at least about 50-fold lower, or at least about 100-fold, or at least about 150-fold less.

In an illustrative embodiment, the modified additional signaling agent is IFN-β. In various embodiments, the IFN-encompasses functional derivatives, analogs, precursors, isoforms, splice variants, or fragments of IFN-β. In various embodiments, the IFN-β encompasses IFN-β derived from any species. In an embodiment, the chimeric proteins or chimeric protein complexes such as Fc-based chimeric protein complex comprises a modified version of mouse IFN-β. In another embodiment, the chimeric proteins or chimeric protein complexes such as Fc-based chimeric protein complex comprises a modified version of human IFN-β. Human IFN-β is a polypeptide with a molecular weight of about 22 kDa comprising 166 amino acid residues. The amino acid sequence of human IFN-β is SEQ ID NO: 277.

In some embodiments, the human IFN-β is IFN-β-1a, which is a glycosylated form of human IFN-β. In some embodiments, the human IFN-β is IFN-β-1b, which is a non-glycosylated form of human IFN-β that has a Met-1 deletion and a Cys-17 to Ser mutation.

In various embodiments, the modified IFN-β has one or more mutations that reduce its binding to or its affinity for the IFNAR1 subunit of IFNAR. In one embodiment, the modified IFN-β has reduced affinity and/or activity at IFNAR1. In various embodiments, the modified IFN-β is human IFN-β and has one or more mutations at positions F67, R71, L88, Y92, 195, N96, K123, and R124. In some embodiments, the one or more mutations are substitutions selected from F67G, F67S, R71A, L88G, L88S, Y92G, Y92S, 195A, N96G, K123G, and R124G. In an embodiment, the modified IFN-β comprises the F67G mutation. In an embodiment, the modified IFN-β comprises the K123G mutation. In an embodiment, the modified IFN-comprises the F67G and R71A mutations. In an embodiment, the modified IFN-comprises the L88G and Y92G mutations. In an embodiment, the modified IFN-β comprises the Y92G, 195A, and N96G mutations. In an embodiment, the modified IFN-β comprises the K123G and R124G mutations. In an embodiment, the modified IFN-β comprises the F67G, L88G, and Y92G mutations. In an embodiment, the modified IFN-β comprises the F67S, L88S, and Y92S mutations.

In some embodiments, the modified IFN-β has one or more mutations that reduce its binding to or its affinity for the IFNAR2 subunit of IFNAR. In one embodiment, the modified IFN-β has reduced affinity and/or activity at IFNAR2. In various embodiments, the modified IFN-β is human IFN-β and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G. In an embodiment, the modified IFN-β comprises the W22G mutation. In an embodiment, the modified IFN-β comprises the L32A mutation. In an embodiment, the modified IFN-comprises the L32G mutation. In an embodiment, the modified IFN-β comprises the R35A mutation. In an embodiment, the modified IFN-comprises the R35G mutation. In an embodiment, the modified IFN-β comprises the V148G mutation. In an embodiment, the modified IFN-β comprises the R152A mutation. In an embodiment, the modified IFN-β comprises the R152G mutation. In an embodiment, the modified IFN-β comprises the Y155G mutation. In an embodiment, the modified IFN-β comprises the W22G and R27G mutations. In an embodiment, the modified IFN-β comprises the L32A and R35A mutation. In an embodiment, the modified IFN-β comprises the L151G and R152A mutations. In an embodiment, the modified IFN-comprises the V148G and R152A mutations.

In some embodiments, the modified IFN-β has one or more of the following mutations: R35A, R35T, E42K, M621, G78S. A141Y, A142T, E149K, and R152H. In some embodiments, the modified IFN-β has one or more of the following mutations: R35A, R35T, E42K, M621, G78S, A141Y, A142T, E149K, and R152H in combination with C17S or C17A.

In some embodiments, the modified IFN-β has one or more of the following mutations: R35A, R35T, E42K, M621, G78S, A141Y, A142T, E149K, and R152H in combination with any of the other IFN-β mutations described herein.

The crystal structure of human IFN-β is known and is described in Karpusas et al., (1998) PNAS, 94 (22): 11813-11818. Specifically, the structure of human IFN-β has been shown to include five α-helices (i.e., A, B, C, D, and E) and four loop regions that connect these helices (i.e., AB, BC, CD, and DE loops). In various embodiments, the modified IFN-β has one or more mutations in the A, B, C, D, E helices and/or the AB, BC, CD, and DE loops which reduce its binding affinity or activity at a therapeutic receptor such as IFNAR. Illustrative mutations are described in WO2000/023114 and US20150011732, the entire contents of which are hereby incorporated by reference. In an illustrative embodiment, the modified IFN-β is human IFN-β comprising alanine substitutions at amino acid positions 15, 16, 18, 19, 22, and/or 23. In an illustrative embodiment, the modified IFN-β is human IFN-β comprising alanine substitutions at amino acid positions 28-30, 32, and 33. In an illustrative embodiment, the modified IFN-β is human IFN-β comprising alanine substitutions at amino acid positions 36, 37, 39, and 42. In an illustrative embodiment, the modified IFN-β is human IFN-β comprising alanine substitutions at amino acid positions 64 and 67 and a serine substitution at position 68. In an illustrative embodiment, the modified IFN-β is human IFN-β comprising alanine substitutions at amino acid positions 71-73. In an illustrative embodiment, the modified IFN-β is human IFN-β comprising alanine substitutions at amino acid positions 92, 96, 99, and 100. In an illustrative embodiment, the modified IFN-β is human IFN-β comprising alanine substitutions at amino acid positions 128, 130, 131, and 134. In an illustrative embodiment, the modified IFN-β is human IFN-β comprising alanine substitutions at amino acid positions 149, 153, 156, and 159. In some embodiments, the mutant IFNβ comprises SEQ ID NO:277 and a mutation at W22, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at W22, the mutation being an aliphatic hydrophobic residue selected from glycine (G), alanine (A), leucine (L), isoleucine (I), methionine (M), and valine (V) and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L32, the mutation being an aliphatic hydrophobic residue selected from glycine (G), alanine (A), isoleucine (I), methionine (M), and valine (V).

In some embodiments, the mutant IFNβ comprises SEQ ID NO:277 and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L32, the mutation being an aliphatic hydrophobic residue selected from glycine (G), alanine (A), isoleucine (I), methionine (M), and valine (V) and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at F67, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at F67, the mutation being an aliphatic hydrophobic residue selected from glycine (G), alanine (A), leucine (L), isoleucine (I), methionine (M), and valine (V) and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L88, the mutation being an aliphatic hydrophobic residue selected from glycine (G), alanine (A), isoleucine (I), methionine (M), and valine (V).

In some embodiments, the mutant IFNβ comprises SEQ ID NO:277 and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at F67, the mutation being an aliphatic hydrophobic residue selected from glycine (G), alanine (A), leucine (L), isoleucine (I), methionine (M), and valine (V) and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at 195, the mutation being an aliphatic hydrophobic residue selected from glycine (G), alanine (A), leucine (L), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO:277 and a mutation at N96, the mutation being an aliphatic hydrophobic residue selected from glycine (G), alanine (A), leucine (L), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at Y92, the mutation being an aliphatic hydrophobic residue selected from glycine (G), alanine (A), leucine (L), isoleucine (I), methionine (M), and valine (V) and a mutation at 195, the mutation being an aliphatic hydrophobic residue selected from glycine (G), alanine (A), leucine (L), methionine (M), and valine (V) and a mutation at N96, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at K123, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at K123, the mutation being an aliphatic hydrophobic residue selected from glycine (G), alanine (A), leucine (L), isoleucine (I), methionine (M), and valine (V) and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at L151, the mutation being an aliphatic hydrophobic residue selected from glycine (G), alanine (A), isoleucine (I), methionine (M), and valine (V).

In some embodiments, the mutant IFNβ comprises SEQ ID NO: 277 and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at L151, the mutation being an aliphatic hydrophobic residue selected from glycine (G), alanine (A), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at V148, the mutation being an aliphatic hydrophobic residue selected from glycine (G), alanine (A), leucine (L), isoleucine (I), and methionine (M).

In some embodiments, the mutant IFNβ comprises SEQ ID NO: 277 and a mutation at V148, the mutation being an aliphatic hydrophobic residue selected from glycine (G), alanine (A), leucine (L), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 277 and a mutation at Y155, the mutation being an aliphatic hydrophobic residue selected from glycine (G), alanine (A), leucine (L), isoleucine (I), methionine (M), and valine (V).

In some embodiments, the present invention relates to a chimeric proteins or chimeric protein complexes such as Fc-based chimeric protein complex comprising: (a) a modified IFN-β, having the amino acid sequence of SEQ ID NO: 277 and a mutation at position W22, wherein the mutation is an aliphatic hydrophobic residue and a modified IL-2 or modified IL-2 variant disclosed here; and (b) one or more targeting moieties, said targeting moieties comprising recognition domains which specifically bind to antigens or receptors of interest, the modified IFN-β and the one or more targeting moieties are optionally connected with one or more linkers. In various embodiments the mutation at position W22 is aliphatic hydrophobic residue is selected from G, A, L, I, M, and V. In various embodiments, the mutation at position W22 is G.

Additional illustrative IFNβ mutants are provided in PCT/EP2017/061544, the entire disclosure of which is incorporated by reference herein.

In some embodiments, the modified additional signaling agent is interferon γ. In such embodiments, the modified interferon γ agent has reduced affinity and/or activity for the interferon-gamma receptor (IFNGR), i.e., IFNGR1 and IFNGR2 chains. In some embodiments, the modified interferon γ agent has substantially reduced or ablated affinity and/or activity for the interferon-gamma receptor (IFNGR), i.e., IFNGR1 and/or IFNGR2 chains.

In some embodiments, the modified additional signaling agent is a consensus interferon. The consensus interferon is generated by scanning the sequences of several human non-allelic IFN-α subtypes and assigning the most frequently observed amino acid in each corresponding position. The consensus interferon differs from IFN-α2b at 20 out of 166 amino acids (88% homology), and comparison with IFN-β shows identity at over 30% of the amino acid positions. In various embodiments, the consensus interferon comprises the following amino acid sequence of SEQ ID NO: 278.

In some embodiments, the consensus interferon comprises the amino acid sequence of SEQ ID NO: 279, which differs from the amino acid sequence of SEQ ID NO: 278 by one amino acid, i.e., SEQ ID NO: 279 lacks the initial methionine residue of SEQ ID NO: 278:

In various embodiments, the consensus interferon comprises a modified version of the consensus interferon, i.e., a consensus interferon variant, as a signaling agent. In various embodiments, the consensus interferon variant encompasses functional derivatives, analogs, precursors, isoforms, splice variants, or fragments of the consensus interferon.

In an embodiment, the consensus interferon variants are selected form the consensus interferon variants disclosed in U.S. Pat. Nos. 4,695,623, 4,897,471, 5,541,293, and 8,496,921, the entire contents of all of which are hereby incorporated by reference. For example, the consensus interferon variant may comprise the amino acid sequence of IFN-CON₂ or IFN-CON₃ as disclosed in U.S. Pat. Nos. 4,695,623, 4,897,471, and 5,541,293. In an embodiment, the consensus interferon variant comprises the amino acid sequence of IFN-CON₂: SEQ ID NO: 280.

In an embodiment, the consensus interferon variant comprises the amino acid sequence of IFN-CON₃: SEQ ID NO: 281.

In an embodiment, the consensus interferon variant comprises the amino acid sequence of any one of the variants disclosed in U.S. Pat. No. 8,496,921. For example, the consensus variant may comprise the amino acid sequence of: SEQ ID NO: 282.

In another embodiment, the consensus interferon variant may comprise the amino acid sequence of: SEQ ID NO: 283.

In some embodiments, the consensus interferon variant may be PEGylated, i.e., comprises a PEG moiety. In an embodiment, the consensus interferon variant may comprise a PEG moiety attached at the S156C position of SEQ ID NO: 283.

In some embodiments, the engineered interferon is a variant of human IFN-α2a, with an insertion of Asp at approximately position 41 in the sequence Glu-Glu-Phe-Gly-Asn-Gln (SEQ ID NO: 284) to yield Glu-Glu-Phe-Asp-Gly-Asn-Gln (SEQ ID NO: 285) (which resulted in a renumbering of the sequence relative to IFN-α2a sequence) and the following mutations of Arg23Lys, Leu26Pro, Glu53Gln, Thr54Ala, Pro56Ser, Asp86Glu, Ile104Thr, Gly106Glu, Thr110Glu, Lys117Asn, Arg125Lys, and Lys136Thr. All embodiments herein that describe consensus interferons apply equally to this engineered interferon In some embodiments, the additional modified signaling agent is vascular endothelial growth factor (VEGF). VEGF is a potent growth factor that plays major roles in physiological but also pathological angiogenesis, regulates vascular permeability and can act as a growth factor on cells expressing VEGF receptors. Additional functions include, among others, stimulation of cell migration in macrophage lineage and endothelial cells. Several members of the VEGF family of growth factors exist, as well as at least three receptors (VEGFR-1, VEGFR-2, and VEGFR-3). Members of the VEGF family can bind and activate more than one VEGFR type. For example, VEGF-A binds VEGFR-1 and -2, while VEGF-C can bind VEGFR-2 and -3. VEGFR-1 and VEGFR-2 activation regulate angiogenesis while VEGFR-3 activation is associated with lymphangiogenesis. The major pro-angiogenic signal is generated from activation of VEGFR-2. VEGFR-1 activation has been reported to be possibly associated with negative role in angiogenesis. It has also been reported that VEGFR-1 signaling is important for progression of tumors in vivo via bone marrow-derived VEGFR-1 positive cells (contributing to formation of premetastatic niche in the bone). Several therapies based on VEGF-A directed/neutralizing therapeutic antibodies have been developed, primarily for use in treatment of various human tumors relying on angiogenesis. These are not without side effects though. This may not be surprising considering that these operate as general, non-cell/tissue specific VEGF/VEGFR interaction inhibitors. Hence, it would be desirable to restrict VEGF (e.g. VEGF-A)/EGFR-2 inhibition to specific target cells (e.g. tumor vasculature endothelial cells).

In some embodiments, the VEGF is VEGF-A, VEGF-B, VEFG-C, VEGF-D, or VEGF-E and isoforms thereof including the various isoforms of VEGF-A such as VEGF₁₂₁, VEGF₁₂₁b, VEGF₁₄₅, VEGF₁₆₅, VEGF₁₆₅b, VEGF₁₈₉, and VEGF₂₀₆. In some embodiments, the modified signaling agent has reduced affinity and/or activity for VEGFR-1 (Flt-1) and/or VEGFR-2 (KDR/FIk-1). In some embodiments, the modified signaling agent has substantially reduced or ablated affinity and/or activity for VEGFR-1 (Flt-1) and/or VEGFR-2 (KDR/FIk-1). In an embodiment, the modified signaling agent has reduced affinity and/or activity for VEGFR-2 (KDR/FIk-1) and/or substantially reduced or ablated affinity and/or activity for VEGFR-1 (Flt-1). Such an embodiment finds use, for example, in wound healing methods or treatment of ischmia-related diseases (without wishing to be bound by theory, mediated by VEGFR-2's effects on endothelial cell function and angiogenesis). In various embodiments, binding to VEGFR-1 (Flt-1), which is linked to cancers and pro-inflammatory activities, is avoided. In various embodiments, VEGFR-1 (Flt-1) acts a decoy receptor and therefore substantially reduces or ablates affinity at this receptor avoids sequestration of the therapeutic agent. In an embodiment, the modified signaling agent has substantially reduced or ablated affinity and/or activity for VEGFR-1 (Flt-1) and/or substantially reduced or ablated affinity and/or activity for VEGFR-2 (KDR/FIk-1). In some embodiments, the VEGF is VEGF-C or VEGF-D. In such embodiments, the modified signaling agent has reduced affinity and/or activity for VEGFR-3. Alternatively, the modified signaling agent has substantially reduced or ablated affinity and/or activity for VEGFR-3.

Proangiogenic therapies are also important in various diseases (e.g. ischemic heart disease, bleeding etc.), and include VEGF-based therapeutics. Activation of VEGFR-2 is proangiogenic (acting on endothelial cells). Activation of VEFGR-1 can cause stimulation of migration of inflammatory cells (including, for example, macrophages) and lead to inflammation associated hypervascular permeability. Activation of VEFGR-1 can also promote bone marrow associated tumor niche formation. Thus, VEGF based therapeutic selective for VEGFR-2 activation would be desirable in this case. In addition, cell specific targeting, e.g. to endothelial cells, would be desirable.

In some embodiments, the additional modified signaling agent has reduced affinity and/or activity (e.g. antagonistic) for VEGFR-2 and/or has substantially reduced or ablated affinity and/or activity for VEGFR-1. When targeted to tumor vasculature endothelial cells via a targeting moiety that binds to a tumor endothelial cell marker (e.g. PSMA and others), such construct inhibits VEGFR-2 activation specifically on such marker-positive cells, while not activating VEGFR-1 en route and on target cells (if activity ablated), thus eliminating induction of inflammatory responses, for example. This would provide a more selective and safe anti-angiogenic therapy for many tumor types as compared to VEGF-A neutralizing therapies.

In some embodiments, the additional modified signaling agent has reduced affinity and/or activity (e.g. agonistic) for VEGFR-2 and/or has substantially reduced or ablated affinity and/or activity for VEGFR-1.

Through targeting to vascular endothelial cells, such construct, in some embodiments, promotes angiogenesis without causing VEGFR-1 associated induction of inflammatory responses. Hence, such a construct would have targeted proangiogenic effects with substantially reduced risk of side effects caused by systemic activation of VEGFR-2 as well as VEGR-1.

In an illustrative embodiment, the modified signaling agent is VEGF₁₆₅, which has the amino acid sequence of SEQ ID NO:235.

In another illustrative embodiment, the additional modified signaling agent is VEGF₁₆₅b, which has the amino acid sequence of SEQ ID NO:236.

In these embodiments, the modified signaling agent has a mutation at amino acid 183 (e.g., a substitution mutation at 183, e.g., 183K, 183R, or 183H). Without wishing to be bound by theory, it is believed that such mutations may result in reduced receptor binding affinity. See, for example, U.S. Pat. No. 9,078,860, the entire contents of which are hereby incorporated by reference.

In an embodiment, the additional modified signaling agent is TNF-α. TNF is a pleiotropic cytokine with many diverse functions, including regulation of cell growth, differentiation, apoptosis, tumorigenesis, viral replication, autoimmunity, immune cell functions and trafficking, inflammation, and septic shock. It binds to two distinct membrane receptors on target cells: TNFR1 (p55) and TNFR2 (p75). TNFR1 exhibits a very broad expression pattern whereas TNFR2 is expressed preferentially on certain populations of lymphocytes, Tregs, endothelial cells, certain neurons, microglia, cardiac myocytes and mesenchymal stem cells. Very distinct biological pathways are activated in response to receptor activation, although there is also some overlap. As a general rule, without wishing to be bound by theory, TNFR1 signaling is associated with induction of apoptosis (cell death) and TNFR2 signaling is associated with activation of cell survival signals (e.g. activation of NFkB pathway). Administration of TNF is systemically toxic, and this is largely due to TNFR1 engagement. However, it should be noted that activation of TNFR2 is also associated with a broad range of activities and, as with TNFR1, in the context of developing TNF based therapeutics, control over TNF targeting and activity is important.

In some embodiments, the additional modified signaling agent has reduced affinity and/or activity for TNFR1 and/or TNFR2. In some embodiments, the modified signaling agent has substantially reduced or ablated affinity and/or activity for TNFR1 and/or TNFR2. TNFR1 is expressed in most tissues, and is involved in cell death signaling while, by contrast, TNFR2 is involved in cell survival signaling.

Accordingly, in embodiments directed to methods of treating cancer, the modified signaling agent has reduced affinity and/or activity for TNFR1 and/or substantially reduced or ablated affinity and/or activity for TNFR2. In these embodiments, the chimeric proteins or chimeric protein complexes such as Fc-based chimeric protein complex may be targeted to a cell for which apoptosis is desired, e.g. a tumor cell or a tumor vasculature endothelial cell. In embodiments directed to methods of promoting cell survival, for example, in neurogenesis for the treatment of neurodegenerative disorders, the modified signaling agent has reduced affinity and/or activity for TNFR2 and/or substantially reduced or ablated affinity and/or activity for TNFR1. Stated another way, the present chimeric proteins or chimeric protein complexes such as Fc-based chimeric protein complex, in some embodiments, comprise modified TNF-α agent that allows of favoring either death or survival signals.

In some embodiments, the chimeric proteins or chimeric protein complexes such as Fc-based chimeric protein complex has a modified TNF having reduced affinity and/or activity for TNFR1 and/or substantially reduced or ablated affinity and/or activity for TNFR2. Such a chimera, in some embodiments, is a more potent inducer of apoptosis as compared to a wild type TNF and/or a chimera bearing only mutation(s) causing reduced affinity and/or activity for TNFR1. Such a chimera, in some embodiments, finds use in inducing tumor cell death or a tumor vasculature endothelial cell death (e.g. in the treatment of cancers). Also, in some embodiments, these chimeras avoid or reduce activation of T_{reg} cells via TNFR2, for example, thus further supporting TNFR1-mediated antitumor activity in vivo.

In some embodiments, the chimeric proteins or chimeric protein complexes such as Fc-based chimeric protein complex has a modified TNF having reduced affinity and/or activity for TNFR2 and/or substantially reduced or ablated affinity and/or activity for TNFR1. Such a chimera, in some embodiments, is a more potent activator of cell survival in some cell types, which may be a specific therapeutic objective in various disease settings, including without limitation, stimulation of neurogenesis. In addition, such a TNFR2-favoring chimeras also are useful in the treatment of autoimmune diseases (e.g. Crohn's, diabetes, MS, colitis etc. and many others described herein). In some embodiments, the chimera is targeted to auto-reactive T cells. In some embodiments, the chimera promotes T_{reg} cell activation and indirect suppression of cytotoxic T cells.

In some embodiments, the chimera causes the death of auto-reactive T cells, e.g. by activation of TNFR2 and/or avoidance of TNFR1 (e.g. a modified TNF having reduced affinity and/or activity for TNFR2 and/or substantially reduced or ablated affinity and/or activity for TNFR1). Without wishing to be bound by theory these auto-reactive T cells, have their apoptosis/survival signals altered e.g. by NFkB pathway activity/signaling alterations.

In some embodiments, a TNFR2 based chimera has additional therapeutic applications in diseases, including various autoimmune diseases, heart disease, de-myelinating and neurodegenerative disorders, and infectious disease, among others.

In an embodiment, the wild type TNF-α has the amino acid sequence of SEQ ID NO:237.

In such embodiments, the modified TNF-α agent has mutations at one or more amino acid positions 29, 31, 32, 84, 85, 86, 87, 88, 89, 145, 146 and 147 which produces a modified TNF-α with reduced receptor binding affinity. See, for example, U.S. Pat. No. 7,993,636, the entire contents of which are hereby incorporated by reference.

In some embodiments, the modified human TNF-α moiety has mutations at one or more amino acid positions R32, N34, Q67, H73, L75, T77, S86, Y87, V91, 197, T105, P106, A109, P113, Y115, E127, N137, D143, and A145, as described, for example, in WO/2015/007903, the entire contents of which is hereby incorporated by reference (numbering according to the human TNF sequence, Genbank accession number BAG70306, version BAG70306.1 GI: 197692685). In some embodiments, the modified human TNF-α moiety has substitution mutations selected from R32G, N34G, Q67G, H73G, L75G, L75A, L75S, T77A, S86G, Y87Q, Y87L, Y87A, Y87F, V91G, V91A, 197A, 197Q, 197S, T105G, P106G, A109Y, P113G, Y115G, Y115A, E127G, N137G, D143N, A145G and A145T. In an embodiment, the human TNF-α moiety has a mutation selected from Y87Q, Y87L, Y87A, and Y87F. In another embodiment, the human TNF-α moiety has a mutation selected from 197A, 197Q, and 197S. In a further embodiment, the human TNF-α moiety has a mutation selected from Y115A and Y115G.

In some embodiments, the modified TNF-α agent has one or more mutations selected from N39Y, S147Y, and Y87H, as described in WO2008/124086, the entire contents of which is hereby incorporated by reference.

In an embodiment, the additional modified signaling agent is TNF-β. TNF-β can form a homotrimer or a heterotrimer with LT-β (LT-α1β2). In some embodiments, the modified signaling agent has substantially reduced or ablated affinity and/or activity for TNFR1 and/or TNFR2 and/or herpes virus entry mediator (HEVM) and/or LT-βR.

In an embodiment, the wild type TNF-β has the amino acid sequence of SEQ ID NO:238.

In such embodiments, the modified TNF-β agent may comprise mutations at one or more amino acids at positions 106-113, which produce a modified TNF-β with reduced receptor binding affinity to TNFR2. In an embodiment, the modified signaling agent has one or more substitution mutations at amino acid positions 106-113. In illustrative embodiments, the substitution mutations are selected from Q107E, Q107D, S106E, S106D, Q107R, Q107N, Q107E/S106E, Q107E/S106D, Q107D/S106E, and Q107D/S106D. In another embodiment, the modified signaling agent has an insertion of about 1 to about 3 amino acids at positions 106-113.

In some embodiments, the additional modified agent is a TNF family member (e.g. TNF-alpha, TNF-beta) which can be a single chain trimeric version as described in WO 2015/007903, the entire contents of which are incorporated by reference.

In some embodiments, the modified agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR1. In these embodiments, the modified agent is a TNF family member (e.g. TNF-alpha, TNF-beta) which also, optionally, has substantially reduced or ablated affinity and/or activity for TNFR2. In some embodiments, the modified agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR2. In these embodiments, the modified agent is a TNF family member (e.g. TNF-alpha, TNF-beta) which also, optionally, has substantially reduced or ablated affinity and/or activity for TNFR1. The constructs of such embodiments find use in, for example, methods of dampening TNF response in a cell specific manner. In some embodiments, the antagonistic TNF family member (e.g. TNF-alpha, TNF-beta) is a single chain trimeric version as described in WO 2015/007903.

In an embodiment, the additional modified signaling agent is TRAIL. In some embodiments, the modified TRAIL agent has reduced affinity and/or activity for DR4 (TRAIL-RI) and/or DR5 (TRAIL-RII) and/or DcR1 and/or DcR2. In some embodiments, the modified TRAIL agent has substantially reduced or ablated affinity and/or activity for DR4 (TRAIL-RI) and/or DR5 (TRAIL-RII) and/or DcR1 and/or DcR2. In an embodiment, the wild type TRAIL has the amino acid sequence of SEQ ID NO:239.

In such embodiments, the modified TRAIL agent may comprise a mutation at amino acid positions T127-R132, E144-R149, E155-H161, Y189-Y209, T214-1220, K224-A226, W231, E236-L239, E249-K251, T261-H264 and H270-E271 (Numbering based on the human sequence, Genbank accession number NP_003801, version 10 NP_003801.1, GI: 4507593; see above).

In an embodiment, the additional modified signaling agent is TGFα. In such embodiments, the modified TGFα agent has reduced affinity and/or activity for the epidermal growth factor receptor (EGFR). In some embodiments, the modified TGFα agent has substantially reduced or ablated affinity and/or activity for the epidermal growth factor receptor (EGFR).

In an embodiment, the additional modified signaling agent is TGFβ. In such embodiments, the modified signaling agent has reduced affinity and/or activity for TGFBR1 and/or TGFBR2. In some embodiments, the modified signaling agent has substantially reduced or ablated affinity and/or activity for TGFBR1 and/or TGFBR2. In some embodiments, the modified signaling agent optionally has reduced or substantially reduced or ablated affinity and/or activity for TGFBR3, which, without wishing to be bound by theory, may act as a reservoir of ligand for TGF-beta receptors. In some embodiments, the TGFß may favor TGFBR1 over TGFBR2 or TGFBR2 over TGFBR1. Similarly, LAP, without wishing to be bound by theory, may act as a reservoir of ligand for TGF-beta receptors. In some embodiments, the modified signaling agent has reduced affinity and/or activity for TGFBR1 and/or TGFBR2 and/or substantially reduced or ablated affinity and/or activity for Latency Associated Peptide (LAP). In some embodiments, such chimeras find use in Camurati-Engelmann disease, or other diseases associated with inappropriate TGFß signaling.

In some embodiments, the additional modified agent is a TGF family member (e.g. TGFα, TGFβ) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at one or more of TGFBR1, TGFBR2, TGFBR3. In these embodiments, the modified agent is a TGF family member (e.g. TGFα, TGFβ) which also, optionally, has substantially reduced or ablated affinity and/or activity at one or more of TGFBR1, TGFBR2, TGFBR3.

In some embodiments, the additional modified agent is a TGF family member (e.g. TGFα, TGFβ) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TGFBR1 and/or TGFBR2. In these embodiments, the modified agent is a TGF family member (e.g. TGFα, TGFβ) which also, optionally, has substantially reduced or ablated affinity and/or activity at TGFBR3.

In an embodiment, the additional modified signaling agent is IL-1. In an embodiment, the modified signaling agent is IL-1α or IL-1β. In some embodiments, the modified signaling agent has reduced affinity and/or activity for IL-1R1 and/or IL-1RAcP. In some embodiments, the modified signaling agent has substantially reduced or ablated affinity and/or activity for IL-1R1 and/or IL-1RAcP. In some embodiments, the modified signaling agent has reduced affinity and/or activity for IL-1RAcP. In some embodiments, the modified signaling agent has substantially reduced or ablated affinity and/or activity for IL-1RACP. For instance, in some embodiments, the present modified IL-1 agents avoid interaction at IL-1RAcP and therefore substantially reduce its function as a decoy and/or sink for therapeutic agents.

In an embodiment, the wild type IL-1β has the amino acid sequence of SEQ ID NO:240.

IL-1β is a proinflammatory cytokine and an important immune system regulator. It is a potent activator of CD4 T cell responses, increases proportion of Th17 cells and expansion of IFNγ and IL-4 producing cells. IL-1β is also a potent regulator of CD8⁺ T cells, enhancing antigen-specific CD8⁺ T cell expansion, differentiation, migration to periphery and memory. IL-1β receptors comprise IL-1R1 and IL-1RAcP. Binding to and signaling through the IL-1R1 constitutes the mechanism whereby IL-1β mediates many of its biological (and pathological) activities. IL1-R2 can function as a decoy receptor, thereby reducing IL-1B availability for interaction and signaling through the IL-1R1.

In some embodiments, the modified IL-1α has reduced affinity and/or activity (e.g. agonistic activity) for IL-1R1. In some embodiments, the modified IL-1α has substantially reduced or ablated affinity and/or activity for IL-1RAcP. In such embodiments, there is inducible and/or restorable IL-1β/IL-1R1 signaling and prevention of loss of therapeutic chimeras at IL-R2 and therefore a reduction in dose of IL-1 that is required (e.g. relative to wild type or a chimera bearing only an attenuation mutation for IL-R1). Such constructs find use in, for example, methods of treating cancer, including, for example, stimulating the immune system to mount an anti-cancer response.

In some embodiments, the modified IL-1β has reduced affinity and/or activity (e.g. antagonistic activity, e.g. natural antagonistic activity or antagonistic activity that is the result of one or more mutations, see, e.g., WO 2015/007520, the entire contents of which are hereby incorporated by reference) for IL-1R1. In some embodiments, the modified IL-1β has substantially reduced or ablated affinity and/or activity for IL-1RAcP. In such embodiments, there is the IL-1β/IL-1R1 signaling is not inducible and/or restorable and prevention of loss of therapeutic chimeras at IL-R2 and therefore a reduction in dose of IL-1β that is required (e.g. relative to wild type or a chimera bearing only an attenuation mutation for IL-R1). Such constructs find use in, for example, methods of treating autoimmune diseases, including, for example, suppressing the immune system.

In such embodiments, the modified signaling agent has a deletion of amino acids 52-54 which produces a modified human IL-1β with reduced binding affinity for type I IL-1R and reduced biological activity. See, for example, WO 1994/000491, the entire contents of which are hereby incorporated by reference. In some embodiments, the modified human IL-1β has one or more substitution mutations selected from A117G/P118G, R120X, L122A, T125G/L126G, R127G, Q130X, Q131G, K132A, S137G/Q138Y, L145G, H146X, L145A/L147A, Q148X, Q148G/Q150G, Q150G/D151A, M152G, F162A, F162A/Q164E, F166A, Q164E/E167K, N169G/D170G, 1172A, V174A, K208E, K209X, K209A/K210A, K219X, E221X, E221 S/N224A, N224S/K225S, E244K, N245Q (where X can be any change in amino acid, e.g., a non-conservative change), which exhibit reduced binding to IL-1R, as described, for example, in WO2015/007542 and WO/2015/007536, the entire contents of which is hereby incorporated by reference (numbering base on the human IL-1 β sequence, Genbank accession number NP_000567, version NP-000567.1, GI: 10835145). In some embodiments, the modified human IL-1β may have one or more mutations selected from R120A, R120G, Q130A, Q130W, H146A, H146G, H146E, H146N, H146R, Q148E, Q148G, Q148L, K209A, K209D, K219S, K219Q, E221S and E221K. In an embodiment, the modified human IL-1β comprises the mutations Q131G and Q148G. In an embodiment, the modified human IL-1β comprises the mutations Q148G and K208E. In an embodiment, the modified human IL-1β comprises the mutations R120G and Q131G. In an embodiment, the modified human IL-1β comprises the mutations R120G and H146G. In an embodiment, the modified human IL-1β comprises the mutations R120G and K208E. In an embodiment, the modified human IL-1β comprises the mutations R120G, F162A, and Q164E.

In an embodiment, the additional modified signaling agent is IL-2. In such an embodiment, the modified signaling agent has reduced affinity and/or activity for IL-2Rα and/or IL-2Rβ and/or IL-2Rγ. In some embodiments, the modified signaling agent has reduced affinity and/or activity for IL-2Rβ and/or IL-2Rγ. In some embodiments, the modified signaling agent has substantially reduced or ablated affinity and/or activity for IL-2Rα. Such embodiments may be relevant for treatment of cancer, for instance when the modified IL-2 is agonistic at IL-2Rβ and/or IL-2Rγ. For instance, the present constructs may favor attenuated activation of CD8⁺ T cells (which can provide an anti-tumor effect), which have IL2 receptors β and γ and disfavor Tregs (which can provide an immune suppressive, pro-tumor effect), which have IL2 receptors α, β, and γ. Further, in some embodiments, the preferences for IL-2Rβ and/or IL-2Rγ over IL-2Rα avoid IL-2 side effects such as pulmonary edema. Also, IL-2-based chimeras are useful for the treatment of autoimmune diseases, for instance when the modified IL-2 is antagonistic (e.g. natural antagonistic activity or antagonistic activity that is the result of one or more mutations, see, e.g., WO 2015/007520, the entire contents of which are hereby incorporated by reference) at IL-2Rβ and/or IL-2Rγ. For instance, the present constructs may favor attenuated suppression of CD8⁺ T cells (and therefore dampen the immune response), which have IL2 receptors β and γ and disfavor Tregs which have IL2 receptors α, β, and γ. Alternatively, in some embodiments, the chimeras bearing IL-2 favor the activation of Tregs, and therefore immune suppression, and activation of disfavor of CD8⁺ T cells. For instance, these constructs find use in the treatment of diseases or diseases that would benefit from immune suppression, e.g. autoimmune disorders.

In some embodiments, the chimeric proteins or chimeric protein complexes such as Fc-based chimeric protein complex has targeting moieties as described herein directed to CD8⁺ T cells as well as a modified IL-2 agent having reduced affinity and/or activity for IL-2Rβ and/or IL-2Rγ and/or substantially reduced or ablated affinity and/or activity for IL-2Rα. In some embodiments, these constructs provide targeted CD8⁺ T cell activity and are generally inactive (or have substantially reduced activity) towards T_{reg} cells. In some embodiments, such constructs have enhanced immune stimulatory effect compared to wild type IL-2 (e.g., without wishing to be bound by theory, by not stimulating Tregs), whilst eliminating or reducing the systemic toxicity associated with IL-2.

In an embodiment, the wild type IL-2 has the amino acid sequence of SEQ ID NO:241.

In such embodiments, the modified IL-2 agent has one or more mutations at amino acids L72 (L72G, L72A, L72S, L72T, L72Q, L72E, L72N, L72D, L72R, or L72K), F42 (F42A, F42G, F42S, F42T, F42Q, F42E, F42N, F42D, F42R, or F42K) and Y45 (Y45A, Y45G, Y45S, Y45T, Y45Q, Y45E, Y45N, Y45D, Y45R or Y45K). Without wishing to be bound by theory, it is believed that these modified IL-2 agents have reduced affinity for the high-affinity IL-2 receptor and preserves affinity to the intermediate-affinity IL-2 receptor, as compared to the wild-type IL-2. See, for example, US Patent Publication No. 2012/0244112, the entire contents of which are hereby incorporated by reference.

In an embodiment, the additional modified signaling agent is IL-3. In some embodiments, the modified signaling agent has reduced affinity and/or activity for the IL-3 receptor, which is a heterodimer with a unique alpha chain paired with the common beta (beta c or CD131) subunit. In some embodiments, the modified signaling agent has substantially reduced or ablated affinity and/or activity for the IL-3 receptor, which is a heterodimer with a unique alpha chain paired with the common beta (beta c or CD131) subunit.

In an embodiment, the additional modified signaling agent is IL-4. In such an embodiment, the modified signaling agent has reduced affinity and/or activity for type 1 and/or type 2 IL-4 receptors. In such an embodiment, the modified signaling agent has substantially reduced or ablated affinity and/or activity for type 1 and/or type 2 IL-4 receptors. Type 1 IL-4 receptors are composed of the IL-4Rα subunit with a common γ chain and specifically bind IL-4. Type 2 IL-4 receptors include an IL-4Rα subunit bound to a different subunit known as IL-13Rα1. In some embodiments, the modified signaling agent has substantially reduced or ablated affinity and/or activity the type 2 IL-4 receptors.

In an embodiment, the wild type IL-4 has the amino acid sequence of SEQ ID NO:242.

In such embodiments, the modified IL-4 agent has one or more mutations at amino acids R121 (R121A, R121D, R121E, R121F, R121H, R1211, R121K, R121N, R121P, R121T, R121W), E122 (E122F), Y124 (Y124A, Y124Q, Y124R, Y124S, Y124T) and S125 (S125A). Without wishing to be bound by theory, it is believed that these modified IL-4 agents maintain the activity mediated by the type I receptor, but significantly reduces the biological activity mediated by the other receptors. See, for example, U.S. Pat. No. 6,433,157, the entire contents of which are hereby incorporated by reference.

In an embodiment, the additional modified signaling agent is IL-6. IL-6 signals through a cell-surface type I cytokine receptor complex including the ligand-binding IL-6R chain (CD126), and the signal-transducing component gp130. IL-6 may also bind to a soluble form of IL-6R (sIL-6R), which is the extracellular portion of IL-6R. The sIL-6R/IL-6 complex may be involved in neurites outgrowth and survival of neurons and, hence, may be important in nerve regeneration through remyelination. Accordingly, in some embodiments, the modified signaling agent has reduced affinity and/or activity for IL-6R/gp130 and/or sIL-6R. In some embodiments, the modified signaling agent has substantially reduced or ablated affinity and/or activity for IL-6R/gp130 and/or sIL-6R.

In an embodiment, the wild type IL-6 has the amino acid sequence of SEQ ID NO:243.

In such embodiments, the modified signaling agent has one or more mutations at amino acids 58, 160, 163, 171 or 177. Without wishing to be bound by theory, it is believed that these modified IL-6 agents exhibit reduced binding affinity to IL-6Ralpha and reduced biological activity. See, for example, WO 97/10338, the entire contents of which are hereby incorporated by reference.

In an embodiment, the additional modified signaling agent is IL-10. In such an embodiment, the modified signaling agent has reduced affinity and/or activity for IL-10 receptor-1 and IL-10 receptor-2. In some embodiments, the modified signaling agent has substantially reduced or ablated affinity and/or activity for IL-10 receptor-1 and IL-10 receptor-2

In an embodiment, the additional modified signaling agent is IL-11. In such an embodiment, the modified signaling agent has reduced affinity and/or activity for IL-11Rα and/or IL-11Rβ and/or gp130. In such an embodiment, the modified signaling agent has substantially reduced or ablated affinity and/or activity for IL-11Rα and/or IL-11Rβ and/or gp130.

In an embodiment, the additional modified signaling agent is IL-12. In such an embodiment, the modified signaling agent has reduced affinity and/or activity for IL-12Rβ1 and/or IL-12Rβ2. In such an embodiment, the modified signaling agent has substantially reduced or ablated affinity and/or activity for IL-12Rβ1 and/or IL-12Rβ2.

In an embodiment, the additional modified signaling agent is IL-13. In such an embodiment, the modified signaling agent has reduced affinity and/or activity for the IL-4 receptor (IL-4Rα) and IL-13Rα1. In some embodiments, the modified signaling agent has substantially reduced or ablated affinity and/or activity for IL-4 receptor (IL-4Rα) or IL-13Rα1.

In an embodiment, the wild type IL-1β has the amino acid sequence of SEQ ID NO:244.

In such embodiments, the modified IL-1β agent has one or more mutations at amino acids 13, 16, 17, 66, 69, 99, 102, 104, 105, 106, 107, 108, 109, 112, 113 and 114. Without wishing to be bound by theory, it is believed that these modified IL-1β agents exhibit reduced biological activity. See, for example, WO 2002/018422, the entire contents of which are hereby incorporated by reference.

In an embodiment, the signaling agent is a wild type or modified IL-15. In embodiments, the modified IL-15 has reduced affinity and/or activity for interleukin 15 receptor.

In an embodiment, the wild type IL-15 has the amino acid sequence of:

(SEQ ID NO: 292)
NWVNVISDLKKIEDLIQSMHIDATLYTESDVHPSCKVTAMKCFLLELQVI
SHESGDTDIHDTVENLIILANNILSSNGNITESGCKECEELEEKNIKEFL
QSFVHIVQMFINTS

In such embodiments, the modified IL-15 agent has one or more mutations at amino acids S7, D8, K10, K11, E46, L47, V49, 150, D61, N65, L66, 167, 168, L69, N72, Q108 with respect to SEQ ID NO: 292.

In an embodiment, the additional modified signaling agent is IL-18. In some embodiments, the modified signaling agent has reduced affinity and/or activity for IL-18Rα and/or IL-18Rβ. In some embodiments, the modified signaling agent has substantially reduced or ablated affinity and/or activity for IL-18Rα and/or IL-18Rβ. In some embodiments, the modified signaling agent has substantially reduced or ablated affinity and/or activity for IL-18Rα type II, which is an isoform of IL-18Rα that lacks the TIR domain required for signaling.

In an embodiment, the wild type IL-18 has the amino acid sequence of SEQ ID NO:245.

In such embodiments, the modified IL-18 agent may comprise one or more mutations in amino acids or amino acid regions selected from Y37-K44, R49-Q54, D59-R63, E67-C74, R80, M87-A97, N 127-K129, Q139-M149, K165-K171, R183 and Q190-N191, as described in WO/2015/007542, the entire contents of which are hereby incorporated by reference (numbering based on the human IL-18 sequence, Genbank accession number AAV38697, version AAV38697.1, GI: 54696650).

In an embodiment, the additional modified signaling agent is IL-33. In such an embodiment, the modified signaling agent has reduced affinity and/or activity for the ST-2 receptor and IL-1RAcP. In some embodiments, the modified signaling agent has substantially reduced or ablated affinity and/or activity for the ST-2 receptor and IL-1RAcP.

In an embodiment, the wild type IL-33 has the amino acid sequence of SEQ ID NO:246.

In such embodiments, the modified IL-33 agent may comprise one or more mutations in amino acids or amino acid regions selected from 1113-Y122, S127-E139, E144-D157, Y163-M183, E200, Q215, L220-C227 and T260-E269, as described in WO/2015/007542, the entire contents of which are hereby incorporated by reference (numbering based on the human sequence, Genbank accession number NP_254274, version NP_254274.1, GI: 15559209).

In an embodiment, the modified signaling agent is epidermal growth factor (EGF). EGF is a member of a family of potent growth factors. Members include EGF, HB-EGF, and others such as TGFalpha, amphiregulin, neuregulins, epiregulin, betacellulin. EGF family receptors include EGFR (ErbB1), ErbB2, ErbB3 and ErbB4. These may function as homodimeric and/or heterodimeric receptor subtypes. The different EGF family members exhibit differential selectivity for the various receptor subtypes. For example, EGF associates with ErbB1/ErbB1, ErbB1/ErbB2, ErbB4/ErbB2 and some other heterodimeric subtypes. HB-EGF has a similar pattern, although it also associates with ErbB4/4. Modulation of EGF (EGF-like) growth factor signaling, positively or negatively, is of considerable therapeutic interest. For example, inhibition of EGFRs signaling is of interest in the treatment of various cancers where EGFR signaling constitutes a major growth-promoting signal. Alternatively, stimulation of EGFRs signaling is of therapeutic interest in, for example, promoting wound healing (acute and chronic), oral mucositis (a major side effect of various cancer therapies, including, without limitation radiation therapy).

In some embodiments, the additional modified signaling agent has reduced affinity and/or activity for ErbB1, ErbB2, ErbB3, and/or ErbB4. Such embodiments find use, for example, in methods of treating wounds. In some embodiments, the modified signaling agent binds to one or more ErbB1, ErbB2, ErbB3, and ErbB4 and antagonizes the activity of the receptor. In such embodiments, the modified signaling agent has reduced affinity and/or activity for ErbB1, ErbB2, ErbB3, and/or ErbB4 that allows for the activity of the receptor to be antagonized in an attenuated fashion. Such embodiments find use in, for example, treatments of cancer. In an embodiment, the modified signaling agent has reduced affinity and/or activity for ErbB1. ErbB1 is the therapeutic target of kinase inhibitors-most have side effects because they are not very selective (e.g., gefitinib, erlotinib, afatinib, brigatinib and icotinib). In some embodiments, attenuated antagonistic ErbB1 signaling is more on-target and has less side effects than other agents targeting receptors for EGF.

In some embodiments, the additional modified signaling agent has reduced affinity and/or activity (e.g. antagonistic e.g. natural antagonistic activity or antagonistic activity that is the result of one or more mutations, see, e.g., WO 2015/007520, the entire contents of which are hereby incorporated by reference) for ErbB1 and/or substantially reduced or ablated affinity and/or activity for ErbB4 or other subtypes it may interact with. Through specific targeting via the targeting moiety, cell-selective suppression (antagonism e.g. natural antagonistic activity or antagonistic activity that is the result of one or more mutations, see, e.g., WO 2015/007520, the entire contents of which are hereby incorporated by reference) of ErbB1/ErbB1 receptor activation would be achieved—while not engaging other receptor subtypes potentially associated with inhibition-associated side effects. Hence, in contrast to EGFR kinase inhibitors, which inhibit EGFR activity in all cell types in the body, such a construct would provide a cell-selective (e.g., tumor cell with activated EGFR signaling due to amplification of receptor, overexpression etc.) anti-EGFR (ErbB1) drug effect with reduced side effects.

In some embodiments, the additional modified signaling agent has reduced affinity and/or activity (e.g. agonistic) for ErbB4 and/or other subtypes it may interact with. Through targeting to specific target cells through the targeting moiety, a selective activation of ErbB1 signaling is achieved (e.g. epithelial cells). Such a construct finds use, in some embodiments, in the treatment of wounds (promoting would healing) with reduced side effects, especially for treatment of chronic conditions and application other than topical application of a therapeutic (e.g. systemic wound healing).

In an embodiment, the modified signaling agent is insulin or insulin analogs. In some embodiments, the modified insulin or insulin analog has reduced affinity and/or activity for the insulin receptor and/or IGF1 or IGF2 receptor. In some embodiments, the modified insulin or insulin analog has substantially reduced or ablated affinity and/or activity for the insulin receptor and/or IGF1 or IGF2 receptor. Attenuated response at the insulin receptor allows for the control of diabetes, obesity, metabolic disorders and the like while directing away from IGF1 or IGF2 receptor avoids pro-cancer effects.

In an embodiment, the modified signaling agent is insulin-like growth factor-I or insulin-like growth factor-II (IGF-1 or IGF-2). In an embodiment, the modified signaling agent is IGF-1. In such an embodiment, the modified signaling agent has reduced affinity and/or activity for the insulin receptor and/or IGF1 receptor. In an embodiment, the modified signaling agent may bind to the IGF1 receptor and antagonize the activity of the receptor. In such an embodiment, the modified signaling agent has reduced affinity and/or activity for IGF1 receptor, which allows for the activity of the receptor to be antagonized in an attenuated fashion. In some embodiments, the modified signaling agent has substantially reduced or ablated affinity and/or activity for the insulin receptor and/or IGF1 receptor. In some embodiments, the modified signaling agent has reduced affinity and/or activity for IGF2 receptor, which allows for the activity of the receptor to be antagonized in an attenuated fashion. In an embodiment, the modified signaling agent has substantially reduced or ablated affinity and/or activity for the insulin receptor and accordingly does not interfere with insulin signaling. In various embodiments, this applies to cancer treatment. In various embodiments, the present agents may prevent IR isoform A from causing resistance to cancer treatments.

In an embodiment, the modified signaling agent is EPO. In various embodiments, the modified EPO agent has reduced affinity and/or activity for the EPO receptor (EPOR) receptor and/or the ephrin receptor (EphR) relative to wild type EPO or other EPO based agents described herein. In some embodiments, the modified EPO agent has substantially reduced or ablated affinity and/or activity for the EPO receptor (EPOR) receptor and/or the Eph receptor (EphR). Illustrative EPO receptors include, but are not limited to, an EPOR homodimer or an EPOR/CD131 heterodimer. Also included as an EPO receptor is beta-common receptor (βcR). Illustrative Eph receptors include, but are not limited to, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, and EPHB6. In some embodiments, the modified EPO protein comprises one or more mutations that cause the EPO protein to have reduced affinity for receptors that comprise one or more different EPO receptors or Eph receptors (e.g. heterodimer, heterotrimers, etc., including by way of non-limitation: EPOR-EPHB4, EPOR-βcR-EPOR). Also provided are the receptors of EP Patent Publication No. 2492355 the entire contents of which are hereby incorporated by reference, including by way of non-limitation, NEPORs.

In an embodiment, the human EPO has the amino acid sequence of SEQ ID NO:247 (first 27 amino acids are the signal peptide).

In an embodiment, the human EPO protein is the mature form of EPO (with the signal peptide being cleaved off) which is a glycoprotein of 166 amino acid residues having the sequence of SEQ ID NO:248.

The structure of the human EPO protein is predicted to comprise four-helix bundles including helices A, B, C, and D. In various embodiments, the modified EPO protein comprises one or more mutations located in four regions of the EPO protein which are important for bioactivity, i.e., amino acid residues 10-20, 44-51, 96-108, and 142-156. In some embodiments, the one or more mutations are located at residues 11-15, 44-51, 100-108, and 147-151. These residues are localized to helix A (Val11, Arg14, and Tyr15), helix C (Ser100, Arg103, Ser104, and Leu108), helix D (Asn147, Arg150, Gly151, and Leu155), and the A/B connecting loop (residues 42-51). In some embodiments, the modified EPO protein comprises mutations in residues between amino acids 41-52 and amino acids 147, 150, 151, and 155. Without wishing to be bound by theory, it is believed that mutations of these residues have substantial effects on both receptor binding and in vitro biological activity. In some embodiments, the modified EPO protein comprises mutations at residues 11, 14, 15, 100, 103, 104, and 108. Without wishing to be bound by theory, it is believed that mutations of these residues have modest effects on receptor binding activity and much greater effects on in vitro biological activity. Illustrative substitutions include, but are not limited to, one or more of Val11Ser, Arg14Ala, Arg14Gln, Tyr15Ile, Pro42Asn, Thr44Ile, Lys45Asp, Val46Ala, Tyr51Phe, Ser100Glu, Ser100Thr, Arg103Ala, Ser104Ile, Ser104Ala, Leu108Lys, Asn147Lys, Arg150Ala, Gly151Ala, and Leu155Ala.

In some embodiments, the modified EPO protein comprises mutations that effect bioactivity and not binding, e.g. those listed in Eliot, et al. Mapping of the Active Site of Recombinant Human Erythropoietin Jan. 15, 1997; Blood: 89 (2), the entire contents of which are hereby incorporated by reference.

In some embodiments, the modified EPO protein comprises one or more mutations involving surface residues of the EPO protein which are involved in receptor contact. Without wishing to be bound by theory, it is believed that mutations of these surface residues are less likely to affect protein folding thereby retaining some biological activity. Illustrative surface residues that may be mutated include, but are not limited to, residues 147 and 150. In illustrative embodiments, the mutations are substitutions including, one or more of N147A, N147K, R150A and R150E.

In some embodiments, the modified EPO protein comprises one or more mutations at residues N59, E62, L67, and L70, and one or more mutations that affect disulfide bond formation. Without wishing to be bound by theory, it is believed that these mutations affect folding and/or are predicted be in buried positions and thus affects biological activity indirectly.

In an embodiment, the modified EPO protein comprises a K20E substitution, which significantly reduces receptor binding. See Elliott, et al., (1997) Blood, 89:493-502, the entire contents of which are hereby incorporated by reference.

Additional EPO mutations that may be incorporated into the chimeric EPO protein of the invention are disclosed in, for example, Elliott, et al., (1997) Blood, 89:493-502, the entire contents of which are hereby incorporated by reference and Taylor et al., (2010) PEDS, 23 (4): 251-260, the entire contents of which are hereby incorporated by reference.

In various embodiments, the signaling agent is a toxin or toxic enzyme. In some embodiments, the toxin or toxic enzyme is derived from plants and bacteria. Illustrative toxins or toxic enzymes include, but are not limited to, the diphtheria toxin, Pseudomonas toxin, anthrax toxin, ribosome-inactivating proteins (RIPs) such as ricin and saporin, modeccin, abrin, gelonin, and poke weed antiviral protein. Additional toxins include those disclosed in Mathew et al., (2009) Cancer Sci 100 (8): 1359-65, the entire disclosures are hereby incorporated by reference. In such embodiments, the chimeric proteins or chimeric protein complexes such as Fc-based chimeric protein complex of the invention may be utilized to induce cell death in cell-type specific manner. In such embodiments, the toxin may be modified, e.g. mutated, to reduce affinity and/or activity of the toxin for an attenuated effect, as described with other signaling agents herein.

### Linkers and Functional Groups

In some embodiments, chimeric protein, chimeric protein complex, vaccine composition, or adjuvant optionally comprises one or more connectors (e.g., linkers and/or an Fc domains). In some embodiments, chimeric protein, chimeric protein complex, vaccine composition, or adjuvant comprise a connector (e.g., linker and/) connecting the targeting moiety and the signaling agent (e.g., IL-1α or a variant thereof). In some embodiments, chimeric protein, chimeric protein complex, vaccine composition, or adjuvant comprise a connector (e.g., linker and/or Fc domain) within the signaling agent (e.g., IL-1α or a variant thereof). In some embodiments, the connector (e.g., linker and/or Fc domain) may be utilized to link various functional groups, residues, or moieties as described herein to chimeric protein, chimeric protein complex, vaccine composition, or adjuvant. In some embodiments, the connector (e.g., linker and/or Fc domain) is a single amino acid or a plurality of amino acids that does not affect or reduce the stability, orientation, binding, neutralization, and/or clearance characteristics of the binding regions and the binding protein. In various embodiments, the linker is selected from a peptide, a protein, a sugar, or a nucleic acid.

In some embodiments, vectors encoding chimeric protein, chimeric protein complex, vaccine composition, or adjuvant are linked as a single nucleotide sequence to any of the connectors (e.g., linkers and/or Fc domains) described herein are provided and may be used to prepare such chimeric protein, chimeric protein complex, vaccine composition, or adjuvant. In embodiments, the substituents of the chimeric protein complex (e.g., Fc-based chimeric protein complex) are expressed as nucleotide sequences in a vector.

In some embodiments, the linker length allows for efficient binding of a targeting moiety and the signaling agent (e.g., IL-1α or a variant thereof) to their receptors. For instance, in some embodiments, the linker length allows for efficient binding of one of the targeting moieties and the signaling agent to receptors on the same cell.

In some embodiments, the linker length is at least equal to the minimum distance between the binding sites of one of the targeting moieties and the signaling agent to receptors on the same cell. In some embodiments the linker length is at least twice, or three times, or four times, or five times, or ten times, or twenty times, or 25 times, or 50 times, or one hundred times, or more the minimum distance between the binding sites of one of the targeting moieties and the signaling agent to receptors on the same cell.

As described herein, the linker length allows for efficient binding of one of the targeting moieties and the signaling agent to receptors on the same cell, the binding being sequential, e.g. targeting moiety/receptor binding preceding signaling agent/receptor binding.

In some embodiments, there are two linkers in a single chimera, each connecting the signaling agent to a targeting moiety. In various embodiments, the linkers have lengths that allow for the formation of a site that has a disease cell and an effector cell without steric hindrance that would prevent modulation of the either cell.

The invention contemplates the use of a variety of linker sequences. In various embodiments, the linker may be derived from naturally-occurring multi-domain proteins or are empirical linkers as described, for example, in Chichili et al., (2013), Protein Sci. 22 (2): 153-167, Chen et al., (2013), Adv Drug Deliv Rev. 65 (10): 1357-1369, the entire contents of which are hereby incorporated by reference. In some embodiments, the linker may be designed using linker designing databases and computer programs such as those described in Chen et al., (2013), Adv Drug Deliv Rev. 65 (10): 1357-1369 and Crasto et al., (2000), Protein Eng. 13 (5): 309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the present chimeric proteins or chimeric protein complexes such as Fc-based chimeric protein complex.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In some embodiments directed to chimeric protein, chimeric protein complex, vaccine composition, or adjuvant having two or more targeting moieties, a linker connects the two targeting moieties to each other and this linker has a short length and a linker connects a targeting moiety and a signaling agent this linker is longer than the linker connecting the two targeting moieties. For example, the difference in amino acid length between the linker connecting the two targeting moieties and the linker connecting a targeting moiety and a signaling agent may be about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids.

In some embodiments, the connector between the signaling moiety and the targeting moiety is a flexible linker. In various embodiments, the linker is substantially comprised of glycine and serine residues (e.g.

about 30%, or about 40%, or about 50%, or about 60%, or about 70%, or about 80%, or about 90%, or about 95%, or about 97% glycines and serines). For example, in some embodiments, the linker is (Gly4Ser)ₙ, where n is from about 1 to about 8, e.g. 1, 2, 3, 4, 5, 6, 7, or 8 (SEQ ID NO: 249-SEQ ID NO: 256, respectively). In an embodiment, the linker sequence is GGSGGSGGGGSGGGGS (SEQ ID NO: 257). Additional illustrative linkers include, but are not limited to, linkers having the sequence LE, GGGGS (SEQ ID NO: 249), (GGGGS)ₙ (n=1-4) (SEQ ID NO: 249-SEQ ID NO: 252). (Gly)₈ (SEQ ID NO: 258), (Gly)₆ (SEQ ID NO: 259), (EAAAK)ₙ (n=1-3) (SEQ ID NO: 260-SEQ ID NO: 262), A(EAAAK)ₙA (n=2-5) (SEQ ID NO: 263-SEQ ID NO: 266), AEAAAKEAAAKA (SEQ ID NO: 263), A(EAAAK)₄ALEA(EAAAK)₄A (SEQ ID NO: 267), PAPAP (SEQ ID NO: 268), KESGSVSSEQLAQFRSLD (SEQ ID NO: 269), EGKSSGSGSESKST (SEQ ID NO: 270), GSAGSAAGSGEF (SEQ ID NO: 271), and (XP)ₙ, with X designating any amino acid, e.g., Ala, Lys, or Glu. In various embodiments, the linker is GGS or a repeat thereof wherein the GGS sequence is repeated 1 to 20 times (SEQ ID NO: 415-434). In some embodiments, the linker is GGGS or a repeat thereof wherein the GGGS sequence is repeated 1 to 8 times (SEQ ID NO: 435-442).

In some embodiments, the linker is one or more of GGGSE (SEQ ID NO: 272), GSESG (SEQ ID NO: 273), GSEGS (SEQ ID NO: 274), GEGGSGEGSSGEGSSSEGGGSEGGGSEGGGSEGGS (SEQ ID NO: 275), and a linker of randomly placed G, S, and E every 4 amino acid intervals.

In some embodiments, the linker is a synthetic linker such as PEG.

In various embodiments, the linker may be functional. For example, without limitation, the linker may function to improve the folding and/or stability, improve the expression, improve the pharmacokinetics, and/or improve the bioactivity of the present chimeric proteins or chimeric protein complexes such as Fc-based chimeric protein complex. In another example, the linker may function to target the chimeric proteins or chimeric protein complexes such as Fc-based chimeric protein complex to a particular cell type or location.

In various embodiments, the present chimeric proteins or chimeric protein complexes such as Fc-based chimeric protein complex may include one or more functional groups, residues, or moieties. In various embodiments, the one or more functional groups, residues, or moieties are attached or genetically fused to any of the signaling agents or targeting moieties described herein. In some embodiments, such functional groups, residues or moieties confer one or more desired properties or functionalities to the chimeric proteins or chimeric protein complexes such as Fc-based chimeric protein complex of the invention. Examples of such functional groups and of techniques for introducing them into the chimeric proteins or chimeric protein complexes such as Fc-based chimeric protein complex are known in the art, for example, see Remington's Pharmaceutical Sciences, 16th ed., Mack Publishing Co., Easton, Pa. (1980).

In various embodiments, each of the chimeric proteins or chimeric protein complexes such as Fc-based chimeric protein complex may by conjugated and/or fused with another agent to extend half-life or otherwise improve pharmacodynamic and pharmacokinetic properties. In some embodiments, the chimeric proteins or chimeric protein complexes such as Fc-based chimeric protein complex may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In various embodiments, each of the individual chimeric proteins or chimeric protein complexes such as Fc-based chimeric protein complex is fused to one or more of the agents described in BioDrugs (2015) 29:215-239, the entire contents of which are hereby incorporated by reference.

In some embodiments, the functional groups, residues, or moieties comprise a suitable pharmacologically acceptable polymer, such as poly(ethyleneglycol) (PEG) or derivatives thereof (such as methoxypoly(ethyleneglycol) or mPEG). In some embodiments, attachment of the PEG moiety increases the half-life and/or reduces the immunogenicity of the chimeric proteins or chimeric protein complexes such as Fc-based chimeric protein complex. Generally, any suitable form of pegylation can be used, such as the pegylation used in the art for antibodies and antibody fragments (including but not limited to single domain antibodies such as VHHs); see, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04/060965, the entire contents of which are hereby incorporated by reference. Various reagents for pegylation of proteins are also commercially available, for example, from Nektar Therapeutics, USA. In some embodiments, site-directed pegylation is used, in particular via a cysteine-residue (see, for example, Yang et al., Protein Engineering, 16, 10, 761-770 (2003), the entire contents of which is hereby incorporated by reference). In some embodiments, the chimeric proteins or chimeric protein complexes such as Fc-based chimeric protein complex of the invention is modified so as to suitably introduce one or more cysteine residues for attachment of PEG, or an amino acid sequence comprising one or more cysteine residues for attachment of PEG may be fused to the amino-and/or carboxy-terminus of the chimeric proteins or chimeric protein complexes such as Fc-based chimeric protein complex, using techniques known in the art.

In some embodiments, the functional groups, residues, or moieties comprise N-linked or O-linked glycosylation. In some embodiments, the N-linked or O-linked glycosylation is introduced as part of a co-translational and/or post-translational modification.

In some embodiments, the functional groups, residues, or moieties comprise one or more detectable labels or other signal-generating groups or moieties. Suitable labels and techniques for attaching, using and detecting them are known in the art and, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels include moieties that can be detected using NMR or ESR spectroscopy. Such labeled VHHs and polypeptides of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label.

In some embodiments, the functional groups, residues, or moieties comprise a tag that is attached or genetically fused to the chimeric proteins or chimeric protein complexes such as Fc-based chimeric protein complex. In some embodiments, the chimeric proteins or chimeric protein complexes such as Fc-based chimeric protein complex may include a single tag or multiple tags. The tag for example is a peptide, sugar, or DNA molecule that does not inhibit or prevent binding of the chimeric proteins or chimeric protein complexes such as Fc-based chimeric protein complex to its target or any other antigen of interest such as tumor antigens. In various embodiments, the tag is at least about: three to five amino acids long, five to eight amino acids long, eight to twelve amino acids long, twelve to fifteen amino acids long, or fifteen to twenty amino acids long. Illustrative tags are described for example, in U.S. Patent Publication No. US2013/0058962. In some embodiment, the tag is an affinity tag such as glutathione-S-transferase (GST) and histidine (His) tag. In an embodiment, the chimeric proteins or chimeric protein complexes such as Fc-based chimeric protein complex comprises a His tag.

In some embodiments, the functional groups, residues, or moieties comprise a chelating group, for example, to chelate one of the metals or metallic cations. Suitable chelating groups, for example, include, without limitation, diethyl-enetriaminepentaacetic acid (DTPA) or ethylenediaminetetraacetic acid (EDTA).

In some embodiments, the functional groups, residues, or moieties comprise a functional group that is one part of a specific binding pair, such as the biotin-(strept) avidin binding pair. Such a functional group may be used to link the chimeric proteins or chimeric protein complexes such as Fc-based chimeric protein complex of the invention to another protein, polypeptide or chemical compound that is bound to the other half of the binding pair, i.e., through formation of the binding pair. For example, a chimeric proteins or chimeric protein complexes such as Fc-based chimeric protein complex of the invention may be conjugated to biotin, and linked to another protein, polypeptide, compound or carrier conjugated to avidin or streptavidin. For example, such a conjugated chimeric proteins or chimeric protein complexes such as Fc-based chimeric protein complex may be used as a reporter, for example, in a diagnostic system where a detectable signal-producing agent is conjugated to avidin or streptavidin. Such binding pairs may, for example, also be used to bind the chimeric proteins or chimeric protein complexes such as Fc-based chimeric protein complex to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the chimeric proteins or chimeric protein complexes such as Fc-based chimeric protein complex of the invention.

### Production of Chimeric Proteins or Chimeric Protein Complexes

Methods for producing chimeric protein, chimeric protein complex, vaccine composition, or adjuvant of the invention are described herein. For example, DNA sequences encoding chimeric protein, chimeric protein complex, vaccine composition, or adjuvant of the invention (e.g., DNA sequences encoding the signaling agent (e.g., IL-1α or a variant thereof) and the targeting moiety and the linker) can be chemically synthesized using methods known in the art.

Synthetic DNA sequences can be ligated to other appropriate nucleotide sequences, including, e.g., expression control sequences, to produce gene expression constructs encoding chimeric protein, chimeric protein complex, vaccine composition, or adjuvant. Accordingly, in various embodiments, the present invention provides for isolated nucleic acids comprising a nucleotide sequence encoding chimeric protein, chimeric protein complex, vaccine composition, or adjuvant of the invention.

Nucleic acids encoding chimeric protein, chimeric protein complex, vaccine composition, or adjuvant of the invention can be incorporated (ligated) into expression vectors, which can be introduced into host cells through transfection, transformation, or transduction techniques. For example, nucleic acids encoding chimeric protein, chimeric protein complex, vaccine composition, or adjuvant of the invention can be introduced into host cells by retroviral transduction. Illustrative host cells are E. coli cells, Chinese hamster ovary (CHO) cells, human embryonic kidney 293 (HEK 293) cells, Hela cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode chimeric protein, chimeric protein complex, vaccine composition, or adjuvant of the invention. Accordingly, in various embodiments, the present invention provides expression vectors comprising nucleic acids that encode chimeric protein, chimeric protein complex, vaccine composition, or adjuvant of the invention. In various embodiments, the present invention additional provides host cells comprising such expression vectors.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. In another example, if the engineered gene is to be expressed in eukaryotic host cells, e.g., CHO cells, it is first inserted into an expression vector containing for example, a suitable eukaryotic promoter, a secretion signal, enhancers, and various introns. The gene construct can be introduced into the host cells using transfection, transformation, or transduction techniques.

The chimeric protein, chimeric protein complex, vaccine composition, or adjuvant of the invention can be produced by growing a host cell transfected with an expression vector encoding chimeric protein, chimeric protein complex, vaccine composition, or adjuvant under conditions that permit expression of the protein. Following expression, the protein can be harvested and purified using techniques well known in the art, e.g., affinity tags such as glutathione-S-transferase (GST) and histidine tags or by chromatography.

Accordingly, in various embodiments, the present invention provides for a nucleic acid encoding a chimeric protein, chimeric protein complex, vaccine composition, or adjuvant of the present invention. In various embodiments, the present invention provides for a host cell comprising a nucleic acid encoding chimeric protein, chimeric protein complex, vaccine composition, or adjuvant of the present invention.

In various embodiments, IL-1α or its variant; or chimeric protein, chimeric protein complex, vaccine composition, or adjuvant comprising the IL-1α or its variant may be expressed in vivo, for instance, in a patient. For example, in various embodiments, the IL-1α, its variant, or chimeric protein, chimeric protein complex, vaccine composition, or adjuvant comprising the IL-1α or its variant may administered in the form of nucleic acid which encodes for the IL-1α or its variant or chimeric protein, chimeric protein complex, vaccine composition, or adjuvant comprising IL-1α or its variant. In various embodiments, the nucleic acid is DNA or RNA. In some embodiments, the IL-1α, its variant, or chimeric protein, chimeric protein complex, vaccine composition, or adjuvant comprising the IL-1α or its variant is encoded by a modified mRNA, i.e. an mRNA comprising one or more modified nucleotides. In some embodiments, the modified mRNA comprises one or modifications found in U.S. Pat. No. 8,278,036, the entire contents of which are hereby incorporated by reference. In some embodiments, the modified mRNA comprises one or more of m5C, m5U, m6A, s2U, Ψ, and 2′-O-methyl-U. In some embodiments, the present invention relates to administering a modified mRNA encoding one or more of chimeric protein, chimeric protein complex, vaccine composition, or adjuvant. In some embodiments, the present invention relates to gene therapy vectors comprising the same. In some embodiments, the present invention relates to gene therapy methods comprising the same. In various embodiments, the nucleic acid is in the form of an oncolytic virus, e.g. an adenovirus, reovirus, measles, herpes simplex, Newcastle disease virus or vaccinia.

In various embodiments, chimeric protein, chimeric protein complex, vaccine composition, or adjuvant comprises a targeting moiety that is a VHH. In various embodiments, the VHH is not limited to a specific biological source or to a specific method of preparation. For example, the VHH can generally be obtained: (1) by isolating the V_{H}H domain of a naturally occurring heavy chain antibody; (2) by expression of a nucleotide sequence encoding a naturally occurring V_{H}H domain; (3) by “humanization” of a naturally occurring V_{H}H domain or by expression of a nucleic acid encoding a such humanized V_{H}H domain; (4) by “camelization” of a naturally occurring VH domain from any animal species, such as from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known in the art; (7) by preparing a nucleic acid encoding a VHH using techniques for nucleic acid synthesis known in the art, followed by expression of the nucleic acid thus obtained; and/or (8) by any combination of one or more of the foregoing.

In an embodiment, chimeric protein, chimeric protein complex, vaccine composition, or adjuvant comprises a VHH that corresponds to the V_{H}H domains of naturally occurring heavy chain antibodies directed against a target of interest. In some embodiments, such V_{H}H sequences can generally be generated or obtained by suitably immunizing a species of Camelid with a molecule of based on the target of interest (e.g., XCR1, Clec9a, CD8, SIRP1α, FAP, etc.) (i.e., so as to raise an immune response and/or heavy chain antibodies directed against the target of interest), by obtaining a suitable biological sample from the Camelid (such as a blood sample, or any sample of B-cells), and by generating V_{H}H sequences directed against the target of interest, starting from the sample, using any suitable known techniques. In some embodiments, naturally occurring V_{H}H domains against the target of interest can be obtained from naive libraries of Camelid V_{H}H sequences, for example, by screening such a library using the target of interest or at least one part, fragment, antigenic determinant or epitope thereof using one or more screening techniques known in the art. Such libraries and techniques are, for example, described in WO 9937681, WO 0190190, WO 03025020 and WO 03035694, the entire contents of which are hereby incorporated by reference. In some embodiments, improved synthetic or semi-synthetic libraries derived from naive V_{H}H libraries may be used, such as V_{H}H libraries obtained from naive V_{H}H libraries by techniques such as random mutagenesis and/or CDR shuffling, as for example, described in WO 0043507, the entire contents of which are hereby incorporated by reference. In some embodiments, another technique for obtaining V_{H}H sequences directed against a target of interest involves suitably immunizing a transgenic mammal that is capable of expressing heavy chain antibodies (i.e., so as to raise an immune response and/or heavy chain antibodies directed against the target of interest), obtaining a suitable biological sample from the transgenic mammal (such as a blood sample, or any sample of B-cells), and then generating V_{H}H sequences directed against XCR1 starting from the sample, using any suitable known techniques. For example, for this purpose, the heavy chain antibody-expressing mice and the further methods and techniques described in WO 02085945 and in WO 04049794 (the entire contents of which are hereby incorporated by reference) can be used.

In an embodiment, chimeric protein, chimeric protein complex, vaccine composition, or adjuvant of the present invention comprises a V_{H}H that has been “humanized” i.e., by replacing one or more amino acid residues in the amino acid sequence of the naturally occurring V_{H}H sequence (and in particular in the framework sequences) by one or more of the amino acid residues that occur at the corresponding position(s) in a VH domain from a conventional 4-chain antibody from a human being. This can be performed using humanization techniques known in the art. In some embodiments, possible humanizing substitutions or combinations of humanizing substitutions may be determined by methods known in the art, for example, by a comparison between the sequence of a VHH and the sequence of a naturally occurring human VH domain. In some embodiments, the humanizing substitutions are chosen such that the resulting humanized VHHs still retain advantageous functional properties. Generally, as a result of humanization, the VHHs of the invention may become more “human-like,” while still retaining favorable properties such as a reduced immunogenicity, compared to the corresponding naturally occurring V_{H}H domains. In various embodiments, the humanized VHHs of the invention can be obtained in any suitable manner known in the art and thus are not strictly limited to polypeptides that have been obtained using a polypeptide that comprises a naturally occurring V_{H}H domain as a starting material.

In an embodiment, chimeric protein, chimeric protein complex, vaccine composition, or adjuvant comprises a VHH that has been “camelized,” i.e., by replacing one or more amino acid residues in the amino acid sequence of a naturally occurring VH domain from a conventional 4-chain antibody by one or more of the amino acid residues that occur at the corresponding position(s) in a V_{H}H domain of a heavy chain antibody of a camelid. In some embodiments, such “camelizing” substitutions are inserted at amino acid positions that form and/or are present at the VH-VL interface, and/or at the so-called Camelidae hallmark residues (see, for example, WO9404678, the entire contents of which are hereby incorporated by reference). In some embodiments, the VH sequence that is used as a starting material or starting point for generating or designing the camelized VHH is a VH sequence from a mammal, for example, the VH sequence of a human being, such as a VH3 sequence. In various embodiments, the camelized VHHs can be obtained in any suitable manner known in the art (i.e., as indicated under points (1)-(8) above) and thus are not strictly limited to polypeptides that have been obtained using a polypeptide that comprises a naturally occurring VH domain as a starting material.

In various embodiments, both “humanization” and “camelization” can be performed by providing a nucleotide sequence that encodes a naturally occurring V_{H}H domain or VH domain, respectively, and then changing, in a manner known in the art, one or more codons in the nucleotide sequence in such a way that the new nucleotide sequence encodes a “humanized” or “camelized” VHH, respectively. This nucleic acid can then be expressed in a manner known in the art, so as to provide the desired VHH of the invention. Alternatively, based on the amino acid sequence of a naturally occurring V_{H}H domain or VH domain, respectively, the amino acid sequence of the desired humanized or camelized VHH of the invention, respectively, can be designed and then synthesized de novo using techniques for peptide synthesis known in the art. Also, based on the amino acid sequence or nucleotide sequence of a naturally occurring V_{H}H domain or VH domain, respectively, a nucleotide sequence encoding the desired humanized or camelized VHH, respectively, can be designed and then synthesized de novo using techniques for nucleic acid synthesis known in the art, after which the nucleic acid thus obtained can be expressed in a manner known in the art, so as to provide the desired VHH of the invention. Other suitable methods and techniques for obtaining the VHHs of the invention and/or nucleic acids encoding the same, starting from naturally occurring VH sequences or V_{H}H sequences, are known in the art, and may, for example, comprise combining one or more parts of one or more naturally occurring VH sequences (such as one or more FR sequences and/or CDR sequences), one or more parts of one or more naturally occurring V_{H}H sequences (such as one or more FR sequences or CDR sequences), and/or one or more synthetic or semi-synthetic sequences, in a suitable manner, so as to provide a VHH of the invention or a nucleotide sequence or nucleic acid encoding the same.

### Vaccines and Adjuvants

In accordance with certain embodiments of the invention, the pharmaceutical composition comprising chimeric protein, chimeric protein complex, vaccine composition, or adjuvant may be administered, for example, more than once daily (e.g., about two times, about three times, about four times, about five times, about six times, about seven times, about eight times, about nine times, or about ten times daily), about once per day, about every other day, about every third day, about once a week, about once every two weeks, about once every month, about once every two months, about once every three months, about once every six months, or about once every year. In an embodiment, the pharmaceutical composition comprising chimeric protein, chimeric protein complex, vaccine composition, or adjuvant is administered about three times a week.

In various embodiments, chimeric protein, chimeric protein complex, vaccine composition, or adjuvant may be administered for a prolonged period. For example, chimeric protein, chimeric protein complex, vaccine composition, or adjuvant may be administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks. For example, chimeric protein, chimeric protein complex, vaccine composition, or adjuvant may be administered for 12 weeks, 24 weeks, 36 weeks or 48 weeks. In some embodiments, chimeric protein, chimeric protein complex, vaccine composition, or adjuvant is administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some embodiments, chimeric protein, chimeric protein complex, vaccine composition, or adjuvant may be administered for at least about 1 year, at least about 2 years, at least about 3 years, at least about 4 years, or at least about 5 years.

One aspect of the present application is related to a vaccine composition comprising: (a) an adjuvant, and (b) an antigen that is suitable for inducing an immune response. The adjuvant comprises a chimeric protein or chimeric protein complex comprising: (i) an IL-1α, pro-IL-1α, or a mutant thereof (which are examples of signaling agents as described herein), (ii) one or more targeting moieties, said targeting moieties comprising recognition domains which specifically bind to an antigen or receptor of interest; and (iii) a connector between (i) and (ii).

In embodiments, the connector comprises: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii); and/or (2) a flexible linker that connects (i) and (ii), wherein the mutant IL-1α or pro-IL-1α is characterized by low or high affinity or activity at the IL-1 receptor.

In embodiments, the adjuvant is a nucleic acid, which encodes the chimeric protein or chimeric protein complex. In embodiments, the nucleic acid is an mRNA, optionally comprising one or more non-canonical nucleotides, optionally selected from pseudouridine and 5-methoxyuridine. In embodiments, the nucleic acid is DNA, optionally selected from linear DNA, DNA fragments, or DNA plasmids.

In some embodiments, the vaccine composition of the present invention further comprises an aluminum gel or salt. In embodiments, the aluminum gel or salt is selected from aluminum hydroxide, aluminum phosphate, and aluminum sulfate. In some embodiments, the adjuvant is a nucleic acid encoding the chimeric protein or chimeric protein complex as described herein.

In some embodiments, the additional adjuvant is selected from, oil-in-water emulsion formulations, saponin adjuvants, ovalbumin, toll like receptors ligands, Freunds Adjuvant, cytokines, and chitosans. Illustrative additional adjuvants include, but are not limited to: (1) ovalbumin (e.g. ENDOFIT), which is often used for biochemical studies; (2) oil-in-water emulsion formulations (with or without other specific immunostimulating agents such as muramyl peptides or bacterial cell wall components), such as for example (a) MF59 (PCT Publ. No. WO 90/14837), containing 5% Squalene, 0.5% Tween 80, and 0.5% Span 85 (optionally containing various amounts of MTP-PE) formulated into submicron particles using a microfluidizer such as, for example, Model HOy microfluidizer (Microfluidics, Newton, Mass.), (b) SAF, containing 10% Squalane, 0.4% Tween 80, 5% pluronic-blocked polymer L121, and thr-MDP either microfluidized into a submicron emulsion or vortexed to generate a larger particle size emulsion, (c) RIBI adjuvant system (RAS), (RIBI IMMUNOCHEM, Hamilton, MO.) containing 2% Squalene, 0.2% Tween 80, and, optionally, one or more bacterial cell wall components from the group of monophosphorylipid A (MPL), trehalose dimycolate (TDM), and cell wall skeleton (CWS), including MPL+CWS (DETOX™); and (d) ADDAVAX (Invitrogen); (3) saponin adjuvants, such as STIMULON (Cambridge Bioscience, Worcester, Mass.) may be used or particles generated therefrom such as ISCOMs (immunostimulating complexes); (4) Complete Freunds Adjuvant (CFA) and Incomplete Freunds Adjuvant (IFA); (5) cytokines, such as interleukins (by way of non-limiting example, IL-1, IL-2, IL-4, IL-5, IL-6, IL-7, IL-12, etc.), interferons (e.g., gamma interferon), macrophage colony stimulating factor (M-CSF), tumor necrosis factor (TNF), etc; (6) chitosans and other derivatives of chitin or poly-N-acetyl-D-glucosamine in which the greater proportion of the N-acetyl groups have been removed through hydrolysis (see, e.g., European Patent Application 460 020, which is hereby incorporated by reference in its entirety, disclosing pharmaceutical formulations including chitosans as mucosal absorption enhancers; and (7) other substances that act as immunostimulating agents to enhance the effectiveness of the composition, e.g., monophosphoryl lipid A. In other embodiments, the additional adjuvant is one or more of an aluminum salt or gel, a pattern recognition receptors (PRR) agonist, CpG ODNs and imidazoquinolines. In some embodiments, the additional adjuvant is one or more of cyclic [G(3′,5′)pA(3′,5′)p] (e.g. 3′3′-cGAMP VACCIGRADE); cyclic [G(2′,5′)pA(3′,5′)p]2′3′ (e.g. 2′3′ cGAMP VACCIGRADE); cyclic [G(2′,5′)pA(2′,5′)p] (e.g. 2′2′-cGAMP VACCIGRADE), cyclic diadenylate monophosphate (e.g. c-di-AMP VACCIGRADE); cyclic diguanylate monophosphate (e.g. c-di-GMP VACCIGRADE); TLR7 agonist-imidazoquinolines compound (e.g. TLR7 agonists, such as, for example, Gardiquimod VACCIGRADE, Imiquimod VACCIGRADE, R848 VACCIGRADE); lipopolysaccharides (e.g. TLR4 agonists), such as that from E. coli 0111:B4 strain (e.g. LPS-EB VACCIGRADE); monophosphoryl lipid A (e.g. MPLA-SM VACCIGRADE and MPLA Synthetic VACCIGRADE); N-glycolylated muramyldipeptide (e.g. N-Glycolyl-MDP VACCIGRADE); CpG ODN, class A and/or CpG ODN, class B and/or CpG ODN, class C (e.g. ODN 1585 VACCIGRADE, ODN 1826 VACCIGRADE, ODN 2006 VACCIGRADE, ODN 2395 VACCIGRADE), a triacylated lipoprotein (e.g. Pam3CSK4 VACCIGRADE); Polyinosine-polycytidylic acid (e.g. Poly(I: C) (HMW) VACCIGRADE); and cord factor (i.e. mycobacterial cell wall component trehalose 6,6′ dimycolate (TDM)) or an analog thereof (e.g. TDB VACCIGRADE, TDB-HS15 VACCIGRADE). In some embodiments, the additional adjuvant is a TLR agonist (e.g. TLR1, and/or TLR2, and/or TLR3, and/or TLR4, and/or TLR5, and/or TLR6, and/or TLR7, and/or TLR8, and/or TLR9, and/or TLR10, and/or TLR11, and/or TLR12, and/or TLR13), a nucleotide-binding oligomerization domain (NOD) agonist, a stimulator of interferon genes (STING) ligand, or related agent. In some embodiments, the adjuvant is a ligand for toll like receptors (TLR) including endotoxin derived compounds, CpG, flagellin.

In some embodiments, the additional adjuvant is one or more of a mineral adjuvant, gel-based adjuvant, tensoactive agent, bacterial product, oil emulsion, particulated adjuvant, fusion protein, and lipopeptide. Other mineral salt adjuvants, besides the aluminum adjuvants described elsewhere, include salts of calcium (e.g. calcium phosphate), iron and zirconium. Other gel-based adjuvants, besides the aluminum gel-based adjuvants described elsewhere, include Acemannan. Tensoactive agents include Quil A, saponin derived from an aqueous extract from the bark of Quillaja saponaria; saponins, tensoactive glycosides containing a hydrophobic nucleus of triterpenoid structure with carbohydrate chains linked to the nucleus, and QS-21. Bacterial products include cell wall peptidoglycan or lipopolysaccharide of Gram-negative bacteria (e.g. from Mycobacterium spp., Corynebacterium parvum, C. granulosum, Bordetella pertussis and Neisseria meningitidis), N-acetyl muramyl-L-alanyl-D-isoglutamine (MDP), different compounds derived from MDP (e.g. threonyl-MDP), lipopolysaccharides (LPS) (e.g. from the cell wall of Gram-negative bacteria), trehalose dimycolate (TDM), and DNA containing CpG motifs. Oil emulsions include FIA, Montanide, Adjuvant 65, Lipovant, the montanide family of oil-based adjuvants, and various liposomes. Among particulated and polymeric systems, poly (DL-lactide-coglycolide) microspheres have been extensively studied and find use herein.

Further, in some embodiments, cytokines are an adjuvant of the present invention (e.g. IFN-γ and granulocyte-macrophage colony stimulating factor (GM-CSF)). Also carbohydrate adjuvants (e.g. inulin-derived adjuvants, such as, gamma inulin, algammulin (a combination of γ-inulin and aluminum hydroxide), and polysaccharides based on glucose and mannose, such as glucans, dextrans, lentinans, glucomannans and galactomannans) find use in the present invention. In some embodiments, adjuvant formulations are useful in the present invention and include alum salts in combination with other adjuvants such as Lipid A, algammulin, immunostimulatory complexes (ISCOMS), which are virus like particles of 30-40 nm and dodecahedric structure, composed of Quil A, lipids, and cholesterol.

In some embodiments, the additional adjuvants are described in Jennings et al. Adjuvants and Delivery Systems for Viral Vaccines-Mechanisms and Potential. In: Brown F, Haaheim L R, (eds). Modulation of the Immune Response to Vaccine Antigens. Dev. Biol. Stand, Vol. 92. Basel: Karger 1998; 19-28 and/or Sayers et al. J Biomed Biotechnol. 2012; 2012:831486, and/or Petrovsky and Aguilar, Immunology and Cell Biology (2004) 82, 488-496 the contents of which are hereby incorporated by reference in their entireties.

In various embodiments, the present adjuvants may be part of live and attenuated, or killed or inactivated, or toxoid, or subunit or conjugate vaccines. In some embodiments, the adjuvant and antigen are administered concurrently. In some embodiments, the adjuvant complex and antigen are co-formulated. In some embodiments, the adjuvant and antigen are administered sequentially. In some embodiments, the adjuvant and antigen are administered in multiple doses. In some embodiments, the adjuvant is administered in multiple booster doses and the antigen is administered once.

In some embodiments, the vaccine or vaccine composition of the present invention causes an improvement in adjuvant properties relative to a vaccine comprising the antigen and the aluminum gel or salt alone. In various embodiments, the vaccine and/or adjuvant described herein causes a broader, more diverse, more robust and longer lasting immunostimulatory effect than the vaccine comprising the antigen and the aluminum gel or salt alone and/or the adjuvant comprising the aluminum gel or salt alone.

In various embodiments, the present vaccine composition is part of the following vaccines (e.g. the antigens of these vaccines may be used as the antigen of the present vaccines): DTP (diphtheria-tetanus-pertussis vaccine), DTaP (diphtheria-tetanus-acellular pertussis vaccine), Hib (Haemophilus influenzae type b) conjugate vaccines, Pneumococcal conjugate vaccine, Hepatitis A vaccines, Poliomyelitis vaccines, Yellow fever vaccines, Hepatitis B vaccines, combination DTaP, Tdap, Hib, Human Papillomavirus (HPV) vaccine, Anthrax vaccine, and Rabies vaccine.

In some embodiments, the adjuvant or vaccine composition as described herein has (a) low toxicity; (b) an ability to stimulate a long-lasting immune response against the antigen; (c) substantial stability; (d) an ability to elicit a humoral immune response and/or a cell-mediated immunity to the antigen; (e) a capability of selectively interacting with populations of antigen presenting cells; (f) an ability to specifically elicit TH1 and/or TH2 cell-specific immune responses to the antigen; and/or (g) an ability to selectively increase appropriate antibody isotype levels against antigens, the isotype optionally being IgA, when administered to a patient.

In embodiments, the adjuvant or vaccine composition does not substantially cause one or more of fever, neutrophilia and the release of acute phase proteins when administered to a patient. In some embodiments, the adjuvant or vaccine composition stimulates activation of the IL-1R, when administered to a patient. In some embodiments, the methods described herein are where the adjuvant or vaccine composition stimulates activation of the IL-1R, when administered to a patient.

In some embodiments, the present invention is related to a composition comprising a wild type IL-1α, e.g. with an amino acid sequence of SEQ ID NO: 1 or 3, or a variant having at least about 95%, or at least about 97%, or at least about 99% identity thereto, and a targeting moiety that comprises a recognition domain that recognizes and/or binds CD8, CD3, CD4, Clec9A, XCR1, or SIRP1α. In some embodiments, this composition is a chimeric protein composition, a chimeric protein complex composition or a vaccine composition as described herein.

In some embodiments, the present invention is related to a composition comprising a wild type pro-IL-1α, e.g. with an amino acid sequence of SEQ ID NO: 2 or 4, or a variant having at least about 95%, or at least about 97%, or at least about 99% identity thereto, and a targeting moiety that comprises a recognition domain that recognizes and/or binds CD8, CD3, CD4, Clec9A, XCR1, or SIRP1α. In some embodiments, this composition is a chimeric protein composition, a chimeric protein complex composition or a vaccine composition as described herein.

In some embodiments, the present invention is related to a composition comprising a mutant IL-1α that comprises one or more substitution mutations selected from N29, S31, P3, M15, R16, 117, 118, L24, N25, D26, L28, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, and Q153 with respect to the amino acid sequence of SEQ ID NO: 1 or 3, or a variant having at least about 95%, or at least about 97%, or at least about 99% identity thereto, and a targeting moiety that comprises a recognition domain that recognizes and/or binds CD8, CD3, CD4, Clec9A, XCR1, or SIRP1α. In some embodiments, one or more of these mutations produce a modified human IL-1α with reduced or increased binding affinity for type I IL-1 and reduced or increased biological activity. In some embodiments, the mutant IL-1α has one or more substitution mutations selected from N29A, N29D, N29G, S31A, S31G, M15S, R16A, R16K, R16G, 118A, 118L, L24K, L24S, N25A, N25G, D26V, L28A, L28G, A44G, A44S, A44T, A44N, A44H H46A, H46G, A58G, A58S, A58T, A58N, A58H, A58F, Y59A, K60A, K60G, D64A, D64G, D65A, 168A, 168G, V70A, Y80A, K100A, K100D, W113F, Q136C, D151K, F152Q, F152N, F152S, Q153A, and Q153G where the residue numbering is based on SEQ ID NO: 1 or 3. In some embodiments, this composition is a chimeric protein composition, a chimeric protein complex composition or a vaccine composition as described herein.

In some embodiments, the present invention is related to a composition comprising a mutant pro-IL-1α that comprises one or more substitution mutations selected from N141, S143, P115, M127, R128, 1129, 1130, L136, N137, D138, L140, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, and Q265, with respect to the amino acid sequence of SEQ ID NO: 2 or 4, or a variant having at least about 95%, or at least about 97%, or at least about 99% identity thereto, and a targeting moiety that comprises a recognition domain that recognizes and/or binds CD8, CD3, CD4, Clec9A, XCR1, or SIRP1α. In some embodiments, one or more of these mutations produce a modified human pro-IL-1α with reduced or increased binding affinity for type I IL-1 and reduced or increased biological activity. In some embodiments, the mutant pro-IL-1α has a one or more substitution mutations selected from N141A, N141D, N141G, S143A, S143G, M127S, R128A, R128K, 1130A, 1130L, L136K, L136S, N137A, N137G, D138V, L140A, L140G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G where the residue numbering is based on SEQ ID NO: 2 or 4. In some embodiments, this composition is a chimeric protein composition, a chimeric protein complex composition or a vaccine composition as described herein.

In some embodiments, the antigen of the present invention is a protein or an antigenic fragment of a protein. In some embodiments, the antigen is a nucleic acid encoding a protein or an antigenic fragment of a protein. In embodiments, the nucleic acid which is an antigen or which encodes a protein or an antigenic fragment of a protein can be an mRNA, optionally comprising one or more non-canonical nucleotides, optionally selected from pseudouridine and 5-methoxyuridine. In embodiments, the nucleic acid is DNA, optionally selected from linear DNA, DNA fragments, or DNA plasmids.

### Pharmaceutically Acceptable Salts and Excipients

Chimeric protein, chimeric protein complex, vaccine composition, or adjuvant described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

### Pharmaceutical Compositions and Formulations

In various embodiments, the present invention pertains to pharmaceutical compositions comprising chimeric protein, chimeric protein complex, vaccine composition, or adjuvant described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients.

Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, to the lung, by inhalation, sublingual, intranasal, intracerebral, intravaginal, transdermal, rectally, by inhalation, or topically. Administration can be local or systemic. In some embodiments, the administering is effected orally. In another embodiment, the administration is by parenteral injection or via aerosol or nebulizer. The mode of administration can be left to the discretion of the practitioner, and depends in-part upon the site of the medical condition. In most instances, administration results in the release of any agent described herein into the bloodstream. In some embodiments, the administration is via liquid nebulization, dry powder dispersion or meter-dose administration.

In one embodiment, chimeric protein, chimeric protein complex, vaccine composition, or adjuvant described herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any chimeric protein, chimeric protein complex, vaccine composition, or adjuvant described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled-or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of chimeric protein, chimeric protein complex, vaccine composition, or adjuvant to be administered according to the present invention will vary according to the particular dosage form, and the mode of administration. Many factors that may modify the action of chimeric protein, chimeric protein complex, vaccine composition, or adjuvant (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In some embodiments, a suitable dosage of chimeric protein, chimeric protein complex, vaccine composition, or adjuvant is in a range of about 0.01 μg/kg to about 100 mg/kg of body weight of the subject, about 0.01 μg/kg to about 10 mg/kg of body weight of the subject, or about 0.01 μg/kg to about 1 mg/kg of body weight of the subject for example, about 0.01 μg/kg, about 0.02 μg/kg, about 0.03 μg/kg, about 0.04 μg/kg, about 0.05 μg/kg, about 0.06 μg/kg, about 0.07 μg/kg, about 0.08 μg/kg, about 0.09 μg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, or about 100 mg/kg body weight, inclusive of all values and ranges therebetween.

Individual doses of chimeric protein, chimeric protein complex, vaccine composition, or adjuvant can be administered in unit dosage forms (e.g., tablets, capsules, or liquid formulations) containing, for example, from about 1 μg to about 100 mg, from about 1 μg to about 90 mg, from about 1 μg to about 80 mg, from about 1 μg to about 70 mg, from about 1 μg to about 60 mg, from about 1 μg to about 50 mg, from about 1 μg to about 40 mg, from about 1 μg to about 30 mg, from about 1 μg to about 20 mg, from about 1 μg to about 10 mg, from about 1 μg to about 5 mg, from about 1 μg to about 3 mg, from about 1 μg to about 1 mg per unit dosage form, or from about 1 μg to about 50 μg per unit dosage form. For example, a unit dosage form can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In one embodiment, chimeric protein, chimeric protein complex, vaccine composition, or adjuvant is administered at an amount of from about 1 μg to about 100 mg daily, from about 1 μg to about 90 mg daily, from about 1 μg to about 80 mg daily, from about 1 μg to about 70 mg daily, from about 1 μg to about 60 mg daily, from about 1 μg to about 50 mg daily, from about 1 μg to about 40 mg daily, from about 1 μg to about 30 mg daily, from about 1 μg to about 20 mg daily, from about 01 μg to about 10 mg daily, from about 1 μg to about 5 mg daily, from about 1 μg to about 3 mg daily, or from about 1 μg to about 1 mg daily. In various embodiments, chimeric protein, chimeric protein complex, vaccine composition, or adjuvant is administered at a daily dose of about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

### Combination Therapy and Additional Therapeutic Agents

In various embodiments, the pharmaceutical composition of the present invention is co-administered in conjunction with additional therapeutic agent(s). Co-administration can be simultaneous or sequential.

In one embodiment, the additional therapeutic agent and chimeric protein, chimeric protein complex, vaccine composition, or adjuvant of the present invention are administered to a subject simultaneously. The term “simultaneously” as used herein, means that the additional therapeutic agent and chimeric protein, chimeric protein complex, vaccine composition, or adjuvant are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and chimeric protein, chimeric protein complex, vaccine composition, or adjuvant can be by simultaneous administration of a single formulation (e.g., a formulation comprising the additional therapeutic agent and chimeric protein, chimeric protein complex, vaccine composition, or adjuvant) or of separate formulations (e.g., a first formulation including the additional therapeutic agent and a second formulation including chimeric protein, chimeric protein complex, vaccine composition, or adjuvant).

Co-administration does not require the therapeutic agents to be administered simultaneously, if the timing of their administration is such that the pharmacological activities of the additional therapeutic agent and chimeric protein, chimeric protein complex, vaccine composition, or adjuvant overlap in time, thereby exerting a combined therapeutic effect. For example, the additional therapeutic agent and chimeric protein, chimeric protein complex, vaccine composition, or adjuvant can be administered sequentially. The term “sequentially” as used herein means that the additional therapeutic agent and chimeric protein, chimeric protein complex, vaccine composition, or adjuvant are administered with a time separation of more than about 60 minutes. For example, the time between the sequential administration of the additional therapeutic agent and chimeric protein, chimeric protein complex, vaccine composition, or adjuvant can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and chimeric protein, chimeric protein complex, vaccine composition, or adjuvant being administered. Either the additional therapeutic agent or chimeric protein, chimeric protein complex, vaccine composition, or adjuvant cell may be administered first.

Co-administration also does not require the therapeutic agents to be administered to the subject by the same route of administration. Rather, each therapeutic agent can be administered by any appropriate route, for example, parenterally or non-parenterally.

In some embodiments, chimeric protein, chimeric protein complex, vaccine composition, or adjuvant described herein acts synergistically when co-administered with another therapeutic agent. In such embodiments, chimeric protein, chimeric protein complex, vaccine composition, or adjuvant and the additional therapeutic agent may be administered at doses that are lower than the doses employed when the agents are used in the context of monotherapy.

In some embodiments, the present invention pertains to chemotherapeutic agents as additional therapeutic agents. For example, without limitation, such combination of chimeric protein, chimeric protein complex, vaccine composition, or adjuvant and chemotherapeutic agent find use in the treatment of cancers, as described elsewhere herein. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In some embodiments, inclusive of, without limitation, infectious disease applications, the present invention pertains to anti-infectives as additional therapeutic agents. In some embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In some embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

In illustrative embodiments, the present invention pertains to the use of hepatitis therapeutics as additional therapeutic agents. In various embodiments, the hepatitis therapeutics include, but are not limited to, IFN-α such as INTRON A or pegylated IFN-α such as Pegasys or PEG-INTRON, ribavirin, boceprevir, simeprevir, sofosbuvir, simeprevir, daclatasvir, ledipasvir/sofosbuvir ((Harvoni), ombitasvir/paritaprevir/ritonavir (Technivie), ombitasvir/paritaprevir/ritonavir/dasabuvir (Viekira Pak), lamivudine, adefovir, entecavir, telbivudine, entecavir, tenofovir, velpatasvir, elbasvir, grazoprevir, dasabuvir, and any combinations thereof. In an embodiment, the additional therapeutic agent is IFN-α (e.g., INTRON A) or pegylated IFN-α (e.g., Pegasys or PEG-INTRON). In another embodiment, the additional therapeutic agent is ribavirin.

In some embodiments, the present invention relates to combination therapies using chimeric protein, chimeric protein complex, vaccine composition, or adjuvant and an immunosuppressive agent. In some embodiments, the present invention relates to administration of the Clec9A binding agent to a patient undergoing treatment with an immunosuppressive agent.

In an embodiment, the immunosuppressive agent is TNF. In illustrative embodiments, chimeric protein, chimeric protein complex, vaccine composition, or adjuvant act synergistically when co-administered with TNF. In an illustrative embodiment, chimeric protein, chimeric protein complex, vaccine composition, or adjuvant act synergistically when co-administered with TNF for use in treating tumor or cancer. For example, co-administration of chimeric protein, chimeric protein complex, vaccine composition, or adjuvant of the present invention and TNF may act synergistically to reduce or eliminate the tumor or cancer, or slow the growth and/or progression and/or metastasis of the tumor or cancer. In some embodiments, the combination of chimeric protein, chimeric protein complex, vaccine composition, or adjuvant and TNF may exhibit improved safety profiles when compared to the agents used alone in the context of monotherapy. In some embodiments, chimeric protein, chimeric protein complex, vaccine composition, or adjuvant and TNF may be administered at doses that are lower than the doses employed when the agents are used in the context of monotherapy.

In some embodiments, inclusive, without limitation, of autoimmune applications, the additional therapeutic agent is an immunosuppressive agent that is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In some embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin). Additional anti-inflammatory agents are described, for example, in U.S. Pat. No. 4,537,776, the entire contents of which is incorporated by reference herein.

In some embodiments, the present invention pertains to various agents used for treating obesity as additional therapeutic agents. Illustrative agents used for treating obesity include, but are not limited to, orlistat (e.g. ALL1, XENICAL), loracaserin (e.g. BELVIQ), phentermine-topiramate (e.g. QSYMIA), sibutramme (e.g. REDUCTIL or MERJDIA), rimonabant (ACOMPLLA), exenatide (e.g. BYETTA), pramlintide (e.g. SYMLIN) phentermine, benzphetamine, diethylpropion, phendimetrazme, bupropion, and metformin. Agents that interfere with the body's ability to absorb specific nutrients in food are among the additional agents, e.g. orlistat (e.g. ALU, XENICAL), glucomannan, and guar gum. Agents that suppress appetite are also among the additional agents, e.g. catecholamines and their derivatives (such as phenteimine and other amphetamine-based drugs), various antidepressants and mood stabilizers (e.g. bupropion and topiramate), anorectics (e.g. dexedrine, digoxin). Agents that increase the body's metabolism are also among the additional agents.

In some embodiments, additional therapeutic agents may be selected from among appetite suppressants, neurotransmitter reuptake inhibitors, dopaminergic agonists, serotonergic agonists, modulators of GABAergic signaling, anticonvulsants, antidepressants, monoamine oxidase inhibitors, substance P (NK1) receptor antagonists, melanocortin receptor agonists and antagonists, lipase inhibitors, inhibitors of fat absorption, regulators of energy intake or metabolism, cannabinoid receptor modulators, agents for treating addiction, agents for treating metabolic syndrome, peroxisome proliferator-activated receptor (PPAR) modulators; dipeptidyl peptidase 4 (DPP-4) antagonists, agents for treating cardiovascular disease, agents for treating elevated triglyceride levels, agents for treating low HDL, agents for treating hypercholesterolemia, and agents for treating hypertension. Some agents for cardiovascular disease include statins (e.g. lovastatin, atorvastatin, fluvastatin, rosuvastatin, simvastatin and pravastatin) and omega-3 agents (e.g. LOVAZA, EPANQVA, VASCEPA, esterified omega-3's in general, fish oils, krill oils, algal oils). In some embodiments, additional agents may be selected from among amphetamines, benzodiazepines, suifonyl ureas, meglitinides, thiazolidinediones, biguanides, beta-blockers, XCE inhibitors, diuretics, nitrates, calcium channel blockers, phenlermine, sibutramine, iorcaserin, cetilistat, rimonabant, taranabant, topiramate, gabapentin, valproate, vigabatrin, bupropion, tiagabine, sertraline, fluoxetine, trazodone, zonisamide, methylphenidate, varenicline, naltrexone, diethylpropion, phendimetrazine, rcpaglini.de, nateglinide, glimepiride, metformin, pioglitazone, rosiglilazone, and sitagliptin.

In some embodiments, the present invention pertains to an agent used for treating diabetes as additional therapeutic agents. Illustrative anti-diabetic agents include, but are not limited to, sulfonylurea (e.g., DYMELOR (acetohexamide), DIABINESE (chlorpropamide), ORINASE (tolbutamide), and TOLINASE (tolazamide), GLUCOTROL (glipizide), GLUCOTROL XL (extended release), DIABETA (glyburide), MICRONASE (glyburide), GLYNASE PRESTAB (glyburide), and AMARYL (glimepiride)); a Biguanide (e.g. metformin (GLUCOPHAGE, GLUCOPHAGE XR, RIOMET, FORTAMET, and GLUMETZA)); a thiazolidinedione (e.g. ACTOS (pioglitazone) and AVANDIA (rosiglitazone); an alpha-glucosidase inhibitor (e.g., PRECOSE (acarbose) and GLYSET (miglitol); a Meglitinide (e.g., PRANDIN (repaglinide) and STARLIX (nateglinide)); a Dipeptidyl peptidase IV (DPP-IV) inhibitor (e.g., JANUVIA (sitagliptin), NESINA (alogliptin), ONGLYZA (saxagliptin), and TRADJENTA (linagliptin)); Sodium-glucose co-transporter 2 (SGLT2) inhibitor (e.g. INVOKANA (canaglifozin)); and a combination pill (e.g. GLUCOVANCE, which combines glyburide (a sulfonylurea) and metformin, METAGLIP, which combines glipizide (a sulfonylurea) and metformin, and AVANDAMET, which uses both metformin and rosiglitazone (AVANDIA) in one pill, KAZANO (alogliptin and metformin), OSENI (alogliptin plus pioglitazone), METFORMIN oral, ACTOS oral, BYETTA subcutaneous, JANUVIA oral, WELCHOL oral, JANUMET oral, glipizide oral, glimepiride oral, GLUCOPHAGE oral, LANTUS subcutaneous, glyburide oral, ONGLYZA oral, AMARYI oral, LANTUS SOLOSTAR subcutaneous, BYDUREON subcutaneous, LEVEMIR FLEXPEN subcutaneous, ACTOPLUS MET oral, GLUMETZA oral, TRADJENTA oral, bromocriptine oral, KOMBIGLYZE XR oral, INVOKANA oral, PRANDIN oral, LEVEMIR subcutaneous, PARLODEL oral, pioglitazone oral, NOVOLOG subcutaneous, NOVOLOG FLEXPEN subcutaneous, VICTOZA 2-PAK subcutaneous, HUMALOG subcutaneous, STARLIX oral, FORTAMET oral, GLUCOVANCE oral, GLUCOPHAGE XR oral, NOVOLOG Mix 70-30 FLEXPEN subcutaneous, GLYBURIDE-METFORMIN oral, acarbose oral, SYMLINPEN 60 subcutaneous, GLUCOTROI XL oral, NOVOLIN R inj, GLUCOTROL oral, DUETACT oral, sitagliptin oral, SYMLINPEN 120 subcutaneous, HUMALOG KWIKPEN subcutaneous, JANUMET XR oral, GLIPIZIDE-METFORMIN oral, CYCLOSET oral, HUMALOG MIX 75-25 subcutaneous, nateglinide oral, HUMALOG Mix 75-25 KWIKPEN subcutaneous, HUMULIN 70/30 subcutaneous, PRECOSE oral, APIDRA subcutaneous, Humulin R inj, Jentadueto oral, Victoza 3-Pak subcutaneous, Novolin 70/30 subcutaneous, NOVOLIN N subcutaneous, insulin detemir subcutaneous, glyburide micronized oral, GLYNASE oral, HUMULIN N subcutaneous, insulin glargine subcutaneous, RIOMET oral, pioglitazone-metformin oral, APIDRA SOLOSTAR subcutaneous, insulin lispro subcutaneous, GLYSET oral, HUMULIN 70/30 Pen subcutaneous, colesevelam oral, sitagliptin-metformin oral, DIABETA oral, insulin regular human inj, HUMULIN N Pen subcutaneous, exenatide subcutaneous, HUMALOG Mix 50-50 KWIKPEN subcutaneous, liraglutide subcutaneous, KAZANO oral, repaglinide oral, chlorpropamide oral, insulin aspart subcutaneous, NOVOLOG Mix 70-30 subcutaneous, HUMALOG Mix 50-50 subcutaneous, saxagliptin oral, ACTOPLUS Met XR oral, miglitol oral, NPH insulin human recomb subcutaneous, insulin NPH and regular human subcutaneous, tolazamide oral, mifepristone oral, insulin aspart protam-insulin aspart subcutaneous, repaglinide-metformin oral, saxagliptin-metformin oral, linagliptin-metformin oral, NESINA oral, OSENI oral, tolbutamide oral, insulin lispro protamine and lispro subcutaneous, pramlintide subcutaneous, insulin glulisine subcutaneous, pioglitazone-glimepiride oral, PRANDIMET oral, NOVOLOG PenFill subcutaneous, linagliptin oral, exenatide microspheres subcutaneous, KORLYM oral, alogliptin oral, alogliptin-pioglitazone oral, alogliptin-metformin oral, canagliflozin oral, Lispro (HUMALOG); Aspart (NOVOLOG); Glulisine (APIDRA); Regular (NOVOLIN R or HUMULIN R); NPH (NOVOLIN N or HUMULIN N); Glargine (LANTUS); Detemir (LEVEMIR); HUMULIN or NOVOLIN 70/30; and NOVOLOG Mix 70/30 HUMALOG Mix 75/25 or 50/50.

In some embodiments, chimeric protein, chimeric protein complex, vaccine composition, or adjuvant of the present invention act synergistically when used in combination with Chimeric Antigen Receptor (CAR) T-cell therapy. In an illustrative embodiment, chimeric protein, chimeric protein complex, vaccine composition, or adjuvant act synergistically when used in combination with CAR T-cell therapy in treating tumor or cancer. In an embodiment, chimeric protein, chimeric protein complex, vaccine composition, or adjuvant act synergistically when used in combination with CAR T-cell therapy in treating blood-based tumors. In an embodiment, chimeric protein, chimeric protein complex, vaccine composition, or adjuvant act synergistically when used in combination with CAR T-cell therapy in treating solid tumors. For example, use of chimeric protein, chimeric protein complex, vaccine composition, or adjuvant and CAR T-cells may act synergistically to reduce or eliminate the tumor or cancer, or slow the growth and/or progression and/or metastasis of the tumor or cancer. In various embodiments, chimeric protein, chimeric protein complex, vaccine composition, or adjuvant of the invention induces CAR T-cell division. In various embodiments, chimeric protein, chimeric protein complex, vaccine composition, or adjuvant of the invention induces CAR T-cell proliferation. In various embodiments, chimeric protein, chimeric protein complex, vaccine composition, or adjuvant of the invention prevents anergy of the CAR T cells.

In various embodiments, the CAR T-cell therapy comprises CAR T cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MUC16, ROR-1, IL13Rα2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, human epidermal growth factor receptor 2 (HER2), mesothelin, EGFRvIII, fibroblast activation protein (FAP), carcinoembryonic antigen (CEA), and vascular endothelial growth factor receptor 2 (VEGF-R2), as well as other tumor antigens well known in the art. Additional illustrative tumor antigens include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, lg-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2.

Illustrative CAR T-cell therapy include, but are not limited to, JCAR014 (Juno Therapeutics), JCAR015 (Juno Therapeutics), JCAR017 (Juno Therapeutics), JCAR018 (Juno Therapeutics), JCAR020 (Juno Therapeutics), JCAR023 (Juno Therapeutics), JCAR024 (Juno Therapeutics), CTL019 (Novartis), KTE-C19 (Kite Pharma), BPX-401 (Bellicum Pharmaceuticals), BPX-501 (Bellicum Pharmaceuticals), BPX-601 (Bellicum Pharmaceuticals), bb2121 (Bluebird Bio), CD-19 Sleeping Beauty cells (Ziopharm Oncology), UCART19 (Cellectis), UCART123 (Cellectis), UCART38 (Cellectis), UCARTCS1 (Cellectis), OXB-302 (Oxford BioMedica, MB-101 (Mustang Bio) and CAR T-cells developed by Innovative Cellular Therapeutics.

In some embodiments, chimeric protein, chimeric protein complex, vaccine composition, or adjuvant of the present invention is used in a method of treating multiple sclerosis (MS) in combination with one or more MS therapeutics including, but not limited to, 3-interferons, glatiramer acetate, T-interferon, IFN-β-2 (U.S. Patent Publication No. 2002/0025304), spirogermaniums (e.g., N-(3-dimethylaminopropyl)-2-aza-8,8-dimethyl-8-germanspiro [4:5] decane, N-(3-dimethylaminopropyl)-2-aza-8,8-diethyl-8-germaspiro [4:5] decane, N-(3-dimethylaminopropyl)-2-aza-8,8-dipropyl-8-germaspiro[4:5] decane, and N-(3-dimethylaminopropyl)-2-aza-8, 8-dibutyl-8-germaspiro [4:5] decane), vitamin D analogs (e.g., 1,25 (OH) 2D3, (see, e.g., U.S. Pat. No. 5,716,946)), prostaglandins (e.g., latanoprost, brimonidine, PGE1, PGE2 and PGE3, see, e.g., U.S. Patent Publication No. 2002/0004525), tetracycline and derivatives (e.g., minocycline and doxycycline, see, e.g., U.S. Patent Publication No. 20020022608), a VLA-4 binding antibody (see, e.g., U.S. Patent Publication No. 2009/0202527), adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, fumarate, anti-CD20 antibody (e.g., rituximab), and tizanidine hydrochloride.

In some embodiments, chimeric protein, chimeric protein complex, vaccine composition, or adjuvant is used in combination with one or more therapeutic agents that treat one or more symptoms or side effects of MS. Such agents include, but are not limited to, amantadine, baclofen, papaverine, meclizine, hydroxyzine, sulfamethoxazole, ciprofloxacin, docusate, pemoline, dantrolene, desmopressin, dexamethasone, tolterodine, phenyloin, oxybutynin, bisacodyl, venlafaxine, amitriptyline, methenamine, clonazepam, isoniazid, vardenafil, nitrofurantoin, psyllium hydrophilic mucilloid, alprostadil, gabapentin, nortriptyline, paroxetine, propantheline bromide, modafinil, fluoxetine, phenazopyridine, methylprednisolone, carbamazepine, imipramine, diazepam, sildenafil, bupropion, and sertraline.

In some embodiments, chimeric protein, chimeric protein complex, vaccine composition, or adjuvant is used in a method of treating multiple sclerosis in combination with one or more of the disease modifying therapies (DMTs) described herein (e.g. the agents of Table 6). In some embodiments, the present invention provides an improved therapeutic effect as compared to use of one or more of the DMTs described herein (e.g. the agents listed in Table 6 below) without the one or more disclosed binding agent. In an embodiment, the combination of chimeric protein, chimeric protein complex, vaccine composition, or adjuvant and the one or more DMTs produces synergistic therapeutic effects.

Illustrative disease modifying therapies include, but are not limited to:

TABLE 6

| Generic Name | Branded Name/Company | Frequency/Route of Delivery/Usual Dose |
|---|---|---|
| teriflunomide | AUBAGIO (GENZYME) | Every day; pill taken orally; 7 mg or 14 mg. |
| interferon beta-1a | AVONEX (BIOGEN IDEC) | Once a week; intramuscular (into the muscle) |
| | | injection; 30 mcg |
| interferon beta-1b | BETASERON (BAYER | Every other day; subcutaneous (under the |
| | HEALTHCARE | skin) injection; 250 mcg. |
| | PHARMACEUTICALS, INC.) | |
| glatiramer acetate | COPAXONE (TEVA | Every day; subcutaneous (under the skin) |
| | NEUROSCIENCE) | injection; 20 mg (20,000 mcg) OR Three |
| | | times a week; subcutaneous (under the skin) |
| | | injection; 40 mg (40,000 mcg) |
| interferon beta-1b | EXTAVIA (NOVARTIS | Every other day; subcutaneous (under the |
| | PHARMACEUTICALS | skin) injection; 250 mcg. |
| | CORP.) | |
| fingolimod | GILENYA (NOVARTIS | Every day; capsule taken orally; 0.5 mg. |
| | PHARMACEUTICALS | |
| | CORP.) | |
| Alemtuzumab (anti-CD52 | LEMTRADA (GENZYME) | Intravenous infusion on five consecutive days, |
| monoclonal antibody) | | followed by intravenous infusion on three |
| | | consecutive days one year later (12 mg) |
| mitoxantrone | NOVANTRONE (EMD | Four times a year by IV infusion in a medical |
| | SERONO) | facility. Lifetime cumulative dose limit of |
| | | approximately 8-12 doses over 2-3 years |
| | | (140 mg/m2). |
| pegylated interferon beta- | PLEGRIDY (BIOGEN IDEC) | Every 14 days; subcutaneous (under the skin) |
| 1a | | injection; 125 mcg |
| interferon beta-1a | REBIF (EMD SERONO, | Three times a week; subcutaneous (under the |
| | INC.) | skin) injection; 44 mcg |
| dimethyl fumarate (BG-12) | TECFIDERA (BIOGEN | Twice a day; capsule taken orally; 120 mg for |
| | IDEC) | one week and 240 mg therafter |
| Natalizumab (humanized | TYSABRI (BIOGEN IDEC) | Every four weeks by IV infusion in a |
| monoclonal antibody VLA- | | registered infusion facility; 300 mg |
| 4 antagonist) | | |

DMTs in Development

| | | |
|---|---|---|
| Amiloride (targets Acid- | PAR PHARMACEUTICAL, | Oral |
| sensing ion channel-1 | PERRIGO COMPANY, | |
| Epithelial sodium channel | SIGMAPHARM | |
| Na+/H+ exchanger) | LABORATORIES | |
| ATX-MS-1467 (targets | APITOPE/MERCK | Intradermal Subcutaneous |
| Major histocompatibility | SERONO | |
| complex class II T cell | | |
| responses to myelin basic | | |
| protein) | | |
| BAF312 (targets | NOVARTIS PHARMA | Oral |
| Sphingosine 1-phosphate | | |
| (S1P) receptor subtypes | | |
| S1P1 and S1P5 B cell | | |
| distrubution T cell | | |
| distribution) | | |
| BGC20-0134 (targets | BTG PLC | Oral |
| Proinflammatory and anti- | | |
| inflammatory cytokines) | | |
| BIIB033 (targets LINGO-1 | BIOGEN | Intravenous infusion used in Phase I and |
| (“leucine-rich repeat and | | Phase II trials Subcutaneous injection used in |
| immunoglobulin-like | | Phase I trial |
| domain-containing, Nogo | | |
| receptor-interacting | | |
| protein”)) | | |
| Cladribine (targets CD4+ | MERCK SERONO | Oral |
| T cells DNA synthesis and | | |
| repair E-selectin | | |
| Intracellular adhesion | | |
| molecule-1 Pro- | | |
| inflammatory cytokines | | |
| interleukin 2 and | | |
| interleukin 2R Pro- | | |
| inflammatory cytokines | | |
| interleukin 8 and RANTES | | |
| Cytokine secretion | | |
| Monocyte and lymphocyte | | |
| migration) | | |
| Cyclophosphamide | BAXTER HEALTHCARE | Oral, monthly intravenous pulses |
| (targets T cells, | CORPORATION | |
| particularly CD4+ helper T | | |
| cells B cells) | | |
| Daclizumab (humanized | BIOGEN IDEC/ABBVIE | Projected to be IM injection once monthly |
| monoclonal antibody | BIOTHERAPEUTICS | |
| targeting CD25 Immune | | |
| modulator of T cells) | | |
| Dalfampridine (targets | ACORDA THERAPEUTICS/ | One tablet every 12 hours (extended release), |
| Voltage-gated potassium | BIOGEN IDEC | 10 mg twice a day |
| channels | | |
| Degenerin/epithelial | | |
| sodium channels L-type | | |
| calcium channels that | | |
| contain subunit Cavbeta3) | | |
| Dronabinol (targets | ABBVIE INC. | Oral |
| Cannabinoid receptor CB1 | | |
| Cannabinoid receptor | | |
| CB2) | | |
| Firategrast (targets | GLAXOSMITHKLINE | Oral |
| Alpha4beta1 integrin) | | |
| GNbAC1MSRV-Env | GENEURO SA/SERVIER | Intravenous infusion |
| (targets envelope protein | | |
| of the MS-associated | | |
| retrovirus) | | |
| Idebenone (targets | SANTHERA | Oral Dose in clinical trial for PPMS is 2250 mg |
| Reactive oxygen species) | PHARMACEUTICALS | per day (750 mg dose, 3 times per day) |
| Imilecleucel-T (targets | OPEXA THERAPEUTICS/ | Subcutaneous Given 5 times per year, |
| Myelin-specific, | MERCK SERONO | according to information from the |
| autoreactive T cells) | | manufacturer |
| Laquinimod | TEVA | Projected to be 0.6 mg or 1.2 mg oral tablet |
| | | taken daily |
| Masitinib (targets KIT (a | AB SCIENCE | Oral |
| stem cell factor, also | | |
| called c-KIT) receptor as | | |
| well as select other | | |
| tyrosine kinases Mast | | |
| cells) | | |
| MEDI-551 (targets CD19, | MEDIMMUNE | Intravenous Subcutaneous |
| a B cell-specific antigen | | |
| that is part of the B cell | | |
| receptor complex and that | | |
| functions in determining | | |
| the threshold for B cell | | |
| activation B cells | | |
| Plasmablasts, B cells that | | |
| express CD19 (but not | | |
| CD20) and that secrete | | |
| large quantities of | | |
| antibodies; depletion of | | |
| plasmablasts may be | | |
| useful in autoimmune | | |
| diseases involving | | |
| pathogenic | | |
| autoantibodies) | | |
| Minocycline (targets T | VARIOUS | Oral Available as pellet-filled capsules and an |
| cells Microglia Leukocyte | | oral suspension |
| migration Matrix | | |
| metalloproteinases) | | |
| MIS416 (targets Innate | INNATE | Intravenous |
| immune system | IMMUNOTHERAPEUTICS | |
| Pathogen-associated | | |
| molecular pattern | | |
| recognition receptors of | | |
| the innate immune system | | |
| Myeloid cells of the innate | | |
| immune system, which | | |
| might be able to remodel | | |
| the deregulated immune | | |
| system activity that occurs | | |
| in SPMS) | | |
| Mycophenolate mofetil | MANUFACTURED BY | Oral |
| (targets Purine synthesis) | GENENTECH | |
| Naltrexone (targets Opioid | VARIOUS | Given at low doses (3 to 4.5 mg per day) in |
| receptors Toll-like | | oral form as “Low-dose naltrexone” |
| receptor 4) | | (or “LDN”) |
| Ocrelizumab and | ROCHE/GSK | Projected to be IV infusion |
| Ofatumumab (humanized | | |
| monoclonal antibodies | | |
| targeting CD20 B cell | | |
| suppression | | |
| ONO-4641 (targets | ONO PHARMACEUTICAL | Oral |
| Sphingosine 1-phosphate | CO. | |
| receptor) | | |
| Phenytoin (targets Sodium | PFIZER | Intravenous Intramuscular (less favored |
| channels) | | option) Oral |
| Ponesimod | ACTELION | To be determined |
| Raltegravir (targets | MERCK | Oral 400 mg tablet twice daily, according to |
| Retroviral integrase | | information from the manufacturer |
| Herpesvirus DNA | | |
| packaging terminase) | | |
| RHB-104 | REDHILL BIOPHARMA | 95 mg clarithromycin, 45 mg rifabutin, and 10 |
| | LIMITED | mg clofazimine |
| Riluzole (targets | COVIS PHARMA/SANOFI | Oral |
| Glutamatergic | | |
| neurotransmission | | |
| Glutamate uptake and | | |
| release Voltage-gated | | |
| sodium channels Protein | | |
| kinase C) | | |

In some embodiments, the present invention relates to combination therapy with a blood transfusion. For instance, the present compositions may supplement a blood transfusion. In some embodiments, the present invention relates to combination therapy with iron supplements.

In some embodiments, the present invention relates to combination therapy with one or more EPO-based agents. For example, the present compositions may be used as an adjuvant to other EPO-based agents. In some embodiments, the present compositions are used as a maintenance therapy to other EPO-based agents. Other EPO-based agents include the following: epoetin alfa, including without limitation, DARBEPOETIN (ARANESP), EPOCEPT (LUPIN PHARMA), NANOKINE (NANOGEN PHARMACEUTICAL), EPOFIT (INTAS PHARMA), EPOGEN (AMGEN), EPOGIN, EPREX, (JANSSEN-CILAG), BINOCRIT7 (SANDOZ), PROCRIT; epoetin beta, including without limitation, NEORECORMON (HOFFMANN-LA ROCHE), RECORMON, Methoxy polyethylene glycol-epoetin beta (MIRCERA, ROCHE); epoetin delta, including without limitation, DYNEPO (erythropoiesis stimulating protein, SHIRE PLC); epoetin omega, including without limitation, EPOMAX; epoetin zeta, including without limitation, SILAPO (STADA) and RETACRIT (HOSPIRA) and other EPOs, including without limitation, EPOCEPT (LUPIN PHARMACEUTICALS), EPOTRUST (PANACEA BIOTEC LTD), ERYPRO SAFE (BIOCON LTD.), REPOITIN (SERUM INSTITUTE OF INDIA LIMITED), VINTOR (EMCURE PHARMACEUTICALS), EPOFIT (INTAS PHARMA), ERYKINE (INTAS BIOPHARMACEUTICA), WEPOX (WOCKHARDT BIOTECH), ESPOGEN (LG LIFE SCIENCES), RELIPOIETIN (RELIANCE LIFE SCIENCES), SHANPOIETIN (SHANTHA BIOTECHNICS LTD), ZYROP (CADILA HEALTHCARE LTD.), EPIAO (RHUEPO) (SHENYANG SUNSHINE PHARMACEUTICAL CO. LTD), CINNAPOIETIN (CINNAGEN).

In some embodiments, the present invention relates to combination therapy with one or more immune-modulating agents, for example, without limitation, agents that modulate immune checkpoint. In various embodiments, the immune-modulating agent targets one or more of PD-1, PD-L1, and PD-L2. In various embodiments, the immune-modulating agent is PD-1 inhibitor. In various embodiments, the immune-modulating agent is an antibody specific for one or more of PD-1, PD-L1, and PD-L2. For instance, in some embodiments, the immune-modulating agent is an antibody such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL3280A (ROCHE). In some embodiments, the immune-modulating agent targets one or more of CD137 or CD137L. In various embodiments, the immune-modulating agent is an antibody specific for one or more of CD137 or CD137L. For instance, in some embodiments, the immune-modulating agent is an antibody such as, by way of non-limitation, urelumab (also known as BMS-663513 and anti-4-1BB antibody). In some embodiments, chimeric protein, chimeric protein complex, vaccine composition, or adjuvant is combined with urelumab (optionally with one or more of nivolumab, lirilumab, and urelumab) for the treatment of solid tumors and/or B-cell non-Hodgkins lymphoma and/or head and neck cancer and/or multiple myeloma. In some embodiments, the immune-modulating agent is an agent that targets one or more of CTLA-4, AP2M1, CD80, CD86, SHP-2, and PPP2R5A. In various embodiments, the immune-modulating agent is an antibody specific for one or more of CTLA-4, AP2M1, CD80, CD86, SHP-2, and PPP2R5A. For instance, in some embodiments, the immune-modulating agent is an antibody such as, by way of non-limitation, ipilimumab (MDX-010, MDX-101, Yervoy, BMS) and/or tremelimumab (Pfizer). In some embodiments, chimeric protein, chimeric protein complex, vaccine composition, or adjuvant is combined with ipilimumab (optionally with bavituximab) for the treatment of one or more of melanoma, prostate cancer, and lung cancer. In various embodiments, the immune-modulating agent targets CD20. In various embodiments, the immune-modulating agent is an antibody specific CD20. For instance, in some embodiments, the immune-modulating agent is an antibody such as, by way of non-limitation, Ofatumumab (GENMAB), obinutuzumab (GAZYVA), AME-133v (APPLIED MOLECULAR EVOLUTION), Ocrelizumab (GENENTECH), TRU-015 (TRUBION/EMERGENT), veltuzumab (IMMU-106).

Additional therapeutic agents, e.g. for coronavirus-related methods, include, for example, one or more of acyclovir, ganciclovir, remdesivir; favipiravir; galidesivir; prezcobix; lopinavir and/or ritonavir and/or arbidol; mRNA-1273; recombinant proteins such as agonists, antagonists, blockers, or decoy mimetics of the viral spike protein, or agonists, antagonists, blockers, or decoy mimetics of the ACE2 protein; stem cell-derived exosomes; lopinavir/ritonavir and/or ribavirin and/or IFN-alpha, IFN-beta, IFN-gamma; xiyanping; anti-VEGF-A agents (e.g. Bevacizumab, ranibizumab, aflibercept, and others); fingolimod; carrimycin; hydroxychloroquine chloroquine; darunavir and cobicistat; prednisone, prednisolone, methylprednisolone; fluocinalone, brilacidin; leronlimab (PRO 140); and thalidomide. In various embodiments, the present adjuvatns are administered to a patient undergoing treatment with one or more additional therapeutic agents. In various embodiments, additional therapeutic agents include anti-virals, anti-inflammatories, agents that reduce vascular leakage and tissue edema, anti-fibrotic agents. Additional therapeutic agents, include, for example, one or more of acyclovir, ganciclovir, remdesivir; favipiravir; galidesivir; prezcobix; lopinavir and/or ritonavir and/or arbidol; mRNA-1273; recombinant proteins such as agonists, antagonists, blockers, or decoy mimetics of the viral spike protein, or agonists, antagonists, blockers, or decoy mimetics of the ACE2 protein; stem cell-derived exosomes; lopinavir/ritonavir and/or ribavirin and/or IFN-alpha, IFN-beta, IFN-gamma; xiyanping; anti-VEGF-A agents (e.g. Bevacizumab, ranibizumab, aflibercept, and others); fingolimod; carrimycin; hydroxychloroquine chloroquine; darunavir and cobicistat; prednisone, prednisolone, methylprednisolone; fluocinalone, brilacidin; leronlimab (PRO 140); and thalidomide.

In some embodiments, the additional therapeutic agents include convalescent plasma, i.e., plasma from a donor subject (e.g. a human subject) who has recovered from the viral infection, e.g., SARS-CoV-2. In some embodiments, the additional therapeutic agents include plasma from a donor subject (e.g. a human subject) comprising IgG and IgM antibodies directed against a virus disclosed herein that causes an infection or a disease, e.g., SARS-CoV-2.

In some embodiments, the present invention relates to combination therapy with one or more chimeric agents described in WO 2013/10779, WO 2015/007536, WO 2015/007520, WO 2015/007542, and WO 2015/007903, the entire contents of which are hereby incorporated by reference in their entireties.

In some embodiments, the present invention relates to combination therapy with one or more chimeric agents described in WO 2013/10779, WO 2015/007536, WO 2015/007520, WO 2015/007542, and WO 2015/007903, the entire contents of which are hereby incorporated by reference in their entireties.

In some embodiments, chimeric protein, chimeric protein complex, vaccine composition, or adjuvant described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc.

In still other embodiments, chimeric protein, chimeric protein complex, vaccine composition, or adjuvant described herein further comprise a cytotoxic agent, comprising, in illustrative embodiments, a toxin, a chemotherapeutic agent, a radioisotope, and an agent that causes apoptosis or cell death. Such agents may be conjugated to a composition described herein.

Chimeric protein, chimeric protein complex, vaccine composition, or adjuvant described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Illustrative cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Toxin conjugated monoclonal antibodies, tumor antigen specific monoclonal antibodies, Erbitux, Avastin, Pertuzumab, anti-CD20 antibodies, Rituxan, ocrelizumab, ofatumumab, DXL625, HERCEPTIN®, or any combination thereof. Toxic enzymes from plants and bacteria such as ricin, diphtheria toxin and Pseudomonas toxin may be conjugated to the therapeutic agents (e.g. antibodies) to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to chimeric protein, chimeric protein complex, vaccine composition, or adjuvant, with or without the use of a complex-forming agent. Such radionuclides include beta-emitters such as Phosphorus-32, Scandium-47, Copper-67, Gallium-67, Yttrium-88, Yttrium-90, lodine-125, lodine-131, Samarium-153, Lutetium-177, Rhenium-186 or Rhenium-188, and alpha-emitters such as Astatine-211, Lead-212, Bismuth-212, Bismuth-213 or Actinium-225.

Illustrative detectable moieties further include, but are not limited to, horseradish peroxidase, acetylcholinesterase, alkaline phosphatase, beta-galactosidase and luciferase. Further illustrative fluorescent materials include, but are not limited to, rhodamine, fluorescein, fluorescein isothiocyanate, umbelliferone, dichlorotriazinylamine, phycoerythrin and dansyl chloride. Further illustrative chemiluminescent moieties include, but are not limited to, luminol. Further illustrative bioluminescent materials include, but are not limited to, luciferin and aequorin. Further illustrative radioactive materials include, but are not limited to, lodine-125, Carbon-14, Sulfur-35, Tritium and Phosphorus-32.

### Methods of Treatment or Vaccination

Methods and compositions described herein have application to treating various diseases and disorders, including, but not limited to cancer and infectious diseases, immune disorders, anemia, autoimmune diseases, cardiovascular diseases, wound healing, ischemia-related diseases, neurodegenerative diseases, metabolic diseases and many other diseases and disorders.

Further, any of the present agents may be for use in the treating, or the manufacture of a medicament for treating, various diseases and disorders, including, but not limited to cancer, infections, immune disorders, inflammatory diseases or conditions, and autoimmune diseases.

In some embodiments, the present invention relates to the treatment of, or a patient having one or more of chronic granulomatous disease, osteopetrosis, idiopathic pulmonary fibrosis, Friedreich's ataxia, atopic dermatitis, Chagas disease, cancer, heart failure, autoimmune disease, sickle cell disease, thalassemia, blood loss, transfusion reaction, diabetes, vitamin B12 deficiency, collagen vascular disease, Shwachman syndrome, thrombocytopenia purpura, Celiac disease, endocrine deficiency state such as hypothyroidism or Addison's disease, autoimmune disease such as Crohn's Disease, systemic lupus erythematosis, rheumatoid arthritis or juvenile rheumatoid arthritis, ulcerative colitis immune disorders such as eosinophilic fasciitis, hypoimmunoglobulinemia, or thymoma/thymic carcinoma, graft versus host disease, preleukemia, Nonhematologic syndrome (e.g. Down's, Dubowwitz, Seckel), Felty syndrome, hemolytic uremic syndrome, myelodysplasic syndrome, nocturnal paroxysmal hemoglobinuria, osteomyelofibrosis, pancytopenia, pure red-cell aplasia, Schoenlein-Henoch purpura, malaria, protein starvation, menorrhagia, systemic sclerosis, liver cirrhosis, hypometabolic states, and congestive heart failure.

In some embodiments, chimeric protein, chimeric protein complex, vaccine composition, or adjuvant described herein are suitable for vaccinating against, preventing, or mitigating a disease or disorder is an infectious disease. In some embodiments, the disease or disorder is selected from diphtheria, tetanus, pertussis, influenza, pneumonia, hepatitis A, hepatitis B, polio, yellow fever, Human Papillomavirus (HPV) infection, anthrax, rabies, Japanese Encephalitis, meningitis, measles, mumps, rubella, gastroenteritis, smallpox, typhoid fever, varicella (chickenpox), rotavirus, and shingles.

One aspect of the present invention is related to a method for vaccinating a subject against an infectious disease, comprising administering: (a) administering an adjuvant comprising a chimeric protein or chimeric protein complex, comprising: (i) a mutant IL-1α or pro-IL-1α, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) or (2) a flexible linker that connects (i) and (ii); wherein the mutant IL-1α or mutant pro-IL-1α is characterized by low or high affinity or activity at the IL-1 receptor; and (b) an antigen which is suitable for inducing an immune response.

In some embodiments, the infectious disease is an infection with a pathogen, optionally selected from a bacterium, virus, fungus, or parasite. In some embodiments, the virus is: (a) an influenza virus, optionally selected from Type A, Type B, Type C, and Type D influenza viruses, or (b) a member of the Coronaviridae family, optionally selected from a betacoronavirus, optionally selected from severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or an alphacoronavirus, optionally selected from HCoV-NL63 and HCoV-229E. In some embodiments, the virus is SARS-CoV-2. In some embodiments, the antigen is a 2019-nCoV protein, an antigenic fragment thereof, or a nucleic acid encoding the same, optionally selected from spike surface glycoprotein, membrane glycoprotein M, envelope protein E, and nucleocapsid phosphoprotein N. In some embodiments, the antigen is the S1 or S2 subunit of the spike surface glycoprotein, or an antigenic fragment thereof.

In an embodiment, the spike surface glycoprotein comprises the amino acid sequence of SEQ ID NO: 500:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASWNIQKEIDRLNEVAKNLNESLIDLQ
ELGKYEQYIKWPWYIWLGFIAGLIAIVMVTIMLCCMTSCCSCLKGCCSCG
SCCKFDEDDSEPVLKGVKLHYT

In an embodiment, the membrane glycoprotein precursor M comprises the amino acid sequence of SEQ ID NO: 501:

MADSNGTITVEELKKLLEQWNLVIGFLFLTWICLLQFAYANRNRFLYIIK
LIFLWLLWPVTLACFVLAAVYRINWITGGIAIAMACLVGLMWLSYFIASF
RLFARTRSMWSFNPETNILLNVPLHGTILTRPLLESELVIGAVILRGHLR
IAGHHLGRCDIKDLPKEITVATSRTLSYYKLGASQRVAGDSGFAAYSRYR
IGNYKLNTDHSSSSDNIALLVQ

In an embodiment, the envelope protein E comprises the amino acid sequence of SEQ ID NO: 502:

MYSFVSEETGTLIVNSVLLFLAFVVFLLVTLAILTALRLCAYCCNIVNVS
LVKPSFYVYSRVKNLNSSRVPDLLV

In an embodiment, the nucleocapeid phosphoprotein N comprises the amino acid sequence of SEQ ID NO: 503:

MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA

In various embodiments, the subject is afflicted with coronavirus disease 2019 (COVID-19). In additional embodiments, the subject is elderly and/or afflicted with one or more comorbidities, including, but not limited to, hypertension and/or diabetes. A subject afflicted with a coronavirus infection can acquire symptoms including, but not limited to, fever, tiredness, dry cough, aches and pains, shortness of breath and other breathing difficulties, diarrhea, upper respiratory symptoms (e.g. sneezing, runny nose, nasal congestion, cough, sore throat), pneumonia, pneumonia respiratory failure, hepatic and renal insufficiency, acute respiratory distress syndrome (ARDS), and a cytokine imbalance.

In some embodiments, the virus is an influenza virus. In some embodiments, the antigen is an influenza viral antigen, optionally selected from hemagglutinin (HA) protein, matrix 2 (M2) protein, and neuraminidase, or an antigenic fragment thereof, or a nucleic acid encoding the same.

In some embodiments, the antigens described herein have at least about 90%, or at least about 95%, or at least about 97%, or at least about 98%, or at least about 99% identity with its wild type sequence. In some embodiments, the spike surface glycoprotein, membrane glycoprotein M, envelope protein E, and nucleocapsid phosphoprotein N have at least about 90%, or at least about 95%, or at least about 97%, or at least about 98%, or at least about 99% identity with their sequences as shown above.

Another aspect of the present invention is related to a method for vaccinating a subject against an influenza infection, comprising administering: (a) administering an adjuvant comprising a chimeric protein or chimeric protein complex, comprising: (i) a mutant IL-1α or mutant pro-IL-1α, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) or (2) a flexible linker that connects (i) and (ii); wherein the mutant IL-1α or mutant pro-IL-1α is characterized by low or high affinity or activity at the IL-1 receptor; and (b) an influenza antigen which is suitable for inducing an immune response.

Yet another aspect of the present invention is related to a method for vaccinating a subject against a severe acute respiratory syndrome coronavirus 2 (SARS-CoV-2) infection comprising administering: (a) administering an adjuvant comprising a chimeric protein or chimeric protein complex, comprising: (i) a mutant IL-1α or mutant pro-IL-1α, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) or (2) a flexible linker that connects (i) and (ii); wherein the mutant IL-1α or mutant pro-IL-1α is characterized by low or high affinity or activity at the IL-1 receptor; and (b) a SARS-CoV-2 antigen which is suitable for inducing an immune response.

In some aspects, the present invention is related to a method for treating a subject afflicted with an infectious disease, comprising administering a chimeric protein or chimeric protein complex, comprising: (i) an IL-1α, pro-IL-1α, or a mutant thereof, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) and/or (2) a flexible linker that connects (i) and (ii); wherein the mutant IL-1α or the mutant pro-IL-1α is characterized by low or high affinity or activity at the IL-1 receptor. For example, in embodiments, the invention is related to a method for treating a subject afflicted with a coronavirus (e.g. SARS-CoV-2) or influenza infection. In such embodiments, the adjuvant is administered to a patient who has a low level or moderate infection and the adjuvant causes a boost to the natural immune response to the infection occurring in the patient.

In some embodiments, the present invention relates to the treatment or vaccination of patients who are naive to antiviral therapy. In other embodiments, the present invention relates to the treatment or vaccination of patients who did not respond to previous antiviral therapy. In some embodiments, the present vaccine compositions may be used to vaccinate relapsed patients.

In some aspects, the present invention is related to a method for treating a subject afflicted with a cancer, comprising administering a chimeric protein or chimeric protein complex, comprising (i) an IL-1α, pro-IL-1α, or a mutant thereof, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) and/or (2) a flexible linker that connects (i) and (ii); wherein the mutant IL-1α or the mutant pro-IL-1α is characterized by low or high affinity or activity at the IL-1 receptor.

In some embodiments, the present invention is related to a method for treating cancer, comprising administering an effective amount of i) the chimeric protein and/or the chimeric protein complex (e.g., Fc-based chimeric protein complex) to a patient in need thereof; ii) a recombinant nucleic acid encoding the chimeric protein, the chimeric protein and/or the chimeric protein complex (e.g., Fc-based chimeric protein complex) to a patient in need thereof; or iii) a host cell comprising the recombinant nucleic acid encoding the chimeric protein, the chimeric protein and/or the chimeric protein complex (e.g., Fc-based chimeric protein complex) to a patient in need thereof.

In some embodiments, the present invention relates to the treatment of, or a patient having one or more of chronic granulomatous disease, osteopetrosis, idiopathic pulmonary fibrosis, Friedreich's ataxia, atopic dermatitis, Chagas disease, mycobacterial infections, cancer, scleroderma, hepatitis, hepatitis C, septic shock, and rheumatoid arthritis.

In some embodiments, the present invention relates to the treatment of, or a patient having cancer. As used herein, cancer refers to any uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

Illustrative cancers that may be treated include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g. astrocytoma, oligodendroglioma, and ependymoma), meningioma, pituitary adenoma, and neuromas, and spinal cord tumors (e.g. meningiomas and neurofibroma).

Illustrative cancers that may be treated include, but are not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In some embodiments, the present invention relates to the treatment of, or a patient having a microbial infection and/or chronic infection. Illustrative infections include, but are not limited to, Chagas disease, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In some embodiments, the disease or disorder is selected from diphtheria, tetanus, pertussis, influenza, pneumonia, hepatitis A, hepatitis B, polio, yellow fever, Human Papillomavirus (HPV) infection, anthrax, rabies, Japanese Encephalitis, meningitis, measles, mumps, rubella, gastroenteritis, smallpox, typhoid fever, varicella (chickenpox), rotavirus, and shingles. In some embodiments, the present invention relates to the treatment of hepatitis. Illustrative hepatitis that may be treated include, but is not limited to, hepatitis A, hepatitis B, hepatitis C, hepatitis D, hepatitis E, autoimmune hepatitis, alcoholic hepatitis, acute hepatitis, and chronic hepatitis.

In an illustrative embodiment, the present invention relates to the treatment of chronic hepatitis C. In an embodiment, the chimeric proteins or chimeric protein complexes such as Fc-based chimeric protein complex of the invention may be utilized to treat a patient infected with any one of the hepatitis C genotypes, including genotype 1 (e.g., 1a, 1b), genotype 2 (e.g. 2a, 2b, 2c and 2d), genotype 3 (e.g., 3a, 3b, 3c, 3d, 3e, and 3f), genotype 4 (e.g., 4a, 4b, 4c, 4d, 4e, 4f, 4g, 4h, 4i and 4j), genotype 5a, and genotype 6a.

In various embodiments, chimeric protein, chimeric protein complex, vaccine composition, or adjuvant of the invention may be utilized to treat patients who are poorly or non-responsive to standard of care antiviral therapy or who are otherwise difficult to treat with standard of care hepatitis C treatment. In an embodiment, the present invention is directed to the treatment of patients infected with hepatitis C genotype 1 or any other genotype who did not respond to previous therapy. In an embodiment, chimeric protein, chimeric protein complex, vaccine composition, or adjuvant of the invention may be used to treat a patient with high baseline viral load (e.g., greater than 800,000 IU/mL). In an embodiment, chimeric protein, chimeric protein complex, vaccine composition, or adjuvant of the invention may be utilized to treat patients with severe liver damage including those patients with advanced liver fibrosis and/or liver cirrhosis.

In some embodiments, the present invention relates to the treatment of patients who are naive to antiviral therapy. In other embodiments, the present invention relates to the treatment of patients who did not respond to previous antiviral therapy. In some embodiments, chimeric protein, chimeric protein complex, vaccine composition, or adjuvant may be used to treat relapsed patients.

In some embodiments, chimeric protein, chimeric protein complex, vaccine composition, or adjuvant may be effective in treating hepatitis infection in all ethnic groups including white, African-American, Hispanic, and Asian.

In various embodiments, chimeric protein, chimeric protein complex, vaccine composition, or adjuvant of the invention provides improved safety compared to, e.g., untargeted IL-1α or an unmodified, wildtype IL-1α or a modified IL-1α (e.g., pegylated IL-1α). In illustrative embodiments, administration of chimeric protein, chimeric protein complex, vaccine composition, or adjuvant is associated with minimal side effects such as those side effects associated with the use of the untargeted IL-1α or an unmodified, wildtype IL-1α or a modified IL-1α.

In some embodiments, chimeric protein, chimeric protein complex, vaccine composition, or adjuvant of the invention shows improved therapeutic activity compared to untargeted IL-1α or an unmodified, wildtype IL-1α, or a modified IL-1α (e.g., pegylated IL-1α). In some embodiments, chimeric protein, chimeric protein complex, vaccine composition, or adjuvant of the invention show improved pharmacokinetic profile (e.g., longer serum half-life and stability) compared to untargeted IL-1α or an unmodified, wildtype IL-1α or a modified IL-1α (e.g., pegylated IL-1α).

Without wishing to be bound by theory, it is believed that due to such advantageous safety and pharmacokinetic and therapeutic profiles, chimeric protein, chimeric protein complex, vaccine composition, or adjuvant may be used to treat patients at high dosages and/or for prolonged periods of time. For example, chimeric protein, chimeric protein complex, vaccine composition, or adjuvant may be used at high dosages for initial induction therapy against chronic hepatitis C infection. In another example, chimeric protein, chimeric protein complex, vaccine composition, or adjuvant may be used for long-term maintenance therapy to prevent disease relapse.

In various embodiments, the present compositions are used to treat or prevent one or more inflammatory diseases or conditions, such as inflammation, acute inflammation, chronic inflammation, respiratory disease, atherosclerosis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconioses.

In various embodiments, the present compositions are used to treat or prevent one or more autoimmune diseases or conditions, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases.

In various embodiments, the present compositions are used to treat, control or prevent cardiovascular disease, such as a disease or condition affecting the heart and vasculature, including but not limited to, coronary heart disease (CHD), cerebrovascular disease (CVD), aortic stenosis, peripheral vascular disease, atherosclerosis, arteriosclerosis, myocardial infarction (heart attack), cerebrovascular diseases (stroke), transient ischemic attacks (TIA), angina (stable and unstable), atrial fibrillation, arrhythmia, valvular disease, and/or congestive heart failure.

In various embodiments, the present compositions are used to treat or prevent one or more metabolic-related disorders. In various embodiments, the present invention is useful for the treatment, controlling or prevention of diabetes, including Type 1 and Type 2 diabetes and diabetes associated with obesity. The compositions and methods of the present invention are useful for the treatment or prevention of diabetes-related disorders, including without limitation diabetic nephropathy, hyperglycemia, impaired glucose tolerance, insulin resistance, obesity, lipid disorders, dyslipidemia, hyperlipidemia, hypertriglyceridemia, hypercholesterolemia, low HDL levels, high LDL levels, atherosclerosis and its sequelae, vascular restenosis, irritable bowel syndrome, inflamatory bowel disease, including Crohn's disease and ulcerative colitis, other inflammatory conditions, pancreatitis, abdominal obesity, neurodegenerative disease, retinopathy, neoplastic conditions, adipose cell tumors, adipose cell carcinomas, such as liposarcoma, prostate cancer and other cancers, including gastric, breast, bladder and colon cancers, angiogenesis, Alzheimer's disease, psoriasis, high blood pressure, Metabolic Syndrome (e.g. a person has three or more of the following disorders: abdominal obesity, hypertriglyceridemia, low HDL cholesterol, high blood pressure, and high fasting plasma glucose), ovarian hyperandrogenism (polycystic ovary syndrome), and other disorders where insulin resistance is a component, such as sleep apnea. The compositions and methods of the present invention are useful for the treatment, control, or prevention of obesity, including genetic or environmental, and obesity-related disorders. The obesity-related disorders herein are associated with, caused by, or result from obesity. Examples of obesity-related disorders include obesity, diabetes, overeating, binge eating, and bulimia, hypertension, elevated plasma insulin concentrations and insulin resistance, dyslipidemia, hyperlipidemia, endometrial, breast, prostate, kidney and colon cancer, osteoarthritis, obstructive sleep apnea, gallstones, heart disease, abnormal heart rhythms and arrythmias, myocardial infarction, congestive heart failure, coronary heart disease, sudden death, stroke, polycystic ovary disease, craniopharyngioma, Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are Metabolic Syndrome, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, lower back pain, gallbladder disease, hyperuricemia, gout, and kidney cancer, and increased anesthetic risk. The compositions and methods of the present invention are also useful to treat Alzheimer's disease.

In various embodiments, the present compositions are used to treat or prevent one or more respiratory diseases, such as idiopathic pulmonary fibrosis (IPF), asthma, chronic obstructive pulmonary disease (COPD), bronchiectasis, allergic rhinitis, sinusitis, pulmonary vasoconstriction, inflammation, allergies, impeded respiration, respiratory distress syndrome, cystic fibrosis, pulmonary hypertension, pulmonary vasoconstriction, emphysema, Hantavirus pulmonary syndrome (HPS), Loeffler's syndrome, Goodpasture's syndrome, Pleurisy, pneumonitis, pulmonary edema, pulmonary fibrosis, Sarcoidosis, complications associated with respiratory syncytial virus infection, and other respiratory diseases.

In some embodiments, the present invention is used to treat or prevent one or more neurodegenerative disease. Illustrative neurodegenerative diseases include, but are not limited to, Friedreich's Ataxia, multiple sclerosis (including without limitation, benign multiple sclerosis; relapsing-remitting multiple sclerosis (RRMS); secondary progressive multiple sclerosis (SPMS); progressive relapsing multiple sclerosis (PRMS); and primary progressive multiple sclerosis (PPMS)), Alzheimer's. disease (including, without limitation, Early-onset Alzheimer's, Late-onset Alzheimer's, and Familial Alzheimer's disease (FAD), Parkinson's disease and parkinsonism (including, without limitation, Idiopathic Parkinson's disease, Vascular parkinsonism, Drug-induced parkinsonism, Dementia with Lewy bodies, Inherited Parkinson's, Juvenile Parkinson's), Huntington's disease, Amyotrophic lateral sclerosis (ALS, including, without limitation, Sporadic ALS, Familial ALS, Western Pacific ALS, Juvenile ALS, Hiramaya Disease).

In various embodiments, chimeric protein, chimeric protein complex, vaccine composition, or adjuvant finds use in treating wounds, e.g., a non-healing wound, an ulcer, a burn, or frostbite, a chronic or acute wound, open or closed wound, internal or external wound (illustrative external wounds are penetrating and non-penetrating wound.

In various embodiments, chimeric protein, chimeric protein complex, vaccine composition, or adjuvant find use in treating ischemia, by way of non-limiting example, ischemia associated with acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, frostbite, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, or wounds to tissues or organs

In various embodiments, the present invention relates to the treatment of one or more of anemia, including anemia resulting from chronic kidney disease (e.g. from dialysis) and/or an anti-cancer agent (e.g. chemotherapy and/or HIV treatment (e.g. Zidovudine (INN) or azidothymidine (AZT)), inflammatory bowel disease (e.g. Crohn's disease and ulcer colitis), anemia linked to inflammatory conditions (e.g. arthritis, lupus, IBD), anemia linked to diabetes, schizophrenia, cerebral malaria, as aplastic anemia, and myelodysplasia from the treatment of cancer (e.g. chemotherapy and/or radiation), and various myelodysplastic syndrome diseases (e.g. sickle cell anemia, hemoglobin SC disease, hemoglobin C disease, alpha- and beta-thalassemias, neonatal anemia after premature birth, and comparable conditions).

In some embodiments, the present invention relates to the treatment of, or a patient having anemia, i.e. a condition in which the number of red blood cells and/or the amount of hemoglobin found in the red blood cells is below normal. In various embodiments, the anemia may be acute or chronic. For example, the present anemias include but are not limited to iron deficiency anemia, renal anemia, anemia of chronic diseases/inflammation, pernicious anemia such as macrocytic achylic anemia, juvenile pernicious anemia and congenital pernicious anemia, cancer-related anemia, anti-cancer-related anemia (e.g. chemotherapy-related anemia, radiotherapy-related anemia), pure red cell aplasia, refractory anemia with excess of blasts, aplastic anemia, X-lined siderobalstic anemia, hemolytic anemia, sickle cell anemia, anemia caused by impaired production of ESA, myelodysplasia syndromes, hypochromic anemia, microcytic anemia, sideroblastic anemia, autoimmune hemolytic anemia, Cooley's anemia, Mediterranean anemia, Diamond Blackfan anemia, Fanconi's anemia and drug-induced immune hemolytic anemia. Anemia may cause serious symptoms, including hypoxia, chronic fatigue, lack of concentration, pale skin, low blood pressure, dizziness and heart failure.

In some embodiments, the present invention relates to the treatment of anemia resulting from chronic renal failure. In some embodiments, the present invention relates to the treatment of anemia resulting from the use of one or more renal replacement therapies, inclusive of dialysis, hemodialysis, peritoneal dialysis, hemofiltration, hemodiafiltration, and renal transplantation.

In some embodiments, the present invention relates to the treatment of anemia in patients with chronic kidney disease who are not on dialysis. For instance, the present invention relates to patients in stage 1 CKD, or stage 2 CKD, or stage 3 CKD, or stage 4 CKD, or stage 5 CKD. In some embodiments, the present patient is stage 4 CKD or stage 5 CKD. In some embodiments, the present patient has undergone a kidney transplant. In some embodiments, the present invention relates to the treatment of anemia is a patient having an acute kidney injury (AKI).

In some embodiments, the anemia is induced by chemotherapy. For instance, the chemotherapy may be any myelosuppressive chemotherapy. In some embodiment, the chemotherapy is one or more of Revlimid, Thalomid, dexamethasone, Adriamycin and Doxil. In some embodiments, the chemotherapy is one or more platinum-based drugs including cisplatin (e.g. PLATINOL) and carboplatin (e.g. PARAPLATIN). In some embodiments, the chemotherapy is any one of the chemotherapeutic agents described herein. In some embodiments, the chemotherapy is any agent described in Groopman et al. J Natl Cancer Inst (1999) 91 (19): 1616-1634, the contents of which are hereby incorporated by reference in their entireties. In some embodiments, the present compositions and methods are used in the treatment of chemotherapy-related anemia in later stage cancer patients (e.g. a stage IV, or stage III, or stage II cancer). In some embodiments, the present compositions and methods are used in the treatment of chemotherapy-related anemia in cancer patients receiving dose-dense chemotherapy or other aggressive chemotherapy regimens.

In some embodiments, the present invention relates to the treatment of anemia in a patient having one or more blood-based cancers, such as leukemia, lymphoma, and multiple myeloma. Such cancers may affect the bone marrow directly. Further, the present invention relates to metastatic cancer that has spread to the bone or bone marrow. In some embodiments, the present invention relates to the treatment of anemia in a patient undergoing radiation therapy. Such radiation therapy may damage the bone marrow, lowering its ability to make red blood cells. In further embodiments, the present invention relates to the treatment of anemia in a patient having a reduction or deficiency of one or more of iron, vitamin B12, and folic acid. In further embodiments, the present invention relates to the treatment of anemia in a patient having excessive bleeding including without limitation, after surgery or from a tumor that is causing internal bleeding. In further embodiments, the present invention relates to the treatment of anemia in a patient having anemia of chronic disease.

In some embodiments, the present methods and compositions stimulate red blood cell production. In some embodiments, the present methods and compositions stimulate division and differentiation of committed erythroid progenitors in the bone marrow.

Certain embodiments of the present invention are particularly useful for treating chemotherapy-induced anemia in cancer patients. In some embodiments, the present methods and compositions allows for continued administration of chimeric protein, chimeric protein complex, vaccine composition, or adjuvant after a cancer patient's chemotherapy is finished. In some embodiments, the present methods and compositions allows for treatment of a cancer patient without dose reduction relative to a non-cancer patient. In some embodiments, the present methods and compositions allows for treatment of a cancer patient receiving chemotherapy and considered curable. In various embodiments, the cancer patient has one or more of a history of blood clots, recent surgery, prolonged periods of bed rest or limited activity, and treatment with a chemotherapeutic agent.

In various embodiments, the vaccine compositions of the invention provide improved safety compared to, e.g., untargeted IL-1α or an unmodified, wild type IL-1α or a modified IL-1α (e.g., pegylated IL-1α). In illustrative embodiments, administration of the vaccine composition is associated with minimal side effects such as those side effects associated with the use of the untargeted IL-1α or an unmodified, wild type IL-1α or a modified IL-1α (e.g., influenza-like symptoms, myalgia, leucopenia, thrombocytopenia, neutropenia, depression, and weight loss).

In some embodiments, the vaccine composition of the invention shows improved therapeutic activity compared to untargeted IL-1α or an unmodified, wild type IL-1α, or a modified IL-1α (e.g., pegylated IL-1α). In some embodiments, the vaccine composition of the invention shows improved pharmacokinetic profile (e.g., longer serum half-life and stability) compared to untargeted IL-1α or an unmodified, wild type IL-1α or a modified IL-1α (e.g., pegylated IL-1α).

### Kits

The invention also provides kits for the administration of any agent described herein (e.g. chimeric protein, chimeric protein complex, vaccine composition, or adjuvant with or without various additional therapeutic agents). The kit is an assemblage of materials or components, including at least one of the inventive pharmaceutical compositions described herein. Thus, in some embodiments, the kit contains at least one of the pharmaceutical compositions described herein.

The exact nature of the components configured in the kit depends on its intended purpose. In one embodiment, the kit is configured for treating human subjects.

Instructions for use may be included in the kit. Instructions for use typically include a tangible expression describing the technique to be employed in using the components of the kit to effect a desired outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label, which indicates the contents and/or purpose of the kit and/or its components.

### Definitions

As used herein, “a,” “an,” or “the” can mean one or more than one.

Unless specifically stated or obvious from context, as used herein, the term “or” is understood to be inclusive and covers both “or” and “and”.

Further, the term “about” when used in connection with a referenced numeric indication means the referenced numeric indication plus or minus up to 10% of that referenced numeric indication, e.g., within (plus or minus) 10%, 9%, 8%, 7%, 6%, 5%, 4%, 3%, 2%, 1%, 0.5%, 0.1%, 0.05%, or 0.01% of the stated value. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein. This invention is further illustrated by the following non-limiting examples.

### EXAMPLES

### Example 1: Construction, Production, and Purification of Human IL-1 Alpha Fusion Proteins

The mature and calpain-processed human IL-1 alpha protein was fused to the C-terminal end of a human Fc to create a heterodimeric, ‘knob-in-hole’ Fc protein containing a single IL-1 alpha copy. To this end, the human IgG1 Fc sequence containing the L234A_L235A_K322Q effector mutations and the ‘knob’ modifications S354C_T366W was fused (via the flexible 20*GGS-linker) to human IL-1 alpha protein (SEQ ID NO: 1 or 3 delta 1-6) in the pcDNA3.4 expression vector. Here, the human IL-1 alpha protein has a deletion of amino acids numbered 1-6 (“del1-6”) with respect to SEQ ID NOs: 1 or 3, which is the equivalent of “SEQ ID NO: 1 or 3 delta 1-6” or “IL1α delta 1-6” or “hIL 1α Δ1-6.”

The second partner of the Fc proteins was a human IgG1 Fc sequence containing the L234A_L235A_K322Q effector mutations and the ‘hole’ modifications Y349C_T366S_L368A_Y407V were also cloned in the pcDNA3.4 expression vector. Alternatively, the second partner was an CD8 VHH fused with a 5*GGS linker to the human IgG1 Fc sequence containing the L234A_L235A_K322Q effector mutations and the ‘hole’ modifications Y349C_T366S_L368A_Y407V.

To produce these ‘knob-in-hole’ Fc AFNs, both a knob and hole plasmid were transfected in ExpiCHO cells (ThermoFisher) according to the manufacturer's instructions. Seven days after transfection, recombinant proteins were purified using protein A High Performance MultiTrap (Cytiva, cat #28-9031-33) according to the manufacturer's instructions. The quantitation of the protein was performed using the Pierce 660 nm Protein Quantitation Assay kit (Thermo. Scientific, Cat #22660) on a SpectraMax multi well plate reader.

### Amino Acid Sequences Used in the Above Example:

Fc-knob_20GGS_IL1α delta 1-6 (underlined sequence
is a leader sequence)
(SEQ ID NO: 443)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVCLAGGPPSITDFQILENQA
Fc_hole (underlined sequence is a leader
sequence)
(SEQ ID NO: 444)
MGWSCIIFFLVATATGVHSDKTHTCPPCPAPEAAGGPSVFLFPPKPKDT
LMISRTPEVTCVVVDVSHEDPEVKFNWYVDGVEVHNAKTKPREEQYNST
YRVVSVLTVLHQDWLNGKEYKCQVSNKALPAPIEKTISKAKGQPREPQV
CTLPPSRDELTKNQVSLSCAVKGFYPSDIAVEWESNGQPENNYKTTPPV
LDSDGSFFLVSKLTVDKSRWQQGNVFSCSVMHEALHNHYTQKSLSLSPG
K
CD8 VHH_5GGS_Fc_hole (underlined sequence is
a leader sequence)
(SEQ ID NO: 445)
MGWSCIIFFLVATATGVHSDVQLQESGGGLVQPGGSLRLSCAASGSIFS
INVMGWYRQTPGKERELVAKITNFGITSYADSAQGRFTISRGNAKNTVY
LQMNSLKPEDTAVYYCNLDTTGWGPPPYQYWGQGTQVTVSSGGSGGSGG
SGGSGGSDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQ
DWLNGKEYKCQVSNKALPAPIEKTISKAKGQPREPQVCTLPPSRDELTK
NQVSLSCAVKGFYPSDIAVEWESNGQPENNYKTTPPVLDSDGSFFLVSK
LTVDKSRWQQGNVFSCSVMHEALHNHYTQKSLSLSPGK

### Example 2: Purified IL-1 Alpha Fusion Proteins Exhibit Specificity and Biologic Activity

To assess biological activity of the purified IL-1 alpha fusion proteins, the NF-KB reporter activity induced by wild type recombinant IL1α (Peprotech cat #200-01A) or the recombinant Fc_IL-1 alpha heterodimeric fusion proteins, was measured on HEK293T cells which lack CD8 expression. To evaluate the potential effect of the targeting in the CD8 VHH containing heterodimeric fusion protein, the HEK293T cells were additionally transiently transfected with a vector coding for human CD8 alpha expression. In brief, HEK293T cells (ATCC, cat #CRL-3216) were transiently transfected (Calcium Phosphate transfection Kit, Millipore, cat #233-140-S) with either a reporter vector only (pGL4.32 [luc2P/NF-κB-RE/Hygro] Vector, Promega, cat #E8491) or with the 1:4 ratio DNA mixture of the reporter vector and the hCD8+ construct (pMET7 derived). At 48 hours post-transfection the transiently transfected cells were stimulated with different concentrations of protein, diluted in DMEM, cell growth media, for 5 hours. Luciferase signaling was then assessed by addition of the read-out solution mixture (Pierce Luciferase Cell Lysis Buffer (2×), Thermo Fisher, cat #16189 and One-Glo Luciferase Assay System, Promega, cat #E6120 at 1:1 ratio). Signal was detected using SpectraMax plate reader within 10 minutes post addition of read-out solution mix to cells.

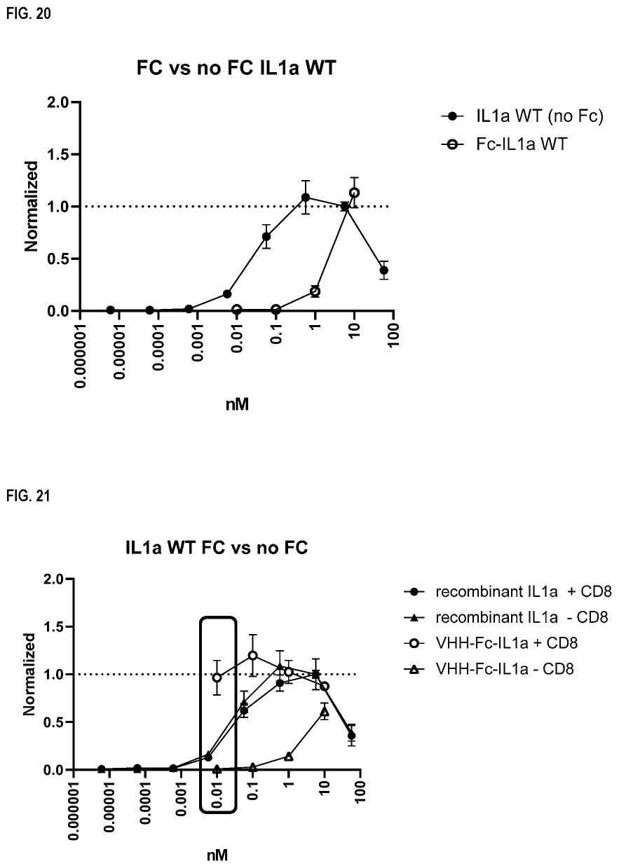

Surprisingly, FIG. 20 shows that the mutant IL-1 alpha A1-6 protein exhibited strongly reduced activity as a fusion protein, compared to wild type recombinant IL-1 alpha. In contrast, the CD8-targeted IL-1 alpha fusion protein surprisingly exhibited significantly more activity than wild type recombinant IL-1 alpha on CD8 positive cells (see FIG. 21). FIG. 20 and FIG. 21, taken together, show that the fusion protein has reduced IL-1α activity, which is restorable, and even increased, upon CD8 targeting. Indeed, the restored activity was shown to be higher than wild type recombinant IL-1 alpha activity.

### Example 3: IL-1 Alpha Fusion Proteins Devoid of Free Cysteine

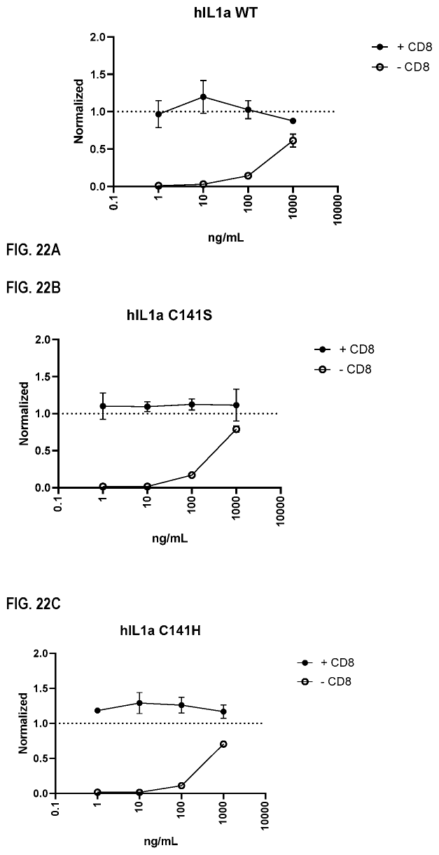

IL-1 alpha is known to contain a free cysteine and this free cysteine was mutated to the polar uncharged amino acid serine or to the positively charged polar amino acid histidine. Heterodimeric fusion proteins with the CD8 VHH-Fc construct were produced, purified, and tested for activity as described above. FIGS. 22A-C shows that both types of mutation of the free cysteine have no substantial impact on the biological activity of the fusion proteins. Specifically, FIGS. 22A-C show that IL1α Δ1-6 and C141 mutants fused to Fc exhibit reduced activity that is rescued by VHH cell targeting.

### Amino Acid Sequences Used in the Above Example:

Fc-knob_20GGS_IL1α delta 1-6_C141S (underlined
sequence is a leader sequence)
(SEQ ID NO: 446)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVSLAGGPPSITDFQILENQA
Fc-knob_20GGS_IL1α delta 1-6_C141H (underlined
sequence is a leader sequence)
(SEQ ID NO: 447)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVHLAGGPPSITDFQILENQA

### Example 4: IL-1 Alpha Fusion Proteins with Additional Mutations

Mutations (e.g., those thought, without wishing to be bound by theory, to be involved with receptor interaction) were introduced in the IL-1 alpha Fc-fusion protein and again produced and purified as heterodimeric fusion proteins with the CD8 VHH-Fc construct as described above. Several of these mutations were shown to restore IL1α activity in Fc fusion, as elucidated below.

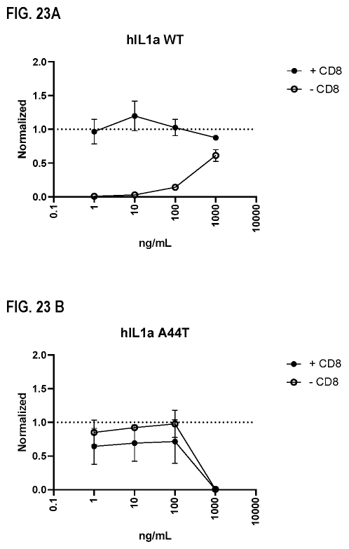

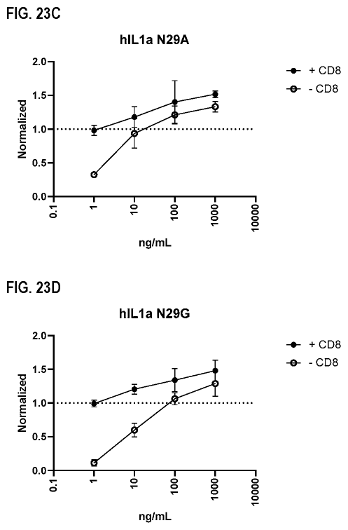

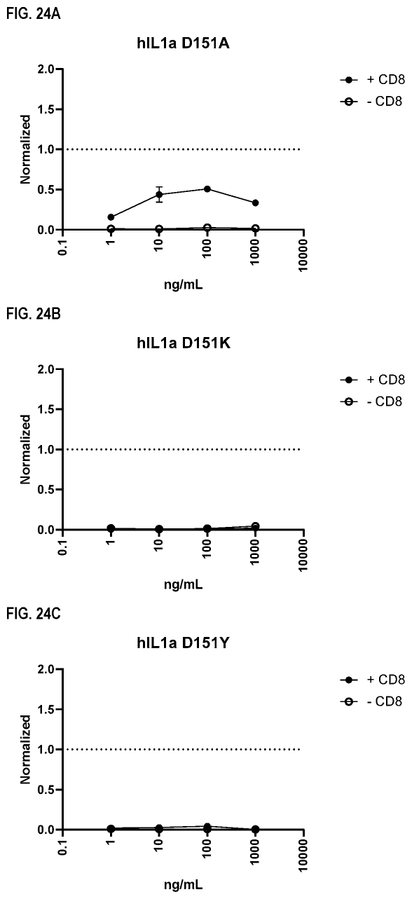

Testing for activity was performed as described above and resulted in surprising findings. Mutations N29A, N29G and A44T enhanced the activity of the fusion protein on the reporter cell line without CD8 expression (FIGS. 23A-D). Mutations at position D151 strongly reduced the activity and could not be restored by CD8 targeting in case of mutation to D151K or D151Y and were only partially restorable in case of mutation to D151A (FIGS. 24A-C). The remaining mutations are summarized in Table 7 below. This table shows the normalized signal (Table 7, columns C-F) for each test condition versus recombinant (unfused) IL1α at 100 ng/ml (i.e., a value of 1 means equally potent as 100 ng/ml of recombinant IL1α).

TABLE 7

| | | | | | | | H |
|---|---|---|---|---|---|---|---|
| | | | | | | G | ratio |
| | | | | | | ratio | “−CD8 cells” |
| A | | | | | | “+CD8 | mutant 100 |
| Anti-CD8- | | C | D | E | F | 1 ng/ml” | ng/ml/CD8- |
| VHH-Fc- | B | ng/ml | | | | “−CD8 | targeted Fc |
| fusions | Cells | 1000 | 100 | 10 | 1 | 1000 ng/ml” | IL1α Δ1-6 |
| hIL1α Δ1-6 | +CD8 | 0.88 | 1.03 | 1.20 | 0.97 | 1.58 | 1.00 |
| hIL1α Δ1-6 | −CD8 | 0.61 | 0.14 | 0.03 | 0.01 | | |
| hIL1α Δ1-6 | +CD8 | 1.11 | 1.12 | 1.09 | 1.10 | 1.39 | 1.21 |
| C141S | | | | | | | |
| hIL1α Δ1-6 | −CD8 | 0.79 | 0.17 | 0.02 | 0.02 | | |
| C141S | | | | | | | |
| hIL1α Δ1-6 | +CD8 | 1.17 | 1.26 | 1.29 | 1.18 | 1.68 | 0.78 |
| C141H | | | | | | | |
| hIL1α Δ1-6 | −CD8 | 0.70 | 0.11 | 0.02 | 0.02 | | |
| C141H | | | | | | | |
| hIL1α Δ1-6 | +CD8 | 1.13 | 1.22 | 1.34 | 1.11 | 1.83 | 0.59 |
| M15A | | | | | | | |
| hIL1α Δ1-6 | −CD8 | 0.60 | 0.08 | 0.02 | 0.01 | | |
| M15A | | | | | | | |
| hIL1α Δ1-6 | +CD8 | 1.31 | 1.20 | 0.95 | 0.72 | 11.09 | 0.09 |
| M15G | | | | | | | |
| hIL1α Δ1-6 | −CD8 | 0.06 | 0.01 | 0.02 | 0.02 | | |
| M15G | | | | | | | |
| hIL1α Δ1-6 | +CD8 | 1.32 | 1.40 | 1.32 | 1.07 | 2.98 | 0.24 |
| R16A | | | | | | | |
| hIL1α Δ1-6 | −CD8 | 0.36 | 0.03 | 0.01 | 0.02 | | |
| R16A | | | | | | | |
| hIL1α Δ1-6 | +CD8 | 1.19 | 1.07 | 0.73 | 0.25 | 12.68 | 0.08 |
| R16G | | | | | | | |
| hIL1α Δ1-6 | −CD8 | 0.02 | 0.01 | 0.02 | 0.01 | | |
| R16G | | | | | | | |
| hIL1α Δ1-6 | +CD8 | 1.10 | 1.24 | 1.26 | 1.04 | 4.13 | 0.26 |
| 118A | | | | | | | |
| hIL1α Δ1-6 | −CD8 | 0.25 | 0.04 | 0.02 | 0.01 | | |
| 118A | | | | | | | |
| hIL1α Δ1-6 | +CD8 | 1.20 | 1.18 | 0.90 | 0.40 | 7.29 | 0.09 |
| 118G | | | | | | | |
| hIL1α Δ1-6 | −CD8 | 0.05 | 0.01 | 0.02 | 0.01 | | |
| 118G | | | | | | | |
| hIL1α Δ1-6 | +CD8 | 1.08 | 1.18 | 1.26 | 1.32 | 1.76 | 0.97 |
| N25A | | | | | | | |
| hIL1α Δ1-6 | −CD8 | 0.75 | 0.14 | 0.01 | 0.02 | | |
| N25A | | | | | | | |
| hIL1α Δ1-6 | +CD8 | 1.30 | 1.00 | 0.80 | 0.72 | 0.87 | 1.03 |
| N25G | | | | | | | |
| hIL1α Δ1-6 | −CD8 | 0.83 | 0.15 | 0.02 | 0.0 | | |
| N25G | | | | | | | |
| hIL1α Δ1-6 | +CD8 | 1.56 | 1.22 | 1.10 | 1.01 | 0.91 | 4.08 |
| L28A | | | | | | | |
| hIL1α Δ1-6 | −CD8 | 1.11 | 0.58 | 0.07 | 0.01 | | |
| L28A | | | | | | | |
| hIL1α Δ1-6 | +CD8 | 1.56 | 1.38 | 1.24 | 0.98 | 0.96 | 1.39 |
| L28G | | | | | | | |
| hIL1α Δ1-6 | −CD8 | 1.02 | 0.20 | 0.03 | 0.01 | | |
| L28G | | | | | | | |
| hIL1α Δ1-6 | +CD8 | 1.46 | 1.35 | 1.09 | 0.83 | 1.30 | 0.52 |
| 133A | | | | | | | |
| hIL1α Δ1-6 | −CD8 | 0.64 | 0.07 | 0.01 | 0.02 | | |
| 133A | | | | | | | |
| hIL1α Δ1-6 | +CD8 | 1.74 | 1.45 | 1.10 | 0.88 | 1.51 | 0.26 |
| 133G | | | | | | | |
| hIL1α Δ1-6 | −CD8 | 0.58 | 0.04 | 0.01 | 0.02 | | |
| 133G | | | | | | | |
| hIL1α Δ1-6 | +CD8 | nd | 1.27 | 1.07 | 0.89 | nd | 0.76 |
| A44G | | | | | | | |
| hIL1α Δ1-6 | −CD8 | nd | 0.11 | 0.02 | 0.01 | | |
| A44G | | | | | | | |
| hIL1α Δ1-6 | +CD8 | 1.64 | 1.31 | 1.18 | 0.84 | 0.86 | 0.84 |
| A44S | | | | | | | |
| hIL1α Δ1-6 | −CD8 | 0.97 | 0.12 | 0.02 | 0.01 | | |
| A44S | | | | | | | |
| hIL1α Δ1-6 | +CD8 | 1.47 | 1.25 | 1.12 | 0.99 | 0.97 | 1.25 |
| A44H | | | | | | | |
| hIL1α Δ1-6 | −CD8 | 1.03 | 0.18 | 0.02 | 0.01 | | |
| A44H | | | | | | | |
| hIL1α Δ1-6 | +CD8 | 1.21 | 1.17 | 1.13 | 1.03 | 1.20 | 0.84 |
| A44N | | | | | | | |
| hIL1α Δ1-6 | −CD8 | 0.86 | 0.12 | 0.01 | 0.01 | | |
| A44N | | | | | | | |
| hIL1α Δ1-6 | +CD8 | 1.35 | 1.24 | 1.02 | 0.73 | 3.14 | 0.10 |
| H46A | | | | | | | |
| hIL1α Δ1-6 | −CD8 | 0.23 | 0.01 | 0.02 | 0.01 | | |
| H46A | | | | | | | |
| hIL1α Δ1-6 | +CD8 | 1.37 | 1.20 | 1.13 | 0.78 | 2.51 | 0.15 |
| H46G | | | | | | | |
| hIL1α Δ1-6 | −CD8 | 0.31 | 0.02 | 0.01 | 0.01 | | |
| H46G | | | | | | | |
| hIL1α Δ1-6 | +CD8 | 1.28 | 1.26 | 1.21 | 1.18 | 1.43 | 0.70 |
| A58G | | | | | | | |
| hIL1α Δ1-6 | −CD8 | 0.83 | 0.10 | 0.02 | 0.02 | | |
| A58G | | | | | | | |
| hIL1α Δ1-6 | +CD8 | 1.07 | 1.27 | 1.25 | 1.12 | 1.44 | 0.65 |
| A58S | | | | | | | |
| hIL1α Δ1-6 | −CD8 | 0.78 | 0.09 | 0.01 | 0.01 | | |
| A58S | | | | | | | |
| hIL1α Δ1-6 | +CD8 | 1.53 | 1.32 | 1.22 | 1.05 | 1.64 | 0.33 |
| A58T | | | | | | | |
| hIL1α Δ1-6 | −CD8 | 0.64 | 0.05 | 0.02 | 0.02 | | |
| A58T | | | | | | | |
| hIL1α Δ1-6 | +CD8 | 0.38 | 1.31 | 1.13 | 0.90 | 5.22 | 0.15 |
| A58N | | | | | | | |
| hIL1α Δ1-6 | −CD8 | 0.17 | 0.02 | 0.01 | 0.01 | | |
| A58N | | | | | | | |
| hIL1α Δ1-6 | +CD8 | 1.30 | 1.33 | 1.12 | 0.79 | 2.84 | 0.20 |
| A58H | | | | | | | |
| hIL1α Δ1-6 | −CD8 | 0.28 | 0.03 | 0.02 | 0.01 | | |
| A58H | | | | | | | |
| hIL1α Δ1-6 | +CD8 | 1.52 | 1.27 | 1.25 | 1.07 | 1.55 | 0.74 |
| A58F | | | | | | | |
| hIL1α Δ1-6 | −CD8 | 0.69 | 0.11 | 0.02 | 0.01 | | |
| A58F | | | | | | | |
| hIL1α Δ1-6 | +CD8 | 1.14 | 0.84 | 0.75 | 0.52 | 1.15 | 0.33 |
| K60A | | | | | | | |
| hIL1α Δ1-6 | −CD8 | 0.45 | 0.05 | 0.02 | 0.03 | | |
| K60A | | | | | | | |
| hIL1α Δ1-6 | +CD8 | 1.19 | 1.12 | 0.88 | 0.54 | 0.68 | 0.90 |
| K60G | | | | | | | |
| hIL1α Δ1-6 | −CD8 | 0.79 | 0.13 | 0.02 | 0.02 | | |
| K60G | | | | | | | |
| hIL1α Δ1-6 | +CD8 | 1.47 | 1.01 | 0.91 | 0.82 | 1.16 | 0.67 |
| D64A | | | | | | | |
| hIL1α Δ1-6 | −CD8 | 0.71 | 0.09 | 0.02 | 0.02 | | |
| D64A | | | | | | | |
| hIL1α Δ1-6 | +CD8 | 1.67 | 1.27 | 1.02 | 0.79 | 0.82 | 0.85 |
| K67A | | | | | | | |
| hIL1α Δ1-6 | −CD8 | 0.97 | 0.12 | 0.01 | 0.01 | | |
| K67A | | | | | | | |
| hIL1α Δ1-6 | +CD8 | 1.66 | 1.21 | 1.06 | 0.86 | 0.94 | 1.61 |
| 168A | | | | | | | |
| hIL1α Δ1-6 | −CD8 | 0.91 | 0.23 | 0.03 | 0.01 | | |
| 168A | | | | | | | |
| hIL1α Δ1-6 | +CD8 | 1.47 | 1.24 | 0.92 | 0.60 | 3.34 | 0.07 |
| 168G | | | | | | | |
| hIL1α Δ1-6 | −CD8 | 0.18 | 0.01 | 0.01 | 0.01 | | |
| 168G | | | | | | | |
| hIL1α Δ1-6 | +CD8 | 1.40 | 1.25 | 1.03 | 0.97 | 1.13 | 0.76 |
| Q136A | | | | | | | |
| hIL1α Δ1-6 | −CD8 | 0.85 | 0.11 | 0.02 | 0.01 | | |
| Q136A | | | | | | | |
| hIL1α Δ1-6 | +CD8 | 0.50 | 1.26 | 1.10 | 0.91 | 1.48 | 1.25 |
| Q153A | | | | | | | |
| hIL1α Δ1-6 | −CD8 | 0.61 | 0.18 | 0.02 | 0.01 | | |
| Q153A | | | | | | | |
| hIL1α Δ1-6 | +CD8 | 1.64 | 1.20 | 1.08 | 1.06 | 1.23 | 0.89 |
| Q153G | | | | | | | |
| hIL1α Δ1-6 | −CD8 | 0.86 | 0.13 | 0.01 | 0.01 | | |
| Q153G | | | | | | | |
| hIL1α Δ1-6 | +CD8 | 1.02 | 1.10 | 1.07 | 0.81 | 0.94 | 2.10 |
| D64G | | | | | | | |
| hIL1α Δ1-6 | −CD8 | 0.85 | 0.30 | 0.02 | 0.01 | | |
| D64G | | | | | | | |
| hIL1α Δ1-6 | +CD8 | 0.90 | 1.05 | 1.05 | 0.89 | 1.08 | 1.79 |
| D65A | | | | | | | |
| hIL1α Δ1-6 | −CD8 | 0.83 | 0.25 | 0.03 | 0.02 | | |
| D65A | | | | | | | |
| hIL1α Δ1-6 | +CD8 | 1.04 | 1.11 | 1.01 | 0.78 | 2.16 | 0.46 |
| D64G D65A | | | | | | | |
| hIL1α Δ1-6 | −CD8 | 0.36 | 0.07 | 0.02 | 0.02 | | |
| D64G D65A | | | | | | | |
| hIL1α Δ1-6 | +CD8 | nd | 1.14 | 1.00 | 0.88 | nd | 0.78 |
| W113F | | | | | | | |
| hIL1α Δ1-6 | −CD8 | nd | 0.11 | 0.02 | 0.01 | | |
| W113F | | | | | | | |
| hIL1α Δ1-6 | +CD8 | nd | 1.17 | 1.04 | 1.01 | nd | 2.80 |
| K100A | | | | | | | |
| hIL1α Δ1-6 | −CD8 | nd | 0.40 | 0.04 | 0.02 | | |
| K100A | | | | | | | |

nd = no or very low signal at 1000 ng/ml not allowing interpretation

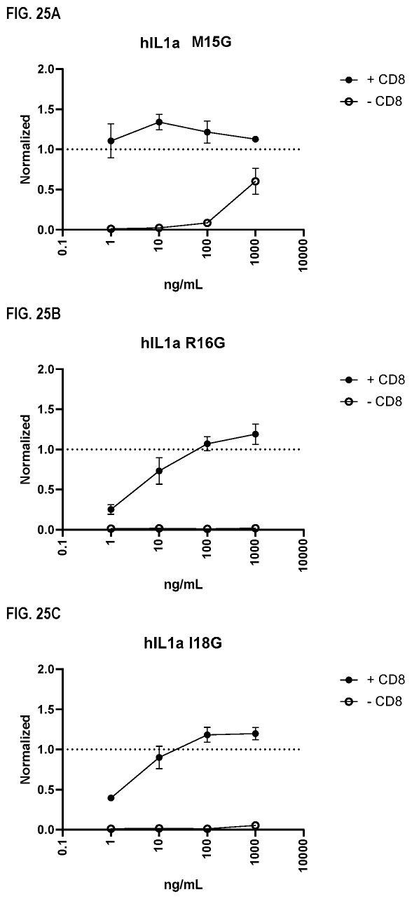

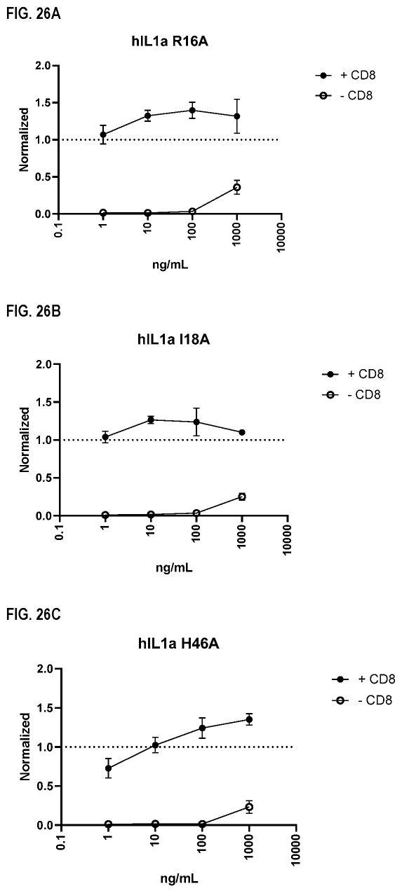

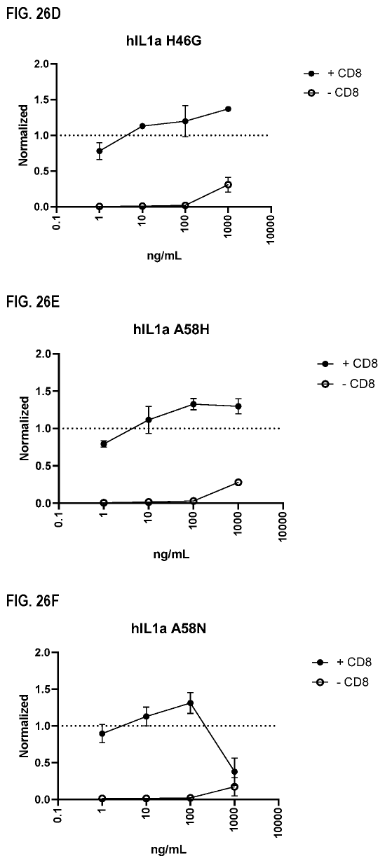

Surprisingly, several mutations could be identified with an increased (versus CD8-targeted IL-1 alpha del1-6, fused to Fc) selectivity window of activity on targeted cells (i.e., a higher ratio of signal at 1 ng/ml on CD8 positive cells over the signal at 1000 ng/ml on CD8 negative cells compared to the ratio for the CD8-targeted Fc IL-1 alpha Δ1-6 fusion) as shown in column G of Table 1. These findings were corroborated by a reduced signal on CD8 negative cells at, for example, 100 ng/ml versus CD8-targeted IL-1 alpha Δ1-6 fused to Fc (see column H of Table 1). These mutations include, for example, R16G, M15G, 118G, A58N, 118A, 168G, H46A, R16A, A58H, H46G and the double mutant D64G/D65A. The responses for these specific mutations are shown in FIGS. 25A-C and FIGS. 26A-H.

### Amino Acid Sequences Used in the Above Example:

Fc-knob_20GGS_IL1α delta 1-6_C141S (underlined sequence is a leader sequence)
(SEQ ID NO: 448)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVSLAGGPPSITDFQILENQA
Fc-knob_20GGS_IL1α delta 1-6_M15A (underlined sequence is a leader sequence)
(SEQ ID NO: 449)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFARIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_M15G (underlined sequence is a leader sequence)
(SEQ ID NO: 450)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFGRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_R16A (underlined sequence is a leader sequence)
(SEQ ID NO: 451)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMAIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_R16G (underlined sequence is a leader sequence)
(SEQ ID NO: 452)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMGIIKYEFILNDALNQSIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_I18A (underlined sequence is a leader sequence)
(SEQ ID NO: 453)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIAKYEFILNDALNQSIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_I18G (underlined sequence is a leader sequence)
(SEQ ID NO: 454)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIGKYEFILNDALNQSIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_N25A (underlined sequence is a leader sequence)
(SEQ ID NO: 455)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILADALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_N25G (underlined sequence is a leader sequence)
(SEQ ID NO: 456)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILGDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_L28A (underlined sequence is a leader sequence)
(SEQ ID NO: 457)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDAANQSIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_L28G (underlined sequence is a leader sequence)
(SEQ ID NO: 458)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDAGNQSIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_N29A (underlined sequence is a leader sequence)
(SEQ ID NO: 459)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALAQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_N92G (underlined sequence is a leader sequence)
(SEQ ID NO: 460)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALGQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_I33A (underlined sequence is a leader sequence)
(SEQ ID NO: 461)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIARANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_I33G (underlined sequence is a leader sequence)
(SEQ ID NO: 462)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIGRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_A44G (underlined sequence is a leader sequence)
(SEQ ID NO: 463)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAGLHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A44S (underlined sequence is a leadersequence)
(SEQ ID NO: 464)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAASLHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A44H (underlined sequence is a leader sequence)
(SEQ ID NO: 465)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAHLHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A44T (underlined sequence is a leader sequence)
(SEQ ID NO: 466)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAATLHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A44N (underlined sequence is a leader sequence)
(SEQ ID NO: 467)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAANLHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_H46A (underlined sequence is a leader sequence)
(SEQ ID NO: 468)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALANLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_H46G (underlined sequence is a leader sequence)
(SEQ ID NO: 469)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALGNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A58G (underlined sequence is a leader sequence)
(SEQ ID NO: 470)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGGYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A58S (underlined sequence is a leader sequence)
(SEQ ID NO: 471)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGSYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A58T (underlined sequence is a leader sequence)
(SEQ ID NO: 472)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGTYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A58N (underlined sequence is a leader sequence)
(SEQ ID NO: 473)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGNYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A58H (underlined sequence is a leader sequence)
(SEQ ID NO: 474)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGHYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_A58F (underlined sequence is a leader sequence)
(SEQ ID NO: 475)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGFYKSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_K60A (underlined sequence is a leader sequence)
(SEQ ID NO: 476)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYASSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_K60G (underlined sequence is a leader sequence)
(SEQ ID NO: 477)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYGSSKDDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_D64A (underlined sequence is a leader sequence)
(SEQ ID NO: 478)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKADAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_K67A (underlined sequence is a leader sequence)
(SEQ ID NO: 479)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAAITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_I68A (underlined sequence is a leader sequence)
(SEQ ID NO: 480)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKATVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_I68G (underlined sequence is a leader sequence)
(SEQ ID NO: 481)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKGTVILRISKTQLYVTAQDEDQPVLLKEMPEIPKTITGSETNLLF
FWETHGTKNYFTSVAHPNLFIATKQDYWVCLAGGPPSITDFQILENQA
Fc-knob_20GGS_IL1α delta 1-6_Q136A (underlined sequence is a leader sequence)
(SEQ ID NO: 482)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKADYWVCLAGGPPSITDFQILENQA
Fc-knob_20GGS_IL1α delta 1-6_Q153A (underlined sequence is a leader sequence)
(SEQ ID NO: 483)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFAILENQA
Fc-knob_20GGS_IL1α delta 1-6_Q153G (underlined sequence is a leader sequence)
(SEQ ID NO: 484)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITDFGILENQA
Fc-knob_20GGS_IL1α delta 1-6_D151A (underlined sequence is a leader sequence)
(SEQ ID NO: 485)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITAFGILENQA
Fc-knob_20GGS_IL1α delta 1-6_D151K (underlined sequence is a leader sequence)
(SEQ ID NO: 486)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITKFGILENQA
Fc-knob_20GGS_IL1α delta 1-6_D151Y (underlined sequence is a leader sequence)
(SEQ ID NO: 487)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVCLAGGPPSITYFGILENQA
Fc-knob_20GGS_IL1α delta 1-6_D64G (underlined sequence is a leader sequence)
(SEQ ID NO: 488)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKGDAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_D65A (underlined sequence is a leader sequence)
(SEQ ID NO: 489)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDAAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_D64AD65A (underlined sequence is a leader sequence)
(SEQ ID NO: 490)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKAAAKITVILRISKTQLYVTAQDEDQPVLLKEMPEIPKTITGSETNLLFF
WETHGTKNYFTSVAHPNLFIATKQDYWVCLAGGPPSITDFQILENQA
Fc-knob_20GGS_IL1α delta 1-6_W113F (underlined sequence is a leader sequence)
(SEQ ID NO: 491)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPKTITGSETNLLFF
FETHGTKNYFTSVAHPNLFIATKQDYWVCLAGGPPSITDFQILENQA
Fc-knob_20GGS_IL1α delta delta 1-6_K100A (underlined sequence is a leader sequence)
(SEQ ID NO: 492)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPATITGSETNLLFF
WETHGTKNYFTSVAHPNLFIATKQDYWVCLAGGPPSITDFQILENQA
Fc-knob_20GGS_IL1α delta 1-6_K100D (underlined sequence is a leader sequence)
(SEQ ID NO: 493)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQPVLLKEMPEIPDTITGSETNLLFF
WETHGTKNYFTSVAHPNLFIATKQDYWVCLAGGPPSITDFQILENQA

### Example 5: Dose-Response Biological Activity of CD8-Targeted Fc-IL-1α Del1-6 with C141 Mutation

CD8-targeted Fc-IL-1α del1-6 and CD8-targeted Fc-IL-1α del1-6 constructs having either a C141A or C141S mutation were produced as described in Example 1. The biological activity was assessed in the assay as described in Example 2 but using an extended dilution series allowing for the calculation of EC50 values. In addition, untargeted molecules were tested.
CD8 VHH_5GGS_Fc_hole+Fc-knob_20GGS_IL1a delta 1-6
CD8 VHH_5GGS_Fc_hole Fc-knob_20GGS_IL1a delta 1-6_C141S
CD8 VHH_5GGS_Fc_hole Fc-knob_20GGS_IL1a delta 1-6_C141A
Fc_hole+Fc-knob_20GGS_IL1a delta 1-6
Fc_hole Fc-knob_20GGS_IL1a delta 1-6_C141S
Fc_hole Fc-knob_20GGS_IL1a delta 1-6_C141A

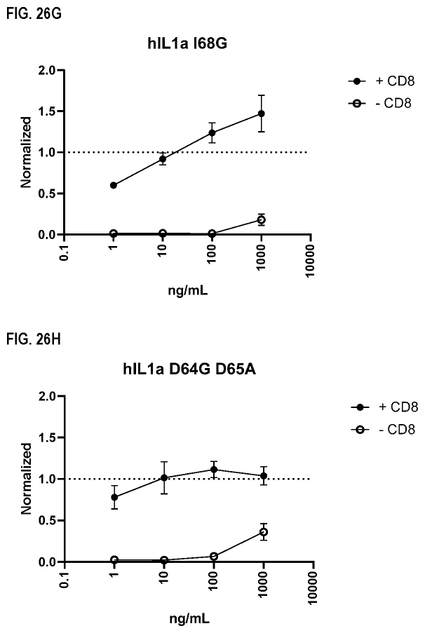

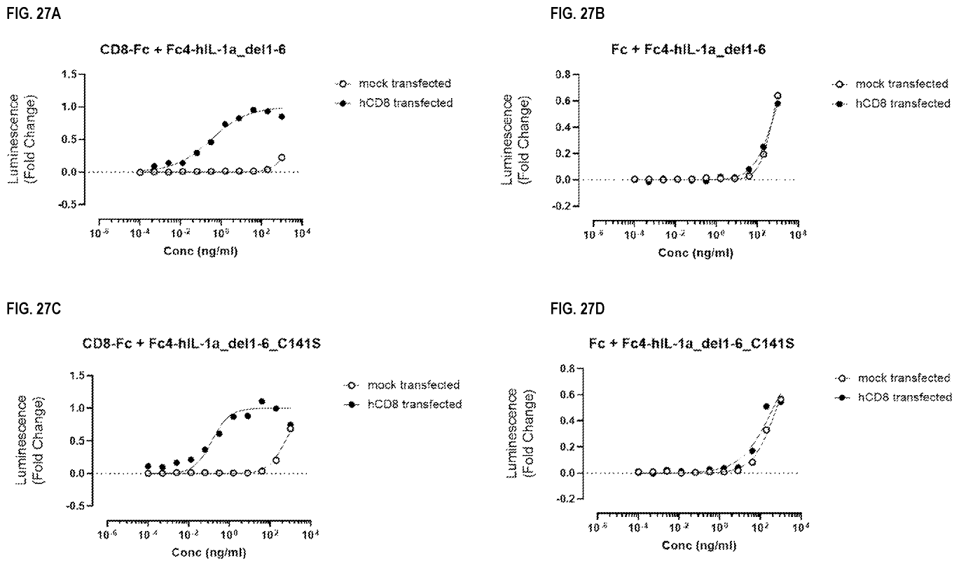

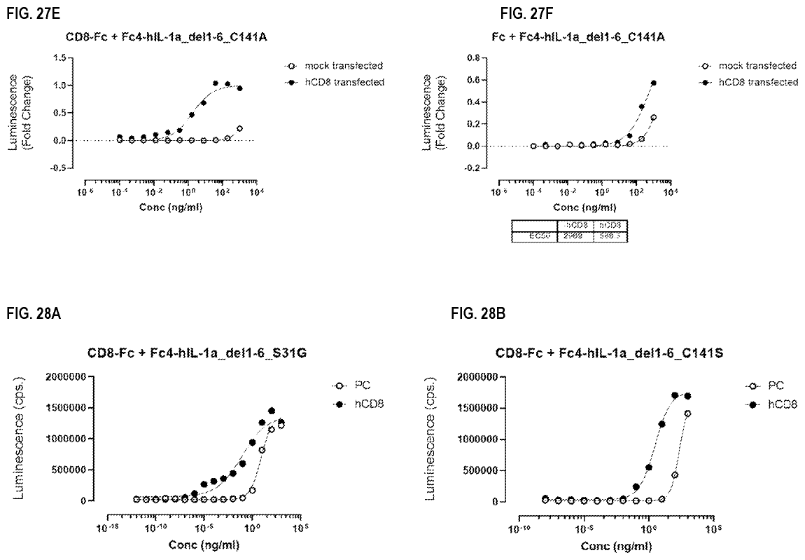

As shown in FIG. 27 and Table 8, the surprising targeting effect of an Fc-fused IL-1α was confirmed showing an about 3-log 10 targeting ratio not only for the IL-1α having only the del1-6 mutation but also of the IL-1α having the additional C141A or C141S mutation. This targeting effect is not only observed when comparing mock transfected cells with CD8 transfected cells but also when comparing untargeted with targeted constructs on CD8 transfected cells.

TABLE 8
biological activity, on transiently transfected HEK293T cells, of CD8 targeted
or untargeted human IL-1a Fc-construct with a del1-6 mutation with or without
C141 mutation. EC50 values in italic are based on extrapolation.

| | EC50 CD8 | EC50 mock | |
|---|---|---|---|
| | transfected | transfected | |
| Construct | (ng/mL) | (ng/mL) | Ratio |
| CD8-Fc + Fc-hIL-1a_del1-6 | 0.33 | 2965 | 8985 |
| Fc + Fc-hIL-1a_del1-6 | 694.7 | 628.6 | 0.9 |
| CD8-Fc + Fc-hIL-1a_del1-6_C141S | 0.14 | 569.2 | 4065 |
| Fc + Fc-hIL-1a_del1-6_C141S | 436.3 | 644.4 | 1.5 |
| CD8-Fc + Fc-hIL-1a_del1-6_C141A | 1.83 | 3082 | 1684 |
| Fc + Fc-hIL-1a_del1-6_C141A | 586.3 | 2969 | 5.1 |

### Amino Acid Sequences Used in the Above Example are as Follows:

CD8 VHH_5GGS_Fc_hole (underlined sequence is a
leader sequence)
(SEQ ID NO: 445)
Fc-knob_20GGS_IL1α delta 1-6 (underlined sequence
is a leader sequence)
(SEQ ID NO: 443)
Fc_hole (underlined sequence is a leader
sequence)
(SEQ ID NO: 444)
Fc-knob_20GGS_IL1α delta 1-6_C141S (underlined
sequence is a leader sequence)
(SEQ ID NO: 446)
Fc-knob_20GGS_IL1α delta 1-6_C141A (underlined
sequence is a leader sequence)
(SEQ ID NO: 494)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALNQSIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVALAGGPPSITDFQILENQA

### Example 6: Dose-Response Biological Activity of CD8-Targeted Fc-IL-1α Del1-6 with N29 or S31 Mutation

CD8-targeted Fc-IL-1α del1-6, optionally with C141 mutations, and CD8-targeted Fc-IL-1α del1-6 mutants having additional mutations at N29 or S31 of IL-1α were produced as described in Example 1.
CD8 VHH_5GGS_Fc_hole+Fc-knob_20GGS_IL1α delta 1-6
CD8 VHH_5GGS_Fc_hole+Fc-knob_20GGS_IL1α delta 1-6_C141S
CD8 VHH_5GGS_Fc_hole+Fc-knob_20GGS_IL1α delta 1-6_C141A
CD8 VHH_5GGS_Fc_hole+Fc-knob_20GGS_IL1α delta 1-6_N29A
CD8 VHH_5GGS_Fc_hole+Fc-knob_20GGS_IL1α delta 1-6_N29G
CD8 VHH_5GGS_Fc_hole+Fc-knob_20GGS_IL1α delta 1-6_N29D
CD8 VHH_5GGS_Fc_hole+Fc-knob_20GGS_IL1α delta 1-6_S31A
CD8 VHH_5GGS_Fc_hole+Fc-knob_20GGS_IL1α delta 1-6_S31G

Biological activity was assessed on the HekBlue-IL-1β cell line (Invivogen; hkb-il1b) with or without stable expression of human CD8. In brief, parental HekBlue-IL-1β cells or hCD8⁺ HekBlue-IL-1β cells were seeded in 96-well plates at 50.000 cells/well and stimulated for 20 hours with a serial dilution of recombinant IL-1α (produced in E. coli; Biolegend; cat. #570004) or the different CD8 V_{H}H-Fc-IL-1α variants. SEAP activity was measured using the PhosphaLight SEAP reporter gene assay system (ThermoFisher; T1017) in the assay described in Example 2 again using an extended dilution series allowing the calculation of EC50 values.

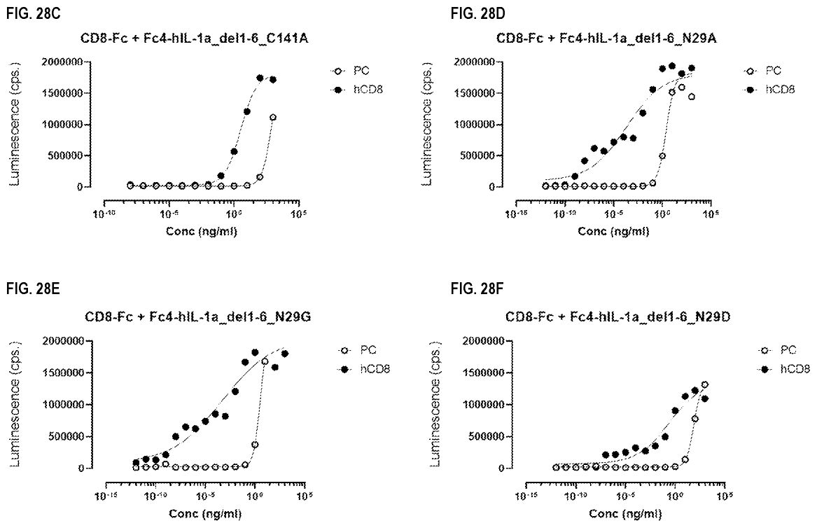

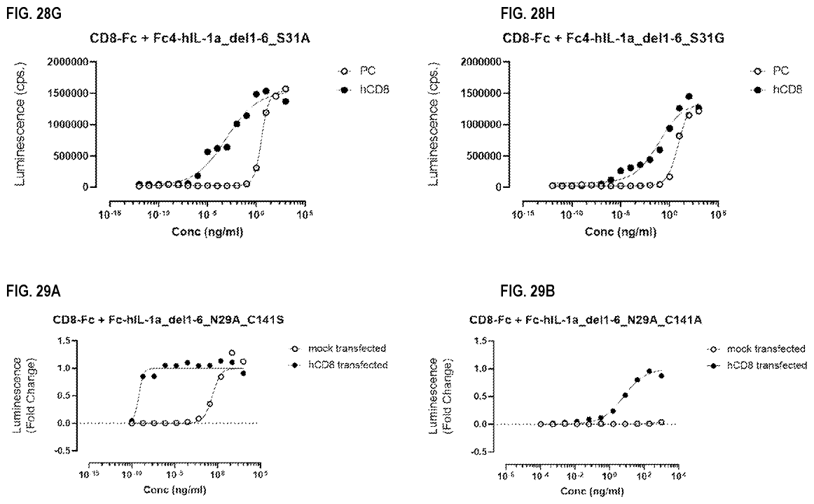

This biological assay results in different EC50 numbers compared to the previous Example (Example 5) for equivalent constructs. This reflects the use of HekBlue cell lines vs. HEK293T cell line, as well stable expression of CD8 and use of a different reporter system. Despite their intrinsic difference in sensitivity, a clear and substantial higher potency (targeting effect, indicated by ratio in Table 9) was observed again in this Example for the CD8-targeted Fc-IL-1α del1-6 constructs, including construct with mutations in C141. Remarkably, the biological activity of the CD8-targeted constructs with N29 and S31 mutations were substantially increased towards the parental cell line (not expressing CD8). Also very surprisingly, the targeting ratio was further enhanced for some of the mutant constructs, as shown in FIG. 28 and Table 9.

TABLE 9
biological activity, on stably transfected HekBlue-IL-1β
cells, of CD8 targeted human IL-1a Fc-constructs with a del1-6
mutation with additional mutations at C141, N29 or S31.

| | EC50 CD8 cell line | EC50 parental cell | |
|---|---|---|---|
| Construct | (ng/mL) | line (ng/mL) | Ratio |
| CD8-Fc + Fc-hIL-1a_del1-6 | 2.2 | 349.2 | 159 |
| CD8-Fc + Fc-hIL-1a_del1-6_C141S | 2.9 | 289.6 | 100 |
| CD8-Fc + Fc-hIL-1a_del1-6_C141A | 3.3 | 684.2 | 196 |
| CD8-Fc + Fc-hIL-1a_del1-6_N29A | 0.0002 | 2.5 | 12500 |
| CD8-Fc + Fc-hIL-1a_del1-6_N29G | 0.0005 | 3.2 | 6038 |
| CD8-Fc + Fc-hIL-1a_del1-6_N29D | 0.27 | 79.2 | 293 |
| CD8-Fc + Fc-hIL-1a_del1-6_S31A | 0.0008 | 3.5 | 4321 |
| CD8-Fc + Fc-hIL-1a_del1-6_S31G | 0.086 | 8.3 | 97 |

### Amino Acid Sequences Used in the Above Example are as Follows:

CD8 VHH_5GGS_Fc_hole (underlined sequence is a
leader sequence)
(SEQ ID NO: 445)
Fc-knob_20GGS_IL1α delta 1-6_N29A (underlined
sequence is a leader sequence)
(SEQ ID NO: 459)
Fc-knob_20GGS_IL1α delta 1-6_N92G (underlined
sequence is a leader sequence)
(SEQ ID NO: 460)
Fc-knob_20GGS_IL1α delta 1-6_N29D (underlined
sequence is a leader sequence)
(SEQ ID NO: 495)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALDQSIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVCLAGGPPSITDFQILENQA
Fc-knob_20GGS_IL1α delta 1-6_S31A (underlined
sequence is a leader sequence)
(SEQ ID NO: 496)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALNQAIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVCLAGGPPSITDFQILENQA
Fc-knob_20GGS_IL1α delta 1-6_S31G (underlined
sequence is a leader sequence)
(SEQ ID NO: 497)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALNQGIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVCLAGGPPSITDFQILENQA

### Example 7: Dose-Response Biological Activity of CD8-Targeted Fc-IL-1α Del1-6 with N29 or S31 Mutations Combined with C141 Mutations

CD8-targeted Fc-IL-1α del1-6 proteins with an N29 or S31 mutation combined with a C141 mutation were produced as described in Example 1 and biological activity assessed as described in Example 2 but using an extended dilution series allowing the calculation of EC₅₀ values.
CD8 VHH_5GGS_Fc_hole+Fc-knob_20GGS_IL1a delta 1-6
CD8 VHH_5GGS_Fc_hole+Fc-knob_20GGS_IL1a delta 1-6_N29A_C141S
CD8 VHH_5GGS_Fc_hole+Fc-knob_20GGS_IL1a delta 1-6_N29A_C141A
CD8 VHH_5GGS_Fc_hole+Fc-knob_20GGS_IL1a delta 1-6_N29G_C141S
CD8 VHH_5GGS_Fc_hole+Fc-knob_20GGS_IL1a delta 1-6_N29G_C141A
CD8 VHH_5GGS_Fc_hole+Fc-knob_20GGS_IL1a delta 1-6_N29D_C141S
CD8 VHH_5GGS_Fc_hole+Fc-knob_20GGS_IL1a delta 1-6_N29D_C141A
CD8 VHH_5GGS_Fc_hole+Fc-knob_20GGS_IL1a delta 1-6_S31A_C141S
CD8 VHH_5GGS_Fc_hole+Fc-knob_20GGS_IL1a delta 1-6_S31A_C141A
CD8 VHH_5GGS_Fc_hole+Fc-knob_20GGS_IL1a delta 1-6_S31G_C141S
CD8 VHH_5GGS_Fc_hole+Fc-knob_20GGS_IL1a delta 1-6_S31G_C141A

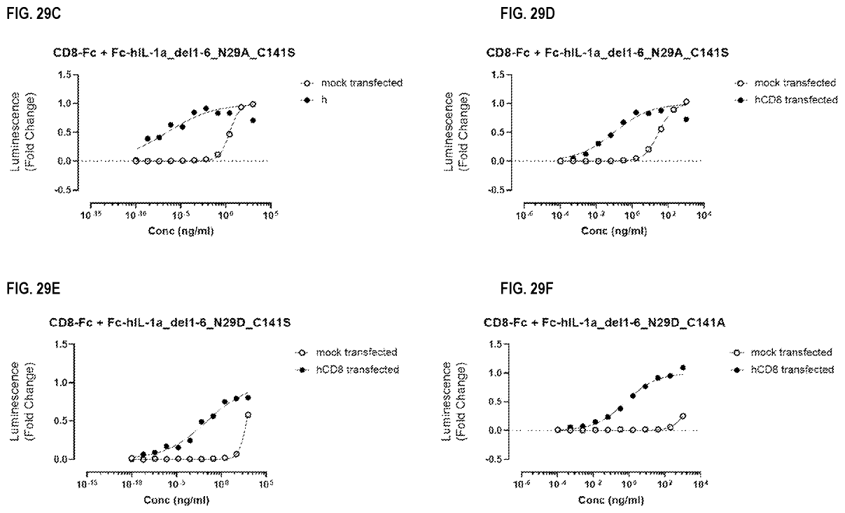

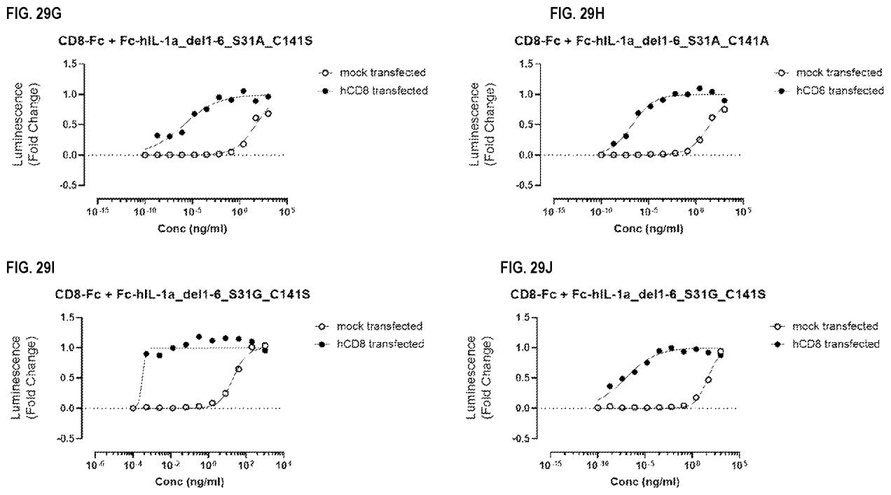

As shown in FIG. 29 and Table 10, most constructs exhibited increased biological activity also when N29 or S31 mutations were combined with a C141 mutation (biological activity on mock and/or CD8 transfected cells). While all constructs still exhibited a two to three log 10 targeting window, surprisingly, most constructs exhibited both enhanced activity and an enlarged targeting window, when compared to the Fc IL-1α delta 1-6.

TABLE 10
biological activity, on transiently transfected HEK293T cells,
of CD8 targeted human IL-1a Fc-construct with a del1-6 mutation
and N29 or S31 mutation with or without C141 mutation.

| | EC50 (ng/ml) | EC50 (ng/ml) | |
|---|---|---|---|
| | mock | CD8 | |
| Construct | transfected | transfected | Ratio |
| CD8-Fc + Fc-hIL-1a_del1-6 | 2965 | 0.33 | 8985 |
| CD8-Fc + Fc-hIL-1a_del1-6_N29A_C141S | 0.24 | 6E−10 | 4E+08 |
| CD8-Fc + Fc-hIL-1a_del1-6_N29A_C141A | >>1000 | 6.64 | >>1000* |
| CD8-Fc + Fc-hIL-1a_del1-6_N29G_C141S | 2.61 | 2.8E−07 | 9.3E+06 |
| CD8-Fc + Fc-hIL-1a_del1-6_N29G_C141A | 29.7 | 0.097 | 306 |
| CD8-Fc + Fc-hIL-1a_del1-6_N29D_C141S | 715 | 0.027 | 26480 |
| CD8-Fc + Fc-hIL-1a_del1-6_N29D_C141A | 3125 | 0.65 | 4807 |
| CD8-Fc + Fc-hIL-1a_del1-6_S31A_C141S | 50.5 | 1.3E−06 | 3.8E+07 |
| CD8-Fc + Fc-hIL-1a_del1-6_S31A_C141A | 29.5 | 1.9E−07 | 1.5E+08 |
| CD8-Fc + Fc-hIL-1a_del1-6_S31G_C141S | 20.6 | 0.0003 | 68666 |
| CD8-Fc + Fc-hIL-1a_del1-6_S31G_C141A | 44.6 | 8.7E−08 | 5.1E+08 |

*For the construct with N29A and C141A mutation, the targeting window could not be determined by comparing EC50s. Hence, the targeting window was estimated by evaluating the lowest concentration resulting in a detectable signal on CD8 transfected cells, i.e. less than 1 ng/m, and the highest concentration having no signal on mock transfected cell, i.e. 1000 ng/mL.

### Amino Acid Sequences Used in the Above Example are as Follows:

CD8 VHH_5GGS_Fc_hole (underlined sequence is a leader sequence)
(SEQ ID NO: 445)
Fc-knob_20GGS_IL1α delta 1-6_N29A_C141A (underlined sequence is a leader sequence)
(SEQ ID NO: 498)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALAQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVALAGGPPSITDFQILENQA
Fc-knob_20GGS_IL1α delta 1-6_N29G_C141A (underlined sequence is a leader sequence)
(SEQ ID NO: 499)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALGQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVALAGGPPSITDFQILENQA
Fc-knob_20GGS_IL1α delta 1-6_N29D_C141A (underlined sequence is a leader sequence)
(SEQ ID NO: 504)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALDQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVALAGGPPSITDFQILENQA
Fc-knob_20GGS_IL1α delta 1-6_S31A_C141A (underlined sequence is a leader sequence)
(SEQ ID NO: 505)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQAIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVALAGGPPSITDFQILENQA
Fc-knob_20GGS_IL1α delta 1-6_S31G_C141A (underlined sequence is a leader sequence)
(SEQ ID NO: 506)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQGIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVALAGGPPSITDFQILENQA
Fc-knob_20GGS_IL1α delta 1-6_N29A_C141S (underlined sequence is a leader sequence)
(SEQ ID NO: 507)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALAQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVSLAGGPPSITDFQILENQA
Fc-knob_20GGS_IL1α delta 1-6_N29G_C141S (underlined sequence is a leader sequence)
(SEQ ID NO: 508)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALGQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVSLAGGPPSITDFQILENQA
Fc-knob_20GGS_IL1α delta 1-6_N29D_C141S (underlined sequence is a leader sequence)
(SEQ ID NO: 509)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALDQSIIRANDQYL
TAAALHNLDEAVKFDMGAYKSSKDDAKITVILRISKTQLYVTAQDEDQPVLLKEMPEIPKTITGSETNLLFF
WETHGTKNYFTSVAHPNLFIATKQDYWVSLAGGPPSITDFQILENQA
Fc-knob_20GGS_IL1α delta 1-6_S31A_C141S (underlined sequence is a leader sequence)
(SEQ ID NO: 510)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQAIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVSLAGGPPSITDFQILENQA
Fc-knob_20GGS_IL1α delta 1-6_S31G_C141S (underlined sequence is a leader sequence)
(SEQ ID NO: 511)
MDMRVPAQLLGLLLLWLRGARCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCQVSNKALPAPIEKTI
SKAKGQPREPQVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSLSNVKYNFMRIIKYEFILNDALNQGIIRANDQY
LTAAALHNLDEAVKFDMGAYKSSKDDAKITVILRISKTQLYVTAQDEDQPVLLKEMPEIPKTITGSETNLLF
FWETHGTKNYFTSVAHPNLFIATKQDYWVSLAGGPPSITDFQILENQA

### Example 8: CD8-Targeted Fc-IL-1α with IL-1α at the N-Terminus

In the above examples, IL-1α was cloned at the C-terminus of the human IgG1 Fc sequence containing the L234A_L235A_K322Q effector mutations and the ‘knob’ modifications S354C_T366W (via the flexible 20×GGS-linker) in the pcDNA3.4 expression vector. In this example, an alternative configuration (see FIG. 7C) was produced in which IL-1α was cloned at the N-terminus.
CD8 VHH_5GGS_Fc_hole+Fc-knob_20GGS_IL1a delta 1-6
CD8 VHH_5GGS_Fc_hole+IL1a delta 1-6_Fc-knob

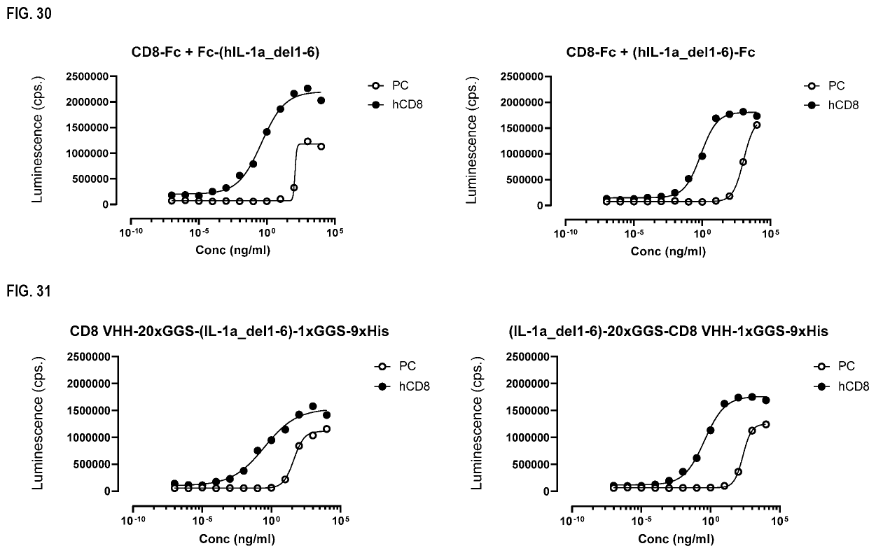

Resulting proteins were tested in the assay described in Example 6. As shown in FIG. 30, both configurations have similar biological activity upon targeting with an EC₅₀ value around 0.5 ng/ml and a similar two to three log 10 targeting window versus CD8 negative cells.

### Amino Acid Sequences Used in the Above Example are as Follows:

CD8 VHH_5GGS_Fc_hole (underlined sequence is a
leader sequence)
(SEQ ID NO: 445)
Fc-knob_20GGS_IL1a delta 1-6 (underlined sequence
is a leader sequence)
(SEQ ID NO: 443)
IL1a delta 1-6_20GGS_Fc-knob (underlined sequence
is a leader sequence)
(SEQ ID NO: 512)
MEFGLSWLFLVAILKGVQCLSNVKYNFMRIIKYEFILNDALNQSIIRAN
DQYLTAAALHNLDEAVKFDMGAYKSSKDDAKITVILRISKTQLYVTAQD
EDQPVLLKEMPEIPKTITGSETNLLFFWETHGTKNYFTSVAHPNLFIAT
KQDYWVCLAGGPPSITDFQILENQAGGSGGSGGSGGSGGSGGSGGSGGS
GGSGGSGGSGGSGGSGGSGGSGGSGGSGGSGGSGGSDKTHTCPPCPAPE
AAGGPSVFLFPPKPKDTLMISRTPEVTCVVVDVSHEDPEVKFNWYVDGV
EVHNAKTKPREEQYNSTYRVVSVLTVLHQDWLNGKEYKCQVSNKALPAP
IEKTISKAKGQPREPQVYTLPPCRDELTKNQVSLWCLVKGFYPSDIAVE
WESNGQPENNYKTTPPVLDSDGSFFLYSKLTVDKSRWQQGNVFSCSVMH
EALHNHYTQKSLSLSPGK

### Example 9: Single-Peptide IL-1α Variants

In this example, IL-1α was cloned in a single-peptide format. To this end, sequences encoding the CD8 VHH 1CDA65 and IL-1α were fused via a flexible 20×GGS-linker in the pcDNA3.4 expression vector for eukaryotic expression. Two variants were created that differ in the orientation of the CD8 VHH and IL-1α. A C-terminal 9×His tag was added for purification purposes. The resulting plasmids were transfected in ExpiCHO cells (ThermoFisher) according to the manufacturer's guidelines. One week after transfection, supernatant was collected, and cells removed by centrifugation. Recombinant proteins were purified based on the His-tag (HisTrap Excel column; Cytiva) and by subsequent size exclusion chromatography (Superdex 200 increase HiScale 16/40 column, Cytiva), both on an Äkta purifier (GE Healthcare). Concentrations were measured with a UV/Vis absorbance spectrometer (Lunatic, Unchained Labs) and purity estimated on SDS-PAGE.
CD8 VHH_20GGS_IL1α delta 1-6_1GGS_9xhis
IL1α delta 1-6_20GGS_CD8 VHH_1GGS_9xhis

Resulting proteins were tested for biological activity on the parental and CD8+ HekBlue-IL-1β cell line as described earlier. Both single peptide variants allowed selective signaling in CD8+ cells with similar potency (EC50 around 0.3 ng/ml) as the previously described Fc-based variant (FIG. 31) and a similar two to three log 10 targeting window versus CD8 negative cells.

Amino Acid Sequences Used in this Example:

hCD8 VHH 1CDA65_20GGS_IL1α delta 1-6_1GGS_9xhis
(underlined sequence is a leader sequence)
(SEQ ID NO: 513)
MEFGLSWLFLVAILKGVQCQVQLQESGGGLVQPGGSLRLSCAASGSIFS
INVMGWYRQTPGKERELVAKITNFGITSYADSAQGRFTISRGNAKNTVY
LQMNSLKPEDTAVYYCNLDTTGWGPPPYQYWGQGTQVTVSSVDGGSGGS
GGSGGSGGSGGSGGSGGSGGSGGSGGSGGSGGSGGSGGSGGSGGSGGSG
GSGGSLSNVKYNFMRIIKYEFILNDALNQSIIRANDQYLTAAALHNLDE
AVKFDMGAYKSSKDDAKITVILRISKTQLYVTAQDEDQPVLLKEMPEIP
KTITGSETNLLFFWETHGTKNYFTSVAHPNLFIATKQDYWVCLAGGPPS
ITDFQILENQAGGSHHHHHHHHH
IL1α delta 1-6_20GGS_hCD8 VHH 1CDA65_1GGS_9xhis
(underlined sequence is a leader sequence)
(SEQ ID NO: 514)
MEFGLSWLFLVAILKGVQCLSNVKYNFMRIIKYEFILNDALNQSIIRAN
DQYLTAAALHNLDEAVKFDMGAYKSSKDDAKITVILRISKTQLYVTAQD
EDQPVLLKEMPEIPKTITGSETNLLFFWETHGTKNYFTSVAHPNLFIAT
KQDYWVCLAGGPPSITDFQILENQAGGSGGSGGSGGSGGSGGSGGSGGS
GGSGGSGGSGGSGGSGGSGGSGGSGGSGGSGGSGGSQVQLQESGGGLVQ
PGGSLRLSCAASGSIFSINVMGWYRQTPGKERELVAKITNFGITSYADS
AQGRFTISRGNAKNTVYLQMNSLKPEDTAVYYCNLDTTGWGPPPYQYWG
QGTQVTVSSVDGGSHHHHHHHHH

### Example 10: Alternative CD8 Targeting Domains

In the above examples human CD8 was targeted via the CD8 VHH 1CDA65. Here, other CD8 VHHs, recognizing an overlapping (2CDA74) or a different CD8 epitope (2CDA5, 3CDA19, 2CDA68, R2HCD26), as well as other types of targeting moieties including scFv's (based on OKT8 antibody sequence from WO2019033043) were compared in the NF-κB reporter assay in transfected Hek293T cells, as described in Example 2. The CD8 targeting moieties were tested in a Fc-IL-1α heterodimeric fusion configuration like the one outlined in FIG. 7B with hIL-1α C141H or M15G.
CD8 VHH 1CDA65_5GGS_Fc_hole+Fc-knob_20GGS_IL1α delta 1-6_C141H
CD8 VHH 1CDA65_5GGS_Fc_hole+Fc-knob_20GGS_IL1α delta 1-6_M15G
CD8 VHH 2CDA74_5GGS_Fc_hole+Fc-knob_20GGS_IL1α delta 1-6_C141H
CD8 VHH 2CDA74_5GGS_Fc_hole+Fc-knob_20GGS_IL1α delta 1-6_M15G
CD8 VHH 2CDA5_5GGS_Fc_hole+Fc-knob_20GGS_IL1α delta 1-6_C141H
CD8 VHH 2CDA5_5GGS_Fc_hole+Fc-knob_20GGS_IL1α delta 1-6_M15G
CD8 VHH 3CDA19_5GGS_Fc_hole+Fc-knob_20GGS_IL1α delta 1-6_C141H
CD8 VHH 3CDA19_5GGS_Fc_hole+Fc-knob_20GGS_IL1α delta 1-6_M15G
CD8 VHH 2CDA68_5GGS_Fc_hole+Fc-knob_20GGS_IL1α delta 1-6_C141H
CD8 VHH 2CDA68_5GGS_Fc_hole+Fc-knob_20GGS_IL1α delta 1-6_M15G
CD8 VHH R2HCD26_5GGS_Fc_hole+Fc-knob_20GGS_IL1α delta 1-6_C141H
CD8 VHH R2HCD26_5GGS_Fc_hole+Fc-knob_20GGS_IL1α delta 1-6_M15G
CD8 OKT8 scFv (V_{H}-V_{L})_20GGS_Fc_hole+Fc-knob_20GGS_IL1α delta 1-6_C141H
CD8 OKT8 scFv (V_{H}-V_{L})_20GGS_Fc_hole+Fc-knob_20GGS_IL1α delta 1-6_M15G
CD8 OKT8 scFv (V_{L}-V_{H})_20GGS_Fc_hole+Fc-knob_20GGS_IL1α delta 1-6_C141H
CD8 OKT8 scFv (V_{L}-V_{H})_20GGS_Fc_hole+Fc-knob_20GGS_IL1α delta 1-6_M15G

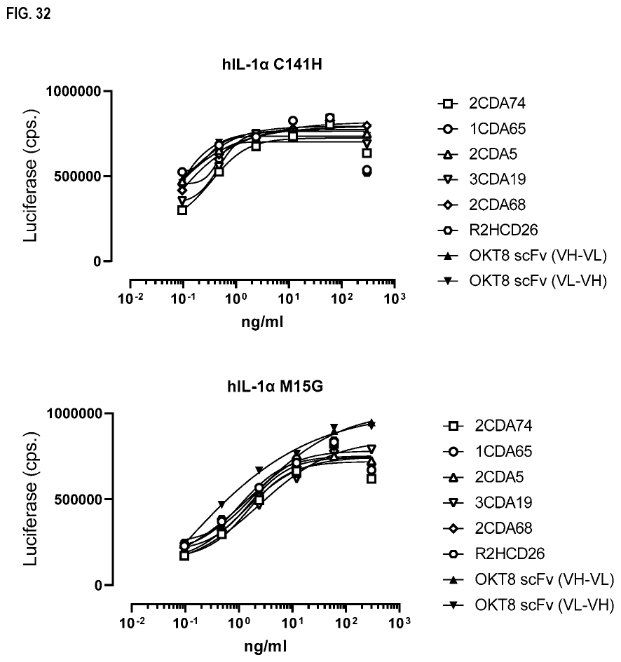

Data in FIG. 32 illustrate that fusions based on CD8 VHHs from the same or a different epitope bin than 1CDA65, show a similar dose-dependent signal compared to 1CDA65-Fc-IL-1α C141H or M15G. Also, the scFv fusions based on the sequence of the OKT8 antibody behave in a similar way.

Amino Acid Sequences Used in this Example:

CD8 VHH 2CDA5_5GGS_Fc_hole (underlined sequence is a leader sequence)
(SEQ ID NO: 515)
MGWSCIIFFLVATATGVHSDVQLQESGGGLVQAGDSLRLTCTASGRTFSNYGIGWFRQAPGKEREFVAGINWSGE
SADYADSVKGRFTISRDNAKNTVYLQMNSLKPEDTAVYYCAAGESGVWVGGLDYWGQGTQVTVSSGGSGGSGG
SGGSGGSDKTHTCPPCPAPEAAGGPSVFLFPPKPKDTLMISRTPEVTCVVVDVSHEDPEVKFNWYVDGVEVHNAK
TKPREEQYNSTYRVVSVLTVLHQDWLNGKEYKCQVSNKALPAPIEKTISKAKGQPREPQVCTLPPSRDELTKNQVSL
SCAVKGFYPSDIAVEWESNGQPENNYKTTPPVLDSDGSFFLVSKLTVDKSRWQQGNVFSCSVMHEALHNHYTQK
SLSLSPGK
CD8 VHH 3CDA19_5GGS_Fc_hole (underlined sequence is a leader sequence)
(SEQ ID NO: 516)
MGWSCIIFFLVATATGVHSDVQLQESGGGLVQAGGSLRLSCAASGFSSDDYTIGWFRQAPGKEREGISCFSSSDGS
TGFADSVKGRFTISSDNATNTVYLEMNSLKPEDTAVYYCAADFNVWSPPICGSRWYGPPPGGMEYWGKGTQVTV
SSGGSGGSGGSGGSGGSDKTHTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCQVSNKALPAPIEKTISKAKGQPREPQVCTLPPS
RDELTKNQVSLSCAVKGFYPSDIAVEWESNGQPENNYKTTPPVLDSDGSFFLVSKLTVDKSRWQQGNVFSCSVMH
EALHNHYTQKSLSLSPGK
CD8 VHH 2CDA74_5GGS_Fc_hole (underlined sequence is a leader sequence)
(SEQ ID NO: 517)
MGWSCIIFFLVATATGVHSDVQLQESGGGLVQAGGSLRLSCAVSGFTFDNYAIGWFRQAPGKEREGVSSISRSDGS
TYYADSVRGRFTISSDNAKNTVYLQMNSLKPEDTAVYYCAAETSADSGEFRFGWVLKPSLYDYWGQGTQVTVSSG
GSGGSGGSGGSGGSDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCQVSNKALPAPIEKTISKAKGQPREPQVCTLPPSRD
ELTKNQVSLSCAVKGFYPSDIAVEWESNGQPENNYKTTPPVLDSDGSFFLVSKLTVDKSRWQQGNVFSCSVMHEA
LHNHYTQKSLSLSPGK
CD8 VHH 2CDA68_5GGS_Fc_hole (underlined sequence is a leader sequence)
(SEQ ID NO: 518)
MGWSCIIFFLVATATGVHSDVQLQESGGGLVQAGGSLRLSCAASGRTFSSYVMGWFRQAPGKEREFVAQISWSA
GSIYYADSVKGRFTISNDNAKRTVYLQMNSLKPEDTAVYYCAERGYAYCSDDGCQRTQDYDYWGQGTQVTVSSG
GSGGSGGSGGSGGSDKTHTCPPCPAPEAAGGPSVFLFPPKPKDTLMISRTPEVTCVVVDVSHEDPEVKFNWYVD
GVEVHNAKTKPREEQYNSTYRVVSVLTVLHQDWLNGKEYKCQVSNKALPAPIEKTISKAKGQPREPQVCTLPPSRD
ELTKNQVSLSCAVKGFYPSDIAVEWESNGQPENNYKTTPPVLDSDGSFFLVSKLTVDKSRWQQGNVFSCSVMHEA
LHNHYTQKSLSLSPGK
CD8 VHH R2HCD26_5GGS_Fc_hole (underlined sequence is a leader sequence)
(SEQ ID NO: 519)
MGWSCIIFFLVATATGVHSDVQLQESGGGLVQAGGSLRLSCAASGFTFDDYAIGWFRQAPGKEREGVSCIRVSDG
STYYADPVKGRFTISSDNAKNTVYLQMNSLKPEDAAVYYCAAGSLYTCVQSIVVVPARPYYDMDYWGKGTQVTVS
SGGSGGSGGSGGSGGSDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCQVSNKALPAPIEKTISKAKGQPREPQVCTLPPSR
DELTKNQVSLSCAVKGFYPSDIAVEWESNGQPENNYKTTPPVLDSDGSFFLVSKLTVDKSRWQQGNVFSCSVMHE
ALHNHYTQKSLSLSPGK
CD8 OKT8 scFv (VH-VL)_20GGS_Fc_hole (underlined sequence is a leader sequence)
(SEQ ID NO: 520)
MEFGLSWLFLVAILKGVQCEVQLVQSGAEVKKPGASVKVSCKASGFNIKDTYIHWVRQAPGQGLEWIGRIDPAND
NTLYASKFQGRATITADTSTSTAYLELSSLRSEDTAVYYCGRGYGYYVFDHWGQGTLVTVSSGGGGSGGGGSGGG
GSDVQITQSPSSLSASVGDRVTITCRTSRSISQYLAWYQEKPGKTNKLLIYSGSTLQSGIPSRFSGSGSGTDFTLTISSLQ
PEDFATYYCQQVNEFPPTFGQGTKVEIKGGSGGSGGSGGSGGSGGSGGSGGSGGSGGSGGSGGSGGSGGSGG
SGGSGGSGGSGGSGGSDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCQVSNKALPAPIEKTISKAKGQPREPQVCTLPPSR
DELTKNQVSLSCAVKGFYPSDIAVEWESNGQPENNYKTTPPVLDSDGSFFLVSKLTVDKSRWQQGNVFSCSVMHE
ALHNHYTQKSLSLSPGK
CD8 OKT8 scFv (VL-VH)_20GGS_Fc_hole (underlined sequence is a leader sequence)
(SEQ ID NO: 521)
MEFGLSWLFLVAILKGVQCDVQITQSPSSLSASVGDRVTITCRTSRSISQYLAWYQEKPGKTNKLLIYSGSTLQSGIPS
RFSGSGSGTDFTLTISSLQPEDFATYYCQQVNEFPPTFGQGTKVEIKGGGGSGGGGSGGGGSEVQLVQSGAEVKKP
GASVKVSCKASGFNIKDTYIHWVRQAPGQGLEWIGRIDPANDNTLYASKFQGRATITADTSTSTAYLELSSLRSEDT
AVYYCGRGYGYYVFDHWGQGTLVTVSSGGSGGSGGSGGSGGSGGSGGSGGSGGSGGSGGSGGSGGSGGSGG
SGGSGGSGGSGGSGGSDKTHTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCQVSNKALPAPIEKTISKAKGQPREPQVCTLPPSR
DELTKNQVSLSCAVKGFYPSDIAVEWESNGQPENNYKTTPPVLDSDGSFFLVSKLTVDKSRWQQGNVFSCSVMHE
ALHNHYTQKSLSLSPGK
Fc-knob_20GGS_IL1α delta 1-6_C141H (underlined sequence is a leader sequence)
(SEQ ID NO: 447)
Fc-knob_20GGS_IL1α delta 1-6_M15G (underlined sequence is a leader sequence)
(SEQ ID NO: 450)

### Example 11: Bivalent Targeting of IL-1α Activity

In this example, targeting of IL-1α activity to CD8 positive cells using formats with one (monovalent) or two CD8 1CDA65 VHHs (bivalent) was evaluated. On a knob-in-hole scaffold, VHHs and IL-1α warhead were cloned in a bivalent configuration like the one outlined in FIG. 16A with TM1 and TM2 being identical and compared to monovalent targeting in the configuration outline in FIG. 10A or FIG. 7B.
CD8 VHH_5GGS_Fc_hole+Fc-knob_20GGS_IL1α delta 1-6 (configuration of FIG. 7B).
Fc_hole+CD8_5GGS_Fc-knob_20GGS_IL1α delta 1-6 (configuration of FIG. 10A)
CD8 VHH_5GGS_Fc_hole+CD8_5GGS_Fc-knob_20GGS_IL1α delta 1-6 (configuration of FIG. 16A)

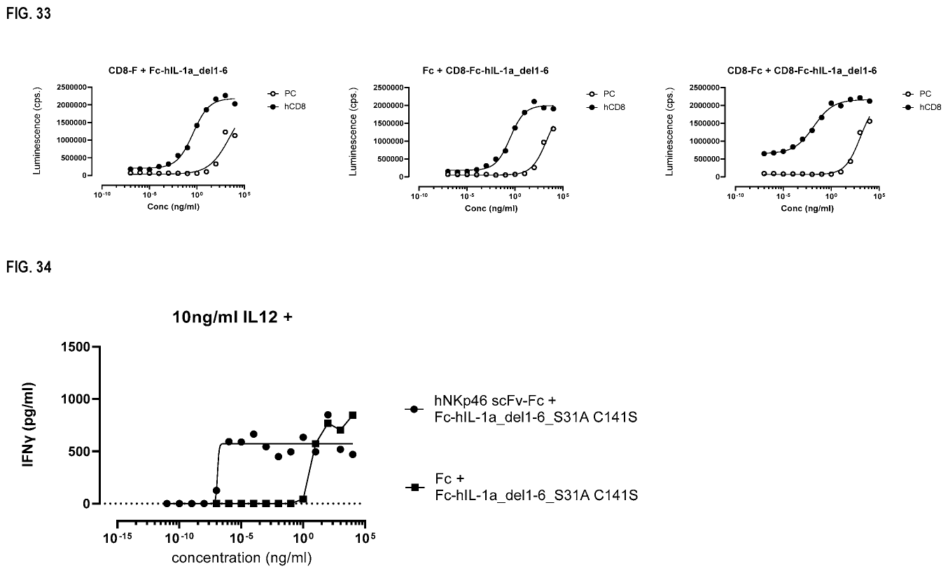

The resulting knob-in-hole Fc combinations were tested for biological activity on the parental and CD8+ HekBlue-IL-1β cell line as described earlier. Data in FIG. 33 illustrate that a bivalent 1CDA65-1CDA65-Fc-IL-1α is more active than its monovalent counterparts which behave in both configurations similarly (EC50 bivalent: 0.015 ng/ml vs EC50 monovalent: 0.3-0.4 ng/ml).

Amino Acid Sequences Used in this Example:

CD8 VHH_5GGS_Fc_hole (underlined sequence is a
leader sequence)
(SEQ ID NO: 445)
Fc_hole (underlined sequence is a leader
sequence)
(SEQ ID NO: 444)
CD8 VHH 1CDA65_5GGS_Fc_knob_20GGS_IL1a delta 1-6
(underlined sequence is a leader sequence)
(SEQ ID NO: 522)
MEFGLSWLFLVAILKGVQCDVQLQESGGGLVQPGGSLRLSCAASGSIF
SINVMGWYRQTPGKERELVAKITNFGITSYADSAQGRFTISRGNAKNT
VYLQMNSLKPEDTAVYYCNLDTTGWGPPPYQYWGQGTQVTVSSGGSGG
SGGSGGSGGSDKTHTCPPCPAPEAAGGPSVFLFPPKPKDTLMISRTPE
VTCVVVDVSHEDPEVKFNWYVDGVEVHNAKTKPREEQYNSTYRVVSVL
TVLHQDWLNGKEYKCQVSNKALPAPIEKTISKAKGQPREPQVYTLPPC
RDELTKNQVSLWCLVKGFYPSDIAVEWESNGQPENNYKTTPPVLDSDG
SFFLYSKLTVDKSRWQQGNVFSCSVMHEALHNHYTQKSLSLSPGKGGS
GGSGGSGGSGGSGGSGGSGGSGGSGGSGGSGGSGGSGGSGGSGGSGGS
GGSGGSGGSLSNVKYNFMRIIKYEFILNDALNQSIIRANDQYLTAAAL
HNLDEAVKFDMGAYKSSKDDAKITVILRISKTQLYVTAQDEDQPVLLK
EMPEIPKTITGSETNLLFFWETHGTKNYFTSVAHPNLFIATKQDYWVC
LAGGPPSITDFQILENQA

### Example 12: Targeting IL-1α to NK Cells Via NKp46

IL-1α was shown to enhance NK cell production of IFNγ when induced by IL-12 (Hunter et al., “The role of the CD28/B7 interaction in the regulation of NK cell responses during infection with Toxoplasma gondii,” J. Immunol. (1997) 158 (5): 2285-93). To demonstrate that IL-1α activity can be specifically targeted to NK cells, a scFv recognizing human NKp46 (based on the NKp46-1 sequence in US 2021/0269523) was fused at the N-terminus of the human IgG1 Fc sequence containing the L234A_L235A_K322Q effector mutations and the ‘hole’ modifications Y349C_T366S_L368A_Y407V (via the flexible 5×GGS-linker) in the pcDNA3.4 expression vector. This construct was co-transfected with a Fc_knob-20×GGS-IL-1α variant for expression in ExpiCHO cells (ThermoFisher) and purified like described above.
NKp46 scFv (VL-VH)_5GGS_Fc_hole+Fc-knob_20GGS_IL1α delta 1-6_S31A_C141S
Fc_hole+Fc-knob_20GGS_IL1α delta 1-6_S31A_C141S

The resulting NKp46 scFv-Fc-IL-1α variant was tested for IFNγ induction on isolated NK cells. In brief, PBMCs from buffy coats of healthy donors were isolated using density gradient centrifugation using Lymphoprep (StemCell technologies). NK cells were enriched using the NK Cell Isolation Kit (130-092-657; Miltenyi Biotec) and incubated overnight in RPMI+10% HI FBS+Pen/strep. The next day, cells were stimulated with 10 ng/ml hIL-12 (573004; BioLegend), combined with a serial dilution of NKp46 scFv or untargeted-Fc-IL-1α variants for 24 hours at 37° C. Cell-free culture supernatant was collected and IFNγ levels were analyzed using the human IFN-gamma DuoSet ELISA (R&D Systems; DY285B).

Data in FIG. 34 illustrate that on isolated NK cells NKp46-targeted IL-1α_del1-6_S31A_C141S mutant has a potency shifted about 7-log when comparing the targeted with the untargeted version (EC50 targeted: 1.2E-07 ng/ml; EC50 untargeted: 5.4 ng/ml). Similar results were obtained when using a NKp46 VHH targeting moiety.

Amino Acid Sequences Used in this Example:

NKp46 scFv (VL-VH)_5GGS_Fc_hole (underlined
sequence is a leader sequence)
(SEQ ID NO: 523)
MGWSCIIFFLVATATGVHSDIQMTQTTSSLSASLGDRVTISCRASQDIS
NYLNWYQQKPDGTVKLLIYYTSRLHSGVPSRFSGSGSGTDYSLTINNLE
QEDIATYFCQQGNTRPWTFGGGTKLEIKGGGGSGGGGSGGGGSQVQLQQ
SGPELVKPGASVKMSCKASGYTFTDYVINWGKQRSGQGLEWIGEIYPGS
GTNYYNEKFKAKATLTADKSSNIAYMQLSSLTSEDSAVYFCARRGRYGL
YAMDYWGQGTSVTVSSGGSGGSGGSGGSGGSDKTHTCPPCPAPEAAGGP
SVFLFPPKPKDTLMISRTPEVTCVVVDVSHEDPEVKFNWYVDGVEVHNA
KTKPREEQYNSTYRVVSVLTVLHQDWLNGKEYKCQVSNKALPAPIEKTI
SKAKGQPREPQVCTLPPSRDELTKNQVSLSCAVKGFYPSDIAVEWESNG
QPENNYKTTPPVLDSDGSFFLVSKLTVDKSRWQQGNVFSCSVMHEALHN
HYTQKSLSLSPGK
Fc-knob_20GGS_IL1α delta 1-6_S31A_C141S
(underlined sequence is a leader sequence)
(SEQ ID NO: 524)
MDMRVPAQLLGLLLLWLRGARCDKTHTCPPCPAPEAAGGPSVFLFPPKP
KDTLMISRTPEVTCVVVDVSHEDPEVKFNWYVDGVEVHNAKTKPREEQY
NSTYRVVSVLTVLHQDWLNGKEYKCQVSNKALPAPIEKTISKAKGQPRE
PQVYTLPPCRDELTKNQVSLWCLVKGFYPSDIAVEWESNGQPENNYKTT
PPVLDSDGSFFLYSKLTVDKSRWQQGNVFSCSVMHEALHNHYTQKSLSL
SPGKGGSGGSGGSGGSGGSGGSGGSGGSGGSGGSGGSGGSGGSGGSGGS
GGSGGSGGSGGSGGSLSNVKYNFMRIIKYEFILNDALNQAIIRANDQYL
TAAALHNLDEAVKFDMGAYKSSKDDAKITVILRISKTQLYVTAQDEDQP
VLLKEMPEIPKTITGSETNLLFFWETHGTKNYFTSVAHPNLFIATKQDY
WVSLAGGPPSITDFQILENQA
Fc_hole (underlined sequence is a leader sequence)
(SEQ ID NO: 444)

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### Embodiments

Embodiment 1. A chimeric protein comprising: (a) an interleukin-1α (IL-1α), pro-IL-1α, or a mutant thereof, and (b) one or more targeting moieties, said targeting moieties comprising recognition domains which specifically bind to an antigen or receptor of interest; wherein the IL-1α, pro-IL-1α, or the mutant thereof, and the one or more targeting moieties are optionally connected with one or more linkers.

Embodiment 2. The chimeric protein of embodiment 1, wherein: (a) the IL-1α comprises an amino acid sequence having at least about 90%, or at least about 93%, or at least about 95%, or at least about 97%, or at least about 98%, or at least about 99% identity with SEQ ID NO: 1 or 3 or (b) the pro-IL-1α comprises an amino acid sequence having at least about 90%, or at least about 93%, or at least about 95%, or at least about 97%, or at least about 98%, or at least about 99% identity with SEQ ID NO: 2 or 4.

Embodiment 3. The chimeric protein of embodiment 1 or 2, wherein the mutant IL-1α or pro-IL-1α comprises one or more mutations.

Embodiment 4. The chimeric protein of embodiment 3, wherein the one or more mutations of the mutant IL-1α or mutant pro-IL-1α confer reduced affinity for IL-1R or IL-1RAcP.

Embodiment 5. The chimeric protein of embodiment 4, wherein the IL-1α or pro-IL-1α exhibits reduced affinity for IL-1R1.

Embodiment 6. The chimeric protein of embodiment 4, wherein the IL-1α or pro-IL-1α exhibits reduced affinity for IL-1RAcP.

Embodiment 7. The chimeric protein of embodiment 4, wherein the one or more mutations of the mutant IL-1α is selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S, Q153A, and Q153G.

Embodiment 8. The chimeric protein of embodiment 4, wherein the amino acid position of the one or more mutations of the mutant pro-IL-1α is selected from P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G.

Embodiment 9. The chimeric protein of any one of embodiments 1-8, wherein the one or more mutations confer reduced affinity that is restorable by attachment to one or more targeting moieties or upon inclusion in the Fc-based chimeric protein complex.

Embodiment 10. The chimeric protein of any one of embodiments 1-9, wherein the targeting moiety is directed against a tumor cell.

Embodiment 11. The chimeric protein of any one of embodiments 1-10, wherein the targeting moiety comprise a recognition domain that recognizes and/or binds an antigen or receptor on a tumor cell, endothelial cell, epithelial cell, mesenchymal cell, tumor stroma or stromal cell, ECM and/or immune cell, organ cells, and/or tissue cells.

Embodiment 12. The chimeric protein of embodiment 11, wherein the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof.

Embodiment 13. The chimeric protein of any one of embodiments 1-12, wherein the targeting moiety comprises a recognition domain that is a full-length antibody or a fragment thereof, a single-domain antibody, a recombinant heavy-chain-only antibody (V_{H}H), a single-chain antibody (scFv), a Humabody, a shark heavy-chain-only antibody (VNAR), a microprotein (e.g. cysteine knot protein, knottin), a darpin, an anticalin, an adnectin, an aptamer, a Fv, a Fab, a Fab′, or a F(ab′)₂.

Embodiment 14. The chimeric protein of any one of embodiments 1-13, wherein the recognition domain is a single-domain antibody (V_{H}H), optionally a V_{H}H, or humanized V_{H}H.

Embodiment 15. The chimeric protein of any one of embodiments 1-14, wherein the recognition domain functionally modulates the antigen or receptor of interest.

Embodiment 16. The chimeric protein of any one of embodiments 1-15, wherein the recognition domain binds but does not functionally modulate the antigen or receptor of interest.

Embodiment 17. The chimeric protein of any one of embodiments 1-16, comprising two or more targeting moieties.

Embodiment 18. The chimeric protein of any one of embodiments 1-17, further comprising one or more additional modified signaling agents.

Embodiment 19. The chimeric protein of any one of embodiments 1-18, wherein the connector between (i) and (ii) is a flexible linker.

Embodiment 20. The chimeric protein of embodiment 19, wherein the flexible linker is substantially comprised of glycine and serine residues, optionally wherein i) the flexible linker comprises (Gly₄Ser)ₙ, where n is from about 1 to about 8; (ii) the flexible linker comprises (Gly₂Ser)ₙ, where n is from about 1 to about 20; or iii) the flexible linker comprises one or more of SEQ ID NOs: 435-442.

Embodiment 21. The chimeric protein of embodiment 19, wherein the flexible linker is substantially comprised of GGSGGSGGGGSGGGGS (SEQ ID NO: 257).

Embodiment 22. The chimeric protein of embodiment 19, wherein the flexible linker is substantially comprised of LE, GGGGS (SEQ ID NO: 249), (GGGGS)n (n=1-4) (SEQ ID NO: 249-SEQ ID NO: 252), (Gly)8 (SEQ ID NO: 258), (Gly)6 (SEQ ID NO: 259), (EAAAK)n (n=1-3) (SEQ ID NO: 260-SEQ ID NO: 262), A(EAAAK)nA (n=2-5) (SEQ ID NO: 263-SEQ ID NO: 266), AEAAAKEAAAKA (SEQ ID NO: 263), A(EAAAK) 4ALEA(EAAAK)4A (SEQ ID NO: 267), PAPAP (SEQ ID NO: 268), KESGSVSSEQLAQFRSLD (SEQ ID NO: 269), EGKSSGSGSESKST (SEQ ID NO: 270), GSAGSAAGSGEF (SEQ ID NO: 271), and (XP)n, with X designating any amino acid, e.g., Ala, Lys, or Glu.

Embodiment 23. The chimeric protein of any one of embodiments 1-18, wherein the chimeric protein is suitable for use in a patient having one or more of: cancer, infections, immune disorders, autoimmune diseases, cardiovascular diseases, wound, ischemia-related diseases, neurodegenerative diseases, and/or metabolic diseases.

Embodiment 24. A recombinant nucleic acid composition encoding one or more chimeric proteins of any one of embodiments 1-23.

Embodiment 25. A host cell comprising a nucleic acid of embodiment 20.

Embodiment 26. A method for treating cancer, comprising administering an effective amount of i) the chimeric protein of any one of the embodiments 1-22 to a patient in need thereof; ii) the recombinant nucleic acid of embodiment 24 to a patient in need thereof; or iii) the host cell of embodiment 25 to a patient in need thereof.

Embodiment 27. The method of embodiment 26, wherein the cancer is selected form one or more of basal cell carcinoma, biliary tract cancer, bladder cancer, bone cancer, brain and central nervous system cancer, breast cancer, cancer of the peritoneum, cervical cancer, choriocarcinoma, colon and rectum cancer, connective tissue cancer, cancer of the digestive system, endometrial cancer, esophageal cancer, eye cancer, cancer of the head and neck, gastric cancer (including gastrointestinal cancer), glioblastoma, hepatic carcinoma, hepatoma, intra-epithelial neoplasm, kidney or renal cancer, larynx cancer, leukemia, liver cancer, lung cancer (e.g., small-cell lung cancer, non-small cell lung cancer, adenocarcinoma of the lung, and squamous carcinoma of the lung), melanoma, myeloma, neuroblastoma, oral cavity cancer (lip, tongue, mouth, and pharynx), ovarian cancer, pancreatic cancer, prostate cancer, retinoblastoma, rhabdomyosarcoma, rectal cancer, cancer of the respiratory system, salivary gland carcinoma, sarcoma (e.g., Kaposi's sarcoma), skin cancer, squamous cell cancer, stomach cancer, testicular cancer, thyroid cancer, uterine or endometrial cancer, cancer of the urinary system, vulval cancer, lymphoma including Hodgkin's and non-Hodgkin's lymphoma, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), hairy cell leukemia, chronic myeloblastic leukemia as well as other carcinomas and sarcomas, post-transplant lymphoproliferative disorder (PTLD) as well as abnormal vascular proliferation associated with phakomatoses, edema (e.g. that associated with brain tumors), and Meigs' syndrome.

Embodiment 28. A method for treating an autoimmune disease or disorder, comprising administering an effective amount of i) the chimeric protein of any one of the embodiments 1-22 to a patient in need thereof; ii) the recombinant nucleic acid of embodiment 24 to a patient in need thereof; or iii) the host cell of embodiment 25 to a patient in need thereof.

Embodiment 29. The method of embodiment 28, wherein the autoimmune disease or disorder is selected from Crohn's disease, diabetes, multiple sclerosis, systemic lupus erythematosis, rheumatoid arthritis or juvenile rheumatoid arthritis, and ulcerative colitis.

Embodiment 30. The chimeric protein of any one of embodiments 1-22 for use as a medicament.

Embodiment 31. Use of the chimeric protein of any one of embodiments 1-22 in the manufacture of a medicament.

Embodiment 32. The chimeric protein of any one of embodiments 1-23 for use in the treatment of cancer, autoimmune diseases, inflammatory diseases, metabolic diseases, cardiovascular diseases, infectious disease, degenerative and neurodegenerative diseases.

Embodiment 33. A Fc-based chimeric protein complex comprising:
(a) an interleukin-1α (IL-1α), pro-IL-1α, or a mutant thereof, and
(b) one or more targeting moieties, said targeting moieties comprising recognition domains which specifically bind to an antigen or receptor of interest; and
(c) a Fc domain, the Fc domain optionally having one or more mutations that reduces or eliminates one or more effector functions of the Fc domain, promotes Fc chain pairing in the Fc domain, and/or stabilizes a hinge region in the Fc domain.

Embodiment 34. The Fc-based chimeric protein complex of embodiment 33, wherein:
(a) the IL-1α comprises an amino acid sequence having at least about 90%, or at least about 93%, or at least about 95%, or at least about 97%, or at least about 98%, or at least about 99% identity with SEQ ID NO: 1 or
(b) the pro-IL-1α comprises an amino acid sequence having at least about 90%, or at least about 93%, or at least about 95%, or at least about 97%, or at least about 98%, or at least about 99% identity with SEQ ID NO:2.

Embodiment 35. The Fc-based chimeric protein complex of embodiment 33 or 34, wherein the mutant IL-1α or pro-IL-1α comprises one or more mutations.

Embodiment 36. The Fc-based chimeric protein complex of embodiment 35, wherein the one or more mutations of the mutant IL-1α or mutant pro-IL-1α confer reduced affinity for IL-1R or IL-1RAcP.

Embodiment 37. The Fc-based chimeric protein complex of embodiment 36, wherein the IL-1α or pro-IL-1α exhibits reduced affinity for IL-1R1.

Embodiment 38. The Fc-based chimeric protein complex of embodiment 36, wherein the IL-1α or pro-IL-1α exhibits reduced affinity for IL-1RAcP.

Embodiment 39. The Fc-based chimeric protein complex of embodiment 36, wherein the one or more mutations of the mutant IL-1α is selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S, Q153A, and Q153G.

Embodiment 40. The Fc-based chimeric protein complex of embodiment 36, wherein the amino acid position of the one or more mutations of the mutant pro-IL-1α is selected from P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G.

Embodiment 41. The Fc-based chimeric protein complex of any one of embodiments 33-40, wherein the one or more mutations confer reduced affinity that is restorable by attachment to one or more targeting moieties or upon inclusion in the Fc-based chimeric protein complex.

Embodiment 42. The Fc-based chimeric protein complex of any one of 33-41, wherein the targeting moiety is directed against a tumor cell.

Embodiment 43. The Fc-based chimeric protein complex of any one of embodiments 33-42, wherein the targeting moiety is directed against an immune cell.

Embodiment 44. The Fc-based chimeric protein complex of embodiment 43, wherein the immune cell is selected from T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof.

Embodiment 45. The Fc-based chimeric protein complex of any one of embodiments 33-44, wherein the targeting moiety comprises a recognition domain that is a single-domain antibody, a recombinant heavy-chain-only antibody (VHH), a single-chain antibody (scFv), a shark heavy-chain-only antibody (VNAR), a microprotein (e.g. cysteine knot protein, knottin), a darpin, an anticalin, an adnectin, an aptamer, a Fv, a Fab, a Fab′, or a F(ab′)₂.

Embodiment 46. The Fc-based chimeric protein complex of any one of embodiments 33-45, wherein the recognition domain is a single-domain antibody (V_{HH}), optionally a V_{HH}, or humanized V_{HH}.

Embodiment 47. The Fc-based chimeric protein complex of any one of embodiments 33-46, wherein the recognition domain functionally modulates the antigen or receptor of interest.

Embodiment 48. The Fc-based chimeric protein complex of any one of embodiments 33-47, wherein the recognition domain binds but does not functionally modulate the antigen or receptor of interest.

Embodiment 49. The Fc-based chimeric protein complex of any one of embodiments 33-48, comprising two or more targeting moieties.

Embodiment 50. The Fc-based chimeric protein complex of any one of embodiments 33-49, further comprising one or more additional modified signaling agents.

Embodiment 51. The Fc-based chimeric protein complex of any one of embodiments 33-50, wherein the Fc-based chimeric protein complex comprises two signaling agents or two targeting moieties or two of both.

Embodiment 52. The Fc-based chimeric protein complex of any one of embodiments 33-51, wherein the Fc-based chimeric protein complex comprises three signaling agents or three targeting moieties or three of both.

Embodiment 53. The Fc-based chimeric protein complex of any one of the embodiments 33-52, wherein the Fc-based chimeric protein complex is suitable for use in a patient having one or more of: cancer, infections, immune disorders, autoimmune diseases, cardiovascular diseases, wound, ischemia-related diseases, neurodegenerative diseases, and/or metabolic diseases.

Embodiment 54. A recombinant nucleic acid composition encoding one or more Fc-based chimeric protein complexes of any one of embodiments 33-53, or a constituent polypeptide thereof.

Embodiment 55. A host cell comprising a nucleic acid of embodiment 54.

Embodiment 56. A method for treating cancer, comprising administering an effective amount of i) the Fc-based chimeric protein of any one of the embodiments 33-53 to a patient in need thereof; ii) the recombinant nucleic acid of embodiment 54 to a patient in need thereof; or iii) the host cell of embodiment 55 to a patient in need thereof.

Embodiment 57. The method of embodiment 56, wherein the cancer is selected form one or more of basal cell carcinoma, biliary tract cancer, bladder cancer, bone cancer, brain and central nervous system cancer, breast cancer, cancer of the peritoneum, cervical cancer, choriocarcinoma, colon and rectum cancer, connective tissue cancer, cancer of the digestive system, endometrial cancer, esophageal cancer, eye cancer, cancer of the head and neck, gastric cancer (including gastrointestinal cancer), glioblastoma, hepatic carcinoma, hepatoma, intra-epithelial neoplasm, kidney or renal cancer, larynx cancer, leukemia, liver cancer, lung cancer (e.g., small-cell lung cancer, non-small cell lung cancer, adenocarcinoma of the lung, and squamous carcinoma of the lung), melanoma, myeloma, neuroblastoma, oral cavity cancer (lip, tongue, mouth, and pharynx), ovarian cancer, pancreatic cancer, prostate cancer, retinoblastoma, rhabdomyosarcoma, rectal cancer, cancer of the respiratory system, salivary gland carcinoma, sarcoma (e.g., Kaposi's sarcoma), skin cancer, squamous cell cancer, stomach cancer, testicular cancer, thyroid cancer, uterine or endometrial cancer, cancer of the urinary system, vulval cancer, lymphoma including Hodgkin's and non-Hodgkin's lymphoma, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), hairy cell leukemia, chronic myeloblastic leukemia as well as other carcinomas and sarcomas, post-transplant lymphoproliferative disorder (PTLD) as well as abnormal vascular proliferation associated with phakomatoses, edema (e.g. that associated with brain tumors), and Meigs' syndrome.

Embodiment 58. A method for treating an autoimmune disease or disorder, comprising administering an effective amount of i) the Fc-based chimeric protein of any one of the embodiments 33-53 to a patient in need thereof; ii) the recombinant nucleic acid of embodiment 54 to a patient in need thereof; or iii) the host cell of embodiment 55 to a patient in need thereof.

Embodiment 59. The method of embodiment 58, wherein the autoimmune disease or disorder is selected from Crohn's disease, diabetes, multiple sclerosis, systemic lupus erythematosis, rheumatoid arthritis or juvenile rheumatoid arthritis, and ulcerative colitis.

Embodiment 60. The Fc-based chimeric protein of any one of embodiments 33-52 for use as a medicament.

Embodiment 61. Use of the chimeric protein of any one of embodiments 33-52 in the manufacture of a medicament.

Embodiment 62. The Fc-based chimeric protein complex of any one of embodiments 33-52 for use as a medicament.

Embodiment 63. The Fc-based chimeric protein complex of any one of embodiments 33-52 for use in the treatment of cancer, autoimmune diseases, inflammatory diseases, metabolic diseases, cardiovascular diseases, infectious disease, degenerative and neurodegenerative diseases.

Embodiment 64. Use of a Fc-based chimeric protein complex of any one of embodiments 33-52 in the manufacture of a medicament.

Embodiment 65. The Fc-based chimeric protein complex of any one of embodiments 33-52, wherein the Fc domain is from IgG, IgA, IgD, IgM or IgE.

Embodiment 66. The Fc-based chimeric protein complex of embodiment 65, wherein the IgG is selected from IgG1, IgG2, IgG3, or IgG4.

Embodiment 67. The Fc-based chimeric protein complex of any one of embodiments 33-53, wherein the Fc domain is from human IgG, IgA, IgD, IgM or IgE.

Embodiment 68. The Fc-based chimeric protein complex of embodiment 67, wherein the human IgG is selected from human IgG1, IgG2, IgG3, or IgG4.

Embodiment 69. The Fc-based chimeric protein complex of any one of embodiments 33-53 or 65-68, wherein the Fc chain pairing is promoted by ionic pairing and/or a knob-in-hole pairing.

Embodiment 70. The Fc-based chimeric protein complex of any one of embodiments 33-53 or 65-69, wherein the one or more mutations to the Fc domain results in an ionic pairing between the Fc chains in the Fc domain.

Embodiment 71. The Fc-based chimeric protein complex of any one of embodiments 33-53 or 65-70, wherein the one or more mutations to the Fc domain results in a knob-in-hole pairing in the Fc domain.

Embodiment 72. The Fc-based chimeric protein complex of any one of embodiments 33-53 or 65-71, wherein the one or more mutations to the Fc domain results in the reduction or elimination of an effector function of the Fc domain.

Embodiment 73. The Fc based chimeric protein complex of any one of embodiments 33-53 or 65-72, wherein the Fo-based chimeric protein complex is a heterodimer and has a trans orientation/configuration, as relates to any targeting moiety and signaling agent, relative to each other, or any targeting moieties relative to each other, or any signaling agents relative to each other.

Embodiment 74. The Fc based chimeric protein complex of any one of embodiments 33-53 or 65-73, wherein the Fc-based chimeric protein complex is a heterodimer and has a cis orientation, as relates to any targeting moiety and signaling agent, relative to each other, or any targeting moieties relative to each other, or any signaling agents relative to each other.

Embodiment 75. The Fc based chimeric protein complex of any one of embodiments 33-53 or 65-74, wherein the Fc comprises L234A, L235A, and K322Q substitutions in human IgG1 (according to EU numbering).

Embodiment 76. The Fc-based chimeric protein complex of any one of embodiments 33-53 or 65-75, wherein the Fc is human IgG1, and optionally contains one or more mutations of L234, L235, K322, D265, P329, and P331 (according to EU numbering).

Embodiment 77. The Fc-based chimeric protein complex of any one of embodiments 33-53 or 65-76, wherein the Fc-based chimeric protein complex has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, and 19A-F.

Embodiment 78. A vaccine composition, comprising (a) an adjuvant, comprising a chimeric protein or chimeric protein complex, comprising: (i) an interleukin-1α (IL-1α), pro-IL-1α, or a mutant thereof, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii); and/or (2) a flexible linker that connects (i) and (ii), wherein the mutant IL-1α or pro-IL-1α are characterized by low affinity or activity at the IL-1 receptor; and (b) an antigen which is suitable for inducing an immune response.

Embodiment 79. The vaccine composition of embodiment 78, wherein the IL-1α, pro-IL-1α, or a mutant thereof are human.

Embodiment 80. The vaccine composition of embodiment 78 or 79, wherein the low affinity or activity at the IL-1 receptor is restorable by attachment to one or more targeting moieties or upon inclusion in the chimeric protein complex.

Embodiment 81. The vaccine composition of any one of embodiments 78-80, wherein: (a) the mutant human IL-1α has an amino acid sequence of at least 95%, or 97% or 98% identity to SEQ ID NO: 1 or 3, or (b) the mutant human pro-IL-1α has an amino acid sequence of at least 95%, or 97% or 98% identity to SEQ ID NO: 2 or 4.

Embodiment 82. The vaccine composition of any one of embodiments 78-81, wherein the one or more mutations of the mutant IL-1α is selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S, Q153A, and Q153G.

Embodiment 83. The vaccine composition of embodiment 78-81, wherein the amino acid position of the one or more mutations of the mutant pro-IL-1α is selected from P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G.

Embodiment 84. The vaccine composition of any one of embodiments 78-83, wherein the targeting moiety is directed against a tumor cell.

Embodiment 85. The vaccine composition of any one of embodiments 78-84, wherein the targeting moiety comprises a recognition domain that recognizes and/or binds an antigen or receptor on a tumor cell, endothelial cell, epithelial cell, mesenchymal cell, stromal cell, ECM and/or immune cell, organ cell, and/or tissue cell.

Embodiment 86. The vaccine composition of embodiment 85, wherein the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof.

Embodiment 87. The vaccine composition of embodiment 86, wherein the immune cell is a T cell.

Embodiment 88. The vaccine composition of any one of embodiments 78-87, wherein the targeting moiety comprises a recognition domain that recognizes and/or binds CD8.

Embodiment 89. The vaccine composition of any one of embodiments 78-88, wherein the targeting moiety comprises a recognition domain that is a full-length antibody or a fragment thereof, a single-domain antibody, a recombinant heavy-chain-only antibody (VHH), a single-chain antibody (scFv), a Humabody, a shark heavy-chain-only antibody (VNAR), a microprotein (e.g. cysteine knot protein, knottin), a darpin, an anticalin, an adnectin, an aptamer, a Fv, a Fab, a Fab′, a F(ab′)₂, a peptide mimetic molecule, a natural ligand for a receptor, or a synthetic molecule.

Embodiment 90. The vaccine composition of any one of embodiments 78-89, wherein the chimeric protein or chimeric protein complex further comprises additional cytokines, wherein the additional cytokines are optionally modified, wherein the modification is optionally a mutation.

Embodiment 91. The vaccine composition of anyone of embodiments 78-90, wherein the chimeric protein or chimeric protein complex further comprises one or more additional targeting moieties.

Embodiment 92. The vaccine composition of any one of embodiments 78-91, wherein the chimeric protein or chimeric protein complex further comprises two signaling agents and/or two targeting moieties or two of both.

Embodiment 93. The vaccine composition of any one of embodiments 78-92, wherein the chimeric protein or chimeric protein complex further comprises three signaling agents and/or three targeting moieties or three of both.

Embodiment 94. The vaccine composition of any one of embodiments 78-93, wherein the targeting moiety comprises a recognition domain that recognizes and/or binds CD8 or CD4 and the mutant IL-1α comprises one or more mutations selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S, Q153A, and Q153G.

Embodiment 95. The vaccine composition of any one of embodiments 78-94, wherein the targeting moiety comprises a recognition domain that recognizes and/or binds CD8 or CD4 and the mutant pro-IL-1α comprises one or more mutations of the mutant pro-IL-1α is selected from P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G.

Embodiment 96. The vaccine composition of any one of embodiments 78-95, further comprising an aluminum gel or salt.

Embodiment 97. The vaccine composition of embodiment 96, wherein the aluminum gel or salt is selected from aluminum hydroxide, aluminum phosphate, and aluminum sulfate.

Embodiment 98. The vaccine composition of any one of embodiments 78-97, wherein the vaccine further comprises an additional adjuvant selected from oil-in-water emulsion formulations, saponin adjuvants, Freunds Adjuvants, toll like receptors ligands, cytokines, and chitosans.

Embodiment 99. The vaccine composition of any one of embodiments 78-98, wherein the vaccine composition is suitable for preventing or mitigating a disease or disorder is an infectious disease, autoimmune disease, or a cancer.

Embodiment 100. The vaccine composition of embodiment 99, wherein the disease or disorder is an infectious disease.

Embodiment 101. The vaccine composition of embodiment 100, wherein the infectious disease is an infection with a pathogen, optionally selected from a bacterium, virus, fungus, or parasite.

Embodiment 102. The vaccine composition of embodiment 101, wherein the pathogen is a virus.

Embodiment 103. The vaccine composition of embodiment 102, wherein the virus is: (a) an influenza virus, optionally selected from Type A, Type B, Type C, and Type D influenza viruses, or (b) a member of the Coronaviridae family, optionally selected from (i) a betacoronavirus, optionally selected from severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or (ii) an alphacoronavirus, optionally selected from HCoV-NL63 and HCoV-229E.

Embodiment 104. The vaccine composition of embodiment 103, wherein the virus is SARS-CoV-2.

Embodiment 105. The vaccine composition of any one of embodiments 78-104, wherein the adjuvant is a nucleic acid encoding the chimeric protein or chimeric protein complex.

Embodiment 106. The vaccine composition of any one of embodiments 78-105, wherein the antigen is a protein or an antigenic fragment of a protein.

Embodiment 107. The vaccine composition of any one of embodiments 78-105, wherein the antigen is a nucleic acid encoding a protein or an antigenic fragment of a protein.

Embodiment 108. The vaccine composition of embodiment 105 or 107, wherein the nucleic acid is mRNA, optionally comprising one or more non-canonical nucleotides, optionally selected from pseudouridine and 5-methoxyuridine.

Embodiment 109. The vaccine composition of embodiment 105 or 107, wherein the nucleic acid is DNA, optionally selected from linear DNA, DNA fragments, or DNA plasmids.

Embodiment 110. The vaccine composition of embodiment 106-109, wherein the antigen is a 2019-nCoV protein, an antigenic fragment thereof, or a nucleic acid encoding the same, wherein the 2019-nCoV protein is optionally selected from spike surface glycoprotein, membrane glycoprotein M, envelope protein E, and nucleocapsid phosphoprotein N.

Embodiment 111. The vaccine composition of embodiment 110, wherein the spike surface glycoprotein is the S1 or S2 subunit, or an antigenic fragment thereof.

Embodiment 112. The vaccine composition of embodiment 111, wherein the spike surface glycoprotein comprises the amino acid sequence of SEQ ID NO: 500, membrane glycoprotein precursor M comprises the amino acid sequence of SEQ ID NO: 501, the envelope protein E comprises the amino acid sequence of SEQ ID NO: 502, and the nucleocapsid phosphoprotein N comprises the amino acid sequence of SEQ ID NO: 503, or an amino acid sequence at least about 90%, or at least about 95%, or at least about 97%, or at least about 98%, or at least about 99% identity with any of the foregoing, or an antigenic fragment of any of the foregoing.

Embodiment 113. The vaccine composition of embodiment 103, wherein the virus is an influenza virus.

Embodiment 114. The vaccine composition of embodiment 113, wherein the antigen is an influenza viral antigen, an antigenic fragment thereof, or a nucleic acid encoding the same, wherein the viral antigen is optionally selected from hemagglutinin (HA) protein, matrix 2 (M2) protein, and neuraminidase, or an antigenic fragment thereof, or a nucleic acid encoding the same.

Embodiment 115. The vaccine composition of embodiment 100, wherein the disease or disorder is selected from diphtheria, tetanus, pertussis, influenza, pneumonia, hepatitis A, hepatitis B, polio, yellow fever, Human Papillomavirus (HPV) infection, anthrax, rabies, Japanese Encephalitis, meningitis, measles, mumps, rubella, gastroenteritis, smallpox, typhoid fever, varicella (chickenpox), rotavirus, and shingles.

Embodiment 116. The vaccine composition of any one of embodiments 78-115, wherein the antigen is that of one or more of the following vaccines: DTP (diphtheria-tetanus-pertussis vaccine), DTaP (diphtheria-tetanus-acellular pertussis vaccine), Hib (Haemophilus influenzae type b) conjugate vaccines, Pneumococcal conjugate vaccine, Hepatitis A vaccines, Poliomyelitis vaccines, Yellow fever vaccines, Hepatitis B vaccines, combination DTaP, Tdap, Hib, Human Papillomavirus (HPV) vaccine, Anthrax vaccine, and Rabies vaccine.

Embodiment 117. The vaccine composition of embodiment 99, wherein the cancer is selected form one or more of basal cell carcinoma, biliary tract cancer, bladder cancer, bone cancer, brain and central nervous system cancer, breast cancer, cancer of the peritoneum, cervical cancer, choriocarcinoma, colon and rectum cancer, connective tissue cancer, cancer of the digestive system, endometrial cancer, esophageal cancer, eye cancer, cancer of the head and neck, gastric cancer (including gastrointestinal cancer), glioblastoma, hepatic carcinoma, hepatoma, intra-epithelial neoplasm, kidney or renal cancer, larynx cancer, leukemia, liver cancer, lung cancer (e.g., small-cell lung cancer, non-small cell lung cancer, adenocarcinoma of the lung, and squamous carcinoma of the lung), melanoma, myeloma, neuroblastoma, oral cavity cancer (lip, tongue, mouth, and pharynx), ovarian cancer, pancreatic cancer, prostate cancer, retinoblastoma, rhabdomyosarcoma, rectal cancer, cancer of the respiratory system, salivary gland carcinoma, sarcoma (e.g., Kaposi's sarcoma), skin cancer, squamous cell cancer, stomach cancer, testicular cancer, thyroid cancer, uterine or endometrial cancer, cancer of the urinary system, vulval cancer, lymphoma including Hodgkin's and non-Hodgkin's lymphoma, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), hairy cell leukemia, chronic myeloblastic leukemia as well as other carcinomas and sarcomas, post-transplant lymphoproliferative disorder (PTLD) as well as abnormal vascular proliferation associated with phakomatoses, edema (e.g. that associated with brain tumors), and Meigs' syndrome.

Embodiment 118. The vaccine composition of embodiments 99, wherein the autoimmune disease or disorder is selected from Crohn's disease, diabetes, multiple sclerosis, systemic lupus erythematosis, rheumatoid arthritis or juvenile rheumatoid arthritis, and ulcerative colitis.

Embodiment 119. The vaccine composition of any one of embodiments 78-118, wherein the connector between (i) and (ii) is a flexible linker.

Embodiment 120. The vaccine composition of embodiment 119, wherein the flexible linker is substantially comprised of glycine and serine residues, optionally wherein i) the flexible linker comprises (Gly₄Ser)ₙ, where n is from about 1 to about 8; (ii) the flexible linker comprises (Gly₂Ser)ₙ, where n is from about 1 to about 20; or iii) the flexible linker comprises one or more of SEQ ID NOs: 435-442.

Embodiment 121. The vaccine composition of embodiment 119, wherein the flexible linker is substantially comprised of GGSGGSGGGGSGGGGS (SEQ ID NO: 257).

Embodiment 122. The vaccine composition of embodiment 119, wherein the flexible linker is substantially comprised of LE, GGGGS (SEQ ID NO: 249), (GGGGS)n (n=1-4) (SEQ ID NO: 249-SEQ ID NO: 252), (Gly)8 (SEQ ID NO: 258), (Gly)6 (SEQ ID NO: 259), (EAAAK)n (n=1-3) (SEQ ID NO: 260-SEQ ID NO: 262), A(EAAAK)nA (n=2-5) (SEQ ID NO: 263-SEQ ID NO: 266), AEAAAKEAAAKA (SEQ ID NO: 263), A(EAAAK) 4ALEA(EAAAK)4A (SEQ ID NO: 267), PAPAP (SEQ ID NO: 268), KESGSVSSEQLAQFRSLD (SEQ ID NO: 269), EGKSSGSGSESKST (SEQ ID NO: 270), GSAGSAAGSGEF (SEQ ID NO: 271), and (XP)n, with X designating any amino acid, e.g., Ala, Lys, or Glu.

Embodiment 123. The vaccine composition of any one of embodiments 78-122, wherein the wherein the Fc domain is from IgG, IgA, IgD, IgM or IgE.

Embodiment 124. The vaccine composition of embodiment 123, wherein the IgG is selected from IgG1, IgG2, IgG3, or IgG4.

Embodiment 125. The vaccine composition of embodiment 123, wherein the Fc domain is from human IgG, IgA, IgD, IgM or IgE.

Embodiment 126. The vaccine composition of embodiment 125, wherein the human IgG is selected from human IgG1, IgG2, IgG3, or IgG4.

Embodiment 127. The vaccine composition of any one of embodiments 78-126, wherein the Fc chain pairing is promoted by ionic pairing and/or a knob-in-hole pairing.

Embodiment 128. The vaccine composition of embodiment 125, wherein the one or more mutations to the Fc domain results in an ionic pairing between the Fc chains in the Fc domain.

Embodiment 129. The vaccine composition of embodiment 125, wherein the one or more mutations to the Fc domain results in a knob-in-hole pairing in the Fc domain.

Embodiment 130. The vaccine composition of any one of embodiments 78-129, wherein the one or more mutations to the Fc domain results in the reduction or elimination of the effector function of the Fc domain.

Embodiment 131. The vaccine composition of any one of embodiments 78-130, wherein the chimeric protein complex is a heterodimer and has a trans orientation.

Embodiment 132. The vaccine composition of any one of embodiments 78-131, wherein the chimeric protein-complex is a heterodimer and has a cis orientation.

Embodiment 133. The vaccine composition of any one of embodiments 78-132, wherein the Fc domain comprises L234A, L235A, and K322Q substitutions in human IgG1 (according to EU numbering).

Embodiment 134. The vaccine composition of embodiment 133, wherein the Fc domain is human IgG1, and optionally contains one or more of L234, L235, K322, D265, P329, and P331 (according to EU numbering).

Embodiment 135. The vaccine composition of any one of embodiments 78-134, wherein the chimeric protein complex has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, and 19A-F.

Embodiment 136. The vaccine composition of any one of embodiments 78-135, wherein the vaccine composition is formulated for administration intravenously.

Embodiment 137. The vaccine composition of any one of embodiments 78-136, wherein the vaccine composition is formulated for administration to the lung.

Embodiment 138. The vaccine composition of any one of embodiments 78-137, wherein the vaccine composition is formulated for administration by inhalation.

Embodiment 139. The vaccine composition of any one of embodiments 78-138, wherein the vaccine composition is formulated for administration via aerosol or nebulizer.

Embodiment 140. The vaccine composition of any one of embodiments 78-139, wherein the vaccine composition is formulated for administration liquid nebulization, dry powder dispersion and meter-dose administration.

Embodiment 141. The vaccine composition of any one of embodiments 78-140, wherein the adjuvant or vaccine composition has
(a) low toxicity;
(b) an ability to stimulate a long-lasting immune response against the antigen;
(c) substantial stability;
(d) an ability to elicit a humoral immune response and/or a cell-mediated immunity to the antigen;
(e) a capability of selectively interacting with populations of antigen presenting cells;
(f) an ability to specifically elicit TH1 and/or TH2 cell-specific immune responses to the antigen; and/or
(g) an ability to selectively increase appropriate antibody isotype levels against antigens, the isotype optionally being IgA,

when administered to a patient.

Embodiment 142. The vaccine composition of any one of embodiments 78-141, wherein the adjuvant or vaccine composition stimulates a CD8⁺ T cell response to the antigen, when administered to a patient.

Embodiment 143. The vaccine composition of any one of embodiments 78-142, wherein the adjuvant or vaccine composition stimulates activation of the IL-1R, when administered to a patient.

Embodiment 144. The vaccine composition of any one of embodiments 78-143, wherein the adjuvant or vaccine composition does not substantially cause one or more of fever, neutrophilia and the release of acute phase proteins when administered to a patient.

Embodiment 145. A method for vaccinating a subject against an infectious disease, comprising administering: (a) an adjuvant comprising a chimeric protein or chimeric protein complex, comprising: (i) an interleukin-1α (IL-1α), pro-IL-1α, or a mutant thereof, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) and/or (2) a flexible linker that connects (i) and (ii); wherein the mutant IL-1α or the mutant pro-IL-1α is characterized by low affinity or activity at the IL-1 receptor; and (b) an antigen which is suitable for inducing an immune response.

Embodiment 146. The method of embodiment 145, wherein the adjuvant and antigen are administered concurrently.

Embodiment 147. The method of embodiment 145, wherein the adjuvant complex and antigen are co-formulated.

Embodiment 148. The method of embodiment 145, wherein the adjuvant and antigen are administered sequentially.

Embodiment 149. The method of embodiment 145, wherein the adjuvant and antigen are administered in multiple doses.

Embodiment 150. The method of embodiment 145, wherein the adjuvant is administered in multiple booster doses and the antigen is administered once.

Embodiment 151. The method of any one of embodiments 145-150, wherein the IL-1α, pro-IL-1α, or a mutant thereof are human.

Embodiment 152. The method of any one of embodiments 145-151, wherein the low affinity or activity at the IL-1 receptor is restorable by attachment to one or more targeting moieties or upon inclusion in the chimeric protein complex.

Embodiment 153. The method of any one of embodiments 145-152, wherein: (a) the mutant human IL-1α has an amino acid sequence of at least 95%, or 97% or 98% identity to SEQ ID NO: 1 or 3, or (b) the mutant human pro-IL-1α has an amino acid sequence of at least 95%, or 97% or 98% identity to SEQ ID NO: 2 or 4.

Embodiment 154. The method of embodiment 153, wherein the mutant IL-1α comprises one or more mutations selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S, Q153A, and Q153G.

Embodiment 155. The method of embodiment 153, wherein the mutant pro-IL-1α comprises one or selected from P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G.

Embodiment 156. The method of any one of embodiments 145-155, wherein the targeting moiety comprises a recognition domain that recognizes and/or binds an antigen or receptor on a tumor cell, endothelial cell, epithelial cell, mesenchymal cell, stromal cell, ECM and/or immune cell, organ cell, and/or tissue cell.

Embodiment 157. The method of embodiment 156, wherein the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof.

Embodiment 158. The method of embodiment 157, wherein the immune cell is a T cell.

Embodiment 159. The method of any one of embodiments 145-158, wherein the targeting moiety comprises a recognition domain that recognizes and/or binds CD8.

Embodiment 160. The method of any one of embodiments 145-159, wherein the targeting moiety comprises a recognition domain that is a full-length antibody or a fragment thereof, a single-domain antibody, a recombinant heavy-chain-only antibody (VHH), a single-chain antibody (scFv), a Humabody, a shark heavy-chain-only antibody (VNAR), a microprotein (e.g. cysteine knot protein, knottin), a darpin, an anticalin, an adnectin, an aptamer, a Fv, a Fab, a Fab′, a F(ab′)₂, a peptide mimetic molecule, a natural ligand for a receptor, or a synthetic molecule.

Embodiment 161. The method of any one of embodiments 145-160, wherein the chimeric protein or chimeric protein complex further comprises additional cytokines, wherein the additional cytokines are optionally modified, wherein the modification is optionally a mutation.

Embodiment 162. The method of any one of embodiments 145-161, wherein the chimeric protein or chimeric protein complex further comprises one or more additional targeting moieties.

Embodiment 163. The method of any one of embodiments 145-162, wherein the chimeric protein or chimeric protein complex further comprises two signaling agents and/or two targeting moieties or two of both.

Embodiment 164. The method of any one of embodiments 145-163, wherein the chimeric protein or chimeric protein complex further comprises three signaling agents and/or three targeting moieties or three of both.

Embodiment 165. The method of any one of embodiments 145-164, wherein the targeting moiety comprises a recognition domain that recognizes and/or binds CD8 or CD4 and the mutant IL-1α comprises one or more mutations selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S, Q153A, and Q153G.

Embodiment 166. The method of any one of embodiments 145-164, wherein the targeting moiety comprises a recognition domain that recognizes and/or binds CD8 or CD4 and the mutant pro-IL-1α comprises one or more mutations of the mutant pro-IL-1α is selected from P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G.

Embodiment 167. The method of any one of embodiments 145-166, further comprising an aluminum gel or salt.

Embodiment 168. The method of embodiment 167, wherein the aluminum gel or salt is selected from aluminum hydroxide, aluminum phosphate, and aluminum sulfate.

Embodiment 169. The method of any one of embodiments 145-168, wherein the vaccine further comprises an additional adjuvant selected from oil-in-water emulsion formulations, saponin adjuvants, Freunds Adjuvants, toll like receptors ligands, cytokines, and chitosans.

Embodiment 170. The method of embodiment 169, wherein the infectious disease is an infection with a pathogen, optionally selected from a bacterium, virus, fungus, or parasite.

Embodiment 171. The method of embodiment 169, wherein the pathogen is a virus.

Embodiment 172. The method of embodiment 171, wherein the virus is: (a) an influenza virus, optionally selected from Type A, Type B, Type C, and Type D influenza viruses, or (b) a member of the Coronaviridae family, optionally selected from (i) a betacoronavirus, optionally selected from severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or (ii) alphacoronavirus, optionally selected from HCoV-NL63 and HCoV-229E.

Embodiment 173. The method of embodiment 172, wherein the virus is SARS-CoV-2.

Embodiment 174. The method of any one of embodiments 145-173, wherein the adjuvant is a nucleic acid encoding the chimeric protein or chimeric protein complex.

Embodiment 175. The method of any one of embodiments 145-173, wherein the antigen is a protein or an antigenic fragment of a protein.

Embodiment 176. The method of any one of embodiments 145-173, wherein the antigen is a nucleic acid encoding a protein or an antigenic fragment of a protein.

Embodiment 177. The method of embodiment 174, wherein the nucleic acid is mRNA, optionally comprising one or more non-canonical nucleotides, optionally selected from pseudouridine and 5-methoxyuridine.

Embodiment 178. The method of embodiment 174, wherein the nucleic acid is DNA, optionally selected from linear DNA, DNA fragments, or DNA plasmids.

Embodiment 179. The method of embodiment 175-178, wherein the antigen is a 2019-nCoV protein, an antigenic fragment thereof or a nucleic acid encoding the same, wherein the 2019-nCoV protein is optionally selected from spike surface glycoprotein, membrane glycoprotein M, envelope protein E, and nucleocapsid phosphoprotein N.

Embodiment 180. The method of embodiment 179, wherein the spike surface glycoprotein is the S1 or S2 subunit, or an antigenic fragment thereof.

Embodiment 181. The method of embodiment 180, wherein the spike surface glycoprotein comprises the amino acid sequence of SEQ ID NO: 500, membrane glycoprotein precursor M comprises the amino acid sequence of SEQ ID NO: 501, the envelope protein E comprises the amino acid sequence of SEQ ID NO: 502, and the nucleocapsid phosphoprotein N comprises the amino acid sequence of SEQ ID NO: 503, or an amino acid sequence at least about 90%, or at least about 95%, or at least about 97%, or at least about 98%, or at least about 99% identity with any of the foregoing, or an antigenic fragment of any of the foregoing.

Embodiment 182. The method of embodiment 172, wherein the virus is an influenza virus. 183. The method of embodiment 182, wherein the antigen is an influenza viral antigen, an antigenic fragment thereof, or a nucleic acid encoding the same, wherein the viral antigen is optionally selected from hemagglutinin (HA) protein, matrix 2 (M2) protein, and neuraminidase, or an antigenic fragment thereof.

Embodiment 184. The method of any one of embodiments 145-183, wherein the connector between (i) and (ii) is a flexible linker.

Embodiment 185. The method of embodiment 184, wherein the flexible linker is substantially comprised of glycine and serine residues, optionally wherein i) the flexible linker comprises (Gly₄Ser)ₙ, where n is from about 1 to about 8; (ii) the flexible linker comprises (Gly₂Ser)ₙ, where n is from about 1 to about 20; or iii) the flexible linker comprises one or more of SEQ ID NOs: 435-442.

Embodiment 186. The method of embodiment 185, wherein the flexible linker is substantially comprised of GGSGGSGGGGSGGGGS (SEQ ID NO: 257).

Embodiment 187. The method of embodiment 184, wherein the flexible linker is substantially comprised of LE, GGGGS (SEQ ID NO: 249), (GGGGS)n (n=1-4) (SEQ ID NO: 249-SEQ ID NO: 252), (Gly)8 (SEQ ID NO: 258), (Gly)6 (SEQ ID NO: 259), (EAAAK)n (n=1-3) (SEQ ID NO: 260-SEQ ID NO: 262), A(EAAAK)nA (n=2-5) (SEQ ID NO: 263-SEQ ID NO: 266), AEAAAKEAAAKA (SEQ ID NO: 263), A(EAAAK)4ALEA(EAAAK)4A (SEQ ID NO: 267), PAPAP (SEQ ID NO: 268), KESGSVSSEQLAQFRSLD (SEQ ID NO: 269), EGKSSGSGSESKST (SEQ ID NO: 270), GSAGSAAGSGEF (SEQ ID NO: 271), and (XP)n, with X designating any amino acid, e.g., Ala, Lys, or Glu.

Embodiment 188. The method of any one of embodiments 145-187, wherein the wherein the Fc domain is from IgG, IgA, IgD, IgM or IgE.

Embodiment 189. The method of embodiment 188, wherein the IgG is selected from IgG1, IgG2, IgG3, or IgG4.

Embodiment 190. The method of embodiment 188, wherein the Fc domain is from human IgG, IgA, IgD, IgM or IgE.

Embodiment 191. The method of embodiment 190, wherein the human IgG is selected from human IgG1, IgG2, IgG3, or IgG4.

Embodiment 192. The method of any one of embodiments 145-191, wherein the Fc chain pairing is promoted by ionic pairing and/or a knob-in-hole pairing.

Embodiment 193. The method of embodiment 192, wherein the one or more mutations to the Fc domain results in an ionic pairing between the Fc chains in the Fc domain.

Embodiment 194. The method of embodiment 192, wherein the one or more mutations to the Fc domain results in a knob-in-hole pairing in the Fc domain.

Embodiment 195. The method of any one of embodiments 145-194, wherein the one or more mutations to the Fc domain results in the reduction or elimination of the effector function of the Fc domain.

Embodiment 196. The method of any one of embodiments 145-195, wherein the Fc-based chimeric protein complex is a heterodimer and has a trans orientation.

Embodiment 197. The method of any one of embodiments 145-196, wherein the Fc-based chimeric protein-complex is a heterodimer and has a cis orientation.

Embodiment 198. The method of any one of embodiments 145-197, wherein the Fc comprises L234A, L235A, and K322Q substitutions in human IgG1 (according to EU numbering).

Embodiment 199. The method of embodiment 198, wherein the Fc is human IgG1, and optionally contains one or more of L234, L235, K322, D265, P329, and P331 (according to EU numbering).

Embodiment 200. The method of any one of embodiments 145-199, wherein the Fc-based chimeric protein has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, and 19A-F.

Embodiment 201. The method of any one of embodiments 145-200, wherein the adjuvant and/or the antigen are formulated for administration intravenously.

Embodiment 202. The method of any one of embodiments 145-200, wherein the adjuvant and/or the antigen are formulated for administration to the lung.

Embodiment 203. The method of any one of embodiments 145-200, wherein the adjuvant and/or the antigen are formulated for administration by inhalation.

Embodiment 204. The method of any one of embodiments 145-200, wherein the adjuvant and/or the antigen are formulated for administration via aerosol or nebulizer.

Embodiment 205. The method of any one of embodiments 145-200, wherein the adjuvant and/or the antigen are formulated for administration liquid nebulization, dry powder dispersion and meter-dose administration.

Embodiment 206. The method of any one of embodiments 145-205, wherein the adjuvant has (a) low toxicity; (b) an ability to stimulate a long-lasting immune response against the antigen; (c) substantial stability; (d) an ability to elicit a humoral immune response and/or a cell-mediated immunity to the antigen; (e) a capability of selectively interacting with populations of antigen presenting cells; (f) an ability to specifically elicit T_{H1} and/or T_{H2} cell-specific immune responses to the antigen; and/or (g) an ability to selectively increase appropriate antibody isotype levels against antigens, the isotype optionally being IgA, when administered to a patient.

Embodiment 207. The method of any one of embodiments 145-206, wherein the adjuvant stimulates a CD8⁺ T cell response to the antigen, when administered to a patient.

Embodiment 208. The method of any one of embodiments 145-207, wherein the adjuvant or vaccine composition stimulates activation of the IL-1R, when administered to a patient.

Embodiment 209. The method of any one of embodiments 145-208, wherein the adjuvant does not substantially cause one or more of fever, neutrophilia and the release of acute phase proteins when administered to a patient.

Embodiment 210. A method for treating a subject afflicted with an infectious disease, comprising administering a chimeric protein or chimeric protein complex, comprising: (i) an interleukin-1α (IL-1α), pro-IL-1α, or a mutant thereof, (ii) one or more targeting moieties, said targeting moieties comprising recognition domains which specifically bind to an antigen or receptor of interest; and (iii) a connector between (i) and (ii), the connector being: (1) an Fc domain, the Fc domain optionally having one or more mutations that reduces or eliminates one or more effector functions of the Fc domain, promotes Fc chain pairing in the Fc domain, and/or stabilizes a hinge region in the Fc domain that connects (i) and (ii) and/or (2) a flexible linker that connects (i) and (ii); wherein the mutant IL-1α or the mutant pro-IL-1α is characterized by low affinity or activity at the IL-1 receptor.

Embodiment 211. The method of embodiment 210, wherein the IL-1α, pro-IL-1α, or a mutant thereof are human.

Embodiment 212. The method of any one of embodiments 210-211, wherein the low affinity or activity at the IL-1 receptor is restorable by attachment to one or more targeting moieties or upon inclusion in the chimeric protein complex.

Embodiment 213. The method of any one of embodiments 210-212, wherein: (a) the mutant human IL-1α has an amino acid sequence of at least 95%, or 97% or 98% identity to SEQ ID NO: 1 or 3 or (b) the mutant human pro-IL-1α has an amino acid sequence of at least 95%, or 97% or 98% identity to SEQ ID NO: 2 or 4.

Embodiment 214. The method of any one of embodiments 210-213, wherein the mutant IL-1α comprises one or more mutations selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S,
Q153A, and Q153G.

Embodiment 215. The method of any one of embodiments 210-213, wherein the mutant pro-IL-1α comprises one or more mutations selected from P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G.

Embodiment 216. The method of any one of embodiments 210-215, wherein the targeting moiety comprises a recognition domain that recognizes and/or binds an antigen or receptor on an endothelial cell, epithelial cell, mesenchymal cell, stromal cell, ECM and/or immune cell, organ cell, and/or tissue cell.

Embodiment 217. The method of embodiment 216, wherein the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof.

Embodiment 218. The method of embodiment 217, wherein the immune cell is a T cell.

Embodiment 219. The method of any one of embodiments 210-218, wherein the targeting moiety comprises a recognition domain that recognizes and/or binds CD8.

Embodiment 220. The method of any one of embodiments 210-218, wherein the targeting moiety comprises a recognition domain that is a full-length antibody or a fragment thereof, a single-domain antibody, a recombinant heavy-chain-only antibody (V_{H}H), a single-chain antibody (scFv), a Humabody, a shark heavy-chain-only antibody (VNAR), a microprotein (e.g. cysteine knot protein, knottin), a darpin, an anticalin, an adnectin, an aptamer, a Fv, a Fab, a Fab′, a F(ab′)₂, a peptide mimetic molecule, a natural ligand for a receptor, or a synthetic molecule.

Embodiment 221. The method of any one of embodiments 210-220, wherein the chimeric protein or chimeric protein complex further comprises additional cytokines, wherein the additional cytokines are optionally modified, wherein the modification is optionally a mutation.

Embodiment 222. The method of any one of embodiments 210-221, wherein the chimeric protein or chimeric protein complex further comprises one or more additional targeting moieties.

Embodiment 223. The method of any one of embodiments 210-222, wherein the chimeric protein or chimeric protein complex further comprises two signaling agents and/or two targeting moieties or two of both.

Embodiment 224. The method of any one of embodiments 210-223, wherein the chimeric protein or chimeric protein complex further comprises three signaling agents and/or three targeting moieties or three of both.

Embodiment 225. The method of any one of embodiments 210-224, wherein the mutant IL-1α comprises one or more mutations selected from a deletion of amino acids 1-6 (del1-6), an amino acid substitution at a position selected from P3, M15, R16, 117, 118, L24, N25, D26, L28, N29, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, C141, L142, D151, F152, Q153 and a combination thereof, wherein the positions are in reference to SEQ ID NO: 1 or 3, optionally selected from M15A, M15G, M15S, R16A, R16K, R16G, 118A, 118G, 118L, L24K, L24S, N25A, N25G, D26V, L28A, L28G, N29A, N29G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, C141S, C141H, D151A, D151K, D151Y, F152Q, F152N, F152S, Q153A, and Q153G and the targeting moiety comprises a recognition domain that recognizes and/or binds CD8 or CD4.

Embodiment 226. The method of any one of embodiments 210-224, wherein the mutant pro-IL-1α comprises P115, M127, R128, 1129, 1130, L136, N137, D138, L140, N141, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from M127S, R128A, R128K, 1130A, 1130L, L136K, L136S, N137A, N137G, D138V, L140A, L140G, N141A, N141G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G and the targeting moiety comprises a recognition domain that recognizes and/or binds CD8 or CD4.

Embodiment 227. The method of any one of embodiments 210-226, further comprising an aluminum gel or salt.

Embodiment 228. The method of embodiment 227, wherein the aluminum gel or salt is selected from aluminum hydroxide, aluminum phosphate, and aluminum sulfate.

Embodiment 229. The method of any one of embodiments 210-228, wherein the vaccine further comprises an additional adjuvant selected from oil-in-water emulsion formulations, saponin adjuvants, Freunds Adjuvants, toll like receptors ligands, cytokines, and chitosans.

Embodiment 230. The method of embodiment 229, wherein the infectious disease is an infection with a pathogen, optionally selected from a bacterium, virus, fungus, or parasite.

Embodiment 231. The method of embodiment 229, wherein the pathogen is a virus.

Embodiment 232. The method of embodiment 231, wherein the virus is: (a) an influenza virus, optionally selected from Type A, Type B, Type C, and Type D influenza viruses, or (b) a member of the Coronaviridae family, optionally selected from (i) a betacoronavirus, optionally selected from severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or (ii) an alphacoronavirus, optionally selected from HCoV-NL63 and HCoV-229E.

Embodiment 233. The method of embodiment 232, wherein the virus is SARS-CoV-2.

Embodiment 234. The method of embodiment 232, wherein the virus is an influenza virus.

Embodiment 235. The method of any one of embodiments 210-234, wherein the connector between (i) and (ii) is a flexible linker.

Embodiment 236. The method of embodiment 235, wherein the flexible linker is substantially comprised of glycine and serine residues, optionally wherein i) the flexible linker comprises (Gly₄Ser)ₙ, where n is from about 1 to about 8; (ii) the flexible linker comprises (Gly₂Ser)ₙ, where n is from about 1 to about 20; or iii) the flexible linker comprises one or more of SEQ ID NOs: 435-442.

Embodiment 237. The method of embodiment 236, wherein the flexible linker is substantially comprised of GGSGGSGGGGSGGGGS (SEQ ID NO: 257).

Embodiment 238. The method of embodiment 237, wherein the flexible linker is substantially comprised of LE, GGGGS (SEQ ID NO: 249), (GGGGS)n (n=1-4) (SEQ ID NO: 249-SEQ ID NO: 252), (Gly)8 (SEQ ID NO: 258), (Gly)6 (SEQ ID NO: 259), (EAAAK)n (n=1-3) (SEQ ID NO: 260-SEQ ID NO: 262), A(EAAAK)nA (n=2-5) (SEQ ID NO: 263-SEQ ID NO: 266), AEAAAKEAAAKA (SEQ ID NO: 263), A(EAAAK)4ALEA(EAAAK)4A (SEQ ID NO: 267), PAPAP (SEQ ID NO: 268), KESGSVSSEQLAQFRSLD (SEQ ID NO: 269), EGKSSGSGSESKST (SEQ ID NO: 270), GSAGSAAGSGEF (SEQ ID NO: 271), and (XP)n, with X designating any amino acid, e.g., Ala, Lys, or Glu.

Embodiment 239. The method of any one of embodiments 210-238, wherein the wherein the Fc domain is from IgG, IgA, IgD, IgM or IgE.

Embodiment 240. The method of embodiment 239, wherein the IgG is selected from IgG1, IgG2, IgG3, or IgG4.

Embodiment 241. The method of embodiment 239, wherein the Fc domain is from human IgG, IgA, IgD, IgM or IgE.

Embodiment 242. The method of embodiment 241, wherein the human IgG is selected from human IgG1, IgG2, IgG3, or IgG4.

Embodiment 243. The method of any one of embodiments 210-242, wherein the Fc chain pairing is promoted by ionic pairing and/or a knob-in-hole pairing.

Embodiment 244. The method of embodiment 243, wherein the one or more mutations to the Fc domain results in an ionic pairing between the Fc chains in the Fc domain.

Embodiment 245. The method of embodiment 243, wherein the one or more mutations to the Fc domain results in a knob-in-hole pairing in the Fc domain.

Embodiment 246. The method of any one of embodiments 210-245, wherein the one or more mutations to the Fc domain results in the reduction or elimination of the effector function of the Fc domain.

Embodiment 247. The method of any one of embodiments 210-246, wherein the Fc-based chimeric protein complex is a heterodimer and has a trans orientation.

Embodiment 248. The method of any one of embodiments 210-247, wherein the Fc-based chimeric protein-complex is a heterodimer and has a cis orientation.

Embodiment 249. The method of any one of embodiments 210-248, wherein the Fc comprises L234A, L235A, and K322Q substitutions in human IgG1 (according to EU numbering).

Embodiment 250. The method of embodiment 249, wherein the Fc is human IgG1, and optionally contains one or more of L234, L235, K322, D265, P329, and P331 (according to EU numbering).

Embodiment 251. The method of any one of embodiments 210-250, wherein the Fc-based chimeric protein complex has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, and 19A-F.

Embodiment 252. The method of any one of embodiments 210-251, wherein the chimeric protein or chimeric protein complex stimulates a CD8⁺ T cell response to the antigen, when administered to a patient.

Embodiment 253. The method of any one of embodiments 210-252, wherein the adjuvant or vaccine composition stimulates activation of the IL-1R, when administered to a patient.

Embodiment 254. The method of any one of embodiments 210-253, wherein the chimeric protein or chimeric protein complex does not substantially cause one or more of fever, neutrophilia and the release of acute phase proteins when administered to a patient.

What is claimed is:

1. A chimeric protein or chimeric protein complex comprising:
(a) a mutant interleukin-1α (IL-1α), wherein the mutation is a deletion of amino acids 1-6 (delta 1-6) with respect to SEQ ID NO: 1 or 3, or a mutant pro-IL-1α, wherein the mutation is a deletion of amino acids 113-118 (delta 113-118) with respect to SEQ ID NO: 2 or 4, and
(b) one or more targeting moieties, said targeting moieties comprising recognition domains which specifically bind to an antigen or receptor of interest; and
(c) a connector between (a) and (b), the connector being:
(i) a flexible linker that connects (a) and (b), and/or
(ii) an Fc domain that connects (a) and (b), the Fc domain optionally having one or more mutations that reduces or eliminates one or more effector functions of the Fc domain, promotes Fc chain pairing in the Fc domain, and/or stabilizes a hinge region in the Fc domain.

2. The chimeric protein or chimeric protein complex of claim 1, wherein the mutant interleukin-1α (IL-1α) further comprises an amino acid substitution at position C141, optionally wherein the substitution is C141S or C141A or C141H, with respect to SEQ ID NO: 1 or 3; or wherein the mutant pro-IL-1α further comprises an amino acid substitution at position C253, optionally wherein the substitution is C253S or C253A or C253H, with respect to SEQ ID NO: 2 or 4.

3. The chimeric protein or chimeric protein complex of claim 1 or 2, wherein the mutant IL-1α comprises one or more additional mutations selected from an amino acid substitution at a position selected from N29, S31, P3, M15, R16, 117, 118, L24, N25, D26, L28, 133, L40, A44, H46, V52, F54, M56, A58, Y59, K60, D64, D65, K67, 168, V70, L72, L79, Y80, P89, L91, E94, P99, K100, E106, F111, W113, K119, S124, P128, 1132, Q136, T134, V140, L142, D151, F152, Q153 and a combination thereof, wherein the positions are in reference to SEQ ID NO: 1 or 3, optionally selected from N29A, N29D, N29G, S31A, S31G, M15A, M15G, M15S, R16A, R16K, R16G, 118A, 118G, 118L, L24K, L24S, N25A, N25G, D26V, L28A, L28G, 133A, 133G, A44G, A44S, A44T, A44N, A44H, H46A, H46G, A58G, A58S, A58T, A58N, A58H, A58F, Y59A, K60A, K60G, D64A, D64G, D65A, K67A, 168A, 168G, V70A, Y80A, K100A, K100D, W113F, Q136A, Q136C, D151A, D151K, D151Y, F152Q, F152N, F152S, Q153A, and Q153G and a combination thereof.

4. The chimeric protein or chimeric protein complex of claim 1 or 2, wherein the mutant pro-IL-1α comprises one or more additional mutations selected from an amino acid substitution at a position selected from N141, S143, P115, M127, R128, 1129, 1130, L136, N137, D138, L140, 1145, L152, A156, H158, V164, F166, M168, A170, A171, K172, D176, D177, K179, 1180, V182, L184, L191, Y192, P201, L203, E206, P211, K212, E218, F223, W225, K231, S236, P240, 1244, Q248, T246, V252, C253, L254, D263, F264, Q265 and a combination thereof, wherein the positions are in reference to SEQ ID NO: 2 or 4, optionally selected from N141A, N141D, N141G, S143A, S143G, M127S, R128A, R128K, 1130A, 1130L, L136K, L136S, N137A, N137G, D138V, L140A, L140G, A156G, A156S, A156T, A156N, A156H, H158A, H158G, A170G, A170S, A170T, A170N, A170H, A170F, Y171A, K172A, K172G, D176A, D176G, D177A, 1180A, 180G, V182A, Y192A, K212A, K212D, W225F, Q248C, D263K, F264Q, F264N, F264S, Q265A, and Q265G and a combination thereof.

5. The chimeric protein or chimeric protein complex of any one of claims 2-4, wherein the one or more additional mutations confer reduced activity as compared to an IL-1α having the amino acid sequence of SEQ ID NO: 1 or 3 having a deletion of amino acids 1-6 (delta 1-6), or compared to a pro-IL-1α having the amino acid sequence of SEQ ID NO: 2 or 4 having a deletion of amino acids 113-118 (delta 113-118).

6. The chimeric protein or chimeric protein complex of claim 5, wherein the one or more mutations confer reduced activity that is inducible and/or restorable by attachment to one or more targeting moieties or upon inclusion in the chimeric protein or chimeric protein complex.

7. The chimeric protein or chimeric protein complex of any one of claims 3-4, wherein the one or more additional mutations confer increased activity as compared to an IL-1α having the amino acid sequence of SEQ ID NO: 1 or 3 having a deletion of amino acids 1-6 (delta 1-6), optionally wherein the one or more additional mutations are selected from an amino acid substitution at a position selected from N29 and S31 with respect to SEQ ID NO: 1 or 3, optionally wherein the substitution is selected from N29A, N29D, N29G, S31A, and S31G; or
wherein the one or more additional mutations confer increased activity as compared to a pro-IL-1α having the amino acid sequence of SEQ ID NO: 2 or 4 having a deletion of amino acids 113-118 (delta 113-118), optionally wherein the one or more additional mutations are selected from an amino acid substitution at a position selected from N141 and S143 with respect to SEQ ID NO: 2 or 4, optionally wherein the substitution is selected from N141A, N141D, N141G, S143A, and S143G.

8. The chimeric protein or chimeric protein complex of any one of the above claims, wherein:
(a) the IL-1α comprises an amino acid sequence having at least about 97%, or at least about 98%, or at least about 99% identity with SEQ ID NO: 1 or 3 or,
(b) the pro-IL-1α comprises an amino acid sequence having at least about 97%, or at least about 98%, or at least about 99% identity with SEQ ID NO: 2 or 4.

9. The chimeric protein or chimeric protein complex of any one of the above claims, wherein the one or more mutations of the mutant IL-1α or mutant pro-IL-1α confer reduced or increased activity and/or reduced or increased affinity for IL-1R or IL-1RAcP.

10. The chimeric protein or chimeric protein complex of claim 9, wherein the IL-1α or pro-IL-1α exhibits reduced or increased activity and/or reduced or increased affinity for IL-1R1.

11. The chimeric protein or chimeric protein complex of claim 9, wherein the IL-1α or pro-IL-1α exhibits reduced or increased activity and/or reduced or increased affinity for IL-1RAcP.

12. The chimeric protein or chimeric protein complex of any one of claims 1-11, wherein the targeting moiety is directed against a tumor cell.

13. The chimeric protein or chimeric protein complex of any one of claims 1-12, wherein the targeting moiety comprises a recognition domain that recognizes and/or binds an antigen or receptor on a tumor cell, endothelial cell, epithelial cell, mesenchymal cell, tumor stroma or stromal cell, ECM and/or immune cell, organ cells, and/or tissue cells.

14. The chimeric protein or chimeric protein complex of claim 13, wherein the immune cell is selected from a T cell, a B cell, a dendritic cell, a macrophage, a neutrophil, a mast cell, a monocyte, a red blood cell, myeloid cell, myeloid derived suppressor cell, a NKT cell, and a NK cell, or derivatives thereof.

15. The chimeric protein or chimeric protein complex of any one of claims 1-14, wherein the targeting moiety comprises a recognition domain that is a full-length antibody or a fragment thereof, a single-domain antibody, a recombinant heavy-chain-only antibody (VHH), a single-chain antibody (scFv), a Humabody, a shark heavy-chain-only antibody (VNAR), a microprotein (e.g. cysteine knot protein, knottin), a darpin, an anticalin, an adnectin, an aptamer, a Fv, a Fab, a Fab′, or a F(ab′)₂.

16. The chimeric protein or chimeric protein complex of claim 15, wherein the recognition domain is a single-domain antibody, optionally a VHH or humanized VHH.

17. The chimeric protein or chimeric protein complex of any one of claims 1-16, wherein the recognition domain functionally modulates the antigen or receptor of interest.

18. The chimeric protein or chimeric protein complex of any one of claims 1-16, wherein the recognition domain binds but does not functionally modulate the antigen or receptor of interest.

19. The chimeric protein or chimeric protein complex of any one of claims 1-18, comprising two or more targeting moieties.

20. The chimeric protein or chimeric protein complex of any one of claims 1-19, further comprising one or more additional modified signaling agents.

21. The chimeric protein or chimeric protein complex of claim 20, wherein the chimeric protein complex comprises two signaling agents or two targeting moieties or two of both.

22. The chimeric protein or chimeric protein complex of any one of claims 1-20, wherein the chimeric protein complex comprises three signaling agents or three targeting moieties or three of both.

23. The chimeric protein of claim 1, wherein the connector between (a) and (b) is a flexible linker.

24. The chimeric protein of claim 23, wherein the flexible linker is substantially comprised of glycine and serine residues, optionally wherein i) the flexible linker comprises (Gly₄Ser)ₙ, where n is from about 1 to about 8; (ii) the flexible linker comprises (Gly₂Ser)ₙ, where n is from about 1 to about 20; or iii) the flexible linker comprises one or more of SEQ ID NOs: 435-442.

25. The chimeric protein of claim 23, wherein the flexible linker is substantially comprised of GGSGGSGGGGSGGGGS (SEQ ID NO: 257).

26. The chimeric protein of claim 23, wherein the flexible linker is substantially comprised of LE, GGGGS (SEQ ID NO: 249), (GGGGS)ₙ (n=1-4) (SEQ ID NO: 249-SEQ ID NO: 252), (Gly)₈ (SEQ ID NO: 258, (Gly)₆ (SEQ ID NO: 259), (EAAAK)ₙ (n=1-3) (SEQ ID NO: 260-SEQ ID NO: 262), A(EAAAK)ₙA (n=2-5) (SEQ ID NO: 263-SEQ ID NO: 266), AEAAAKEAAAKA (SEQ ID NO: 263), A(EAAAK)₄ALEA(EAAAK)₄A (SEQ ID NO: 267), PAPAP (SEQ ID NO: 268), KESGSVSSEQLAQFRSLD (SEQ ID NO: 269), EGKSSGSGSESKST (SEQ ID NO: 270), GSAGSAAGSGEF (SEQ ID NO: 271), and (XP)ₙ, with X designating any amino acid, e.g., Ala, Lys, or Glu.

27. The chimeric protein complex of claim 1, comprising a Fc domain, the Fc domain optionally having one or more mutations that reduces or eliminates one or more effector functions of the Fc domain, promotes Fc chain pairing in the Fc domain, and/or stabilizes a hinge region in the Fc domain.

28. The chimeric protein complex of claim 27, wherein the Fc domain is selected from IgG, IgA, IgD, IgM or IgE.

29. The chimeric protein complex of claim 27 or 28, wherein the IgG is selected from IgG1, IgG2, IgG3, or IgG4.

30. The chimeric protein complex of any one of claims 27-29, wherein the Fc domain is from human IgG, IgA, IgD, IgM or IgE.

31. The chimeric protein complex of claim 30, wherein the human IgG is selected from human IgG1, IgG2, IgG3, or IgG4.

32. The chimeric protein complex of any one of claims 27-31, wherein the Fc chain pairing is promoted by ionic pairing and/or a knob-in-hole pairing.

33. The chimeric protein complex of any one of claims 27-32, wherein the one or more mutations to the Fc domain result in an ionic pairing between the Fc chains in the Fc domain.

34. The chimeric protein complex of any one of claims 27-33, wherein the one or more mutations to the Fc domain result in a knob-in-hole pairing in the Fc domain.

35. The chimeric protein complex of any one of claims 27-34, wherein the one or more mutations to the Fc domain result in the reduction or elimination of an effector function of the Fc domain.

36. The chimeric protein complex of any one of claims 27-35, wherein the chimeric protein complex is a heterodimer and has a trans orientation/configuration, as relates to any targeting moiety and IL-1α or pro-IL-1α, relative to each other, or any targeting moieties relative to each other, or any IL-1α or pro-IL-1α relative to each other.

37. The chimeric protein complex of any one of claims 27-35, wherein the chimeric protein complex is a heterodimer and has a cis orientation, as relates to any targeting moiety and IL-1α or pro-IL-1α, relative to each other, or any targeting moieties relative to each other, or any IL-1α or pro-IL-1α relative to each other.

38. The chimeric protein complex of any one of claims 27-37, wherein the Fc comprises L234A, L235A, and K322Q substitutions in human IgG1 (according to EU numbering).

39. The chimeric protein complex of any one of claims 27-38, wherein the Fc is human IgG1, and optionally contains one or more mutations of L234, L235, K322, D265, P329, and P331 (according to EU numbering).

40. The chimeric protein complex of any one of claims 27-39, wherein the chimeric protein complex has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, and 19A-F.

41. The chimeric protein or chimeric protein complex of any one of claims 1-40, wherein the chimeric protein or chimeric protein complex is suitable for use in a patient having one or more of: cancer, infections, immune disorders, autoimmune diseases, cardiovascular diseases, wound, ischemia-related diseases, neurodegenerative diseases, and/or metabolic diseases.

42. A recombinant nucleic acid composition encoding one or more chimeric proteins or chimeric protein complexes of any one of claims 1-40, or a constituent polypeptide thereof.

43. A host cell comprising a nucleic acid of claim 42.

44. A method for treating cancer, comprising administering an effective amount of i) the chimeric protein or chimeric protein complex of any one of the claims 1-40 to a patient in need thereof; ii) the recombinant nucleic acid of claim 42 to a patient in need thereof; or iii) the host cell of claim 43 to a patient in need thereof.

45. The method of claim 44, wherein the cancer is selected form one or more of basal cell carcinoma, biliary tract cancer, bladder cancer, bone cancer, brain and central nervous system cancer, breast cancer, cancer of the peritoneum, cervical cancer, choriocarcinoma, colon and rectum cancer, connective tissue cancer, cancer of the digestive system, endometrial cancer, esophageal cancer, eye cancer, cancer of the head and neck, gastric cancer, gastrointestinal cancer, glioblastoma, hepatic carcinoma, hepatoma, intra-epithelial neoplasm, kidney or renal cancer, larynx cancer, leukemia, liver cancer, lung cancer, small-cell lung cancer, non-small cell lung cancer, adenocarcinoma of the lung, squamous carcinoma of the lung, melanoma, myeloma, neuroblastoma, oral cavity cancer, ovarian cancer, pancreatic cancer, prostate cancer, retinoblastoma, rhabdomyosarcoma, rectal cancer, cancer of the respiratory system, salivary gland carcinoma, sarcoma, Kaposi's sarcoma, skin cancer, squamous cell cancer, stomach cancer, testicular cancer, thyroid cancer, uterine or endometrial cancer, cancer of the urinary system, vulval cancer, lymphoma, Hodgkin's lymphoma, non-Hodgkin's lymphoma, B-cell lymphoma, low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), hairy cell leukemia, chronic myeloblastic leukemia, carcinoma, sarcoma, post-transplant lymphoproliferative disorder (PTLD), abnormal vascular proliferation associated with phakomatoses, edema, and Meigs' syndrome.

46. A method for treating an autoimmune disease or disorder, comprising administering an effective amount of i) the chimeric protein or chimeric protein complex of any one of the claims 1-40 to a patient in need thereof; ii) the recombinant nucleic acid of claim 42 to a patient in need thereof; or iii) the host cell of claim 43 to a patient in need thereof.

47. The method of claim 46, wherein the autoimmune disease or disorder is selected from Crohn's disease, diabetes, multiple sclerosis, systemic lupus erythematosis, rheumatoid arthritis or juvenile rheumatoid arthritis, and ulcerative colitis.

48. The chimeric protein or chimeric protein complex of any one of claims 1-40, for use as a medicament.

49. Use of the chimeric protein or chimeric protein complex of any one of claims 1-40, in the manufacture of a medicament.

50. The chimeric protein or chimeric protein complex of any one of claims 1-40, for use in the treatment of cancer, autoimmune diseases, inflammatory diseases, metabolic diseases, cardiovascular diseases, infectious disease, degenerative and neurodegenerative diseases.

51. A vaccine composition, comprising
(a) an adjuvant comprising a chimeric protein or chimeric protein complex of any one of claims 1-40; and
(b) an antigen which is suitable for inducing an immune response.

52. The vaccine composition of claim 51, wherein the targeting moiety comprises a recognition domain that recognizes and/or binds an antigen or receptor on an immune cell, and the immune cell is a T cell.

53. The vaccine composition of claim 51, wherein the targeting moiety comprises a recognition domain that recognizes and/or binds CD8, CD3, CD4, Clec9A, XCR1, SIRP1α.

54. The vaccine composition of any one of claims 51-53, further comprising an aluminum gel or salt.

55. The vaccine composition of claim 54, wherein the aluminum gel or salt is selected from aluminum hydroxide, aluminum phosphate, and aluminum sulfate.

56. The vaccine composition of any one of claims 51-55, wherein the vaccine further comprises an additional adjuvant selected from oil-in-water emulsion formulations, saponin adjuvants, Freunds Adjuvants, toll like receptors ligands, cytokines, and chitosans.

57. The vaccine composition of any one of claims 51-56, wherein the vaccine composition is suitable for preventing or mitigating a disease or disorder is an infectious disease, autoimmune disease, or a cancer.

58. The vaccine composition of claim 57, wherein the disease or disorder is an infectious disease.

59. The vaccine composition of claim 58, wherein the infectious disease is an infection with a pathogen, optionally selected from a bacterium, virus, fungus, or parasite.

60. The vaccine composition of claim 59, wherein the pathogen is a virus.

61. The vaccine composition of claim 60, wherein the virus is:
(a) an influenza virus, optionally selected from Type A, Type B, Type C, and Type D influenza viruses, or
(b) a member of the Coronaviridae family, optionally selected from
(i) a betacoronavirus, optionally selected from severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or
(ii) an alphacoronavirus, optionally selected from HCoV-NL63 and HCoV-229E.

62. The vaccine composition of claim 61, wherein the virus is SARS-CoV-2.

63. The vaccine composition of any one of claims 51-62, wherein the adjuvant is a nucleic acid encoding the chimeric protein or chimeric protein complex, or constituent thereof.

64. The vaccine composition of any one of claims 51-63, wherein the antigen is a protein or an antigenic fragment of a protein.

65. The vaccine composition of any one of claims 51-63, wherein the antigen is a nucleic acid encoding a protein or an antigenic fragment of a protein, or constituent thereof.

66. The vaccine composition of claim 63 or 65, wherein the nucleic acid is mRNA, optionally comprising one or more non-canonical nucleotides, optionally selected from pseudouridine and 5-methoxyuridine.

67. The vaccine composition of claim 63 or 65, wherein the nucleic acid is DNA, optionally selected from linear DNA, DNA fragments, or DNA plasmids.

68. The vaccine composition of any one of claims 64-67, wherein the antigen is a 2019-nCoV protein, an antigenic fragment thereof, or a nucleic acid encoding the same, wherein the 2019-nCoV protein is optionally selected from spike surface glycoprotein, membrane glycoprotein M, envelope protein E, and nucleocapsid phosphoprotein N.

69. The vaccine composition of claim 68, wherein the spike surface glycoprotein is the S1 or S2 subunit, or an antigenic fragment thereof.

70. The vaccine composition of claim 69, wherein the spike surface glycoprotein comprises the amino acid sequence of SEQ ID NO: 500, membrane glycoprotein precursor M comprises the amino acid sequence of SEQ ID NO: 501, the envelope protein E comprises the amino acid sequence of SEQ ID NO: 502, and the nucleocapsid phosphoprotein N comprises the amino acid sequence of SEQ ID NO: 503, or an amino acid sequence at least about 90%, or at least about 95%, or at least about 97%, or at least about 98%, or at least about 99% identity with any of the foregoing, or an antigenic fragment of any of the foregoing.

71. The vaccine composition of claim 61, wherein the virus is an influenza virus.

72. The vaccine composition of claim 71, wherein the antigen is an influenza viral antigen, an antigenic fragment thereof, or a nucleic acid encoding the same, wherein the viral antigen is optionally selected from hemagglutinin (HA) protein, matrix 2 (M2) protein, and neuraminidase, or an antigenic fragment thereof, or a nucleic acid encoding the same.

73. The vaccine composition of claim 58, wherein the disease or disorder is selected from diphtheria, tetanus, pertussis, influenza, pneumonia, hepatitis A, hepatitis B, polio, yellow fever, Human Papillomavirus (HPV) infection, anthrax, rabies, Japanese Encephalitis, meningitis, measles, mumps, rubella, gastroenteritis, smallpox, typhoid fever, varicella (chickenpox), rotavirus, and shingles.

74. The vaccine composition of any one of claims 51-73, wherein the antigen is that of one or more of the following vaccines: DTP (diphtheria-tetanus-pertussis vaccine), DTaP (diphtheria-tetanus-acellular pertussis vaccine), Hib (Haemophilus influenza type b) conjugate vaccines, Pneumococcal conjugate vaccine, Hepatitis A vaccines, Poliomyelitis vaccines, Yellow fever vaccines, Hepatitis B vaccines, combination DTaP, Tdap, Hib, Human Papillomavirus (HPV) vaccine, Anthrax vaccine, and Rabies vaccine.

75. The vaccine composition of claim 57, wherein the cancer is selected form one or more of basal cell carcinoma, biliary tract cancer, bladder cancer, bone cancer, brain and central nervous system cancer, breast cancer, cancer of the peritoneum, cervical cancer, choriocarcinoma, colon and rectum cancer, connective tissue cancer, cancer of the digestive system, endometrial cancer, esophageal cancer, eye cancer, cancer of the head and neck, gastric cancer, gastrointestinal cancer, glioblastoma, hepatic carcinoma, hepatoma, intra-epithelial neoplasm, kidney or renal cancer, larynx cancer, leukemia, liver cancer, lung cancer, small-cell lung cancer, non-small cell lung cancer, adenocarcinoma of the lung, squamous carcinoma of the lung, melanoma, myeloma, neuroblastoma, oral cavity cancer, ovarian cancer, pancreatic cancer, prostate cancer, retinoblastoma, rhabdomyosarcoma, rectal cancer, cancer of the respiratory system, salivary gland carcinoma, sarcoma, Kaposi's sarcoma, skin cancer, squamous cell cancer, stomach cancer, testicular cancer, thyroid cancer, uterine or endometrial cancer, cancer of the urinary system, vulval cancer, lymphoma, Hodgkin's lymphoma, non-Hodgkin's lymphoma, B-cell lymphoma, low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, chronic lymphocytic leukemia (CLL), acute lymphoblastic leukemia (ALL), hairy cell leukemia, chronic myeloblastic leukemia, carcinoma, sarcoma, post-transplant lymphoproliferative disorder (PTLD), abnormal vascular proliferation associated with phakomatoses, edema, and Meigs' syndrome.

76. The vaccine composition of claim 57, wherein the autoimmune disease or disorder is selected from Crohn's disease, diabetes, multiple sclerosis, systemic lupus erythematosis, rheumatoid arthritis or juvenile rheumatoid arthritis, and ulcerative colitis.

77. The vaccine composition of any one of claims 51-76, wherein the vaccine composition is formulated for administration intravenously.

78. The vaccine composition of any one of claims 51-76, wherein the vaccine composition is formulated for administration to the lung.

79. The vaccine composition of claim 78, wherein the vaccine composition is formulated for administration by inhalation.

80. The vaccine composition of claim 78 or 79, wherein the vaccine composition is formulated for administration via aerosol or nebulizer.

81. The vaccine composition of claim 80, wherein the vaccine composition is formulated for administration liquid nebulization, dry powder dispersion and meter-dose administration.

82. The vaccine composition of any one of claims 51-81, wherein the adjuvant or vaccine composition has:
(a) low toxicity;
(b) an ability to stimulate a long-lasting immune response against the antigen;
(c) substantial stability;
(d) an ability to elicit a humoral immune response and/or a cell-mediated immunity to the antigen;
(e) a capability of selectively interacting with populations of antigen presenting cells;
(f) an ability to specifically elicit T_{H1} and/or T_{H2} cell-specific immune responses to the antigen; and/or
(g) an ability to selectively increase appropriate antibody isotype levels against antigens, the isotype optionally being IgA,
when administered to a patient.

83. The vaccine composition of any one of claims 51-82, wherein the adjuvant or vaccine composition stimulates a CD8⁺ T cell response to the antigen, when administered to a patient.

84. The vaccine composition of any one of claims 51-83, wherein the adjuvant or vaccine composition stimulates activation of the IL-1R, when administered to a patient.

85. The vaccine composition of any one of claims 51-84, wherein the adjuvant or vaccine composition does not substantially cause one or more of fever, neutrophilia, and the release of acute phase proteins, when administered to a patient.

86. A method for vaccinating a subject against an infectious disease, comprising administering the vaccine composition of any one of claims 51-85.

87. The method of claim 86, wherein the adjuvant and antigen of the vaccine composition are administered concurrently.

88. The method of claim 86, wherein the adjuvant complex and antigen of the vaccine composition are co-formulated.

89. The method of claim 86, wherein the adjuvant and antigen of the vaccine composition are administered sequentially.

90. The method of claim 86, wherein the adjuvant and antigen of the vaccine composition are administered in multiple doses.

91. The method of claim 86, wherein the adjuvant of the vaccine composition is administered in multiple booster doses and the antigen of the vaccine composition is administered once.

92. A method for treating a subject afflicted with an infectious disease, comprising administering the chimeric protein or chimeric protein complex of any one of claims 1-40.

93. The method of claim 92, wherein the targeting moiety of the chimeric protein or chimeric protein complex comprises a recognition domain that recognizes and/or binds an antigen or receptor on an immune cell, optionally wherein the immune cell is a T cell.

94. The method of claim 92, wherein the targeting moiety of the chimeric protein or chimeric protein complex comprises a recognition domain that recognizes and/or binds CD8, CD3, CD4, Clec9A, XCR1, SIRP1α.

95. The method of any one of claims 92-94, wherein the chimeric protein or chimeric protein complex further comprises an aluminum gel or salt.

96. The method of claim 95, wherein the aluminum gel or salt is selected from aluminum hydroxide, aluminum phosphate, and aluminum sulfate.

97. The method of any one of claims 92-96, wherein the chimeric protein or chimeric protein complex further comprises an additional adjuvant selected from oil-in-water emulsion formulations, saponin adjuvants, Freunds Adjuvants, toll like receptors ligands, cytokines, and chitosans.

98. The method of claim 92, wherein the infectious disease is an infection with a pathogen, optionally selected from a bacterium, virus, fungus, or parasite.

99. The method of claim 98, wherein the pathogen is a virus.

100. The method of claim 99, wherein the virus is:
(a) an influenza virus, optionally selected from Type A, Type B, Type C, and Type D influenza viruses, or
(b) a member of the Coronaviridae family, optionally selected from
(i) a betacoronavirus, optionally selected from severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or
(ii) an alphacoronavirus, optionally selected from HCoV-NL63 and HCoV-229E.

101. The method of claim 100, wherein the virus is SARS-CoV-2.

102. The method of claim 100, wherein the virus is an influenza virus.

103. The method of any one of claims 92-102, wherein the chimeric protein or chimeric protein complex stimulates a CD8⁺ T cell response to the antigen, when administered to a patient.

104. The method of any one of claims 92-103, wherein the adjuvant or vaccine composition stimulates activation of the IL-1R, when administered to a patient.

105. The method of any one of claims 92-104, wherein the chimeric protein or chimeric protein complex does not substantially cause one or more of fever, neutrophilia and the release of acute phase proteins when administered to a patient.


SEQUENCE LISTING (524 sequences)

SEQ ID NO: 1 - protein, 159 aa, synthetic construct
  REGION 1..159: note = Synthetic Sequence
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLIFFWETHGTKNYFTSVAHPNLFIATKQDYWVCLAGGPPSITDFQILENQA

SEQ ID NO: 2 - protein, 271 aa, synthetic construct
  REGION 1..271: note = Synthetic Sequence
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLIFFWETHGTKNYFTSVAHPNLFIATKQDYWVCLAGGPPSITDFQILENQA

SEQ ID NO: 3 - protein, 159 aa, synthetic construct
  REGION 1..159: note = Synthetic Sequence
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 4 - protein, 271 aa, synthetic construct
  REGION 1..271: note = Synthetic Sequence
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 5 - protein, 166 aa, synthetic construct
  REGION 1..166: note = Synthetic polymer.
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 6 - skipped

SEQ ID NO: 7 - protein, 447 aa, synthetic construct
  REGION 1..447: note = Synthetic Sequence
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 218 aa, synthetic construct
  REGION 1..218: note = Synthetic Sequence
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 440 aa, synthetic construct
  REGION 1..440: note = Synthetic Sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
  REGION 1..214: note = Synthetic Sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 106 aa, synthetic construct
  REGION 1..106: note = Synthetic Sequence
EIVLTQSPSSLSASVGDRVTITCSARSSVSYMHWYQQKPGKAPKLLIYRTSNLASGVPSRFSGSGSGTDFTLTINSLQPEDFATYYCQQRSSFPLTFGGGTKLEIK

SEQ ID NO: 12 - protein, 106 aa, synthetic construct
  REGION 1..106: note = Synthetic Sequence
EIVLTQSPSSLSASVGDRVTITCSARSSVSYMHWFQQKPGKAPKLWIYRTSNLASGVPSRFSGSGSGTDYTLTINSLQPEDFATYYCQQRSSFPLTFGGGTKLEIK

SEQ ID NO: 13 - protein, 106 aa, synthetic construct
  REGION 1..106: note = Synthetic Sequence
EIVLTQSPSSLSASVGDRVTITCSARSSVSYMHWFQQKPGKAPKLWIYRTSNLASGVPSRFSGSGSGTDYCLTINSLQPEDFATYYCQQRSSFPLTFGGGTKLEIK

SEQ ID NO: 14 - protein, 106 aa, synthetic construct
  REGION 1..106: note = Synthetic Sequence
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 15 - protein, 117 aa, synthetic construct
  REGION 1..117: note = Synthetic Sequence
QVQLVQSGSELKKPGASVKISCKASGYSFSNYGMNWVRQAPGQGLQWMGWINTDSGESTYAEEFKGRFVFSLDTSVSTAYLQITSLTAEDTGMYFCAKVGYDALDYWGQGTLVTVSS

SEQ ID NO: 16 - protein, 117 aa, synthetic construct
  REGION 1..117: note = Synthetic Sequence
QVQLVQSGSELKKPGASVKISCKASGYTFTNYGMNWVRQAPGQGLQWMGWINTDSGESTYAEEFKGRFVFSLDTSVSTAYLQITSLTAEDTGMYFCAKVGYDALDYWGQGTLVTVSS

SEQ ID NO: 17 - protein, 117 aa, synthetic construct
  REGION 1..117: note = Synthetic Sequence
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 18 - protein, 117 aa, synthetic construct
  REGION 1..117: note = Synthetic Sequence
QIQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 19 - protein, 117 aa, synthetic construct
  REGION 1..117: note = Synthetic Sequence
QIQLVQSGSELKKPGASVKISCKASGYTFTNYGMNWVKQAPGQGLKWMGWINTDSGESTYAEEFKGRFAFSLDTSVNTAYLQITSLNAEDTGMYFCVRVGYDALDYWGQGTLVTVSS

SEQ ID NO: 20 - protein, 254 aa, synthetic construct
  REGION 1..254: note = Synthetic Sequence
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 21 - protein, 453 aa, synthetic construct
  REGION 1..453: note = Synthetic Sequence
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 22 aa, synthetic construct
  REGION 1..22: note = Synthetic Sequence
  SITE 8: note = MISC_FEATURE - A branch with sequence SNTSESF
SNTSESFKFRVTQLAPKAQIKE

SEQ ID NO: 23 - protein, 119 aa, synthetic construct
  REGION 1..119: note = Synthetic Sequence
EVQLQQSGPVLVKPGASVKMSCKASGYTFTDYYMNWVKQSHGKSLEWIGNINPYNGGTTYNQKFKGKATLTVDKSSRTAYMEINSLTSEDSAVYYCARGRIYDGSLDYWGQGTALTVSS

SEQ ID NO: 24 - protein, 106 aa, synthetic construct
  REGION 1..106: note = Synthetic Sequence
DIQMTQFPSSLCASQGGKVTVTCKASQDINNYMAWYQHKPGKGPRLLIHYTSTLLSGIPSRFSGSGSGRDYSFSISNLEPEDIATYYCLQYDNLWTFGGGTKLEIK

SEQ ID NO: 25 - protein, 116 aa, synthetic construct
  REGION 1..116: note = Synthetic Sequence
QVQLQQSGAELAKPGASVRLSCKASGYTFTNYWMHWVKQRPGQGLEWIGHINPSSGFTTYNQNFKDKATLTADKSSNTAYMQLSSLTYEDSAVYFCAREDYDVDYWGQGTTLTVSS

SEQ ID NO: 26 - protein, 107 aa, synthetic construct
  REGION 1..107: note = Synthetic Sequence
DIVMTQSQKFMSTSVGDRVSVTCKASQSVDTNVAWYQQKPGQSPKALIFSASYRYSGVPDRFTGSGSGTDFTLTINSVQSEDLAEYFCQQYNSYPYTFGSGTKLEIK

SEQ ID NO: 27 - protein, 122 aa, synthetic construct
  REGION 1..122: note = Synthetic Sequence
EVQLVESGGGLVKPGGSLKLSCAASGFTFSDYGMHWVRQAPEKGLEWVAYISSGSYTIYYTDTVKGRFTISRDNAKNTLFLQMTSLRSEDTAMYYCARRGYGSFYEYYFDYWGQGTTLTVSS

SEQ ID NO: 28 - protein, 106 aa, synthetic construct
  REGION 1..106: note = Synthetic Sequence
QIVLTQSPALMSASPGEKVTMTCSASSSVSYMYWYQQKPRSSPKPWIYLTSNLASGVPARFSGSGSGTSYSLTISSMEAEDAATYYCQQWSSNPFTFGSGTKLEIK

SEQ ID NO: 29 - protein, 119 aa, synthetic construct
  REGION 1..119: note = Synthetic Sequence
EVQLVESGGGLVQAGKSLRLSCAASGSIFSIHAMGWFRQAPGKEREFVAAITWSGGITYYEDSVKGRFTISRDNAKNTVYLQMNSLKPEDTAIYYCAADRAESSWYDYWGQGTQVTVSS

SEQ ID NO: 30 - protein, 119 aa, synthetic construct
  REGION 1..119: note = Synthetic Sequence
EVQLVESGGGLVQAGGSLRLSCAASGSIASIHAMGWFRQAPGKEREFVAVITWSGGITYYADSVKGRFTISRDNAKNTVYLQMNSLKPEDTAIYYCAGDKHQSSWYDYWGQGTQVTVSS

SEQ ID NO: 31 - protein, 119 aa, synthetic construct
  REGION 1..119: note = Synthetic Sequence
EVQLVESGGGLVQAGGSLRLSCAASGSISSIHAMGWFRQAPGKEREFVAAITWSGGITYYADSLKGRFTISRDNAKNTGYLQMNSLKPEDTAIYYCAADRAQSSWYDYWGQGTQVTVSS

SEQ ID NO: 32 - protein, 119 aa, synthetic construct
  REGION 1..119: note = Synthetic Sequence
EVQLVESGGGLVQAGGSLGLSCAASGSIFSINAMAWFRQAPGKEREFVALISWSGGSTYYEDSVKGRFTISRDNAKNTVYLQMNSLKPEDTAIYYCAADRVDSNWYDYWGQGTQVTVSS

SEQ ID NO: 33 - protein, 120 aa, synthetic construct
  REGION 1..120: note = Synthetic Sequence
EVQLVESGGGLVQAGGSLRLSCAASGRAFSSGTMGWFRRAPGKEREFVASIPWSGGRIYYADSVKGRFTISRDNAQNTVYLQMNSLKPEDTAVYYCAVKERSTGWDFASWGQCTQVTVSS

SEQ ID NO: 34 - protein, 121 aa, synthetic construct
  REGION 1..121: note = Synthetic Sequence
QVQLVQSGAELKQPGASVKMSCKASGYSFTSSWIHWVKQAPGQGLEWIGYIYPSTGFTEYNQKFKDRATLTADKSTSTAYMELSSLRSEDSAVYYCARWRDSSGYHAMDYWGQGTSVTVSS

SEQ ID NO: 35 - protein, 121 aa, synthetic construct
  REGION 1..121: note = Synthetic Sequence
QVQLVQSGAEVKQPGASVKMSCKASGYSFTSSWIHWVKQAPGQGLEWIGYIYPSTGFTEYNQKFKDRATLTADKSTSTAYMELSSLRSEDTAVYYCARWRDSSGYHAMDYWGQGTSVTVSS

SEQ ID NO: 36 - protein, 121 aa, synthetic construct
  REGION 1..121: note = Synthetic Sequence
QVQLVQSGHEVKQPGASVKMSCKASGYSFTSSWIHWVKQAPGQGLEWIGYIYPSTGFTEYNQKFKDRATLTADKSTSTAYMELSSLRSEDTAVYYCARWRDSSGYHAMDYWGQGTLVTVSS

SEQ ID NO: 37 - protein, 121 aa, synthetic construct
  REGION 1..121: note = Synthetic Sequence
QVQLVQSGHEVKQPGASVKMSCKASGYSFTSSWIHWVRQAPGQGLEWIGYIYPSTGFTEYNQKFKDRATLTADKSTSTAYMELSSLRSEDTAVYYCARWRDSSGYHAMDYWGQGTLVTVSS

SEQ ID NO: 38 - protein, 121 aa, synthetic construct
  REGION 1..121: note = Synthetic Sequence
QVQLVQSGHEVKQPGASVKVSCKASGYSFTSSWIHWVRQAPGQGLEWIGYIYPSTGFTEYNQKFKDRATITADKSTSTAYMELSSLRSEDTAVYYCARWRDSSGYHAMDYWGQGTLVTVSS

SEQ ID NO: 39 - protein, 111 aa, synthetic construct
  REGION 1..111: note = Synthetic Sequence
DIVLTQSPASLTLSPGQRLTISCRASQSVSTSGYSYMHWYQQKPDQSPKLLIKFGSNLESGIPARFSGSGSGTDFTLTISSLEEEDFATYYCQHSWEIPYTFGQGTKLEIK

SEQ ID NO: 40 - protein, 111 aa, synthetic construct
  REGION 1..111: note = Synthetic Sequence
DIVLTQSPATLSLSPGQRLTISCRASQSVSTSGYSYMHWYQQKPDQSPKLLIKFGSNLESGIPARFSGSGSGTDFTLTISSLEPEDFATYYCQHSWEIPYTFGQGTKLEIK

SEQ ID NO: 41 - protein, 111 aa, synthetic construct
  REGION 1..111: note = Synthetic Sequence
EIVLTQSPATLSLSPGQRLTISCRASQSVSTSGYSYMHWYQQKPDQSPKLLIKFGSNLESGIPARFSGSGSGTDFTLTISSLEPEDFATYYCQHSWEIPYTFGQGTKLEIK

SEQ ID NO: 42 - protein, 111 aa, synthetic construct
  REGION 1..111: note = Synthetic Sequence
DIVLTQSPATLSLSPGQRLTISCRASQSVSTSGYSYMHWYQQKPDQSPKLLIKFGSNLESGIPARFSGSGSGTDFTLTISSLEPEDFAVYYCQHSWEIPYTFGQGTKLEIK

SEQ ID NO: 43 - protein, 451 aa, synthetic construct
  REGION 1..451: note = Synthetic Sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 215 aa, synthetic construct
  REGION 1..215: note = Synthetic Sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 121 aa, synthetic construct
  REGION 1..121: note = Synthetic Sequence
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 46 - protein, 108 aa, synthetic construct
  REGION 1..108: note = Synthetic Sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 47 - protein, 448 aa, synthetic construct
  REGION 1..448: note = Synthetic Sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
  REGION 1..214: note = Synthetic Sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 450 aa, synthetic construct
  REGION 1..450: note = Synthetic Sequence
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 216 aa, synthetic construct
  REGION 1..216: note = Synthetic Sequence
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 51 - protein, 123 aa, synthetic construct
  REGION 1..123: note = Synthetic Sequence
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 52 - protein, 106 aa, synthetic construct
  REGION 1..106: note = Synthetic Sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 53 - protein, 117 aa, synthetic construct
  REGION 1..117: note = Synthetic Sequence
QVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSS

SEQ ID NO: 54 - protein, 107 aa, synthetic construct
  REGION 1..107: note = Synthetic Sequence
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIK

SEQ ID NO: 55 - protein, 118 aa, synthetic construct
  REGION 1..118: note = Synthetic Sequence
QVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSS

SEQ ID NO: 56 - protein, 107 aa, synthetic construct
  REGION 1..107: note = Synthetic Sequence
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 57 - protein, 120 aa, synthetic construct
  REGION 1..120: note = Synthetic Sequence
QVQLVQSGAEVKKPGSSVKVSCKVSGGIFSTYAINWVRQAPGQGLEWMGGIIPIFGTANHAQKFQGRVTITADESTSTAYMELSSLRSEDTAVYYCARDQGIAAALFDYWGQGTLVTVSS

SEQ ID NO: 58 - protein, 108 aa, synthetic construct
  REGION 1..108: note = Synthetic Sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 59 - protein, 113 aa, synthetic construct
  REGION 1..113: note = Synthetic Sequence
EVQLVESGGGLVQPGRSLRLSCAVSGFTFDDYVVHWVRQAPGKGLEWVSGISGNSGNIGYADSVKGRFTISRDNAKNSLYLQMNSLRAEDTALYYCAVPFDYWGQGTLVTVSS

SEQ ID NO: 60 - protein, 107 aa, synthetic construct; identical to SEQ ID NO: 56
  REGION 1..107: note = Synthetic Sequence

SEQ ID NO: 61 - protein, 123 aa, synthetic construct
  REGION 1..123: note = Synthetic Sequence
QVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSS

SEQ ID NO: 62 - protein, 106 aa, synthetic construct; identical to SEQ ID NO: 52
  REGION 1..106: note = Synthetic Sequence

SEQ ID NO: 63 - protein, 123 aa, synthetic construct
  REGION 1..123: note = Synthetic Sequence
QVQLVQSGAEVKKPGSSVKVSCKTSGGTFSSYAISWVRQAPGQGLEWMGGIIPIFGKAHYAQKFQGRVTITADESTTTAYMELSSLRSEDTAVYYCARKYDYVSGSPFGMDVWGQGTTVTVSS

SEQ ID NO: 64 - protein, 106 aa, synthetic construct; identical to SEQ ID NO: 52
  REGION 1..106: note = Synthetic Sequence

SEQ ID NO: 65 - protein, 121 aa, synthetic construct
  REGION 1..121: note = Synthetic Sequence
QVQLVQSGAEVKKPGSSVKVSCKASGGTFSSYAINWVRQAPGQGLEWMGGIIPIFGSANYAQKFQDRVTITADESTSAAYMELSSLRSEDTAVYYCARDSSGWSRYYMDVWGQGTTVTVSS

SEQ ID NO: 66 - protein, 106 aa, synthetic construct
  REGION 1..106: note = Synthetic Sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGGGTKVEIK

SEQ ID NO: 67 - protein, 123 aa, synthetic construct
  REGION 1..123: note = Synthetic Sequence
QVQLVQSGAEVKEPGSSVKVSCKASGGTFNSYAISWVRQAPGQGLEWMGGIIPLFGIAHYAQKFQGRVTITADESTNTAYMDLSSLRSEDTAVYYCARKYSYVSGSPFGMDVWGQGTTVTVSS

SEQ ID NO: 68 - protein, 106 aa, synthetic construct
  REGION 1..106: note = Synthetic Sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTRLEIK

SEQ ID NO: 69 - protein, 121 aa, synthetic construct
  REGION 1..121: note = Synthetic Sequence
EVQLVESGGGLVQPGRSLRLSCAASGITFDDYGMHWVRQAPGKGLEWVSGISWNRGRIEYADSVKGRFTISRDNAKNSLYLQMNSLRAEDTALYYCAKGRFRYFDWFLDYWGQGTLVTVSS

SEQ ID NO: 70 - protein, 107 aa, synthetic construct
  REGION 1..107: note = Synthetic Sequence
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 71 - protein, 118 aa, synthetic construct
  REGION 1..118: note = Synthetic Sequence
EVKLQESGPSLVKPSQTLSLTCSVTGYSITSDYWNWIRKFPGNKLEYVGYISYTGSTYYNPSLKSRISITRDTSKNQYYLQLNSVTSEDTATYYCARYGGWLSPFDYWGQGTTLTVSS

SEQ ID NO: 72 - protein, 107 aa, synthetic construct
  REGION 1..107: note = Synthetic Sequence
DIVMTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 73 - protein, 120 aa, synthetic construct
  REGION 1..120: note = Synthetic Sequence
EVQLQESGPGLVAPSQSLSITCTVSGFSLTTYSINWIRQPPGKGLEWLGVMWAGGGTNSNSVLKSRLIISKDNSKSQVFLKMNSLQTDDTARYYCARYYGNSPYYAIDYWGQGTSVTVSS

SEQ ID NO: 74 - protein, 107 aa, synthetic construct
  REGION 1..107: note = Synthetic Sequence
DIVTTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 75 - protein, 118 aa, synthetic construct
  REGION 1..118: note = Synthetic Sequence
EVKLQESGPSLVKPSQTLSLTCSVTGYSIISDYWNWIRKFPGNKLEYLGYISYTGSTYYNPSLKSRISITRDTSKNQYYLQLNSVTTEDTATYYCARRGGWLLPFDYWGQGTTLTVSS

SEQ ID NO: 76 - protein, 113 aa, synthetic construct
  REGION 1..113: note = Synthetic Sequence
DIVMTQSPSSLAVSVGEKVSMGCKSSQSLLYSSNQKNSLAWYQQKPGQSPKLLIDWASTRESGVPDRFTGSGSGTDFTLTISSVKAEDLAVYYCQQYYGYPLTFGAGTKLELK

SEQ ID NO: 77 - protein, 118 aa, synthetic construct
  REGION 1..118: note = Synthetic Sequence
EVKLQESGPSLVKPGASVKLSCKASGYTFTSYDINWVKQRPGQGLEWIGWIFPRDNNTKYNENFKGKATLTVDTSSTTAYMELHSLTSEDSAVYFCTKENWVGDFDYWGQGTTLTLSS

SEQ ID NO: 78 - protein, 106 aa, synthetic construct
  REGION 1..106: note = Synthetic Sequence
DIVMTQSPAIMSASPGEKVTMTCSASSSIRYMHWYQQKPGTSPKRWISDTSKLTSGVPARFSGSGSGTSYALTISSMEAEDAATYYCHQRSSYPWTFGGGTKLEIK

SEQ ID NO: 79 - protein, 114 aa, synthetic construct
  REGION 1..114: note = Synthetic Sequence
EVQLQQSGPDLVTPGASVRISCQASGYTFPDYYMNWVKQSHGKSLEWIGDIDPNYGGTTYNQKFKGKAILTVDRSSSTAYMELRSLTSEDSAVYYCARGALTDWGQGTSLTVSS

SEQ ID NO: 80 - protein, 106 aa, synthetic construct
  REGION 1..106: note = Synthetic Sequence
QIVLSQSPAILSASPGEKVTMTCRASSSVSYIYWFQQKPGSSPKPWIYATFNLASGVPARFSGSGSGTSYSLTISRVETEDAATYYCQQWSNNPLTFGAGTKLELK

SEQ ID NO: 81 - protein, 115 aa, synthetic construct
  REGION 1..115: note = Synthetic Sequence
EVQLVQSGPELKKPGASVKMSCKASGYTFTSYVMHWVKQAPGQRLEWIGYVNPFNDGTKYNEMFKGRATLTSDKSTSTAYMELSSLRSEDSAVYYCARQAWGYPWGQGTLVTVSS

SEQ ID NO: 82 - protein, 115 aa, synthetic construct
  REGION 1..115: note = Synthetic Sequence
EVQLVQSGAEVKKPGASVKMSCKASGYTFTSYVMHWVKQAPGQRLEWIGYVNPFNDGTKYNEMFKGRATLTSDKSTSTAYMELSSLRSEDTAVYYCARQAWGYPWGQGTLVTVSS

SEQ ID NO: 83 - protein, 115 aa, synthetic construct
  REGION 1..115: note = Synthetic Sequence
EVQLVQSGAEVKKPGASVKMSCKASGYTFTSYVMHWVRQAPGQRLEWIGYVNPFNDGTKYNEMFKGRATLTSDKSTSTAYMELSSLRSEDTAVYYCARQAWGYPWGQGTLVTVSS

SEQ ID NO: 84 - protein, 115 aa, synthetic construct
  REGION 1..115: note = Synthetic Sequence
EVQLVQSGAEVKKPGASVKVSCKASGYTFTSYVMHWVRQAPGQRLEWIGYVNPFNDGTKYNEMFKGRATLTSDKSTSTAYMELSSLRSEDTAVYYCARQAWGYPWGQGTLVTVSS

SEQ ID NO: 85 - protein, 115 aa, synthetic construct
  REGION 1..115: note = Synthetic Sequence
EVQLVQSGAEVKKPGASVKVSCKASGYTFTSYVMHWVRQAPGQRLEWIGYVNPFNDGTKYNEMFKGRATITSDKSTSTAYMELSSLRSEDTAVYYCARQAWGYPWGQGTLVTVSS

SEQ ID NO: 86 - protein, 111 aa, synthetic construct
  REGION 1..111: note = Synthetic Sequence
DIVLTQSPASLALSPGERATLSCRATESVEYYGTSLVQWYQQKPGQPPKLLIYAASSVDSGVPSRFSGSGSGTDFTLTINSLEEEDAAMYFCQQSRRVPYTFGQGTKLEIK

SEQ ID NO: 87 - protein, 111 aa, synthetic construct
  REGION 1..111: note = Synthetic Sequence
DIVLTQSPATLSLSPGERATLSCRATESVEYYGTSLVQWYQQKPGQPPKLLIYAASSVDSGVPSRFSGSGSGTDFTLTINSLEAEDAAMYFCQQSRRVPYTFGQGTKLEIK

SEQ ID NO: 88 - protein, 111 aa, synthetic construct
  REGION 1..111: note = Synthetic Sequence
EIVLTQSPATLSLSPGERATLSCRATESVEYYGTSLVQWYQQKPGQPPKLLIYAASSVDSGVPSRFSGSGSGTDFTLTINSLEAEDAAMYFCQQSRRVPYTFGQGTKLEIK

SEQ ID NO: 89 - protein, 111 aa, synthetic construct
  REGION 1..111: note = Synthetic Sequence
DIVLTQSPATLSLSPGERATLSCRATESVEYYGTSLVQWYQQKPGQPPKLLIYAASSVDSGVPSRFSGSGSGTDFTLTINSLEAEDAATYFCQQSRRVPYTFGQGTKLEIK

SEQ ID NO: 90 - protein, 120 aa, synthetic construct
  REGION 1..120: note = Synthetic Sequence
EVQLVESGGGLVKPGGSLRLSCAASGFTFSTYSMNWVRQAPGKGLEWVSSISSSGDYIYYADSVKGRFTISRDNAKNSLFLQMNSLKAEDTAVYYCARDLVTSMVAFDYWGQGTLVTVSS

SEQ ID NO: 91 - protein, 108 aa, synthetic construct
  REGION 1..108: note = Synthetic Sequence
SYELTQPPSVSVSPGQAARITCSGDALPQKYVFWYQQKSGQAPVLVIYEDSKRPSGIPERFSGSSSGTMATLTISGAQVEDEADYYCYSTDRSGNHRVFGGGTRLTVL

SEQ ID NO: 92 - protein, 120 aa, synthetic construct
  REGION 1..120: note = Synthetic Sequence
EVQLVESGGGLVQPGGSLRLSCAASGFTFSSYWMSWVRQAPGKGLEWVANIKQDGGEQYYVDSVKGRFTISRDNAKNSLYLQMNSLRAEDTAVYYCARDWNYGYYDMDVWGQGTTVTVSS

SEQ ID NO: 93 - protein, 108 aa, synthetic construct
  REGION 1..108: note = Synthetic Sequence
EIVLTQSPGTLSLSPGERATLSCRASQSVSSNYLAWFQQKPGQAPRLLIFGTSSRATGIPDRFSGSGSGTDFTLTISRLEPEDFAVYYCQQYGSSIFTFGPGTKVDIK

SEQ ID NO: 94 - protein, 121 aa, synthetic construct; identical to SEQ ID NO: 45
  REGION 1..121: note = Synthetic Sequence

SEQ ID NO: 95 - protein, 108 aa, synthetic construct
  REGION 1..108: note = Synthetic Sequence
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTEVEIK

SEQ ID NO: 96 - protein, 121 aa, synthetic construct
  REGION 1..121: note = Synthetic Sequence
EVQLLESGGGLVQPGGSLRLSCAASGFTFSNYAMSWVRQAPGKGLEWVSAIRGSGGSTYYADSVKGRFTISRDNSKNTLYLQMNSLRAEDTAVYYCAKDLHYDSSGYLDYWGQGTLVTVSS

SEQ ID NO: 97 - protein, 107 aa, synthetic construct
  REGION 1..107: note = Synthetic Sequence
DIQMTQSPSSVSASVGDRVTITCRASQGIRSWLAWYQQKPGKAPKLLIYAISRLQSGVPSRFSGSGSGTDFTLTISSLQPEDFATYYCQQANSFPLTFGGGTKVEIK

SEQ ID NO: 98 - protein, 119 aa, synthetic construct
  REGION 1..119: note = Synthetic Sequence
EVQLVESGGGLVQPGGSLRLSCAASGFTFSSYWMSWVRQAPGKGLEWVANIKQDGGEKYYVDSVKGRFTISRDNAKNSLFLQMNSLRAEDTAVYYCARVQLYSDYFDYWGQGTLVTVSS

SEQ ID NO: 99 - protein, 107 aa, synthetic construct
  REGION 1..107: note = Synthetic Sequence
DIQMTQSPSSVSASVGDRVTITCRASQGISSWLAWYQQKSGKAPKLLIYAASGLQSGVPSRFSGSGSGTDFTLTISSLQPEDLATYYCQQSHSLPPTFGQGTKVEIK

SEQ ID NO: 100 - protein, 121 aa, synthetic construct
  REGION 1..121: note = Synthetic Sequence
EVQLLESGGDLVQPGGSLRLSCAASGFTFNSYAMSWVRQAPGKGLEWVSTISGSGGFTFSADSVKGRFTISRDNSKNTLFLQMNSLRVEDSAVYSCAKVLVGFNNGCWDYWGQGTLVTVSS

SEQ ID NO: 101 - protein, 108 aa, synthetic construct
  REGION 1..108: note = Synthetic Sequence
SYVLTQPPSVSVAPGQTARITCGGNNIGSKSVHWYQQKPGQAPVLVVYDDSDRPSGIPERFSGSNSGNTATLTISRVEAGDEADYYCQVWDSSNDHVVFGGGTKLTVL

SEQ ID NO: 102 - protein, 120 aa, synthetic construct
  REGION 1..120: note = Synthetic Sequence
EVQLVESGGGLVKPGGSLRLSCAASGFTFSTYSMNWVRQAPGKGLEWVSSISSSGDYIYYADSVKGRFTISRDNAKNSLYLQMNSLRAEDTAVYYCARDLVTSMVAFDYWGQGTLVTVSS

SEQ ID NO: 103 - protein, 108 aa, synthetic construct
  REGION 1..108: note = Synthetic Sequence
SYELTQPPSVSVSPGQTARITCSGDALPQKYVFWYQQKSGQAPVLVIYEDSKRPSGIPERFSGSSSGTMATLTISGAQVEDEADYYCYSTDRSGNHRVFGGGTKLTVL

SEQ ID NO: 104 - protein, 121 aa, synthetic construct; identical to SEQ ID NO: 45
  REGION 1..121: note = Synthetic Sequence

SEQ ID NO: 105 - protein, 108 aa, synthetic construct; identical to SEQ ID NO: 46
  REGION 1..108: note = Synthetic Sequence

SEQ ID NO: 106 - protein, 120 aa, synthetic construct
  REGION 1..120: note = Synthetic Sequence
QVQLVQSGAEVKKPGASVKVSCKASGYTFTSYWMYWVRQATGQGLEWMGRIDPNSGSTKYNEKFKNRFTISRDDSKNTAYLQMNSLKTEDTAVYYCARDYRKGLYAMDYWGQGTTVTVSS

SEQ ID NO: 107 - protein, 120 aa, synthetic construct
  REGION 1..120: note = Synthetic Sequence
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 108 - protein, 120 aa, synthetic construct
  REGION 1..120: note = Synthetic Sequence
EVQLVQSGAEVKKPGESLRISCKGSGYTFTSYWMYWVRQAPGQGLEWMGRIDPNSGSTKYNEKFKNRVTISVDTSKNQFSLKLSSVTAADTAVYYCARDYRKGLYAMDYWGQGTTVTVSS

SEQ ID NO: 109 - protein, 120 aa, synthetic construct
  REGION 1..120: note = Synthetic Sequence
EVQLVQSGAEVKKPGATVKISCKVSGYTFTSYWMYWIRQSPSRGLEWLGRIDPNSGSTKYNEKFKNRLTISKDTSKNQVVLTMTNMDPVDTATYYCARDYRKGLYAMDYWGQGTTVTVSS

SEQ ID NO: 110 - protein, 120 aa, synthetic construct
  REGION 1..120: note = Synthetic Sequence
EVQLVQSGAEVKKPGESLRISCKGSGYTFTSYWMYWIRQPPGKGLEWIGRIDPNSGSTKYNEKFKNRVTITADKSTSTAYMELSSLRSEDTAVYYCARDYRKGLYAMDYWGQGTTVTVSS

SEQ ID NO: 111 - protein, 120 aa, synthetic construct
  REGION 1..120: note = Synthetic Sequence
QVQLVQSGAEVKKPGASVKVSCKASGYTFTSYWMYWIRQSPSRGLEWLGRIDPNSGSTKYNEKFKNRFTISRDDSKNTAYLQMNSLKTEDTAVYYCARDYRKGLYAMDYWGQGTTVTVSS

SEQ ID NO: 112 - protein, 120 aa, synthetic construct
  REGION 1..120: note = Synthetic Sequence
EVQLVQSGAEVKKPGESLRISCKGSGYTFTSYWMYWVRQARGQRLEWIGRIDPNSGSTKYNEKFKNRLTISKDTSKNQVVLTMTNMDPVDTATYYCARDYRKGLYAMDYWGQGTTVTVSS

SEQ ID NO: 113 - protein, 120 aa, synthetic construct
  REGION 1..120: note = Synthetic Sequence
QITLKESGPTLVKPTQTLTLTCTFSGYTFTSYWMYWVRQAPGKGLEWVSRIDPNSGSTKYNEKFKNRVTITADKSTSTAYMELSSLRSEDTAVYYCARDYRKGLYAMDYWGQGTTVTVSS

SEQ ID NO: 114 - protein, 120 aa, synthetic construct
  REGION 1..120: note = Synthetic Sequence
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 115 - protein, 107 aa, synthetic construct
  REGION 1..107: note = Synthetic Sequence
DIVMTQTPLSLPVTPGEPASISCKASQDVGTAVAWYLQKPGQSPQLLIYWASTRHTGIPARFSGSGSGTEFTLTISSLQSEDFAVYYCQQYNSYPLTFGQGTKVEIK

SEQ ID NO: 116 - protein, 107 aa, synthetic construct
  REGION 1..107: note = Synthetic Sequence
DIQMTQSPSSLSASVGDRVTITCKASQDVGTAVAWYLQKPGQSPQLLIYWASTRHTGVPSRFSGSGSGTDFTLTISSLQPEDFATYYCQQYNSYPLTFGQGTKVEIK

SEQ ID NO: 117 - protein, 107 aa, synthetic construct
  REGION 1..107: note = Synthetic Sequence
EIVLTQSPDFQSVTPKEKVTITCKASQDVGTAVAWYLQKPGQSPQLLIYWASTRHTGVPDRFSGSGSGTDFTLKISRVEAEDVGVYYCQQYNSYPLTFGQGTKVEIK

SEQ ID NO: 118 - protein, 107 aa, synthetic construct
  REGION 1..107: note = Synthetic Sequence
EIVLTQSPDFQSVTPKEKVTITCKASQDVGTAVAWYLQKPGQSPQLLIYWASTRHTGVPSRFSGSGSGTDFTFTISSLQPEDIATYYCQQYNSYPLTFGQGTKVEIK

SEQ ID NO: 119 - protein, 107 aa, synthetic construct
  REGION 1..107: note = Synthetic Sequence
EIVLTQSPATLSLSPGERATLSCKASQDVGTAVAWYLQKPGQSPQLLIYWASTRHTGIPPRFSGSGYGTDFTLTINNIESEDAAYYFCQQYNSYPLTFGQGTKVEIK

SEQ ID NO: 120 - protein, 107 aa, synthetic construct
  REGION 1..107: note = Synthetic Sequence
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 121 - protein, 107 aa, synthetic construct
  REGION 1..107: note = Synthetic Sequence
DIQMTQSPSSLSASVGDRVTITCKASQDVGTAVAWYQQKPGQAPRLLIYWASTRHTGVPSRFSGSGSGTDFTFTISSLQPEDIATYYCQQYNSYPLTFGQGTKVEIK

SEQ ID NO: 122 - protein, 107 aa, synthetic construct
  REGION 1..107: note = Synthetic Sequence
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 123 - protein, 107 aa, synthetic construct
  REGION 1..107: note = Synthetic Sequence
EIVLTQSPDFQSVTPKEKVTITCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 124 - protein, 116 aa, synthetic construct
  REGION 1..116: note = Synthetic Sequence
EVKLVESGGGLVKPGGSLKLSCAASGFIFRSYGMSWVRQTPEKRLEWVASISSGGSTYYPDSVKGRFTISRDNARNILYLQMSSLRSEDTAMYDCARGYDSGFAYWGQGTLVTVSE

SEQ ID NO: 125 - protein, 116 aa, synthetic construct
  REGION 1..116: note = Synthetic Sequence
EVKLVESGGGLVKPGGSLKLSCAASGFTFRSYGMSWVRQTPEKRLEWVASISSGGTTYYPDSVKGRFIISRDNARNILYLQMSSLRSEDTAMYYCAKGYDSGFAYWGQGTLVIVSA

SEQ ID NO: 126 - protein, 116 aa, synthetic construct
  REGION 1..116: note = Synthetic Sequence
QVQLKQSGPGLVQPSQSLSITCTVSGFSLTTYGVHWVRQSPGKGLEWLGVIWRGVTTDYNAAFMSRLTITKDNSKSQVFFKMNSLQANDTAIYYCARLGFYAMDYWGQGTSVTVSS

SEQ ID NO: 127 - protein, 116 aa, synthetic construct
  REGION 1..116: note = Synthetic Sequence
QVQLKQSGPGLVQPSQSLSITCTVSGFSLTSYGVHWVRQSPGKGLEWLGVIWSGGVTDYNAAFISRLSISKDNSKSQVFFKMNSLQANDTAIYYCARLGFYAMDYWGQGTSVTVSS

SEQ ID NO: 128 - protein, 119 aa, synthetic construct
  REGION 1..119: note = Synthetic Sequence
EVKLFESGGGLVQPGGSLKLSCVASGFDFSTYWMHWVRQAPGQGLEWIGQINPDSTTINYAPSLKDRFIISRDNAKNTLFLQMSKVRSEDTALYYCAKPGDYGYDFDCWGQGTTLTVSS

SEQ ID NO: 129 - protein, 119 aa, synthetic construct
  REGION 1..119: note = Synthetic Sequence
EVQLQESGPSLVKPSQTLSLTCSVTGDSITSGYWNWIRKFPGNKLEYMGYISYSGSTYYNPSLKSRISITRDTSKNQYYLQLNSVTTEDTATYYCARSLLWFSTGFAYWGQGTLVTVSA

SEQ ID NO: 130 - protein, 116 aa, synthetic construct
  REGION 1..116: note = Synthetic Sequence
QVQLKQSGPGLVQPSQSLSITCTVSGFSLTSYGVHWVRQSPGKGLEWLGVIWSGGITDYNAAFKSRLSISKDNSKSQVFFKMNSLQANDTAIYFCARLGFYAMDYWGQGTSVTVSS

SEQ ID NO: 131 - protein, 116 aa, synthetic construct
  REGION 1..116: note = Synthetic Sequence
EVKLVESGGGLVKPGGSLKLSCAASGFTFRSYGMSWARQIPEKRLEWVASISSGGTTYYLGSVQGRFTISRDNARNILYLQMSSLRSEDTAMYYCARGYDAGFAYWGQGTLVSVSE

SEQ ID NO: 132 - protein, 117 aa, synthetic construct
  REGION 1..117: note = Synthetic Sequence
EVQLQESGPSLVKPSQTLSLTCSVTGDSITSGYWTWIRKFPGNKLEYMGYISYTGSTYYNPSLKSRISISRDTSKSQYYLQLNSVTTEDTATYYCARQRDWLGFAYWGQGTLVTVSA

SEQ ID NO: 133 - protein, 116 aa, synthetic construct
  REGION 1..116: note = Synthetic Sequence
EEKLVESGGGLVKPGGSLKLSCAASGFSFSSYGMSWVRQTPEKRLEWVASISSGGSIYYPDSVKGRFTISRDNARNILYLQMSSLRSEDTAMYYCARGYDAGFAFWGQGTLVTASA

SEQ ID NO: 134 - protein, 116 aa, synthetic construct
  REGION 1..116: note = Synthetic Sequence
QITLKESGPTLVKPTQTLTLTCTVSGFSLSTYGVHWIRQPPGKALEWLGVIWRGVTTDYNAAFMSRLTITKDNSKNQVVLTMNNMDPVDTATYYCARLGFYAMDYWGQGTLVTVSS

SEQ ID NO: 135 - protein, 116 aa, synthetic construct
  REGION 1..116: note = Synthetic Sequence
EVQLVESGGGLVKPGGSLRLSCAASGFIFRSYGMSWVRQAPGKGLEWVASISSGGSTYYPDSVKGRFTISRDNAKNSLYLQMNSLRAEDTAVYDCARGYDSGFAYWGQGTLVTVSS

SEQ ID NO: 136 - protein, 112 aa, synthetic construct
  REGION 1..112: note = Synthetic Sequence
DIVLTQSPASLAVSLGQRATISCRASQSVSTSSSSFMHWYQQKPGQPPKLLIKYASNLESGVPARFSGSGSGTDFTLNIHPVEEEDTATYYCQHSWEIPYTFGGGTKLEIKR

SEQ ID NO: 137 - protein, 111 aa, synthetic construct
  REGION 1..111: note = Synthetic Sequence
DIVLTQSPPSLAVSLGQRATISCRASQSVSTSSSSYMHWYQQKPGQPPKLLIKYASNLESGVPARFSGSGSGTDFTLNIHPVEEEDTATYYCQHSWEIPYTFGGGTKLEIK

SEQ ID NO: 138 - protein, 107 aa, synthetic construct
  REGION 1..107: note = Synthetic Sequence
SIVMTQTPKFLLVSAGDRVTITCKASQSVSNDVAWYQQKPGQSPKLLIYYAANRYTGVPDRFTGSGYGTDFTFTISIVQAEDLAVYFCQQDYTSPYTFGGGTKLEIK

SEQ ID NO: 139 - protein, 107 aa, synthetic construct
  REGION 1..107: note = Synthetic Sequence
SIVMTQTPKFLLVSAGDRVTITCKASQSVSNDVGWYQQKPGQSPKLLIYYASNRYSGVPDRFTGSGYGTDFTFTISTVQAEDLAVYFCQQDYTSPYTFGGGTKLEIK

SEQ ID NO: 140 - protein, 112 aa, synthetic construct
  REGION 1..112: note = Synthetic Sequence
DVLMTQTPLYLPVSLGDQASISCRSSQIIVHSNANTYLEWFLQKPGQSPKLLIYKVSNRFSGVPDRFSGSGSGTDFTLKISRVEAEDLGVYYCFQGSHVPYTFGGGTKLEIK

SEQ ID NO: 141 - protein, 108 aa, synthetic construct
  REGION 1..108: note = Synthetic Sequence
QIVLTQSPAIMSASPGEKVTLTCSASSSVSSSYLYWNQQKPGSSPKVWIYNTSNLASGVPARFSGSGSGTSYSLTISSMEAEDAASYFCHQWRSYPPTLGAGTKLELK

SEQ ID NO: 142 - protein, 106 aa, synthetic construct
  REGION 1..106: note = Synthetic Sequence
QIVLTQSPAIMSASPGEKVTMTCSANSSVSYMHWYQQKSGTSPKRWIYDTSKLASGVPARFSGSGSGTSYSLTISSMGAEDAATYYCQQWSSNPWTFGGGTKLEIK

SEQ ID NO: 143 - protein, 111 aa, synthetic construct
  REGION 1..111: note = Synthetic Sequence
DIVLTQSPASLAVSLGQRATISCRASQSVSTSSYSYMHWYQQKPGQPPKLLIKYASNLESGVPARFSGSGSGTDFTLNIHPVEEEDTATYYCQNSWEIPYTFGGGTKLEIK

SEQ ID NO: 144 - protein, 113 aa, synthetic construct
  REGION 1..113: note = Synthetic Sequence
DIVMTQTPSSLAVSLGEKVTMSCKSSQSLLYSSNQKNSLAWYQQKPGQSPKLLIYWASNRESGVPDRFTGSSSGTDFTLTISSVKAEDLAVYYCQQYYSYPLTFGAGTKLELK

SEQ ID NO: 145 - protein, 111 aa, synthetic construct
  REGION 1..111: note = Synthetic Sequence
DIVLTQSPASLAVSLGQRATISCRASQSVSTSSYSYVHWYQQKPGQPPKLLIKYASNLESGVPARFSGSGSGTDFTLNIHPVEEEDTATYYCQHSWEIPYTFGGGTKLEIK

SEQ ID NO: 146 - protein, 107 aa, synthetic construct
  REGION 1..107: note = Synthetic Sequence
DIQMTQSPSSLSASVGDRVTITCKASQSVSNDVAWYQQKPGKAPKLLIYYAANRYTGVPDRFSGSGYGTDFTFTISSLQPEDIATYFCQQDYTSPYTFGQGTKLEIK

SEQ ID NO: 147 - protein, 111 aa, synthetic construct
  REGION 1..111: note = Synthetic Sequence
DIVLTQSPASLAVSPGQRATITCRASQSVSTSSSSFMHWYQQKPGQPPKLLIKYASNLESGVPARFSGSGSGTDFTLTINPVEANDTANYYCQHSWEIPYTFGQGTKLEIK

SEQ ID NO: 148 - protein, 117 aa, synthetic construct
  REGION 1..117: note = Synthetic Sequence
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 149 - protein, 117 aa, synthetic construct
  REGION 1..117: note = Synthetic Sequence
EVQLVQSGAEVKKPGESLRISCKGSGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSS

SEQ ID NO: 150 - protein, 117 aa, synthetic construct
  REGION 1..117: note = Synthetic Sequence
QVQLVQSGAEVKKPGASVKVSCKASGYTFTTYWMHWIRQSPSRGLEWLGNIYPGTGGSNFDEKFKNRFTISRDNSKNTLYLQMNSLRAEDTAVYYCTRWTTGTGAYWGQGTTVTVSS

SEQ ID NO: 151 - protein, 117 aa, synthetic construct
  REGION 1..117: note = Synthetic Sequence
EVQLVQSGAEVKKPGESLRISCKGSGYTFTTYWMHWVRQAPGQGLEWMGNIYPGTGGSNFDEKFKNRFTISRDNSKNTLYLQMNSLRAEDTAVYYCTRWTTGTGAYWGQGTTVTVSS

SEQ ID NO: 152 - protein, 113 aa, synthetic construct
  REGION 1..113: note = Synthetic Sequence
EIVLTQSPATLSLSPGERATLSCKSSQSLLDSGNQKNFLTWYQQKPGQAPRLLIYWASTRESGVPSRFSGSGSGTEFTLTISSLQPDDFATYYCQNDYSYPYTFGQGTKVEIK

SEQ ID NO: 153 - protein, 113 aa, synthetic construct
  REGION 1..113: note = Synthetic Sequence
DIQMTQSPSSLSASVGDRVTITCKSSQSLLDSGNQKNFLTWYQQKPGQAPRLLIYWASTRESGIPPRFSGSGYGTDFTLTINNIESEDAAYYFCQNDYSYPYTFGQGTKVEIK

SEQ ID NO: 154 - protein, 113 aa, synthetic construct
  REGION 1..113: note = Synthetic Sequence
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 155 - protein, 113 aa, synthetic construct
  REGION 1..113: note = Synthetic Sequence
DIVMTQTPLSLPVTPGEPASISCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 156 - protein, 113 aa, synthetic construct
  REGION 1..113: note = Synthetic Sequence
EIVLTQSPATLSLSPGERATLSCKSSQSLLDSGNQKNFLTWYQQKPGKAPKLLIYWASTRESGVPSRFSGSGSGTDFTFTISSLEAEDAATYYCQNDYSYPYTFGQGTKVEIK

SEQ ID NO: 157 - protein, 113 aa, synthetic construct
  REGION 1..113: note = Synthetic Sequence
EIVLTQSPDFQSVTPKEKVTITCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 158 - protein, 113 aa, synthetic construct
  REGION 1..113: note = Synthetic Sequence
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 159 - protein, 113 aa, synthetic construct
  REGION 1..113: note = Synthetic Sequence
DIQMTQSPSSLSASVGDRVTITCKSSQSLLDSGNQKNFLTWYLQKPGQSPQLLIYWASTRESGVPSRFSGSGSGTDFTFTISSLEAEDAATYYCQNDYSYPYTFGQGTKVEIK

SEQ ID NO: 160 - protein, 113 aa, synthetic construct
  REGION 1..113: note = Synthetic Sequence
DVVMTQSPLSLPVTLGQPASISCKSSQSLLDSGNQKNFLTWYQQKPGKAPKLLIYWASTRESGVPSRFSGSGSGTDFTFTISSLEAEDAATYYCQNDYSYPYTFGQGTKVEIK

SEQ ID NO: 161 - protein, 118 aa, synthetic construct
  REGION 1..118: note = Synthetic Sequence
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 162 - protein, 108 aa, synthetic construct
  REGION 1..108: note = Synthetic Sequence
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 163 - protein, 126 aa, synthetic construct
  REGION 1..126: note = Synthetic Sequence
EVQLVESGGGLVQPGGSLRLSCAASGFTLDYYAIGWFRQAPGKEREWASSISSSDGSTYYADSVKGRFTISRDNAKNTVFLQMNSLKPEDTAVYSCAASQAPITIATMMKPFYDYWGQGTQVTVSS

SEQ ID NO: 164 - protein, 123 aa, synthetic construct
  REGION 1..123: note = Synthetic Sequence
EVQLVESGGGLVQPGGSLRLSCAASGFTLDYYAKCWFRQAPGKEREWVSCISSSDGSTYYADSVKGRFTISRDNAKNTVYLQMNSLKPEDTAVYFCAARHGGPLTVEYFFDYWGQGTQVTVSS

SEQ ID NO: 165 - protein, 124 aa, synthetic construct
  REGION 1..124: note = Synthetic Sequence
EVQLVESGGGLVQPGGSLRLSCAASGFTFDYYAIGWFRQAPGKAREGVSCISGGDNSTYYADSVKGRFTISRDNAKNTVYLQMNSLKPEDTAVYYCATGGWKYCSGYDPEYIYWGQGTQVTVSS

SEQ ID NO: 166 - protein, 115 aa, synthetic construct
  REGION 1..115: note = Synthetic Sequence
EVQLVESGGGLVQAGGSLRLSCAASGSTFSQYDVGWYRQAPGKQRELVAFSSSGGRTIYPDSVKGRFTFSRDNTKNTVYLQMTSLKPEDTAVYYCKIDWYLNSYWGQGTQVTVSS

SEQ ID NO: 167 - protein, 114 aa, synthetic construct
  REGION 1..114: note = Synthetic Sequence
EVQLVESGGGLVQAGGSLRLSCAASGVDASNSAMGWYRQAPGKQREWVARITGGGLIAYTDSVKGRFTISRDNAKSTVYLQMNSLEPEDTAVYYCNTINSRDGWGQGTQVTVSS

SEQ ID NO: 168 - protein, 111 aa, synthetic construct
  REGION 1..111: note = Synthetic Sequence
EVQLVESGGGLVQAGGSLTISCAASGITFSDSIVSWYRRARGKQREWVAGISNGGTTKYAESVLGRFTISRDNAKNNVYLQMNGLNPEDTAVYLCKVRQYWGQGTQVTVSS

SEQ ID NO: 169 - protein, 130 aa, synthetic construct
  REGION 1..130: note = Synthetic Sequence
EVQLVESGGGLVQAGGSLRLSCAASESTVLINAMGWYRQAPGKQRELVASISSGGSTNYADSVKGRFTISRDNAKNTVYLQMNSLKPEDTAVYYCNADVYPQDYGLGYVEGKVYYGHDYWGTGTLVTVSS

SEQ ID NO: 170 - protein, 119 aa, synthetic construct
  REGION 1..119: note = Synthetic Sequence
EVQLVESGGGLVQAGGSLRLSCAASGSTFSNYVSNYAMGWGRQAPGTQRELVASISNGDTTNYADSVKGRFTISRDNAKNTVYLQMNSLKPEDTAVYYCFEHQVAGLTWGQGTQVTVSS

SEQ ID NO: 171 - protein, 118 aa, synthetic construct
  REGION 1..118: note = Synthetic Sequence
  SITE 27: note = misc_feature - Xaa can be any naturally occurring amino acid
  SITE 32: note = misc_feature - Xaa can be any naturally occurring amino acid
  SITE 75: note = misc_feature - Xaa can be any naturally occurring amino acid
EVQLVESGGGLVQAGGSLRLSCVASGXALKIXVMGWYRQAPGKQRELVAAITSGGRTNYSDSVKGRFTISGDNAXNTVYLQMNSLKSEDTAVYYCREWNSGYPPVDYWGQGTQVTVSS

SEQ ID NO: 172 - protein, 120 aa, synthetic construct
  REGION 1..120: note = Synthetic Sequence
EVQLVESGGGLVQAGGSLRLSCAASGRTFSSGTMGWFRRAPGKEREFVASIPWSGGRTYYADSVKDRFTISRDNAQNTVFLQMNSLKPEDTAVYYCAFKERSTGWDFASWGQGIQVTVSS

SEQ ID NO: 173 - protein, 127 aa, synthetic construct
  REGION 1..127: note = Synthetic Sequence
EVQLVESGGGLVQTGGSLRLSCAASGFTLDYYGIGWFRQAPGKEREGVSFISGSDGSTYYAESVKGRFTISRDKAKNTVYLQMNSLKPEDTAVYYCAADPWGPPSIATMTSYEYKHWGQGTQVTVSS

SEQ ID NO: 174 - protein, 113 aa, synthetic construct
  REGION 1..113: note = Synthetic Sequence
EVQLVESGGGLVQPGGSLRLSCAASGFTFSTYTMIWLRRAPGKGFEWVSTIDKDGNTNYVDSVKGRFAVSRDNTKNTLYLQMNSLKPEDTAMYYCTKHGSSARGQGTRVTVSS

SEQ ID NO: 175 - protein, 114 aa, synthetic construct
  REGION 1..114: note = Synthetic Sequence
EVQLVESGGGLVEPGGSLRLSCVASGFTFSSYDMSWVRQAPGKGLEWVSTINSGGGITYRGSVKGRFTISRDNAKNTLYLQMNSLKPEDTAVYYCENGGSSYRRGQGTQVTVSS

SEQ ID NO: 176 - protein, 114 aa, synthetic construct
  REGION 1..114: note = Synthetic Sequence
QVQLVQSGAELKKPGASVKMSCKASGYTFTGYTMHWVKQAPGQGLEWIGYINPRSGYTEYNQKFKDRTTLTADKSTSTAYMELSSLRSEDSAVYYCARPWFAYWGQGTLVTVSS

SEQ ID NO: 177 - protein, 114 aa, synthetic construct
  REGION 1..114: note = Synthetic Sequence
QVQLVQSGAEVKKPGASVKMSCKASGYTFTGYTMHWVKQAPGQGLEWIGYINPRSGYTEYNQKFKDRTTLTADKSTSTAYMELSSLRSEDTAVYYCARPWFAYWGQGTLVTVSS

SEQ ID NO: 178 - protein, 114 aa, synthetic construct
  REGION 1..114: note = Synthetic Sequence
QVQLVQSGAEVKKPGASVKMSCKASGYTFTGYTMHWVRQAPGQGLEWIGYINPRSGYTEYNQKFKDRTTLTADKSTSTAYMELSSLRSEDTAVYYCARPWFAYWGQGTLVTVSS

SEQ ID NO: 179 - protein, 114 aa, synthetic construct
  REGION 1..114: note = Synthetic Sequence
QVQLVQSGAEVKKPGASVKVSCKASGYTFTGYTMHWVRQAPGQGLEWIGYINPRSGYTEYNQKFKDRTTLTADKSTSTAYMELSSLRSEDTAVYYCARPWFAYWGQGTLVTVSS

SEQ ID NO: 180 - protein, 114 aa, synthetic construct
  REGION 1..114: note = Synthetic Sequence
QVQLVQSGAEVKKPGASVKVSCKASGYTFTGYTMHWVRQAPGQGLEWIGYINPRSGYTEYNQKFKDRTTITADKSTSTAYMELSSLRSEDTAVYYCARPWFAYWGQGTLVTVSS

SEQ ID NO: 181 - protein, 113 aa, synthetic construct
  REGION 1..113: note = Synthetic Sequence
DIVMTQSPASLTVTPGEKVTITCKSSQSLLNSGNQKNYLTWYQQKPGQPPKLLIYWASTRESGVPDRFTGSGSGTDFTLTISSLQAEDVAVYYCQNDYSYPLTFGQGTKLEIK

SEQ ID NO: 182 - protein, 113 aa, synthetic construct
  REGION 1..113: note = Synthetic Sequence
DIVMTQSPASLSVTPGEKVTITCKSSQSLLNSGNQKNYLTWYQQKPGQPPKLLIYWASTRESGVPDRFTGSGSGTDFTLTISSLQAEDVAVYYCQNDYSYPLTFGQGTKLEIK

SEQ ID NO: 183 - protein, 113 aa, synthetic construct
  REGION 1..113: note = Synthetic Sequence
DIVMTQSPAFLSVTPGEKVTITCKSSQSLLNSGNQKNYLTWYQQKPGQPPKLLIYWASTRESGVPDRFTGSGSGTDFTLTISSLQAEDVAVYYCQNDYSYPLTFGQGTKLEIK

SEQ ID NO: 184 - protein, 113 aa, synthetic construct
  REGION 1..113: note = Synthetic Sequence
DIVMTQSPAFLSVTPGEKVTITCKSSQSLLNSGNQKNYLTWYQQKPGQPPKLLIYWASTRESGVPDRFSGSGSGTDFTLTISSLQAEDVAVYYCQNDYSYPLTFGQGTKLEIK

SEQ ID NO: 185 - protein, 450 aa, synthetic construct
  REGION 1..450: note = Synthetic Sequence
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 213 aa, synthetic construct
  REGION 1..213: note = Synthetic Sequence
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 187 - protein, 449 aa, synthetic construct
  REGION 1..449: note = Synthetic Sequence
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 216 aa, synthetic construct
  REGION 1..216: note = Synthetic Sequence
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 189 - protein, 449 aa, synthetic construct
  REGION 1..449: note = Synthetic Sequence
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 213 aa, synthetic construct
  REGION 1..213: note = Synthetic Sequence
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 120 aa, synthetic construct [sequence omitted]

SEQ ID NO: 192 - protein, 106 aa, synthetic construct [sequence omitted]

SEQ ID NO: 193 - protein, 118 aa, synthetic construct [sequence omitted]

SEQ ID NO: 194 - protein, 118 aa, synthetic construct
  REGION 1..118: note = Synthetic Sequence
QVQLVQSGGGVVQSGRSLRLSCAASGFKFSGYGMHWVRQAPGKGLEWVAVIWYDGSKKYYVDSVKGRFTISRDNSKNTLYLQMNSLRGEDTAVYYCARQMGYWHFDLWGRGTLVTVSS

SEQ ID NO: 195 - protein, 108 aa, synthetic construct [sequence omitted]

SEQ ID NO: 196 - protein, 108 aa, synthetic construct [sequence omitted]

SEQ ID NO: 197 - protein, 125 aa, synthetic construct
  REGION 1..125: note = Synthetic Sequence
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 198 - protein, 125 aa, synthetic construct
  REGION 1..125: note = Synthetic Sequence
EVKLVESGGGLVKPGRSLRLSCAASGFTFNTYAMNWVRQAPGKGLEWVARIRSKYNNYATYYADSVKDRFTISRDDSKSILYLQMNNLKTEDTAMYYCVRHGNFGNSYVSWFAYWGQGTLVTVSS

SEQ ID NO: 199 - protein, 125 aa, synthetic construct
  REGION 1..125: note = Synthetic Sequence
EVKLVESGGGLVKPGRSLRLSCAASGFTFNTYAMNWVRQAPGKGLEWVARIRSKYNNYATYYADSVKDRFTISRDDSKSILYLQMNSLKTEDTAMYYCVRHGNFGNSYVSWFAYWGQGTLVTVSS

SEQ ID NO: 200 - protein, 125 aa, synthetic construct
  REGION 1..125: note = Synthetic Sequence
EVKLVESGGGLVKPGRSLRLSCAASGFTFNTYAMNWVRQAPGKGLEWVARIRSKYNNYATYYADSVKDRFTISRDDSKSILYLQMNSLKTEDTAMYYCVRHGNFGNSYVSWFAYWGQGTMVTVSS

SEQ ID NO: 201 - protein, 109 aa, synthetic construct
  REGION 1..109: note = Synthetic Sequence
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 202 - protein, 109 aa, synthetic construct
  REGION 1..109: note = Synthetic Sequence
QAVVTQEPSFSVSPGGTVTLTCRSSTGAVTTSNYANWVQQTPGQAFRGLIGGTNKRAPGVPARFSGSILGNKAALTITGAQADDESIYFCALWYSNLWVFGGGTKLTVL

SEQ ID NO: 203 - protein, 109 aa, synthetic construct
  REGION 1..109: note = Synthetic Sequence
QAVVTQEPSFSVSPGGTVTLTCRSSTGAVTTSNYANWVQQTPGQAFRGLIGGTNKRAPGVPARFSGSILGNKAALTITGAQADDESDYYCALWYSNLWVFGGGTKLTVL

SEQ ID NO: 204 - protein, 119 aa, synthetic construct
  REGION 1..119: note = Synthetic Sequence
QVQLVQSGSELKKPGASVKMSCKASGYTFTRYTMHWVRQAPGKGLEWIGYINPSRGYTNYNQKFKDRATLTTDKSTSTAYMQLSSLRSEDTAVYYCARYYDDHYSLDYWGQGTLVTVSS

SEQ ID NO: 205 - protein, 106 aa, synthetic construct
  REGION 1..106: note = Synthetic Sequence
QIVLTQSPATLSLSPGERATMSCSASSSVSYMNWYQQKPGKAPKRWIYDTSKLASGVPSRFRGSGSGTDYTLTISSLQPEDFATYYCQQWSSNPFTFGGGTKVEIK

SEQ ID NO: 206 - protein, 125 aa, synthetic construct
  REGION 1..125: note = Synthetic Sequence
ELQLVESGGGVVQPGRSLRLSCAASGFTFSSYGMHWVRQAPGKGLEWVAVISYDGSNKYYADSVKGRFTISRDNSKNTLYLQMNSLRSEDTAVYYCARLSPYCTNGVCWDAFDIWGQGTMVTVSS

SEQ ID NO: 207 - protein, 122 aa, synthetic construct
  REGION 1..122: note = Synthetic Sequence
ELQLVESGGGLVKPGRSLRLSCTASGFTFGDYAMSWFRQAPGKGLEWVGFIRSKAYGGTTEYAASVKGRFTISRDDSKSIAYLQMNSLKTEDTAVYYCTPQLWLLQDAFDIWGQGTMVTVSS

SEQ ID NO: 208 - protein, 120 aa, synthetic construct
  REGION 1..120: note = Synthetic Sequence
ELQLVESGPGLVKPSGTLSLTCAVSGGSISSRNWWSWVRQPPGKGLEWIGDIYHSGSTNYNPSLKSRVTISVDKSKNQFSLKLSSVTAADTAVYYCASGYTSCRDAFDIWGQGTMVTVSS

SEQ ID NO: 209 - protein, 126 aa, synthetic construct
  REGION 1..126: note = Synthetic Sequence
ELQLVEWGAGLLKPSETLSLTCAVYGGSFSGYYWSWIRQPPGKGLEWIGEINHSGSTNYNPSLKSRVTISVDTSKNQFSLKLSSVTAADTAVYYCARGRGRFLGWLLGGSNWFDPWGQGTLVTVSS

SEQ ID NO: 210 - protein, 119 aa, synthetic construct
  REGION 1..119: note = Synthetic Sequence
ELQLVEWGAGLLKPSETLSLTCAVYGGSFSGYYWSWIRQPPGKGLEWIGEINHSGSTNYNPSLKSRVTISVDTSKNQFSLKLSSVTAADTAVYYCARGPDRMGHGFDIWGQGTMVTVSS

SEQ ID NO: 211 - protein, 127 aa, synthetic construct
  REGION 1..127: note = Synthetic Sequence
ELQLVESGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDRRRIAARQYYGMDVWGQGTTVTVSS

SEQ ID NO: 212 - protein, 121 aa, synthetic construct
  REGION 1..121: note = Synthetic Sequence
ELQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKAKFLGHYYGMDVWGQGTTVTVSS

SEQ ID NO: 213 - protein, 123 aa, synthetic construct
  REGION 1..123: note = Synthetic Sequence
ELQLVESGPVLVKPTDTLTLTCTVSGFSLNNPRMGVSWIRQPPGKTLEWLAHIFPSDAKAHSASLKSRLTISKDTSKSQVVPTMTNMDPVDTATYYCARILGEYYPPAWFDPWGQGTLVTVSS

SEQ ID NO: 214 - protein, 107 aa, synthetic construct
  REGION 1..107: note = Synthetic Sequence
ELQMTQSPSSLSASVGDRVSITCRASQTISNYLNWYQLKPGKAPKLLIYAASTLQSEVPTRFSGSGSGTDFTLTISGLHPEDFATYYCQQFNSYPRTFGQGTKVEIK

SEQ ID NO: 215 - protein, 107 aa, synthetic construct
  REGION 1..107: note = Synthetic Sequence
ELQMTQSPSSLSASVGDRVTITCRASQGISNYLAWYQQKPGKVPKLLIYAASTLQSGVPSRFSGSGSGTDFTLTISSLQPEDFATYYCQQSYSTPPTFGQGTKLEIK

SEQ ID NO: 216 - protein, 107 aa, synthetic construct
  REGION 1..107: note = Synthetic Sequence
ELVMTQSPSSLSASVGDRVTITCRASQGIGNYLAWYQQKPGQPPKMLIYWASIRESGVPDRFSGSGSGTDFTLTISSLQAEDVAVYYCQQYYSNPQTFGQGTKVEIK

SEQ ID NO: 217 - protein, 107 aa, synthetic construct
  REGION 1..107: note = Synthetic Sequence
ELVMTQSPSSLSASVGDRVTITCRASQGISNYLNWYQQKPGKAPKLLIYDASNLETGVPSRFSGSGSGTDFTLTISSLQPEDFATYYCQQSYSTPYTFGQGTKVDIK

SEQ ID NO: 218 - protein, 107 aa, synthetic construct
  REGION 1..107: note = Synthetic Sequence
ELQMTQSPSSLSASVGDRVTITCRASQSISSYLNWYQQKSGKAPKLLIYAASSLQSGVPSRFSGSGSGTDFTLTISSLQPEDFATYYCQQSYSSPWTFGQGTKVEIK

SEQ ID NO: 219 - protein, 108 aa, synthetic construct
  REGION 1..108: note = Synthetic Sequence
ELVLTQSPGTLSLSPGERATLSCRASQSVSSNYLAWYQQKPGQAPRLLIYGASSRATGIPDRFSGSGSGTDFTLTISSLQPEDVATYYCQKYNSAPLTFGGGTKVEIK

SEQ ID NO: 220 - protein, 107 aa, synthetic construct
  REGION 1..107: note = Synthetic Sequence
ELQMTQSPSSLSASVGDRVTITCRASQSISSYLNWYQQKPGKAPKLLIYAASSLQSGVPSRFSGSGSGTEFTLTISSLQPEDFATYYCLQHNAYPYTFGQGTKVEIK

SEQ ID NO: 221 - protein, 113 aa, synthetic construct
  REGION 1..113: note = Synthetic Sequence
ELVMTQSPDSLAVSLGERATINCKSSQSVLYSSNNKNYLAWYQQKPGQPPKLLIYWASTRESGVPDRFSGSGSGTDFTLTISSLQAEDVAVYYCQQYLKIPYTFGQGTKVEIK

SEQ ID NO: 222 - protein, 119 aa, synthetic construct
  REGION 1..119: note = Synthetic Sequence
QVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYCLDYWGQGTLVTVSS

SEQ ID NO: 223 - protein, 119 aa, synthetic construct
  REGION 1..119: note = Synthetic Sequence
QVQLVQSGAEVKKPGASVKVSCKASGYTFTRYTMHWVRQAPGQGLEWMGYINPSRGYTNYNQKFKDRVTMTTDTSISTAYMELSRLRSDDTAVYYCARYYDDHYSLDYWGQGTLVTVSS

SEQ ID NO: 224 - protein, 106 aa, synthetic construct
  REGION 1..106: note = Synthetic Sequence
EIVLTQSPATLSLSPGERATLSCSASSSVSYMNWYQQKPGQAPRLLIYDTSKLASGVPAHFRGSGSGTDYTLTISSLEPEDFAVYYCQQWSSNPFTFGQGTKVEIK

SEQ ID NO: 225 - protein, 125 aa, synthetic construct
  REGION 1..125: note = Synthetic Sequence
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSWFAYWGQGTLVTVSS

SEQ ID NO: 226 - protein, 125 aa, synthetic construct; identical to SEQ ID NO: 225
  REGION 1..125: note = Synthetic Sequence

SEQ ID NO: 227 - protein, 125 aa, synthetic construct
  REGION 1..125: note = Synthetic Sequence
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSYFAYWGQGTLVTVSS

SEQ ID NO: 228 - protein, 125 aa, synthetic construct
  REGION 1..125: note = Synthetic Sequence
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCARHGNFGNSYVSHFAYWGQGTLVTVSS

SEQ ID NO: 229 - protein, 110 aa, synthetic construct
  REGION 1..110: note = Synthetic Sequence
DIQMTQSPSSLSASVGDRVTITCRSSTGAVTTSNYANWVQQKPGKAPKGLIGGTNKRAPGVPSRFSGSLIGDKATLTISSLQPEDFATYYCALWYSNLWVFGQGTKVEIK

SEQ ID NO: 230 - protein, 110 aa, synthetic construct
  REGION 1..110: note = Synthetic Sequence
DIQMTQSPSSLSASVGDRVTITCRSSTGAVTTSNYANWVQQKPGKAPKGLIGGTNKRAPGVPARFSGSGSGTDFTLTISSLQPEDFATYYCALWYSNLWVFGQGTKVEIK

SEQ ID NO: 231 - protein, 110 aa, synthetic construct
  REGION 1..110: note = Synthetic Sequence
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIK

SEQ ID NO: 232 - protein, 110 aa, synthetic construct; identical to SEQ ID NO: 229
  REGION 1..110: note = Synthetic Sequence

SEQ ID NO: 233 - protein, 165 aa, synthetic construct
  REGION 1..165: note = Synthetic Sequence
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 234 - protein, 165 aa, synthetic construct [sequence omitted]

SEQ ID NO: 235 - protein, 165 aa, synthetic construct
  REGION 1..165: note = Synthetic Sequence
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 236 - protein, 165 aa, synthetic construct
  REGION 1..165: note = Synthetic Sequence
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 237 - protein, 157 aa, synthetic construct [sequence omitted]

SEQ ID NO: 238 - protein, 171 aa, synthetic construct
  REGION 1..171: note = Synthetic Sequence
LPGVGLTPSAAQTARQHPKMHLAHSNLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 239 - protein, 281 aa, synthetic construct [sequence omitted]

SEQ ID NO: 240 - protein, 153 aa, synthetic construct
  REGION 1..153: note = Synthetic Sequence
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 241 - protein, 133 aa, synthetic construct [sequence omitted]

SEQ ID NO: 242 - protein, 129 aa, synthetic construct [sequence omitted]

SEQ ID NO: 243 - protein, 185 aa, synthetic construct
  REGION 1..185: note = Synthetic Sequence
APVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLTTKLQAQNQWLQDMTTHLILRSFKEFLQSSLRALRQM

SEQ ID NO: 244 - protein, 114 aa, synthetic construct
  REGION 1..114: note = Synthetic Sequence
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 245 - protein, 194 aa, synthetic construct
  REGION 1..194: note = Synthetic Sequence
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDL

SEQ ID NO: 246 - protein, 270 aa, synthetic construct
  REGION 1..270: note = Synthetic Sequence
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 247 - protein, 193 aa, synthetic construct
  REGION 1..193: note = Synthetic Sequence
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 248 - protein, 166 aa, synthetic construct
  REGION 1..166: note = Synthetic Sequence
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 249 - protein, 5 aa, synthetic construct
  REGION 1..5: note = Synthetic Sequence
GGGGS

SEQ ID NO: 250 - protein, 10 aa, synthetic construct
  REGION 1..10: note = Synthetic Sequence
GGGGSGGGGS

SEQ ID NO: 251 - protein, 15 aa, synthetic construct
  REGION 1..15: note = Synthetic Sequence
GGGGSGGGGSGGGGS

SEQ ID NO: 252 - protein, 20 aa, synthetic construct
  REGION 1..20: note = Synthetic Sequence
GGGGSGGGGSGGGGSGGGGS

SEQ ID NO: 253 - protein, 25 aa, synthetic construct
  REGION 1..25: note = Synthetic Sequence
GGGGSGGGGSGGGGSGGGGSGGGGS

SEQ ID NO: 254 - protein, 30 aa, synthetic construct
  REGION 1..30: note = Synthetic Sequence
GGGGSGGGGSGGGGSGGGGSGGGGSGGGGS

SEQ ID NO: 255 - protein, 35 aa, synthetic construct
  REGION 1..35: note = Synthetic Sequence
GGGGSGGGGSGGGGSGGGGSGGGGSGGGGSGGGGS

SEQ ID NO: 256 - protein, 40 aa, synthetic construct
  REGION 1..40: note = Synthetic Sequence
GGGGSGGGGSGGGGSGGGGSGGGGSGGGGSGGGGSGGGGS

SEQ ID NO: 257 - protein, 16 aa, synthetic construct
  REGION 1..16: note = Synthetic Sequence
GGSGGSGGGGSGGGGS

SEQ ID NO: 258 - protein, 8 aa, synthetic construct
  REGION 1..8: note = Synthetic Sequence
GGGGGGGG

SEQ ID NO: 259 - protein, 6 aa, synthetic construct
  REGION 1..6: note = Synthetic Sequence
GGGGGG

SEQ ID NO: 260 - protein, 5 aa, synthetic construct
  REGION 1..5: note = Synthetic Sequence
EAAAK

SEQ ID NO: 261 - protein, 10 aa, synthetic construct
  REGION 1..10: note = Synthetic Sequence
EAAAKEAAAK

SEQ ID NO: 262 - protein, 15 aa, synthetic construct
  REGION 1..15: note = Synthetic Sequence
EAAAKEAAAKEAAAK

SEQ ID NO: 263 - protein, 12 aa, synthetic construct
  REGION 1..12: note = Synthetic Sequence
AEAAAKEAAAKA

SEQ ID NO: 264 - protein, 17 aa, synthetic construct
  REGION 1..17: note = Synthetic Sequence
AEAAAKEAAAKEAAAKA

SEQ ID NO: 265 - protein, 22 aa, synthetic construct
  REGION 1..22: note = Synthetic Sequence
AEAAAKEAAAKEAAAKEAAAKA

SEQ ID NO: 266 - protein, 27 aa, synthetic construct
  REGION 1..27: note = Synthetic Sequence
AEAAAKEAAAKEAAAKEAAAKEAAAKA

SEQ ID NO: 267 - protein, 46 aa, synthetic construct
  REGION 1..46: note = Synthetic Sequence
AEAAAKEAAAKEAAAKEAAAKALEAEAAAKEAAAKEAAAKEAAAKA

SEQ ID NO: 268 - protein, 5 aa, synthetic construct
  REGION 1..5: note = Synthetic Sequence
PAPAP

SEQ ID NO: 269 - protein, 18 aa, synthetic construct
  REGION 1..18: note = Synthetic Sequence
KESGSVSSEQLAQFRSLD

SEQ ID NO: 270 - protein, 14 aa, synthetic construct
  REGION 1..14: note = Synthetic Sequence
EGKSSGSGSESKST

SEQ ID NO: 271 - protein, 12 aa, synthetic construct
  REGION 1..12: note = Synthetic Sequence
GSAGSAAGSGEF

SEQ ID NO: 272 - protein, 5 aa, synthetic construct
  REGION 1..5: note = Synthetic Sequence
GGGSE

SEQ ID NO: 273 - protein, 5 aa, synthetic construct
  REGION 1..5: note = Synthetic Sequence
GSESG

SEQ ID NO: 274 - protein, 5 aa, synthetic construct
  REGION 1..5: note = Synthetic Sequence
GSEGS

SEQ ID NO: 275 - protein, 35 aa, synthetic construct
  REGION 1..35: note = Synthetic Sequence
GEGGSGEGSSGEGSSSEGGGSEGGGSEGGGSEGGS

SEQ ID NO: 276 - protein, 4 aa, synthetic construct
  REGION 1..4: note = Synthetic Sequence
CPPC

SEQ ID NO: 277 - protein, 166 aa, synthetic construct
  REGION 1..166: note = Synthetic Sequence (Human IFN-beta)
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 278 - protein, 167 aa, synthetic construct
  REGION 1..167: note = Synthetic Sequence (Consensus interferon)
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 279 - protein, 166 aa, synthetic construct
  REGION 1..166: note = Synthetic polymer.
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 280 - protein, 166 aa, synthetic construct
  REGION 1..166: note = Synthetic Sequence (IFN-Con2 (Consensus Interferon Variant 2))
CDLPQTHSLGNRRTLMLLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 281 - protein, 166 aa, synthetic construct
  REGION 1..166: note = Synthetic Sequence (IFN-Con3 (Consensus Interferon Variant 3))
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNEDSILAVRKYFQRITLYLTEKKYSPCAWEVVRAEIMRSFSLSTNLQERLRRKE

SEQ ID NO: 282 - protein, 167 aa, synthetic construct
  REGION 1..167: note = Synthetic Sequence (Consensus Interferon Variant)
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNEDSILAVRKYFQRITLYLTEKKYSPCAWEVVRAEIMRSFSLSTNLQERLRRKE

SEQ ID NO: 283 - protein, 167 aa, synthetic construct
  REGION 1..167: note = Synthetic Sequence (Consensus Interferon Variant)
MCDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNEDSILAVRKYFQRITLYLTEKKYSPCAWEVVRAEIMRSFSLCTNLQERLRRKE

SEQ ID NO: 284 - protein, 6 aa, synthetic construct
  REGION 1..6: note = Synthetic Sequence (human IFN-alpha2a)
EEFGNQ

SEQ ID NO: 285 - protein, 7 aa, synthetic construct
  REGION 1..7: note = Synthetic Sequence (human IFN-alpha2a)
EEFDGNQ

SEQ ID NO: 286-287 - skipped (2 entries)

SEQ ID NO: 288 - protein, 118 aa, synthetic construct
  REGION 1..118: note = Synthetic Sequence
QVQLQESGGGLAQAGGSLRLSCAASGRTFSMGWFRQAPGKEREFVAAITYSGGSPYYASSVRGRFTISRDNAKNTVYLQMNSLKPEDTAVYYCAANPTYGSDWNAENWGQGTQVTVSS

SEQ ID NO: 289-291 - skipped (3 entries)

SEQ ID NO: 292 - protein, 114 aa, synthetic construct
  REGION 1..114: note = Synthetic Sequence (wild type IL-15)
NWVNVISDLKKIEDLIQSMHIDATLYTESDVHPSCKVTAMKCFLLELQVISHESGDTDIHDTVENLIILANNILSSNGNITESGCKECEELEEKNIKEFLQSFVHIVQMFINTS

SEQ ID NO: 293 - protein, 10 aa, synthetic construct
  REGION 1..10: note = Synthetic polymer.
GFNIKDTYIH

SEQ ID NO: 294 - protein, 17 aa, synthetic construct
  REGION 1..17: note = Synthetic polymer.
RIDPANDNTLYASKFQG

SEQ ID NO: 295 - protein, 17 aa, synthetic construct
  REGION 1..17: note = Synthetic polymer.
RIDPANDNTLYARKFQG

SEQ ID NO: 296 - protein, 11 aa, synthetic construct
  REGION 1..11: note = Synthetic polymer.
GRGYGYYVFDH

SEQ ID NO: 297 - protein, 11 aa, synthetic construct
  REGION 1..11: note = Synthetic polymer.
TRGYGYYVFDT

SEQ ID NO: 298 - protein, 5 aa, synthetic construct
  REGION 1..5: note = Synthetic polymer.
SISQY

SEQ ID NO: 299 - protein, 5 aa, synthetic construct
  REGION 1..5: note = Synthetic polymer.
SISKY

SEQ ID NO: 300 - protein, 6 aa, synthetic construct
  REGION 1..6: note = Synthetic polymer.
SGSTLQ

SEQ ID NO: 301 - protein, 6 aa, synthetic construct
  REGION 1..6: note = Synthetic polymer.
HNENPL

SEQ ID NO: 302 - protein, 6 aa, synthetic construct
  REGION 1..6: note = Synthetic polymer.
HNEFPV

SEQ ID NO: 303 - protein, 6 aa, synthetic construct
  REGION 1..6: note = Synthetic polymer.
HNEFPP

SEQ ID NO: 304 - protein, 6 aa, synthetic construct
  REGION 1..6: note = Synthetic polymer.
VNEFPP

SEQ ID NO: 305 - protein, 6 aa, synthetic construct
  REGION 1..6: note = Synthetic polymer.
VNEFPV

SEQ ID NO: 306 - protein, 8 aa, synthetic construct
  REGION 1..8: note = Synthetic polymer.
GFIFSNYG

SEQ ID NO: 307 - protein, 8 aa, synthetic construct
  REGION 1..8: note = Synthetic polymer.
IWYDGSNK

SEQ ID NO: 308 - protein, 16 aa, synthetic construct
  REGION 1..16: note = Synthetic polymer.
ARSYDMLTGSGDYYGL

SEQ ID NO: 309 - protein, 6 aa, synthetic construct
  REGION 1..6: note = Synthetic polymer.
QDITNY

SEQ ID NO: 310 - protein, 9 aa, synthetic construct
  REGION 1..9: note = Synthetic polymer.
QQYNNYPLT

SEQ ID NO: 311 - protein, 11 aa, synthetic construct
  REGION 1..11: note = Synthetic polymer.
SGYTGTDYNMH

SEQ ID NO: 312 - protein, 17 aa, synthetic construct
  REGION 1..17: note = Synthetic polymer.
YIYPYTGGTGYNQKFKN

SEQ ID NO: 313 - protein, 5 aa, synthetic construct
  REGION 1..5: note = Synthetic polymer.
DFGMN

SEQ ID NO: 314 - protein, 11 aa, synthetic construct
  REGION 1..11: note = Synthetic polymer.
LIYYDGSNKFY

SEQ ID NO: 315 - protein, 12 aa, synthetic construct
  REGION 1..12: note = Synthetic polymer.
PHYDGYYHFFDS

SEQ ID NO: 316 - protein, 15 aa, synthetic construct
  REGION 1..15: note = Synthetic polymer.
RASESVDSYDNSLMH

SEQ ID NO: 317 - protein, 7 aa, synthetic construct
  REGION 1..7: note = Synthetic polymer.
LASNLES

SEQ ID NO: 318 - protein, 9 aa, synthetic construct
  REGION 1..9: note = Synthetic polymer.
QQNNEDPYT

SEQ ID NO: 319 - protein, 11 aa, synthetic construct
  REGION 1..11: note = Synthetic polymer.
KGSQDINNYLA

SEQ ID NO: 320 - protein, 7 aa, synthetic construct
  REGION 1..7: note = Synthetic polymer.
NTDILHT

SEQ ID NO: 321 - protein, 8 aa, synthetic construct
  REGION 1..8: note = Synthetic polymer.
YQYNNGYT

SEQ ID NO: 322 - protein, 7 aa, synthetic construct
  REGION 1..7: note = Synthetic polymer.
GYTFTSY

SEQ ID NO: 323-332 - skipped (10 entries)

SEQ ID NO: 333 - protein, 6 aa, synthetic construct
  REGION 1..6: note = Synthetic polymer.
DPSDNY

SEQ ID NO: 334 - protein, 16 aa, synthetic construct
  REGION 1..16: note = Synthetic polymer.
PKSAYAFDVGGYAMDY

SEQ ID NO: 335 - protein, 11 aa, synthetic construct
  REGION 1..11: note = Synthetic polymer.
RTSENIDSYLT

SEQ ID NO: 336 - protein, 7 aa, synthetic construct
  REGION 1..7: note = Synthetic polymer.
AATLLAD

SEQ ID NO: 337 - protein, 9 aa, synthetic construct
  REGION 1..9: note = Synthetic polymer.
QHYYSTPWT

SEQ ID NO: 338 - protein, 6 aa, synthetic construct
  REGION 1..6: note = Synthetic polymer.
GFNIKD

SEQ ID NO: 339 - protein, 9 aa, synthetic construct
  REGION 1..9: note = Synthetic polymer.
RIDPANDNT

SEQ ID NO: 340 - protein, 9 aa, synthetic construct
  REGION 1..9: note = Synthetic polymer.
GYGYYVFDH

SEQ ID NO: 341 - protein, 11 aa, synthetic construct
  REGION 1..11: note = Synthetic polymer.
RTSRSISQYLA

SEQ ID NO: 342 - protein, 7 aa, synthetic construct
  REGION 1..7: note = Synthetic polymer.
SGSTLQS

SEQ ID NO: 343 - protein, 9 aa, synthetic construct
  REGION 1..9: note = Synthetic polymer.
QQHNENPLT

SEQ ID NO: 344 - protein, 8 aa, synthetic construct
  REGION 1..8: note = Synthetic polymer.
GYTFTAHI

SEQ ID NO: 345 - protein, 8 aa, synthetic construct
  REGION 1..8: note = Synthetic polymer.
IKPQYGAV

SEQ ID NO: 346 - protein, 6 aa, synthetic construct
  REGION 1..6: note = Synthetic polymer.
QGVGSD

SEQ ID NO: 347 - protein, 5 aa, synthetic construct
  REGION 1..5: note = Synthetic polymer.
QVLQF

SEQ ID NO: 348 - protein, 7 aa, synthetic construct
  REGION 1..7: note = Synthetic polymer.
GYTFTSN

SEQ ID NO: 349 - protein, 6 aa, synthetic construct
  REGION 1..6: note = Synthetic polymer.
YPRSGN

SEQ ID NO: 350 - protein, 7 aa, synthetic construct
  REGION 1..7: note = Synthetic polymer.
RVPYFDH

SEQ ID NO: 351 - protein, 11 aa, synthetic construct
  REGION 1..11: note = Synthetic polymer.
KASQSVGNNVA

SEQ ID NO: 352 - protein, 7 aa, synthetic construct
  REGION 1..7: note = Synthetic polymer.
YASNRYT

SEQ ID NO: 353 - protein, 9 aa, synthetic construct
  REGION 1..9: note = Synthetic polymer.
QQHYSSPFT

SEQ ID NO: 354 - protein, 5 aa, synthetic construct
  REGION 1..5: note = Synthetic polymer.
GYWMY

SEQ ID NO: 355 - protein, 5 aa, synthetic construct
  REGION 1..5: note = Synthetic polymer.
SYSMG

SEQ ID NO: 356 - protein, 5 aa, synthetic construct
  REGION 1..5: note = Synthetic polymer.
FNAMG

SEQ ID NO: 357 - protein, 16 aa, synthetic construct
  REGION 1..16: note = Synthetic polymer.
AISPGGGSTYYPDSVK

SEQ ID NO: 358 - protein, 16 aa, synthetic construct
  REGION 1..16: note = Synthetic polymer.
AISWSGDETSYADSVK

SEQ ID NO: 359 - protein, 16 aa, synthetic construct
  REGION 1..16: note = Synthetic polymer.
TIARAGATKYADSVKG

SEQ ID NO: 360 - protein, 16 aa, synthetic construct
  REGION 1..16: note = Synthetic polymer.
AVRWSSTGIYYTQYAD

SEQ ID NO: 361 - protein, 12 aa, synthetic construct
  REGION 1..12: note = Synthetic polymer.
SLTATHTYEYDY

SEQ ID NO: 362 - protein, 13 aa, synthetic construct
  REGION 1..13: note = Synthetic polymer.
DRWWRPAGLQWDY

SEQ ID NO: 363 - protein, 9 aa, synthetic construct
  REGION 1..9: note = Synthetic polymer.
RVFDLPNDY

SEQ ID NO: 364 - protein, 15 aa, synthetic construct
  REGION 1..15: note = Synthetic polymer.
DTYNSNPARWDGYDF

SEQ ID NO: 365 - protein, 5 aa, synthetic construct
  REGION 1..5: note = Synthetic polymer.
AYVIS

SEQ ID NO: 366 - protein, 17 aa, synthetic construct
  REGION 1..17: note = Synthetic polymer.
EIYPGSGSSYYNEKFKG

SEQ ID NO: 367 - protein, 9 aa, synthetic construct
  REGION 1..9: note = Synthetic polymer.
SGDGSKFVY

SEQ ID NO: 368 - protein, 15 aa, synthetic construct
  REGION 1..15: note = Synthetic polymer.
KASQSVDYCGDSYMN

SEQ ID NO: 369 - protein, 7 aa, synthetic construct
  REGION 1..7: note = Synthetic polymer.
VASNLES

SEQ ID NO: 370 - protein, 9 aa, synthetic construct
  REGION 1..9: note = Synthetic polymer.
QQSLQDPPT

SEQ ID NO: 371 - protein, 10 aa, synthetic construct
  REGION 1..10: note = Synthetic polymer.
GYTFTSYVIH

SEQ ID NO: 372 - protein, 16 aa, synthetic construct
  REGION 1..16: note = Synthetic polymer.
YINPYNDGTDYDEKFK

SEQ ID NO: 373 - protein, 13 aa, synthetic construct
  REGION 1..13: note = Synthetic polymer.
EKDNYATGAWFAY

SEQ ID NO: 374 - protein, 15 aa, synthetic construct
  REGION 1..15: note = Synthetic polymer.
KSSQSLLYSTNQKNY

SEQ ID NO: 375 - protein, 7 aa, synthetic construct
  REGION 1..7: note = Synthetic polymer.
WASTRES

SEQ ID NO: 376 - protein, 8 aa, synthetic construct
  REGION 1..8: note = Synthetic polymer.
QQYYSYRT

SEQ ID NO: 377 - protein, 5 aa, synthetic construct
  REGION 1..5: note = Synthetic polymer.
SYVIH

SEQ ID NO: 378 - protein, 10 aa, synthetic construct
  REGION 1..10: note = Synthetic polymer.
GFTFSNYAMS

SEQ ID NO: 379 - protein, 12 aa, synthetic construct
  REGION 1..12: note = Synthetic polymer.
AISDHSTNTYYP

SEQ ID NO: 380 - protein, 13 aa, synthetic construct
  REGION 1..13: note = Synthetic polymer.
EKDNYATGAWFAY

SEQ ID NO: 381 - protein, 11 aa, synthetic construct
  REGION 1..11: note = Synthetic polymer.
ARKYGGDYDPF

SEQ ID NO: 382 - protein, 16 aa, synthetic construct
  REGION 1..16: note = Synthetic polymer.
KSSQSLLYSTNQKNYL

SEQ ID NO: 383 - protein, 16 aa, synthetic construct
  REGION 1..16: note = Synthetic polymer.
KSSGSLLYSTNQKNYL

SEQ ID NO: 384 - protein, 9 aa, synthetic construct
  REGION 1..9: note = Synthetic polymer.
KASQDINNY

SEQ ID NO: 385 - protein, 7 aa, synthetic construct
  REGION 1..7: note = Synthetic polymer.
WASTRES

SEQ ID NO: 386 - protein, 11 aa, synthetic construct
  REGION 1..11: note = Synthetic polymer.
YTSTLQPGVPS

SEQ ID NO: 387 - protein, 8 aa, synthetic construct
  REGION 1..8: note = Synthetic polymer.
QQYYSYRT

SEQ ID NO: 388 - protein, 6 aa, synthetic construct
  REGION 1..6: note = Synthetic polymer.
YDNLLF

SEQ ID NO: 389 - protein, 5 aa, synthetic construct
  REGION 1..5: note = Synthetic polymer.
TFGVH

SEQ ID NO: 390 - protein, 5 aa, synthetic construct
  REGION 1..5: note = Synthetic polymer.
TAGVH

SEQ ID NO: 391 - protein, 5 aa, synthetic construct
  REGION 1..5: note = Synthetic polymer.
TFGVA

SEQ ID NO: 392 - protein, 16 aa, synthetic construct
  REGION 1..16: note = Synthetic polymer.
VIWRSGITDYNVPFMS

SEQ ID NO: 393 - protein, 16 aa, synthetic construct
  REGION 1..16: note = Synthetic polymer.
VIARSGITDYNVPFMS

SEQ ID NO: 394 - protein, 16 aa, synthetic construct
  REGION 1..16: note = Synthetic polymer.
VIWASGITDYNVPFMS

SEQ ID NO: 395 - protein, 9 aa, synthetic construct
  REGION 1..9: note = Synthetic polymer.
NDPGTGFAY

SEQ ID NO: 396 - protein, 9 aa, synthetic construct
  REGION 1..9: note = Synthetic polymer.
NDPGTGAAY

SEQ ID NO: 397 - protein, 9 aa, synthetic construct
  REGION 1..9: note = Synthetic polymer.
NDPGTGFAA

SEQ ID NO: 398 - protein, 11 aa, synthetic construct
  REGION 1..11: note = Synthetic polymer.
RASENIYSYLA

SEQ ID NO: 399 - protein, 11 aa, synthetic construct
  REGION 1..11: note = Synthetic polymer.
RASENIYSALA

SEQ ID NO: 400 - protein, 7 aa, synthetic construct
  REGION 1..7: note = Synthetic polymer.
DAKTLAE

SEQ ID NO: 401 - protein, 9 aa, synthetic construct
  REGION 1..9: note = Synthetic polymer.
QHHYGNPPT

SEQ ID NO: 402 - protein, 9 aa, synthetic construct
  REGION 1..9: note = Synthetic polymer.
QHAYGNPPT

SEQ ID NO: 403 - protein, 9 aa, synthetic construct
  REGION 1..9: note = Synthetic polymer.
QHHAGNPPT

SEQ ID NO: 404 - protein, 5 aa, synthetic construct
  REGION 1..5: note = Synthetic polymer.
DYVIN

SEQ ID NO: 405 - protein, 17 aa, synthetic construct
  REGION 1..17: note = Synthetic polymer.
EIYPGSGSDYYNENLKD

SEQ ID NO: 406 - protein, 12 aa, synthetic construct
  REGION 1..12: note = Synthetic polymer.
KGENGNSLAFAY

SEQ ID NO: 407 - protein, 12 aa, synthetic construct
  REGION 1..12: note = Synthetic polymer.
QSVDYDGDSYMN

SEQ ID NO: 408 - protein, 7 aa, synthetic construct
  REGION 1..7: note = Synthetic polymer.
AASNLES

SEQ ID NO: 409 - protein, 9 aa, synthetic construct
  REGION 1..9: note = Synthetic polymer.
QQSIQDPCT

SEQ ID NO: 410 - protein, 471 aa, synthetic construct
  REGION 1..471: note = Synthetic polymer.
MEWSGVFMFLLSVTAGVHSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 411 - protein, 237 aa, synthetic construct
  REGION 1..237: note = Synthetic polymer.
MEWSGVFIFLLSVTAGVHSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 412 - protein, 122 aa, synthetic construct
  REGION 1..122: note = Synthetic polymer.
EEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAFAYGQGTLVTVSS

SEQ ID NO: 413 - protein, 110 aa, synthetic construct
  REGION 1..110: note = Synthetic polymer.
DIVMTQSPDSLAVSLGERATINCRASKSVSTSGYSYIYWYQQKPGQPPKLLIYLASILESGVPDRFSGSGSGTDFTLTISSLQAEDVAVYYCQHSRELPTFGQGTKVEIK

SEQ ID NO: 414-415 - skipped (2 entries)

SEQ ID NO: 416 - protein, 6 aa, synthetic construct
  REGION 1..6: note = Synthetic polymer.
GGSGGS

SEQ ID NO: 417 - protein, 9 aa, synthetic construct
  REGION 1..9: note = Synthetic polymer.
GGSGGSGGS

SEQ ID NO: 418 - protein, 12 aa, synthetic construct
  REGION 1..12: note = Synthetic polymer.
GGSGGSGGSGGS

SEQ ID NO: 419 - protein, 15 aa, synthetic construct
  REGION 1..15: note = Synthetic polymer.
GGSGGSGGSGGSGGS

SEQ ID NO: 420 - protein, 18 aa, synthetic construct
  REGION 1..18: note = Synthetic polymer.
GGSGGSGGSGGSGGSGGS

SEQ ID NO: 421 - protein, 21 aa, synthetic construct
  REGION 1..21: note = Synthetic polymer.
GGSGGSGGSGGSGGSGGSGGS

SEQ ID NO: 422 - protein, 24 aa, synthetic construct
  REGION 1..24: note = Synthetic polymer.
GGSGGSGGSGGSGGSGGSGGSGGS

SEQ ID NO: 423 - protein, 27 aa, synthetic construct
  REGION 1..27: note = Synthetic polymer.
GGSGGSGGSGGSGGSGGSGGSGGSGGS

SEQ ID NO: 424 - protein, 30 aa, synthetic construct
  REGION 1..30: note = Synthetic polymer.
GGSGGSGGSGGSGGSGGSGGSGGSGGSGGS

SEQ ID NO: 425 - protein, 33 aa, synthetic construct
  REGION 1..33: note = Synthetic polymer.
GGSGGSGGSGGSGGSGGSGGSGGSGGSGGSGGS

SEQ ID NO: 426 - protein, 36 aa, synthetic construct
  REGION 1..36: note = Synthetic polymer.
GGSGGSGGSGGSGGSGGSGGSGGSGGSGGSGGSGGS

SEQ ID NO: 427 - protein, 39 aa, synthetic construct
  REGION 1..39: note = Synthetic polymer.
GGSGGSGGSGGSGGSGGSGGSGGSGGSGGSGGSGGSGGS

SEQ ID NO: 428 - protein, 42 aa, synthetic construct
  REGION 1..42: note = Synthetic polymer.
GGSGGSGGSGGSGGSGGSGGSGGSGGSGGSGGSGGSGGSGGS

SEQ ID NO: 429 - protein, 45 aa, synthetic construct
  REGION 1..45: note = Synthetic polymer.
GGSGGSGGSGGSGGSGGSGGSGGSGGSGGSGGSGGSGGSGGSGGS

SEQ ID NO: 430 - protein, 48 aa, synthetic construct
  REGION 1..48: note = Synthetic polymer.
GGSGGSGGSGGSGGSGGSGGSGGSGGSGGSGGSGGSGGSGGSGGSGGS

SEQ ID NO: 431 - protein, 51 aa, synthetic construct
  REGION 1..51: note = Synthetic polymer.
GGSGGSGGSGGSGGSGGSGGSGGSGGSGGSGGSGGSGGSGGSGGSGGSGGS

SEQ ID NO: 432 - protein, 54 aa, synthetic construct
  REGION 1..54: note = Synthetic polymer.
GGSGGSGGSGGSGGSGGSGGSGGSGGSGGSGGSGGSGGSGGSGGSGGSGGSGGS

SEQ ID NO: 433 - protein, 57 aa, synthetic construct
  REGION 1..57: note = Synthetic polymer.
GGSGGSGGSGGSGGSGGSGGSGGSGGSGGSGGSGGSGGSGGSGGSGGSGGSGGSGGS

SEQ ID NO: 434 - protein, 60 aa, synthetic construct
  REGION 1..60: note = Synthetic polymer.
GGSGGSGGSGGSGGSGGSGGSGGSGGSGGSGGSGGSGGSGGSGGSGGSGGSGGSGGSGGS

SEQ ID NO: 435 - protein, 4 aa, synthetic construct
  REGION 1..4: note = Synthetic polymer.
GGGS

SEQ ID NO: 436 - protein, 8 aa, synthetic construct
  REGION 1..8: note = Synthetic polymer.
GGGSGGGS

SEQ ID NO: 437 - protein, 12 aa, synthetic construct
  REGION 1..12: note = Synthetic polymer.
GGGSGGGSGGGS

SEQ ID NO: 438 - protein, 16 aa, synthetic construct
  REGION 1..16: note = Synthetic polymer.
GGGSGGGSGGGSGGGS

SEQ ID NO: 439 - protein, 20 aa, synthetic construct
  REGION 1..20: note = Synthetic polymer.
GGGSGGGSGGGSGGGSGGGS

SEQ ID NO: 440 - protein, 24 aa, synthetic construct
  REGION 1..24: note = Synthetic polymer.
GGGSGGGSGGGSGGGSGGGSGGGS

SEQ ID NO: 441 - protein, 28 aa, synthetic construct
  REGION 1..28: note = Synthetic polymer.
GGGSGGGSGGGSGGGSGGGSGGGSGGGS

SEQ ID NO: 442 - protein, 32 aa, synthetic construct
  REGION 1..32: note = Synthetic polymer.
GGGSGGGSGGGSGGGSGGGSGGGSGGGSGGGS

SEQ ID NO: 443 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 444 - protein, 246 aa, synthetic construct
  REGION 1..246: note = Synthetic polymer.
MGWSCIIFFLVATATGVHSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 445 - protein, 381 aa, synthetic construct
  REGION 1..381: note = Synthetic polymer.
MGWSCIIFFLVATATGVHSDVQLQESGGGLVQPGGSLRLSCAASGSIFSINVMGWYRQTPGKERELVAKITNFGITSYADSAQGRFTISRGNAKNTVYLQMNSLKPEDTAVYYCNLDTTGWGPPPYQYWGQGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 446 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVSLAGGPPSITDFQILENQA

SEQ ID NO: 447 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVHLAGGPPSITDFQILENQA

SEQ ID NO: 448 - protein, 462 aa, synthetic construct; identical to SEQ ID NO: 446
  REGION 1..462: note = Synthetic polymer.

SEQ ID NO: 449 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFARIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 450 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFGRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 451 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMAIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 452 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMGIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 453 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIAKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 454 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIGKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 455 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILADALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 456 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILGDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 457 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDAANQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 458 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDAGNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 459 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALAQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 460 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALGQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 461 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIARANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 462 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIGRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 463 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAGLHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 464 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAASLHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 465 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAHLHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 466 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAATLHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 467 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAANLHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 468 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALANLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 469 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALGNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 470 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGGYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 471 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGSYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 472 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGTYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 473 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGNYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 474 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGHYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 475 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGFYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 476 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYASSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 477 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYGSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 478 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKADAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 479 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAAITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 480 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKATVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 481 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKGTVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 482 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKADYWVCLAGGPPSITDFQILENQA

SEQ ID NO: 483 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFAILENQA

SEQ ID NO: 484 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFGILENQA

SEQ ID NO: 485 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITAFGILENQA

SEQ ID NO: 486 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITKFGILENQA

SEQ ID NO: 487 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITYFGILENQA

SEQ ID NO: 488 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKGDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 489 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDAAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 490 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKAAAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 491 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFFETHGTKNYFTSVAHPNLFIATKQDYWVCLAGGPPSITDFQILENQA

SEQ ID NO: 492 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPATITGSETNLLFFWETHGTKNYFTSVAHPNLFIATKQDYWVCLAGGPPSITDFQILENQA

SEQ ID NO: 493 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPDTITGSETNLLFFWETHGTKNYFTSVAHPNLFIATKQDYWVCLAGGPPSITDFQILENQA

SEQ ID NO: 494 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVALAGGPPSITDFQILENQA

SEQ ID NO: 495 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALDQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 496 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQAIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 497 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQGIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 498 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALAQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVALAGGPPSITDFQILENQA

SEQ ID NO: 499 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALGQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVALAGGPPSITDFQILENQA

SEQ ID NO: 500 - protein, 1273 aa, synthetic construct
  REGION 1..1273: note = Synthetic polymer.
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 501 - protein, 222 aa, synthetic construct
  REGION 1..222: note = Synthetic polymer.
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 502 - protein, 75 aa, synthetic construct
  REGION 1..75: note = Synthetic polymer.
MYSFVSEETGTLIVNSVLLFLAFVVFLLVTLAILTALRLCAYCCNIVNVSLVKPSFYVYSRVKNLNSSRVPDLLV

SEQ ID NO: 503 - protein, 419 aa, synthetic construct
  REGION 1..419: note = Synthetic polymer.
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 504 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALDQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVALAGGPPSITDFQILENQA

SEQ ID NO: 505 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQAIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVALAGGPPSITDFQILENQA

SEQ ID NO: 506 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQGIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVALAGGPPSITDFQILENQA

SEQ ID NO: 507 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALAQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVSLAGGPPSITDFQILENQA

SEQ ID NO: 508 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALGQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVSLAGGPPSITDFQILENQA

SEQ ID NO: 509 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALDQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVSLAGGPPSITDFQILENQA

SEQ ID NO: 510 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQAIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVSLAGGPPSITDFQILENQA

SEQ ID NO: 511 - protein, 462 aa, synthetic construct
  REGION 1..462: note = Synthetic polymer.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQGIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVSLAGGPPSITDFQILENQA

SEQ ID NO: 512 - protein, 459 aa, synthetic construct
  REGION 1..459: note = Synthetic polymer.
MEFGLSWLFLVAILKGVQCLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQAGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 513 - protein, 366 aa, synthetic construct
  REGION 1..366: note = Synthetic polymer.
MEFGLSWLFLVAILKGVQCQVQLQESGGGLVQPGGSLRLSCAASGSIFSINVMGWYRQTPGKERELVAKITNFGITSYADSAQGRFTISRGNAKNTVYLQMNSLKPEDTAVYYCNLDTTGWGPPPYQYWGQGTQVTVSSVDGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQAGGSHHHHHHHHH

SEQ ID NO: 514 - protein, 366 aa, synthetic construct
  REGION 1..366: note = Synthetic polymer.
MEFGLSWLFLVAILKGVQCLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQAGGSGGSGGSGGSGGSGGSGGSGGSGGSGGSGGSGGSGGSGGSGGSGGSGGSGGSGGSGGSQVQLQESGGGLVQPGGSLRLSCAASGSIFSINVMGWYRQTPGKERELVAKITNFGITSYADSAQGRFTISRGNAKNTVYLQMNSLKPEDTAVYYCNLDTTGWGPPPYQYWGQGTQVTVSSVDGGSHHHHHHHHH

SEQ ID NO: 515 - protein, 382 aa, synthetic construct
  REGION 1..382: note = Synthetic polymer.
MGWSCIIFFLVATATGVHSDVQLQESGGGLVQAGDSLRLTCTASGRTFSNYGIGWFRQAPGKEREFVAGINWSGESADYADSVKGRFTISRDNAKNTVYLQMNSLKPEDTAVYYCAAGESGVWVGGLDYWGQGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 516 - protein, 394 aa, synthetic construct
  REGION 1..394: note = Synthetic polymer.
MGWSCIIFFLVATATGVHSDVQLQESGGGLVQAGGSLRLSCAASGFSSDDYTIGWFRQAPGKEREGISCFSSSDGSTGFADSVKGRFTISSDNATNTVYLEMNSLKPEDTAVYYCAADFNVWSPPICGSRWYGPPPGGMEYWGKGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 517 - protein, 392 aa, synthetic construct
  REGION 1..392: note = Synthetic polymer.
MGWSCIIFFLVATATGVHSDVQLQESGGGLVQAGGSLRLSCAVSGFTFDNYAIGWFRQAPGKEREGVSSISRSDGSTYYADSVRGRFTISSDNAKNTVYLQMNSLKPEDTAVYYCAAETSADSGEFRFGWVLKPSLYDYWGQGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 518 - protein, 389 aa, synthetic construct
  REGION 1..389: note = Synthetic polymer.
MGWSCIIFFLVATATGVHSDVQLQESGGGLVQAGGSLRLSCAASGRTFSSYVMGWFRQAPGKEREFVAQISWSAGSIYYADSVKGRFTISNDNAKRTVYLQMNSLKPEDTAVYYCAERGYAYCSDDGCQRTQDYDYWGQGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 519 - protein, 393 aa, synthetic construct
  REGION 1..393: note = Synthetic polymer.
MGWSCIIFFLVATATGVHSDVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVVVPARPYYDMDYWGKGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 520 - protein, 546 aa, synthetic construct
  REGION 1..546: note = Synthetic polymer.
MEFGLSWLFLVAILKGVQCEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGGGSGGGGSGGGGSDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 521 - protein, 546 aa, synthetic construct
  REGION 1..546: note = Synthetic polymer.
MEFGLSWLFLVAILKGVQCDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKGGGGSGGGGSGGGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 522 - protein, 594 aa, synthetic construct
  REGION 1..594: note = Synthetic polymer.
MEFGLSWLFLVAILKGVQCDVQLQESGGGLVQPGGSLRLSCAASGSIFSINVMGWYRQTPGKERELVAKITNFGITSYADSAQGRFTISRGNAKNTVYLQMNSLKPEDTAVYYCNLDTTGWGPPPYQYWGQGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 523 - protein, 503 aa, synthetic construct
  REGION 1..503: note = Synthetic polymer.
MGWSCIIFFLVATATGVHSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGGGSGGGGSGGGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 524 - protein, 462 aa, synthetic construct; identical to SEQ ID NO: 510
  REGION 1..462: note = Synthetic polymer.